(19) United States
(12) Patent Application Publication
(10) Pub. No.: US 2010/0179109 A1
(43) Pub. Date: Jul. 15, 2010

(54) RENIN INHIBITORS

(76) Inventors: Jonh J. Baldwin, Gwynedd Valley, PA (US); Salvacion Cacatian, Blue Bell, PA (US); David Claremon, Maple Glen, PA (US); Lawrence W. Dillard, Yardley, PA (US); Patrick T. Flaherty, Pittsburgh, PA (US); Bahman Ghavimi-Alagha, Wilmington, DE (US); Damiano Ghirlanda, Legnago (IT); Alexey V. Ishchenko, Somerville, MA (US); Lara S. Kallander, King of Prussia, PA (US); Brian Lawhorn, King of Prussia, PA (US); Colin A. Leach, Collegeville, PA (US); Qing Lu, King of Prussia, PA (US); Gerard McGeehan, Garnet Valley, PA (US); Jaclyn R. Patterson, King of Prussia, PA (US); Robert D. Simpson, Wilmington, DE (US); Suresh B. Singh, Kendall Park, NJ (US); Patrick Stoy, King of Prussia, PA (US); Lamont R. Terrell, King of Prussia, PA (US); Colin Tice, Ambler, PA (US); Zhenrong Xu, Horsham, PA (US); Jing Yuan, Lansdale, PA (US); Catherine C. K. Yuan, King of Prussia, PA (US); Jing Zhang, Waltham, MA (US); Wei Zhao, Eagleville, PA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/450,631

(22) PCT Filed: Apr. 4, 2008
(86) PCT No.: PCT/US08/59384

§ 371 Date: Feb. 5, 2010

Related U.S. Application Data
(60) Provisional application No. 60/910,221, filed on Apr. 5, 2007.

(51) Int. Cl. A61K 31/675; C07D 211/34; A61K 31/445; C07D 265/30; A61K 31/5375; C07D 413/06; A61K 31/5377; A61K 31/535; A61K 31/517; A61K 31/554; A61K 31/4965; A61K 31/5415; A61K 31/55; A61K 31/551; A61K 31/47; A61K 31/566; A61P 25/28; A61P 33/06; A61P 31/18; A61P 9/12; A61P 9/10; A61P 25/22; A61P 27/06

(52) U.S. Cl. 514/91; 546/226; 514/330; 544/162; 514/237.5; 544/131; 514/235.5; 514/233.5; 544/146; 514/252.17; 514/211.07; 514/255.06; 514/224.2; 514/212.07; 514/221; 514/307; 514/211.05; 514/171

(57) ABSTRACT

Disclosed are compounds of Formula (I) wherein the R, R¹, R², R³, X, Y, A, Q, E, and G are defined herein. These compounds bind to aspartic proteases to inhibit their activity and are useful in the treatment or amelioration of diseases associated with aspartic protease activity. Also disclosed are methods of use of the compounds of Formula I for ameliorating or treating aspartic protease related disorders in a subject in need thereof.

### BACKGROUND OF THE INVENTION

Aspartic proteases, including renin, β-secretase (BACE), Candida albicans secreted aspartyl proteases, HIV protease, HTLV protease and plasmepsins I and II, are implicated in a number of disease states. In hypertension elevated levels of angiotensin I, the product of renin catalyzed cleavage of angioteninogen are present. Elevated levels of βamyloid, the product of BACE activity on amyloid precursor protein, are widely believed to be responsible for the amyloid plaques present In the brains of Alzheimer's disease patients. Secreted aspartyl proteases play a role in the virulence of the pathogen Candida albicans. The viruses HIV and HTLV depend on their respective aspartic proteases for viral maturation. Plasmodium falciparum uses plasmepsins I and II to degrade hemoglobin.

In the renin-angiotensin-aldosterone system (RAAS) the biologically active peptide angiotensin II (Ang II) is generated by a two-step mechanism. The highly specific aspartic protease renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAAS represents a major advance in the treatment of cardiovascular diseases (Zaman, M. A. et al Nature Reviews Drug Discovery 2002, 1, 621-636). ACE inhibitors and AT₁ blockers have been accepted as treatments of hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N Engl. J: Med, 1992, 327, 669).

Interest in the development of renin inhibitors stems from the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be bypassed by chymase, a serine protease (Husain A., J. Hypertens., 1993, 11, 1155). In patients, inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the AT1 receptor (e.g., by losartan) on the other hand overexposes other AT-receptor subtypes to Ang II, whose concentration is dramatically increased by the blockade of AT1 receptors. In summary, renin inhibitors are not only expected to be superior to ACE inhibitors and AT₁ blockers with regard to safety, but more importantly also with regard to their efficacy in blocking the RAAS.

Only limited clinical experience (Azizi M. et al., J. Hypertens., 1994, 12, 419; Neutel J. M. et al., Am. Heart, 1991, 122, 1094) has been generated with renin inhibitors because their peptidomimetic character imparts insufficient oral activity (Kleinert H. D., Cardiovasc. Drugs, 1995, 9, 645). The clinical development of several compounds has been stopped because of this problem together with the high cost of goods. It appears as though only one compound has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are not available. Recently, the first non-peptide renin inhibitors were described which show high in vitro activity (Oefner C. et al., Chem. Biol., 1999, 6, 127; Patent Application WO 97/09311; Maerki H. P. et al., Il Farmaco, 2001, 56, 21). The present invention relates to the unexpected identification of renin inhibitors of a non-peptidic nature and of low molecular weight. Orally active renin inhibitors which are active in indications beyond blood pressure regulation where the tissular renin-chymase system may be activated leading to pathophysiologically altered local functions such as renal, cardiac and vascular remodeling, atherosclerosis, and restenosis, are described.

All documents cited herein are incorporated by reference.

### SUMMARY OF THE INVENTION

Compounds have now been found which are orally active and bind to aspartic proteases to inhibit their activity. They are useful in the treatment or amelioration of diseases associated with aspartic protease activity.

In one embodiment the present invention is directed to compounds represented by Formula I:

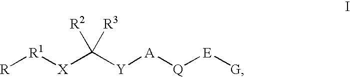

or an enantiomer, diastereomer or salt thereof.

R is:

a) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cycloalkoxy, (C₅-C₇)cyclo-alkenyloxy, (C₃-C₇)cycloalkoxy(C₁-C₃)alkyl, (C₃-C₇)cycloalkyl(C₁-C₃)alkoxy, (C₅-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁-C₃)alkylthio, (C₅-C₇)cycloalkenyl(C₁-C₃)alkylthio, (C₁-C₈)alkylamino, di(C₁-C₈)alkylamino, azepano, azetidino, piperidino, pyrrolidino, (C₃-C₇)cycloalkylamino, ((C₃-C₇)cycloalkyl(C₁-C₃)alkyl)amino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy and oxo;

b) aryl, heteroaryl, arylheterocyclyl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃))alkenyl, or heteroaryl(C₂-C₃))alkynyl, each optionally substituted with up to three substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkyl, halo(C₁-C₆)alkoxy(C₁-C₆)alkyl, (C₃-C₆)cycloalkoxy(C₁-C₆)alkyl, (C₄-C₇)cycloalkylalkoxy(C₁-C₆)alkyl, halo(C₃-C₆)cycloalkoxy(C₁-C₆)alkyl, halo(C₄-C₇)cycloalkylalkoxy(C₁-C₆)alkyl, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or

c) a divalent radical selected from —(CH₂)₃—, —(CH₂)₄—, —(CH₂)₅— and —(CH₂)₆—, which is attached to R¹ to form a fused or spirofused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo;

R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally substituted with up to four substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylamino carbonyl and di(C₁-C₆)alkylamino carbonyl;

X and Y are each independently CH₂ or a single bond;

R² is a) —H; or b) (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, (C₁-C₄)alkoxy(C₁-C₄)alkoxy(C₁-C₄)alkyl, amino carbonylamino(C₁-C₁₂)alkyl, aminocarbonylamino(C₁-C₁₂)alkoxy, aminocarbonylamino(C₁-C₁₂)alkylthio, aminocarbonylamino(C₁-C₁₂)alkylamino, (C₁-C₆)-alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxy-carbonyl(C₁-C₆)alkylamino, (C₁-C₆)acyloxy(C₁-C₆)alkyl, (C₁-C₆)acyloxy(C₁-C₆)alkoxy, (C₁-C₆)acyloxy(C₁-C₆)alkylthio, (C₁-C₆)acyloxy(C₁-C₆)alkylamino, aminosulfonylamino(C₁-C₁₂)alkyl, aminosulfonylamino(C₁-C₁₂)alkoxy, aminosulfonylamino(C₁-C₁₂)alkylthio, aminosulfonyl-amino(C₁-C₁₂)alkylamino, (C₁-C₆)alkanesulfonylamino(C₁-C₆)alkyl, (C₁-C₆)alkanesulfonyl-amino(C₁-C₆)alkoxy, (C₁-C₆)alkane sulfonylamino(C₁-C₆)alkylthio, (C₁-C₆)alkanesulfonyl-amino(C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkyl, (C₁-C₆)alkylaminocarbonylamino(C₁-C₆)alkoxy, (C₁-C₆)alkylamino carbonyl-amino(C₁-C₆)alkylthio, (C₁-C₆)alkylaminocarbonylamino(C₁-C₆)alkylamino, amino carbonyl(C₁-C₆)alkyl, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkyl, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkyl, (C₁-C₆)alkylamino-carboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:

1) 1 to 5 halogen atoms; and

2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy;

The divalent sulfur atoms in R² are independently optionally oxidized to sulfoxide or sulfone and wherein the carbonyl groups are optionally independently changed to a thiocarbonyl groups;

R³ is —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)-alkoxycarbonylamino, (C₁-C₆)alkylamino-carbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonyl-amino, phenylamino or heteroarylamino in which each phenylamino or heteroarylamino group is optionally substituted with 1 to 5 groups independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)-cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)-cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl, provided that

i) R² and R³ are not both hydrogen; and

ii) when R³ is hydroxyl, halogen, or optionally substituted phenylamino or heteroarylamino, R² is not (C₁-C₁₂)alkoxy, (C₁-C₁₂)alkylthio, (C₁-C₁₂)alkylamino, oxo(C₁-C₁₂)alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)-alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, aminocarbonylamino(C₁-C₁₂)alkoxy, aminocarbonyl-amino(C₁-C₁₂)alkylthio, aminocarbonylamino(C₁-C₁₂)alkylamino, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl-(C₁-C₆)alkylamino, (C₁-C₆)acyloxy(C₁-C₆)alkoxy, (C₁-C₆)acyloxy(C₁-C₆)alkylthio, (C₁-C₆)-acyloxy(C₁-C₆)alkylamino, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkoxy, (C₁-C₆)alkylaminocarbonylamino(C₁-C₆)alkylthio, (C₁-C₆)alkylamino-carbonylamino(C₁-C₆)alkylamino, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkoxy, aminocarboxy(C₁-C₆)alkylthio, amino carboxy(C₁-C₆)alkylamino, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:

1) 1 to 5 halogen atoms; and

2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy;

the divalent sulfur atoms in R³ are independently optionally oxidized to sulfoxide or sulfone and wherein the carbonyl groups in R³ are optionally independently changed to thiocarbonyl groups;

A is a saturated or unsaturated 4-, 5-, 6-, or 7-membered ring which is optionally bridged by (CH₂)ₘ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally substituted with up to four independently selected halogen atoms, (C₁-C₆)alkyl groups, halo(C₁-C₆)alkyl groups or oxo groups such that when there is substitution with one oxo group on a carbon atom it forms a carbonyl group and when there is substitution of one or two oxo groups on sulfur it forms sulfoxide or sulfone groups, respectively, where m is 1 to 3;

Q and Y are attached to carbon or nitrogen atoms in ring A in a 1,2 or 1,3, or 1,4 relationship;

Q is a divalent radical:

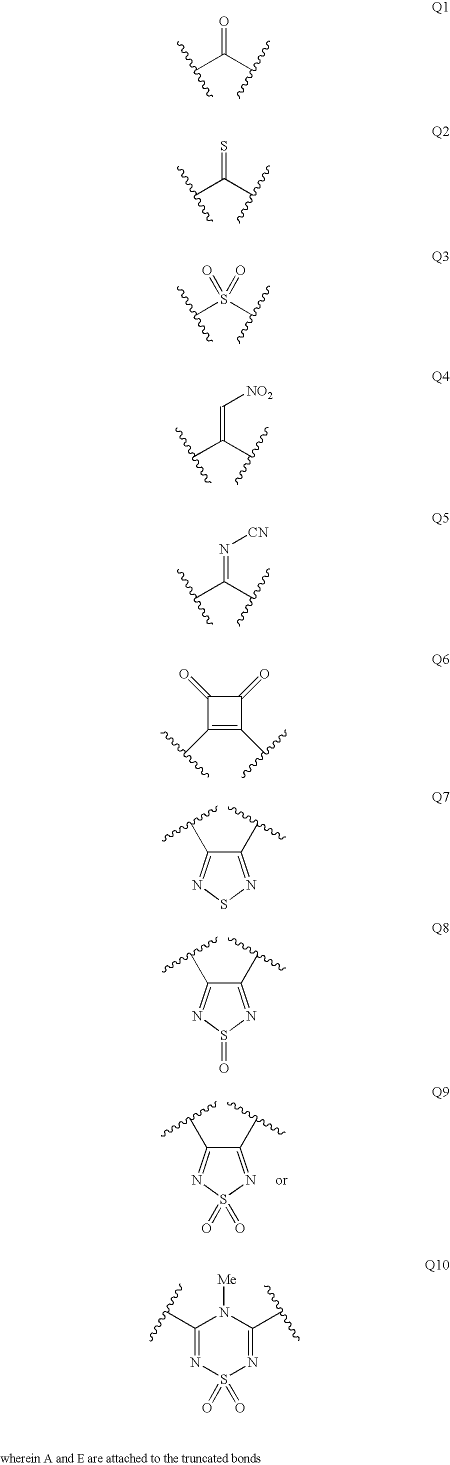

E is an optionally substituted (C₁-C₄)alkyl naphthyl, (C₁-C₄)alkyl phenyl, naphthyl or phenyl group, wherein said group is optionally substituted with up to four groups independently selected from halogen, hydroxy, aryl, (C₁-C₆)alkyl, halo(C₁-C₆)alkyl, hydroxy(C₁-C₆)alkyl-, heterocyclyl(C₁-C₆)alkyl-, (C₄-C₆)cycloalkyl(C₁-C₆)alkyl-, cyano(C₁-C₆)alkyl-, (C₁-C₆)alkoxy-, —C(═O)(C₁-C₆)alkyl, —C(═O)OH, —C(═O)O(C₁-C₆)alkyl, —C(═O)NH₂, —(C₁-C₆)alkyl —C(═O)NH₂, and —CN; When E is an optionally substituted (C₁-C₄)alkyl naphthyl or (C₁-C₄)alkyl phenyl, the bonding arrangement of Q to E is via the (C₁-C₄)alkyl moiety, for example—Q-(C₁-C₄)alkyl-phenyl-G;

G is hydroxy, —NR⁴R^{4a}, —O(C₂-C₆)alkyl-NR⁴R^{4a}, heterocyclyl, —(C₁-C₆)alkyl-OH, —(C₁-C₆)haloalkyl-OH, —(C₁-C₆)alkyl-NR⁴R^{4a}, —(C₁-C₆)alkyl-N⁺(C₁-C₆)alkylR⁴R^{4a}, —(C₁-C₆)alkylSO₂(C₁-C₆)alkyl, —C(═O)(C₁-C₆)alkyl-NR⁴R^{4a}, —C(═O)OH, —C(═O)NH₂, —C(═NH)NR⁴R^{4a}, —NHC(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino, where R^{4a} is H or (C₁-C₃)alkyl and R⁴ is selected from H, optionally substituted (C₁-C₆)alkyl, —C(═NH)NH₂, (C₃-C₇)cycloalkyl, (C₄-C₇)heterocyclyl, (C₃-C₇)cycloalkyl(C₁-C₆)alkyl, and (C₄-C₇)heterocyclyl(C₁-C₆)alkyl, wherein the optionally substituted (C₁-C₆)alkyl is optionally substituted by hydroxy, (C₁-C₆)alkoxy, —NH₂, —NH(C₁-C₆)alkyl, —N(C₁-C₆)alkyl(C₁-C₆)alkyl, —NHSO₂(C₁-C₆)alkyl, (C₁-C₆)alkylthio, (C₁-C₆)alkanesulfonyl, —C(═O)OH, —C(═O)NH₂, —CN, or R⁴ and R^{4a}, taken together with the nitrogen atom to which they are attached, form a 5-6 membered saturated heterocyclic ring composed of carbon atoms and 1-3 nitrogen atoms, specifically 2-3 nitrogen atoms, more specifically 2 nitrogen atoms; said ring being optionally substituted with up to four groups independently selected from halogen, hydroxy, amino, (C₁-C₆)alkyl, (C₁-C₆)alkylamino, halo(C₁-C₆)alkyl, hydroxy(C₁-C₆)alkyl, amino(C₁-C₆)alkyl, and oxo groups such that when there is substitution with one oxo group on a carbon atom it forms a carbonyl group.

In another embodiment the present invention is directed to pharmaceutical compositions comprising a compound described herein or enantiomers, diastereomers, or salts thereof and a pharmaceutically acceptable carrier therefor.

In another embodiment the present invention is directed to a method of inhibiting an aspartic protease in a subject in need thereof comprising administering to the subject a therapeutically effective amount of a compound described herein or an enantiomer, diastereomer, or salt thereof.

In another embodiment the present invention is directed to method for treating or ameliorating an aspartic protease mediated disorder in a subject in need thereof comprising administering to said subject a therapeutically effective amount of a compound described herein or an enantiomer, diastereomer, or salt thereof.

In another embodiment the present invention is directed to a method for treating or ameliorating a renin mediated disorder in a subject in need thereof comprising administering to the subject an effective amount of a compound described herein or an enantiomer, diastereomer, or salt thereof.

In another embodiment the present invention is directed to a method for the treatment of hypertension in a subject in need thereof comprising administering to the subject a compound described herein in combination therapy with one or more additional agents said additional agent selected from the group consisting of α-blockers, β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, and endothelin receptor antagonists.

### DETAILED DESCRIPTION OF THE INVENTION

A description of embodiments of the compounds of Formula I of the invention follows. It is understood that the invention encompasses all combinations of the substituent variables (i.e., R, R¹, R², R³, X, Y, A, Q, E and G) defined herein.

In one embodiment the present invention is directed to compounds represented by Formula I:

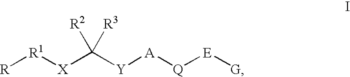

or an enantiomer, diastereomer or salt thereof.

R is:

a) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cycloalkoxy, (C₅-C₇)cyclo-alkenyloxy, (C₃-C₇)cycloalkoxy(C₁-C₃)alkyl, (C₃-C₇)cycloalkyl(C₁-C₃)alkoxy, (C₅-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁-C₃)alkylthio, (C₅-C₇)cycloalkenyl(C₁-C₃)alkylthio, (C₁-C₈)alkylamino, di(C₁-C₈)alkylamino, azepano, azetidino, piperidino, pyrrolidino, (C₃-C₇)cycloalkylamino, ((C₃-C₇)cycloalkyl(C₁-C₃)alkyl)amino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy and oxo;

b) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃))alkenyl, or heteroaryl(C₂-C₃))alkynyl, each optionally substituted with up to three substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or

c) a divalent radical selected from —(CH₂)₃—, —(CH₂)₄—, —(CH₂)₅— and —(CH₂)₆—, which is attached to R¹ to form a fused or spirofused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo.

R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally substituted with up to four substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylaminocarbonyl and di(C₁-C₆)alkylaminocarbonyl.

X and Y are each independently CH₂ or a single bond.

R² is a) —H; or b) (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, aminocarbonylamino(C₁-C₁₂)alkylamino, (C₁-C₆)-alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxy-carbonyl(C₁-C₆)alkylamino, (C₁-C₆)acyloxy(C₁-C₆)alkyl, (C₁-C₆)acyloxy(C₁-C₆)alkoxy, (C₁-C₆)acyloxy(C₁-C₆)alkylthio, (C₁-C₆)acyloxy(C₁-C₆)alkylamino, aminosulfonylamino(C₁-C₁₂)alkyl, aminosulfonylamino(C₁-C₁₂)alkoxy, aminosulfonylamino(C₁-C₁₂)alkylthio, aminosulfonyl-amino(C₁-C₁₂)alkylamino, (C₁-C₆)alkanesulfonylamino(C₁-C₆)alkyl, (C₁-C₆)alkanesulfonyl-amino(C₁-C₆)alkoxy, (C₁-C₆)alkanesulfonylamino(C₁-C₆)alkylthio, (C₁-C₆)alkanesulfonyl-amino(C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkyl, (C₁-C₆)alkylaminocarbonylamino(C₁-C₆)alkoxy, (C₁-C₆)alkylamino carbonyl-amino(C₁-C₆)alkylthio, (C₁-C₆)alkylaminocarbonylamino(C₁-C₆)alkylamino, aminocarbonyl(C₁-C₆)alkyl, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkyl, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkyl, (C₁-C₆)alkylamino-carboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:

1) 1 to 5 halogen atoms; and

2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy.

The divalent sulfur atoms in R² are independently optionally oxidized to sulfoxide or sulfone and wherein the carbonyl groups are optionally independently changed to a thiocarbonyl groups;

R³ is —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)-alkoxycarbonylamino, (C₁-C₆)alkylamino-carbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonyl-amino, phenylamino or heteroarylamino in which each phenylamino or heteroarylamino group is optionally substituted with 1 to 5 groups independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)-cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)-cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl, provided that

i) R² and R³ are not both hydrogen; and

ii) when R³ is hydroxyl, halogen, or optionally substituted phenylamino or heteroarylamino, R² is not (C₁-C₁₂)alkoxy, (C₁-C₁₂)alkylthio, (C₁-C₁₂)alkylamino, oxo(C₁-C₁₂)alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)-alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, aminocarbonylamino(C₁-C₁₂)alkoxy, aminocarbonyl-amino(C₁-C₁₂)alkylthio, aminocarbonylamino(C₁-C₁₂)alkylamino, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl-(C₁-C₆)alkylamino, (C₁-C₆)acyloxy(C₁-C₆)alkoxy, (C₁-C₆) acyloxy(C₁-C₆)alkylthio, (C₁-C₆)-acyloxy(C₁-C₆)alkylamino, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkoxy, (C₁-C₆)alkylaminocarbonylamino(C₁-C₆)alkylthio, (C₁-C₆)alkylamino-carbonylamino(C₁-C₆)alkylamino, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkoxy, aminocarboxy(C₁-C₆)alkylthio, amino carboxy(C₁-C₆)alkylamino, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:

1) 1 to 5 halogen atoms; and

2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy.

The divalent sulfur atoms in R³ are independently optionally oxidized to sulfoxide or sulfone and wherein the carbonyl groups in R³ are optionally independently changed to thiocarbonyl groups.

A is a saturated or unsaturated 4-, 5-, 6-, or 7-membered ring which is optionally bridged by (CH₂)ₘ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally substituted with up to four independently selected halogen atoms, (C₁-C₆)alkyl groups, halo(C₁-C₆)alkyl groups or oxo groups such that when there is substitution with one oxo group on a carbon atom it forms a carbonyl group and when there is substitution of one or two oxo groups on sulfur it forms sulfoxide or sulfone groups, respectively, where m is 1 to 3.

Q and Y are attached to carbon or nitrogen atoms in ring A in a 1, 2 or 1,3, or 1,4 relationship.

Q is a divalent radical:

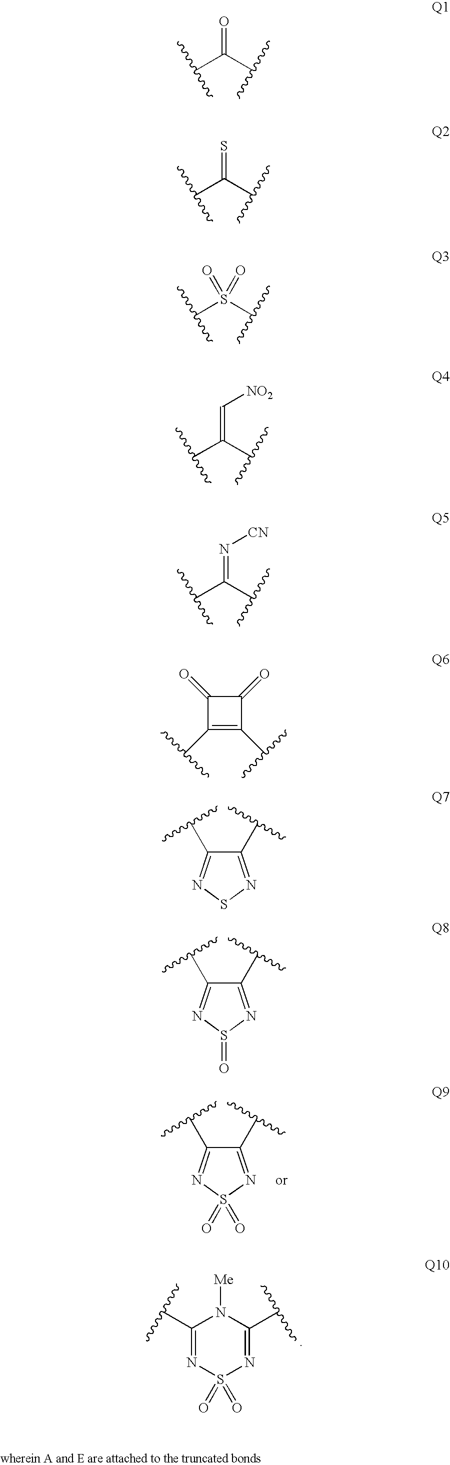

E is phenyl, optionally substituted with up to four groups independently selected from halogen, hydroxy, (C₁-C₆)alkyl, halo(C₁-C₆)alkyl, hydroxy(C₁-C₆)alkyl, heterocyclyl(C₁-C₆)alkyl, (C₄-C₆)cycloalkyl(C₁-C₆)alkyl, (C₁-C₆)alkoxy, —C(═O)(C₁-C₆)alkyl, —C(═O)OH, —C(═O)O(C₁-C₆)alkyl, —C(═O)NH₂, and —CN.

G is hydroxy, —NR⁴R^{4a}, —O(C₂-C₆)alkyl-NR⁴R^{4a}, heterocyclyl, —(C₁-C₆)alkyl-OH, —(C₁-C₆)alkyl-NR⁴R^{4a}, —C(═O)(C₁-C₆)alkyl-NR⁴R^{4a}, —C(═NH)NR⁴R^{4a}, —NHC(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino, where R^{4a} is H or (C₁-C₃)alkyl and R⁴ is selected from H, (C₁-C₃)alkyl, (C₃-C₇)cycloalkyl(C₁-C₆)alkyl, and (C₄-C₇)heterocyclyl(C₁-C₆)alkyl, or R⁴ and R^{4a}, taken together with the nitrogen atom to which they are attached, form a 5-6 membered saturated heterocyclic ring composed of carbon atoms and 1-3 heteroatoms selected from 1, 2, or 3 nitrogen atoms, 0 or 1 oxygen atoms, and 0 or 1 sulfur atoms, said ring being optionally substituted with up to four groups independently selected from halogen, hydroxy, amino, (C₁-C₆)alkyl, (C₁-C₆)alkylamino, halo(C₁-C₆)alkyl, hydroxy(C₁-C₆)alkyl, amino(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively.

In another embodiment of this invention, R is (1) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cyclo alkoxy, (C₅-C₇)cyclo-alkenyloxy, (C₃-C₇)cycloalkoxy(C₁-C₃)alkyl, (C₃-C₇)cyclo alkyl(C₁-C₃)alkoxy, (C₅-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁-C₃)alkylthio, (C₅-C₇)cycloalkenyl(C₁-C₃)alkylthio, (C₁-C₈)alkylamino, di(C₁-C₈)alkylamino, azepano, azetidino, piperidino, pyrrolidino, (C₃-C₇)cycloalkylamino, ((C₃-C₇)cycloalkyl(C₁-C₃)alkyl)amino or tri(C₁-C₄)alkylsilyl, each optionally substituted with up to four substituents independently selected from: fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy, and oxo; or

(2) aryl, heteroaryl, arylheterocyclyl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃))alkenyl, or heteroaryl(C₂-C₃))alkynyl, each optionally substituted with up to three substituents independently selected from: fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or

(3) R is a divalent radical selected from —(CH₂)₃—, —(CH₂)₄—, —(CH₂)₅— and —(CH₂)₆—, which is attached to R¹ to form a fused or spirofused ring system, and is optionally substituted with up to four substituents independently selected from: fluorine, hydroxy, (C₁-C₆)alkyl, halo(C₁-C₆)alkyl, (C₁-C₆)alkoxy and oxo.

In a particular embodiment of this invention, R is (1) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)-alkoxy, (C₃-C₇)cycloalkoxy, (C₃-C₇)cycloalkoxy(C₁-C₃)alkyl, (C₃-C₇)cycloalkyl(C₁-C₃)alkoxy, (C₁-C₈)alkylthio, (C₃-C₇)cycloalkylthio, (C₃-C₇)cycloalkylthio(C₁-C₃)alkyl, (C₃-C₇)cycloalkyl(C₁-C₃)alkylthio, azepano, azetidino, piperidino, pyrrolidino or tri(C₁-C₄)alkylsilyl, each optionally substituted with up to four substituents independently selected from the group consisting of: fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy, and oxo; or

(2) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, arylethenyl, heteroarylethenyl, or arylethynyl, heteroarylethynyl, each optionally substituted with up to three substituents independently selected from the group consisting of: fluorine, chlorine, cyano, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cyclo-alkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NCO, H₂NSO₂₅ (C₁-C₆)alkylaminocarbonyl, and (C₁-C₆)alkylaminosulfonyl; or

(3) R is a divalent radical selected from —(CH₂)₄— and —(CH₂)₅—, which is attached to R¹ to form a fused or spirofused ring system, and is optionally substituted with up to four substituents independently selected from: fluorine, hydroxy, (C₁-C₆)alkyl, halo(C₁-C₆)alkyl, (C₁-C₆)alkoxy and oxo.

In another particular embodiment, R is phenyl, naphthyl, monocyclic heteroaryl, bicyclic heteroaryl, phenoxy, monocyclic heteroaryloxy, phenyl(C₁-C₃)alkoxy, or monocyclic heteroaryl(C₁-C₃)alkoxy, each optionally substituted with up to three substituents independently selected from halogen, cyano, (C₁-C₃)alkyl, (C₃-C₅)cycloalkyl, halo(C₁-C₃)alkyl, (C₁-C₃)alkoxy, halo(C₁-C₃)alkoxy, (C₁-C₃)alkylthio, and H₂NCO; or a divalent radical selected from —(CH₂)₄— or —(CH₂)₅—, which is attached to R¹ to form a fused or spirofused ring system.

In a further particular embodiment of this invention, R is phenyl, naphthyl, monocyclic heteroaryl, bicyclic heteroaryl, phenoxy, monocyclic heteroaryloxy, phenyl(C₁-C₃)alkoxy, phenyl(C₁-C₃)alkyl, and monocyclic heteroaryl(C₁-C₃)alkoxy, each optionally substituted with up to 3 substituents/independently selected from fluorine, chlorine, cyano, (C₁-C₃)alkyl, (C₃-C₆)cycloalkyl, halo(C₁-C₃)alkyl, (C₁-C₃)alkoxy, (C₁-C₃)alkylthio, and H₂NCO.

In another particular embodiment of this invention, R is phenyl, naphthyl, monocyclic heteroaryl, bicyclic heteroaryl, arylheterocyclyl, phenoxy, monocyclic heteroaryloxy, phenyl(C₁-C₃)alkoxy, phenyl(C₁-C₃)alkyl, and monocyclic heteroaryl(C₁-C₃)alkoxy, each optionally substituted with up to 3 substituents/independently selected from fluorine, chlorine, (C₁-C₃)alkyl, (C₃-C₆)cycloalkyl, halo(C₁-C₃)alkyl, and (C₁-C₃)alkoxy.

In specific embodiments of this invention, R is 3-ethylphenyl, 3-methylphenyl, 3-isopropylphenyl, 3-methoxyphenyl, 3-methoxymethyl-phenyl, 2-ethoxyphenyl, 3-methoxy-5-methyl-phenyl, phenoxy, 2-methyl-phenoxy, 2-ethyl-phenoxy, 3-methyl-phenoxy, 2-chloro-6-methyl-phenoxy, 3-methyl-4-fluoro-phenyl, 2-fluoro-5-methyl-phenyl, 3-methylbenzyl-, 2-methylbenzyl-, quinolin-3-yl, or naphthalene-2-yl.

R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally substituted with up to four substituents independently selected from the group consisting of: fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylaminocarbonyl, and di(C₁-C₆)alkylaminocarbonyl.

In a particular embodiment of this invention, R¹ is a phenyl, monocyclic heteroaryl, bicyclic heteroaryl, benzo-1,3-dioxole, or (C₃-C₇)cycloalkyl ring optionally substituted with up to four substituents independently selected from the group consisting of: fluorine, chlorine, bromine, cyano, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NSO₂, H₂NCO, (C₁-C₃)alkylaminosulfonyl, and (C₁-C₃)alkylaminocarbonyl.

In another particular embodiment of this invention, R¹ is a phenyl or a monocyclic heteroaryl ring, optionally substituted with up to four substituents independently selected from: halogen, cyano, (C₁-C₃)alkyl, halo(C₁-C₃)alkyl, (C₁-C₃)alkoxy, halo(C₁-C₃)alkoxy, and H₂NCO. In another particular embodiment of this invention, R¹ is a phenyl ring or a pyridinyl ring, optionally substituted with up to 1-2 substituents independently selected from: halogen, (C₁-C₃)alkyl, halo(C₁-C₃)alkyl, (C₁-C₃)alkoxy, and halo(C₁-C₃)alkoxy.

In specific embodiments of this invention, R¹ is phenyl, 3-fluorophenyl, 3-chlorophenyl, 3,5-difluorophenyl, 3-fluoro-4-methoxyphenyl, 2-chloropyridinyl, 3-chloropyridinyl, or 4-chloropyridinyl. In the above embodiment, when R¹ is an optionally substituted phenyl ring, the relative bonding arrangement of R and X to R¹ and the convention used to number substituents is as follows:

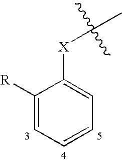

R² is (1) hydrogen or (2) (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, aminocarbonylamino(C₁-C₁₂)alkylamino, (C₁-C₆)-alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxy-carbonyl(C₁-C₆)alkylamino, (C₁-C₆)acyloxy(C₁-C₆)alkyl, (C₁-C₆)acyloxy(C₁-C₆)alkoxy, (C₁-C₆)acyloxy(C₁-C₆)alkylthio, (C₁-C₆)acyloxy(C₁-C₆)alkylamino, aminosulfonylamino(C₁-C₁₂)alkyl, aminosulfonylamino(C₁-C₁₂)alkoxy, aminosulfonylamino(C₁-C₁₂)alkylthio, aminosulfonyl-amino(C₁-C₁₂)alkylamino, (C₁-C₆)alkanesulfonylamino(C₁-C₆)alkyl, (C₁-C₆)alkanesulfonyl-amino(C₁-C₆)alkoxy, (C₁-C₆)alkanesulfonylamino(C₁-C₆)alkylthio, (C₁-C₆)alkanesulfonyl-amino(C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkyl, (C₁-C₆)alkylaminocarbonylamino(C₁-C₆)alkoxy, (C₁-C₆)alkylamino carbonyl-amino(C₁-C₆)alkylthio, (C₁-C₆)alkylaminocarbonylamino(C₁-C₆)alkylamino, amino carbonyl(C₁-C₆)alkyl, amino carbonyl(C₁-C₆)alkoxy, amino carbonyl(C₁-C₆)alkylthio, amino carbonyl(C₁-C₆)alkylamino, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, amino carboxy(C₁-C₆)alkyl, amino carboxy(C₁-C₆)alkoxy, amino carboxy(C₁-C₆)alkylthio, amino carboxy(C₁-C₆)alkylamino, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, wherein (1) each optionally substituted by (a) 1 to 5 halogen atoms and (b) by 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy and wherein (2) divalent sulfur atoms are optionally oxidized to sulfoxide or sulfone and wherein (3) a carbonyl group is optionally changed to a thiocarbonyl group.

In a particular embodiment of this invention, R² is, (C₁-C₁₀)alkoxy, (C₁-C₁₀)alkylthio, (C₁-C₁₀)alkylamino, (C₁-C₅)alkoxy(C₁-C₅)alkyl, (C₁-C₅)alkylthio(C₁-C₅)alkyl, (C₁-C₅)alkylamino(C₁-C₅)alkyl, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)alkylthio, (C₁-C₅)alkylamino(C₁-C₅)alkylamino, (C₁-C₃)alkoxy(C₁-C₃)alkoxy(C₁-C₃)alkyl, aminocarbonylamino(C₁-C₁₀)alkyl, aminocarbonylamino(C₁-C₁₀)alkoxy, aminocarbonylamino-(C₁-C₁₀)alkylthio, aminocarbonylamino(C₁-C₁₀)alkylamino, (C₁-C₅)alkanoylamino(C₁-C₅)alkyl, (C₁-C₅)alkanoylamino(C₁-C₅)alkoxy, (C₁-C₅)alkanoylamino(C₁-C₅)alkylthio, (C₁-C₅)-alkanoylamino(C₁-C₅)alkylamino, aminosulfonylamino(C₁-C₁₀)alkyl, aminosulfonylamino(C₁-C₁₀)alkoxy, aminosulfonylamino(C₁-C₁₀)alkylthio, aminosulfonylamino(C₁-C₁₀)alkylamino, (C₁-C₅)alkanesulfonylamino(C₁-C₅)alkyl, (C₁-C₅)alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkyl, formylamino(C₁-C₅)alkoxy, formylamino(C₁-C₅)alkylthio, formylamino(C₁-C₅)alkylamino, (C₁-C₅)alkoxycarbonylamino(C₁-C₅)alkyl, (C₁-C₅)alkoxy-carbonylamino(C₁-C₅)alkoxy, (C₁-C₅)alkoxycarbonylamino(C₁-C₅)alkylthio, (C₁-C₅)alkoxy-carbonylamino(C₁-C₅)alkylamino, (C₁-C₅)alkylaminocarbonylamino(C₁-C₅)alkyl, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkyl, aminocarbonyl(C₁-C₅)alkoxy, aminocarbonyl(C₁-C₅)alkylthio, amino carbonyl(C₁-C₅)alkylamino, (C₁-C₅)alkylaminocarbonyl(C₁-C₅)alkyl, (C₁-C₅)alkylaminocarbonyl(C₁-C₅)alkoxy, (C₁-C₅)alkylaminocarbonyl(C₁-C₅)alkylthio, (C₁-C₅)alkylaminocarbonyl(C₁-C₅)alkylamino, aminocarboxy(C₁-C₅)alkyl, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkyl, (C₁-C₅)alkylaminocarboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁)alkoxycarbonylamino, (C₁-C₁₀)alkylaminocarbonylamino, or (C₁-C₁₀)-alkanoylamino, wherein (1) each are optionally substituted by (a) 1 to 5 fluorine atoms and/or (b) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy and wherein (2) divalent sulfur atoms are optionally oxidized to sulfoxide or sulfone.

In another particular embodiment of this invention, R² is (C₁-C₈)alkoxy, (C₄-C₉)cycloalkylalkoxy, fluoro(C₁-C₈)alkoxy, hydroxy(C₁-C₈)alkyl, (C₁-C₅)alkoxy(C₁-C₅)alkyl, halo(C₁-C₅)alkylamino(C₁-C₅)alkyl, (C₁-C₅)alkoxy(C₁-C₅)hydroxyalkyl, (C₃-C₆)cycloalkoxy(C₁-C₅)alkyl, fluoro(C₁-C₅)alkoxy(C₁-C₅)alkyl, fluoro(C₃-C₆)cycloalkoxy(C₁-C₅)alkyl, (C₁-C₅)alkylthio(C₁-C₅)alkyl, (C₁-C₅)alkoxy(C₁-C₅)alkoxy, hydroxy(C₁-C₈)alkoxy, (C₃-C₆)cycloalkoxy(C₁-C₅)alkoxy, fluoro(C₁-C₅)alkoxy(C₁-C₅)alkoxy, fluoro(C₃-C₆)cycloalkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, hydroxy-(C₁-C₃)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₃-C₄)-cycloalkanecarbonylamino(C₁-C₅)alkyl, (C₃-C₄)cycloalkanecarbonylamino(C₁-C₅)alkoxy, aminosulfonylamino(C₁-C₈)alkyl, aminosulfonylamino(C₁-C₈)alkoxy, (C₁-C₅)alkane-sulfonylamino(C₁-C₅)alkyl, (C₁-C₅)alkanesulfonylamino(C₁-C₅)alkoxy, formylamino(C₁-C₅)alkyl, formylamino(C₁-C₅)alkoxy, (C₁-C₅)alkoxycarbonylamino(C₁-C₅)alkyl, (C₁-C₅)alkoxycarbonyl-amino(C₁-C₅)alkoxy, (C₁-C₅)alkylaminocarbonylamino(C₁-C₅)alkyl, (C₁-C₅)alkylamino-carbonylamino(C₁-C₅)alkyl, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylamino-carboxy(C₁-C₅)alkoxy, (C₁-C₈)alkoxycarbonylamino, (C₁-C₈)alkylamino carbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino.

In a further particular embodiment of this invention, R² is (C₁-C₃)alkoxy(C₁-C₅)alkyl, (C₁-C₃)alkoxy(C₁-C₅)alkoxy, (C₃-C₆)cycloalkyl(C₁-C₅)alkyl, (C₃-C₆)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, fluoro(C₁-C₃)alkanoylamino(C₁-C₅)alkyl, hydroxy-(C₁-C₃)alkanoylamino(C₁-C₅)alkyl, formylamino(C₁-C₅)alkyl, (C₁-C₃)-alkanoylamino(C₁-C₅)alkoxy, (C₁-C₃)alkylaminocarbonyl(C₁-C₅)alkyl or (C₁-C₃)alkylaminocarbonyl(C₁-C₅)alkoxy. In specific embodiments of this invention, R² is 3-(acetamido)propyl-(CH₃C(═O)NHCH₂CH₂CH₂—), 3-(trifluoro-acetamido)propyl-(CF₃C(═O)NHCH₂CH₂CH₂—), 3-(hydroxy-acetamido)propyl-(HO—CH₂C(═O)NHCH₂CH₂CH₂—), 3-(propanamido)propyl-(CH₃CH₂C(═O)NHCH₂CH₂CH₂—), 3-(formamido)propyl-(H—C(═O)NHCH₂CH₂CH₂—), 3-(methoxycarbonylamino)propyl, 3-(ethoxycarbonylamino)propyl, 3-(isopropoxycarbonylamino)propyl, or 4-methoxybutyl. 3-(methoxycarbonylamino)propyl.

R³ is H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)-alkoxycarbonylamino, (C₁-C₆)alkylamino-carbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonyl-amino, or phenylamino or heteroarylamino in which each phenylamino and heteroarylamino group is optionally substituted with 1 to 5 groups independently selected from the group consisting of: fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)-cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)-cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl; provided that (i) R² and R³ are not both hydrogen and (ii) when R³ is hydroxyl, halogen, or optionally substituted phenylamino or heteroarylamino, R² is not (C₁-C₁₂)alkoxy, (C₁-C₁₂)alkylthio, (C₁-C₁₂)alkylamino, oxo(C₁-C₁₂)alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)-alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, aminocarbonylamino(C₁-C₁₂)alkoxy, amino carbonyl-amino(C₁-C₁₂)alkylthio, aminocarbonylamino(C₁-C₁₂)alkylamino, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl-(C₁-C₆)alkylamino, (C₁-C₆)acyloxy(C₁-C₆)alkoxy, (C₁-C₆) acyloxy(C₁-C₆)alkylthio, (C₁-C₆)-acyloxy(C₁-C₆)alkylamino, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylamino carbonyl-amino(C₁-C₆)alkoxy, (C₁-C₆)alkylaminocarbonylamino(C₁-C₆)alkylthio, (C₁-C₆)alkylamino-carbonylamino(C₁-C₆)alkylamino, aminocarbonyl(C₁-C₆)alkoxy, aminocarbonyl(C₁-C₆)alkylthio, amino carbonyl(C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkoxy, aminocarboxy(C₁-C₆)alkylthio, amino carboxy(C₁-C₆)alkylamino, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, wherein (1) each optionally substituted by (a) 1 to 5 halogen atoms and (b) by 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy and wherein (2) divalent sulfur atoms are optionally oxidized to sulfoxide or sulfone and wherein (3) a carbonyl group is optionally changed to a thiocarbonyl group.

In another particular embodiment of this invention, R³ is hydroxyl, hydroxy(C₁-C₃)alkyl, hydroxy(C₁-C₃)alkoxy, (C₁-C₄)alkanoylamino, (C₁-C₃)alkoxycarbonylamino, (C₁-C₃)alkylamino-carbonylamino, di(C₁-C₃)alkylaminocarbonylamino, (C₁-C₃)alkanesulfonylamino, (C₁-C₃)alkylaminosulfonylamino, di(C₁-C₃)alkylaminosulfonylamino, or phenylamino or heteroarylamino in which each phenylamino and heteroarylamino group is optionally substituted with 1 to 3 groups independently selected from: fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)alkoxycarbonyl;

provided that when R³ is hydroxyl, or optionally substituted phenylamino or heteroarylamino, R² is not (C₁-C₁₀)alkoxy, (C₁-C₁₀)alkylthio, (C₁-C₁₀)alkylamino, (C₁-C₅)alkylthio(C₁-C₅)alkyl, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)alkylthio, (C₁-C₅)alkylamino(C₁-C₅)alkylamino, aminocarbonylamino(C₁-C₁₀)alkoxy, aminocarbonylamino(C₁-C₁₀)alkylthio, amino carbonyl-amino(C₁-C₁₀)alkylamino, (C₁-C₅)alkanoylamino(C₁-C₅)alkoxy, (C₁-C₅)alkanoylamino(C₁-C₅)alkylthio, (C₁-C₅)alkanoylamino(C₁-C₅)alkylamino, aminosulfonylamino(C₁-C₁₀)alkoxy, aminosulfonylamino(C₁-C₁₀)alkylthio, aminosulfonylamino(C₁-C₁₀)alkylamino, (C₁-C₅)-alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkoxy, formylamino(C₁-C₅)alkylthio, formylamino(C₁-C₅)alkylamino, (C₁-C₅)alkoxycarbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl-(C₁-C₅)alkoxy, (C₁-C₅)alkylaminocarbonyl(C₁-C₅)alkylthio, (C₁-C₅)alkylaminocarbonyl(C₁-C₅)alkylamino, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, wherein (1) each are optionally substituted by (a) 1 to 5 fluorine atoms and (b) by 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy and wherein (2) divalent sulfur atoms are optionally oxidized to sulfoxide or sulfone.

In a further particular embodiment of this invention, R³ is OH, (C₁-C₄)alkanoylamino, or (C₁-C₃)alkoxy; provided that when R³ is OH, R² is not (C₃-C₆)cycloalkyl(C₁-C₅)alkoxy, (C₁-C₃)alkoxy(C₁-C₅)alkoxy, (C₁-C₃)-alkanoylamino(C₁-C₅)alkoxy, (C₁-C₃)alkoxycarbonylamino(C₁-C₅)alkoxy, or (C₁-C₃)alkylaminocarbonyl(C₁-C₅)alkoxy. In other embodiments of this invention, R³ is hydrogen or hydroxyl provided that when R³ is hydroxyl, R² is not 3-methoxypropoxy, 2-(acetylamino)ethoxy, or 2-(methoxycarbonylamino)ethoxy. In specific embodiments of this invention, R³ is hydroxyl.

A is a saturated or unsaturated 4-, 5-, 6-, or 7-membered ring which is optionally bridged by (CH₂)ₘ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring atoms being substituted with the appropriate number of hydrogen atoms, said ring being optionally substituted with up to four independently selected halogen atoms, (C₁-C₆)alkyl groups, halo(C₁-C₆)alkyl groups or oxo groups such that when there is substitution with one oxo group on a carbon atom it forms a carbonyl group and when there is substitution of one or two oxo groups on sulfur it forms sulfoxide or sulfone groups, respectively; and, wherein m is 1 to 3. In one embodiment of the compounds of this invention, Ring A is a piperidine (piperidinyl) ring, a morpholine (morpholinyl) ring or a benzene (phenyl) ring. In specific embodiments of this invention, Ring A is a piperidine ring or a morpholine ring.

Q and Y are attached to carbon or nitrogen atoms in ring A in a 1, 2 or 1,3, or 1,4 relationship. In specific embodiments of this invention, Q and Y are attached to carbon or nitrogen atoms in ring A in a 1, 3 or 1,4 relationship. In other specific embodiments, Q and Y are attached to carbon or nitrogen atoms in ring A in a 1,3 relationship. X and Y are each independently CH₂ or a single bond. In the specific embodiments of this invention, X and Y are each a single bond.

In one particular embodiment of this invention, Q is a divalent radical:

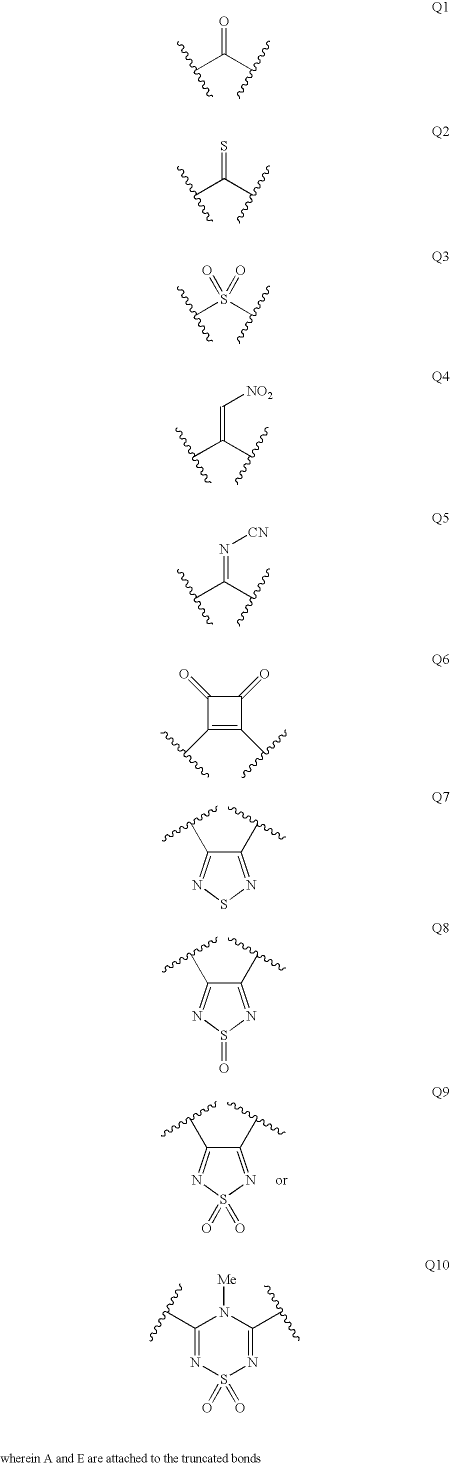

In another embodiment of this invention, Q is a divalent radical selected from Q1, Q2, Q3, Q4, Q5, Q6, and Q7. In a further embodiment of this invention, Q is Q1, Q2 or Q6. In specific embodiments of this invention, Q is Q1.

In another particular embodiment of this invention, E is a phenyl, methyl-phenyl, or naphthyl group, optionally substituted on the phenyl or naphthyl moiety thereof with up to four groups independently selected from halogen, hydroxy, (C₁-C₆)alkyl, halo(C₁-C₆)alkyl, hydroxy(C₁-C₆)alkyl, cyano(C₁-C₆)alkyl, NH₂C(═O)—(C₁-C₆)alkyl, (C₁-C₆)alkoxy, aryl, heterocyclyl(C₁-C₆)alkyl, (C₄-C₆)cycloalkyl(C₁-C₆)alkyl, —C(═O)(C₁-C₆)alkyl, —C(═O)OH, —C(═O)O(C₁-C₆)alkyl, —C(═O)NH₂, and —CN.

In another particular embodiment of this invention, E is phenyl, optionally substituted with up to four groups independently selected from halogen, hydroxy, (C₁-C₆)alkyl, halo(C₁-C₆)alkyl, hydroxy(C₁-C₆)alkyl, heterocyclyl(C₁-C₆)alkyl, (C₄-C₆)cycloalkyl(C₁-C₆)alkyl(C₁-C₆)alkoxy, —C(═O)(C₁-C₆)alkyl, —C(═O)OH, —C(═O)O(C₁-C₆)alkyl, —C(═O)NH₂, and —CN. In specific embodiments of this invention, E is phenyl, optionally substituted with 0 or 1 fluoro groups. In more specific embodiments, E is phenyl or fluoro-phenyl.

In specific embodiments of this invention, E is phenyl, 2-fluorophenyl, 2-bromophenyl, 2-phenylphenyl, 2-methylphenyl, 2-ethylphenyl, 2-n-butylphenyl, 2-iso-butylphenyl, 2-methoxyphenyl, 3-methoxyphenyl, 2-cyanophenyl, 2-carboxyphenyl, 2-[H₂N(C═O)]phenyl, 2-[H₂N(C═O)CH₂]phenyl, 2-[H₂N(C═O)(CH₂)₂]phenyl, 2-(cyanoethyl)phenyl, 2-[(1H-tetrazol-5-yl)ethyl]phenyl, 2-[2-(1,3-dioxolan-2-yl)ethyl]phenyl, naphthyl, or benzyl. In the above embodiment, when R¹ is a substituted phenyl ring, the relative bonding arrangement of Q and G to E and the convention used to number substituents is as follows:

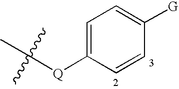

In the embodiments of the compounds of this invention, Q and G may be attached to ring E in a 1, 3 or a 1,4 relationship. In the specific embodiments of this invention, Q and G are attached to ring E in a 1,4 relationship.

In one embodiment of the compounds of this invention, G is hydroxy, —NR⁴R^{4a}, —O—(C₂-C₄)alkyl-NR⁴R^{4a}, —(C₁-C₄)alkyl-OH, —(C₁-C₄)alkyl-NR⁴R^{4a}, —C(═O)(C₁-C₄)alkyl-NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₇)cycloalkyl or —(C₁-C₄)alkyl(C₄-C₇)heterocyclyl, wherein the (C₁-C₄)alkyl moiety of said —C(═O)(C₁-C₄)alkylaryl, —C(═O)(C₁-C₄)alkyl(C₄-C₇)heterocyclyl, —(C₁-C₄)alkyl(C₃-C₇)cycloalkyl and —(C₁-C₄)alkyl(C₄-C₇)heterocyclyl is optionally substituted by amino, hydroxy, or (C₁-C₃)alkylamino, and where R⁴ is H or (C₁-C₃)alkyl and R^{4a} is selected from H, (C₁-C₃)alkyl, (C₃-C₆)cycloalkyl(C₁-C₄)alkyl and (C₄-C₆)heterocyclyl(C₁-C₄)alkyl or R⁴ and R^{4a}, taken together with the nitrogen atom to which they are attached, form a 5-6 membered saturated heterocyclic ring composed of carbon atoms and 1-2, specifically 2, nitrogen atoms, said ring being optionally substituted with up to four groups independently selected from halogen, hydroxy, amino, (C₁-C₆)alkyl, (C₁-C₆)alkylamino, halo(C₁-C₆)alkyl, hydroxy(C₁-C₆)alkyl, amino(C₁-C₆)alkyl, and oxo groups such that when there is substitution with one oxo group on a carbon atom it forms a carbonyl group. In another embodiment of this invention, at least one of R⁴ and R^{4a} is H.

In another embodiment of the compounds of this invention, G is, —(C₁-C₄)alkyl-OH, —(C₁-C₄)alkyl-NR⁴R^{4a}, —O(C₂-C₄)alkyl-NR⁴R^{4a}, —C(═O)(C₁-C₄)alkyl-NR⁴R^{4a}, —C(═O)(C₁-C₄)alkylphenyl, —C(═O)(C₁-C₄)alkyl(C₄-C₆)heterocyclyl, —(C₁-C₄)alkyl(C₃-C₆)cycloalkyl, or —(C₁-C₄)alkyl(C₄-C₆)heterocyclyl, wherein the (C₁-C₄)alkyl moiety of said —C(═O)(C₁-C₄)alkylphenyl, —C(═O)(C₁-C₄)alkyl(C₄-C₆)heterocyclyl, —(C₁-C₄)alkyl(C₃-C₆)cycloalkyl and —(C₁-C₄)alkyl(C₄-C₆)heterocyclyl is optionally substituted by amino, hydroxy, or (C₁-C₃)alkylamino, and where R⁴ is H or (C₁-C₃)alkyl and R^{4a} is selected from H, (C₁-C₃)alkyl, (C₃-C₆)cycloalkyl(C₁-C₄)alkyl and (C₄-C₆)heterocyclyl(C₁-C₄)alkyl, or R⁴ and R^{4a}, taken together with the nitrogen atom to which they are attached, form a 5-6 membered saturated heterocyclic ring composed of carbon atoms and 2 nitrogen atoms.

In specific embodiments of this invention, G is —(C₁-C₃)alkyl-NR⁴R^{4a} or —O(C₂-C₃)alkyl-NR⁴R^{4a}, where R⁴ is H or (C₁-C₃)alkyl and R^{4a} is selected from H, (C₁-C₃)alkyl and (C₅-C₆)cycloalkyl(C₁-C₃)alkyl, or R⁴ and R^{4a}, taken together with the nitrogen atom to which they are attached, form a 5-6 membered saturated heterocyclic ring composed of carbon atoms and 1-2 nitrogen atoms. In more specific embodiments, G is —CH₂NH₂, —CH₂CH₂NH₂, —OCH₂CH₂NH₂, —CH₂NHCH₃, —CH₂NHCH₂CH₃, —CH₂NHCH(CH₃)₂, —CH₂N(CH₃)₂, —CH₂NHCH₂-cyclohexyl, or —CH₂— (piperazin-1-yl).

An embodiment of the invention is a compound of Formula Ia:

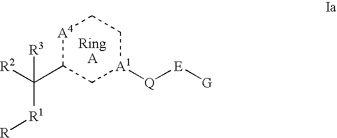

wherein R, R¹, R², R³, Q, E and G are as defined hereinabove, and Ring A is a benzene ring (A¹ and A⁴ are CH and the bonds in ring A are aromatic bonds); or Ring A is a piperidine ring (A¹ is N, A⁴ is CH₂ and the bonds in ring A are single bonds); or Ring A is a morpholine ring (A¹ is N, A⁴ is O and the bonds in ring A are single bonds), or an enantiomer, diastereomer or salt thereof.

An embodiment of the invention is a compound of Formula Ia:

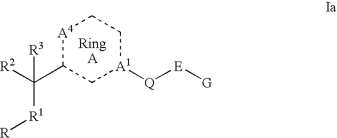

wherein R, R¹, R², R³, Q, E and G are as defined above for Formula I, and Ring A is a benzene ring (A¹ and A⁴ are CH and the bonds in ring A are aromatic bonds); or Ring A is a piperidine ring (A¹ is N, A⁴ is CH₂ and the bonds in ring A are single bonds); or Ring A is a morpholine ring (A¹ is N, A⁴ is O and the bonds in ring A are single bonds), or an enantiomer, diastereomer or salt thereof.

Another embodiment of the invention is a compound of Formula Ia with the stereochemical configuration shown in Formula Ib:

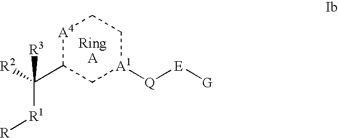

wherein R, R¹, R², R³, Ring A, A¹, A⁴, Q, E and G are as defined above, or an enantiomer, diastereomer or salt thereof. Specific and particular values for each variable in Formula Ib are as described above.

A further embodiment of invention is a compound of Formula Ic:

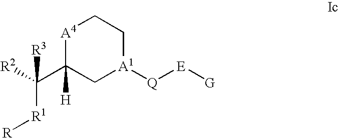

wherein R, R¹, R², R³, A, A¹, A⁴, Q, E and G are as defined above, or an enantiomer, diastereomer or salt thereof. Specific and particular values for each variable in Formula Ic are as described above.

One particular embodiment of the invention is a compound of Formula I, Ia, Ib, or Ic wherein:

E is phenyl, optionally substituted with 1-2 groups independently selected from halogen, hydroxy, (C₁-C₆)alkyl, halo(C₁-C₆)alkyl and hydroxy(C₁-C₆)alkyl;

R is phenyl, naphthyl, monocyclic heteroaryl, bicyclic heteroaryl, phenoxy, monocyclic heteroaryloxy, phenyl(C₁-C₃)alkoxy, phenyl(C₁-C₃)alkyl, and monocyclic heteroaryl(C₁-C₃)alkoxy, each optionally substituted with up to 3 substituents/independently selected from fluorine, chlorine, (C₁-C₃)alkyl, (C₃-C₆)cycloalkyl, halo(C₁-C₃)alkyl, and (C₁-C₃)alkoxy; R¹ is a phenyl ring, optionally substituted with up to 1-2 substituents independently selected from: halogen, (C₁-C₃)alkyl, halo(C₁-C₃)alkyl, (C₁-C₃)alkoxy, and halo(C₁-C₃)alkoxy;

R² is (C₁-C₃)alkoxy(C₁-C₅)alkyl, (C₁-C₃)alkoxy(C₁-C₅)alkoxy, (C₃-C₆)cycloalkyl(C₁-C₅)alkyl, (C₃-C₆)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, fluoro(C₁-C₃)alkanoylamino(C₁-C₅)alkyl, hydroxy-(C₁-C₃)alkanoylamino(C₁-C₅)alkyl, formylamino(C₁-C₅)alkyl, (C₁-C₃)-alkanoylamino(C₁-C₅)alkoxy, (C₁-C₃)alkylaminocarbonyl(C₁-C₅)alkyl or (C₁-C₃)alkylaminocarbonyl(C₁-C₅)alkoxy; and

R³ is OH, (C₁-C₄)alkanoylamino, or (C₁-C₃)alkoxy; provided that when R³ is OH, R² is not (C₃-C₆)cycloalkyl(C₁-C₅)alkoxy, (C₁-C₃)alkoxy(C₁-C₅)alkoxy, (C₁-C₃)-alkanoylamino(C₁-C₅)alkoxy, (C₁-C₃)alkoxycarbonylamino(C₁-C₅)alkoxy, or (C₁-C₃)alkylaminocarbonyl(C₁-C₅)alkoxy, or a salt thereof.

Further embodiments of this invention, comprise compounds of Formula I, Ia, Ib, or Ic wherein E, R, R¹, R² and R³ are as defined hereinabove, and:

A or Ring A is a piperidine ring, a morpholine ring or a benzene ring, where for Formulas I, Ia and Ib: A¹ and A⁴ are CH and the bonds in ring A are aromatic bonds, or for Formulas I, Ia, Ib and Ic: Ring A is a piperidine ring, where A¹ is N, A⁴ is CH₂ and the bonds in ring A are single bonds, or Ring A is a morpholine ring, where A¹ is N, A⁴ is O and the bonds in ring A are single bonds);

Q is Q1; and

G is —(C₁-C₃)alkyl-NR⁴R^{4a} or —O(C₂-C₃)alkyl-NR⁴R^{4a}, where R⁴ is H or (C₁-C₃)alkyl and R^{4a} is selected from H, (C₁-C₃)alkyl, and (C₅-C₆)cycloalkyl(C₁-C₃)alkyl, or R⁴ and R^{4a}, taken together with the nitrogen atom to which they are attached, form a 5-6 membered saturated heterocyclic ring composed of carbon atoms and 1-2 nitrogen atoms, or a salt thereof.

Another particular embodiment of the invention is a compound of Formula I, Ia or Ib wherein, wherein R is 3-ethylphenyl, 3-methylphenyl, 3-isopropylphenyl, 3-methoxyphenyl, 3-methoxymethyl-phenyl, 2-ethoxyphenyl, 3-methoxy-5-methyl-phenyl, phenoxy, 2-methyl-phenoxy, 2-ethyl-phenoxy, 3-methyl-phenoxy, 2-chloro-6-methyl-phenoxy, 3-methyl-4-fluoro-phenyl, 2-fluoro-5-methyl-phenyl, 3-methylbenzyl-, 2-methylbenzyl-, quinolin-3-yl, or naphthalene-2-yl; R¹ is phenyl, 3-fluorophenyl or 3-chlorophenyl; R² is 3-(acetamido)propyl-, 3-(trifluoro-acetamido)propyl-, 3-(hydroxy-acetamido)propyl-, 3-(propanamido)propyl-, 3-(formamido)propyl-, 3-(methoxycarbonylamino)propyl, 3-(ethoxycarbonylamino)propyl, 3-(isopropoxycarbonylamino)propyl, or 4-methoxybutyl; R³ is hydroxyl; Ring A is a piperidine ring or a morpholine ring; Q is Q1; E is phenyl or fluoro-phenyl; G is —CH₂NH₂, —CH₂CH₂NH₂, —OCH₂CH₂NH₂, —CH₂NHCH₃, —CH₂NHCH₂CH₃, —CH₂NHCH(CH₃)₂, —CH₂N(CH₃)₂, —CH₂NHCH₂-cyclohexyl, —CH₂— (piperazin-1-yl); or a salt thereof.

It will be appreciated by those skilled in the art, that the compounds of this invention contain 1, 2 or more chiral centers and may exist in different enantiomeric and/or diastereomeric forms. The following compounds are recited without reference to the relative or absolute configuration of any of the chiral centers present therein, but such recitation is intended to encompass each enantiomeric and/or diastereomeric form of these compounds and all mixtures thereof, such as enantiomerically and/or diastereomerically enriched mixtures and racemic mixtures. The following are compounds of the invention:

| Cpd. | |
|---|---|
| No. | Name |
| I-1 | N-(1-(4-(aminomethyl)benzoyl)piperidin-3-yl)-4-hydroxy-4-(2-(o- |
| | tolyloxy)phenyl)butyl)acetamide |
| I-2 | (4-(aminomethyl)phenyl)(3-(1-(3′-ethyl-6-fluorobiphenyl-2-yl)-1-hydroxy-5- |
| | methoxypentyl)piperidin-1-yl)methanone |
| I-3 | (3-(aminomethyl)phenyl)(4-(1-(3′-ethyl-6-fluorobiphenyl-2-yl)-1-hydroxy-5- |
| | methoxypentyl)piperidin-1-yl)methanone |
| I-4 | (4-(aminomethyl)phenyl)(2-(1-(4′,6-difluoro-3′-methylbiphenyl-2-yl)-1-hydroxy- |
| | 5-methoxypentyl)morpholino)methanone |
| I-5 | methyl 4-(1-(4-(aminomethyl)benzoyl)piperidin-3-yl)-4-hydroxy-4-(2-(o- |
| | tolyloxy)phenyl)butylcarbamate |
| I-6 | (4-(2-aminoethyl)phenyl)(4-(1-(3′-ethyl-6-fluorobiphenyl-2-yl)-1-hydroxy-5- |
| | methoxypentyl)piperidin-1-yl)methanone |
| I-7 | (4-(aminomethyl)phenyl)(3-(1-(6-fluoro-3′-methoxy-5′-methylbiphenyl-2-yl)-1- |
| | hydroxy-5-methoxypentyl)piperidin-1-yl)methanone |
| I-8 | (4-(aminomethyl)phenyl)(3-(1-(6-chloro-3′-ethylbiphenyl-2-yl)-1-hydroxy-5- |
| | methoxypentyl)piperidin-1-yl)methanone |
| I-9 | (4-(aminomethyl)phenyl)(3-(1-(3-chloro-2-(2-methylbenzyl)phenyl)-1-hydroxy- |
| | 5-methoxypentyl)piperidin-1-yl)methanone |
| I-10 | (4-(aminomethyl)phenyl)(3-(1-(3-chloro-2-(3-methylbenzyl)phenyl)-1-hydroxy- |
| | 5-methoxypentyl)piperidin-1-yl)methanone |
| I-11 | (3-(1-(6-chloro-3′-ethylbiphenyl-2-yl)-1-hydroxy-5-methoxypentyl)piperidin-1- |
| | yl)(4-(piperazin-1-ylmethyl)phenyl)methanone |
| I-12 | (4-(aminomethyl)phenyl)(2-(1-(6-fluoro-3′-methoxy-5′-methylbiphenyl-2-yl)-1- |
| | hydroxy-5-methoxypentyl)morpholino)methanone |
| I-13 | (3-(aminomethyl)phenyl)(2-(1-(6-chloro-3′-ethylbiphenyl-2-yl)-1-hydroxy-5- |
| | methoxypentyl)morpholino)methanone |
| I-14 | (4-(aminomethyl)phenyl)(2-(1-(6-chloro-3′-ethylbiphenyl-2-yl)-1-hydroxy-5- |
| | methoxypentyl)morpholino)methanone |
| I-15 | (4-(aminomethyl)phenyl)(3-(1-(3-chloro-2-(o-tolyloxy)phenyl)-1-hydroxy-5- |
| | methoxypentyl)piperidin-1-yl)methanone |
| I-16 | (4-(aminomethyl)phenyl)(3-(1-(2-(2-chloro-6-methylphenoxy)phenyl)-1- |
| | hydroxy-5-methoxypentyl)piperidin-1-yl)methanone |
| I-17 | methyl 4-(6-chloro-3′-ethylbiphenyl-2-yl)-4-(1-(4- |
| | ((dimethylamino)methyl)benzoyl)piperidin-3-yl)-4-hydroxybutylcarbamate |
| I-18 | (4-(aminomethyl)phenyl)(2-(1-(6-chloro-2′-fluoro-5′-methylbiphenyl-2-yl)-1- |
| | hydroxy-5-methoxypentyl)morpholino)methanone |
| I-19 | methyl 4-hydroxy-4-(1-(4-((methylamino)methyl)benzoyl)piperidin-3-yl)-4-(2- |
| | (o-tolyloxy)phenyl)butylcarbamate |
| I-20 | N-(4-(6-chloro-3′-ethylbiphenyl-2-yl)-4-hydroxy-4-(1-(4- |
| | ((methylamino)methyl)benzoyl)piperidin-3-yl)butyl)formamide |
| I-21 | (4-(2-aminoethyl)phenyl)(1-(6-chloro-3′-ethylbiphenyl-2-yl)-1-hydroxy-5- |
| | methoxypentyl)piperidin-1-yl)methanone |
| I-22 | (3-(1-(3-chloro-2-(2-methylbenzyl)phenyl)-1-hydroxy-5- |
| | methoxypentyl)piperidin-1-yl)(4-((methylamino)methyl)phenyl)methanone |
| I-23 | methyl 4-(1-(4-(aminomethyl)benzoyl)piperidin-3-yl)-4-(6-fluoro-3′- |
| | methoxybiphenyl-2-yl)-4-hydroxybutylcarbamate |
| I-24 | (4-(aminomethyl)phenyl)(3-(1-(3-chloro-2-(2-ethylphenoxy)phenyl)-1-hydroxy- |
| | 5-methoxypentyl)piperidin-1-yl)methanone |
| I-25 | (4-(aminomethyl)phenyl)(3-(1-(3-chloro-2-(3-ethylphenoxy)phenyl)-1-hydroxy- |
| | 5-methoxypentyl)piperidin-1-yl)methanone |
| I-26 | (4-(aminomethyl)phenyl)(2-(1-(6-chloro-3′-(methoxymethyl)biphenyl-2-yl)-1- |
| | hydroxy-5-methoxypentyl)morpholino)methanone |
| I-27 | (4-(aminomethyl)phenyl)(3-(1-(3-chloro-2-(quinolin-3-yl)phenyl)-1-hydroxy-5- |
| | methoxypentyl)piperidin-1-yl)methanone |
| I-28 | (4-(aminomethyl)phenyl)(2-(1-(3-chloro-2-(naphthalen-2-yl)phenyl)-1-hydroxy- |
| | 5-methoxypentyl)morpholino)methanone |
| I-29 | (4-(aminomethyl)phenyl)(2-(1-(3-chloro-2-(quinolin-3-yl)phenyl)-1-hydroxy-5- |
| | methoxypentyl)morpholino)methanone |
| I-30 | N-(4-(6-chloro-3′-ethylbiphenyl-2-yl)-4-hydroxy-4-(1-(4- |
| | ((methylamino)methyl)benzoyl)piperidin-3-yl)butyl)acetamide |
| I-31 | methyl 4-(6-fluoro-3′-methoxybiphenyl-2-yl)-4-hydroxy-4-(1-(4- |
| | ((methylamino)methyl)benzoyl)piperidin-3-yl)butylcarbamate |
| I-32 | methyl 4-(1-(4-(aminomethyl)benzoyl)piperidin-3-yl)-4-(6-chloro-3′- |
| | ethylbiphenyl-2-yl)-4-hydroxybutylcarbamate |
| I-33 | methyl 4-(6-chloro-3′-methylbiphenyl-2-yl)-4-hydroxy-4-(1-(4- |
| | ((methylamino)methyl)benzoyl)piperidin-3-yl)butylcarbamate |
| I-34 | (4-(2-aminoethoxy)phenyl)(3-(1-(6-chloro-3′-ethylbiphenyl-2-yl)-1-hydroxy-5- |
| | methoxypentyl)piperidin-1-yl)methanone |
| I-35 | methyl 4-(4-(4-(aminomethyl)benzoyl)morpholin-2-yl)-4-(6-chloro-3′- |
| | ethylbiphenyl-2-yl)-4-hydroxybutylcarbamate |
| I-36 | methyl 4-(1-(4-(aminomethyl)benzoyl)piperidin-3-yl)-4-(6-chloro-3′- |
| | methoxybiphenyl-2-yl)-4-hydroxybutylcarbamate |
| I-37 | N-(4-(6-chloro-3′-ethylbiphenyl-2-yl)-4-hydroxy-4-(1-(4- |
| | ((methylamino)methyl)benzoyl)piperidin-3-yl)butyl)propionamide |
| I-38 | ethyl 4-(1-(4-(aminomethyl)benzoyl)piperidin-3-yl)-4-(6-chloro-3′- |
| | ethylbiphenyl-2-yl)-4-hydroxybutylcarbamate |
| I-39 | methyl 4-(6-chloro-3′-ethylbiphenyl-2-yl)-4-hydroxy-4-(1-(4- |
| | ((methylamino)methyl)benzoyl)piperidin-3-yl)butylcarbamate |
| I-40 | methyl 4-(1-(4-(aminomethyl)benzoyl)piperidin-3-yl)-4-(6-chloro-3′- |
| | isopropylbiphenyl-2-yl)-4-hydroxybutylcarbamate |
| I-41 | N-(4-(6-chloro-3′-ethylbiphenyl-2-yl)-4-hydroxy-4-(1-(4- |
| | ((methylamino)methyl)benzoyl)piperidin-3-yl)butyl)-2-hydroxyacetamide |
| I-42 | methyl 4-(6-chloro-3′-methoxybiphenyl-2-yl)-4-hydroxy-4-(1-(4- |
| | ((methylamino)methyl)benzoyl)piperidin-3-yl)butylcarbamate |
| I-43 | methyl 4-(1-(4-(aminomethyl)-2-fluorobenzoyl)piperidin-3-yl)-4-(6-chloro-3′- |
| | ethylbiphenyl-2-yl)-4-hydroxybutylcarbamate |
| I-44 | isopropyl 4-(1-(4-(aminomethyl)benzoyl)piperidin-3-yl)-4-(6-chloro-3′- |
| | ethylbiphenyl-2-yl)-4-hydroxybutylcarbamate |
| I-45 | methyl 4-(6-chloro-3′-ethylbiphenyl-2-yl)-4-(1-(4- |
| | ((ethylamino)methyl)benzoyl)piperidin-3-yl)-4-hydroxybutylcarbamate |
| I-46 | methyl 4-(6-chloro-3′-ethylbiphenyl-2-yl)-4-hydroxy-4-(1-(4- |
| | ((isopropylamino)methyl)benzoyl)piperidin-3-yl)butylcarbamate |
| I-47 | N-(4-(6-chloro-3′-ethylbiphenyl-2-yl)-4-hydroxy-4-(1-(4- |
| | ((methylamino)methyl)benzoyl)piperidin-3-yl)butyl)-2,2,2-trifluoroacetamide |
| I-48 | methyl {4-(3′-ethyl-6-fluoro-2-biphenylyl)-4-hydroxy-4-[(3R)-1-({4- |
| | [(methylamino)methyl]phenyl}carbonyl)-3-piperidinyl]butyl}carbamate |
| I-49 | methyl {4-(2′,6-difluoro-5′-methyl-2-biphenylyl)-4-hydroxy-4-[(3R)-1-({4- |
| | [(methylamino)methyl]phenyl}carbonyl)-3-piperidinyl]butyl}carbamate |
| I-50 | N-{4-(2′,6-difluoro-5′-methyl-2-biphenylyl)-4-hydroxy-4-[(3R)-1-({4- |
| | [(methylamino)methyl]phenyl}carbonyl)-3-piperidinyl]butyl}-2-hydroxyacetamide |
| I-51 | methyl 4-(6-chloro-3′-ethylbiphenyl-2-yl)-4-(1-(4- |
| | ((cyclohexylmethylamino)methyl)benzoyl)piperidin-3-yl)-4-hydroxybutylcarbamate |
| I-52 | 1-(6-chloro-3′-ethyl-2-biphenylyl)-1-[4-({4- |
| | [(methylamino)methyl]phenyl}carbonyl)-2-morpholinyl]-5-(methyloxy)-1-pentanol |
| I-53 | 1-(6-chloro-3′-ethyl-2-biphenylyl)-1-[1-({4- |
| | [(methylamino)methyl]phenyl}carbonyl)-3-piperidinyl]-5-(methyloxy)-1-pentanol |
| I-54 | methyl [4-(1-{[4-(aminomethyl)-3-(methyloxy)phenyl]carbonyl}-3-piperidinyl)- |
| | 4-(6-chloro-3′-ethyl-2-biphenylyl)-4-hydroxybutyl]carbamate |
| I-55 | N-{4-(6-chloro-3′-methyl-2-biphenylyl)-4-hydroxy-4-[1-({4- |
| | [(methylamino)methyl]phenyl}carbonyl)-3-piperidinyl]butyl}acetamide |
| I-56 | N-[4-(1-{[4-(aminomethyl)-2-fluorophenyl]carbonyl}-3-piperidinyl)-4-(6- |
| | chloro-3′-methyl-2-biphenylyl)-4-hydroxybutyl]acetamide |
| I-57 | N-[4-(1-{[4-(aminomethyl)phenyl]carbonyl}-3-piperidinyl)-4-(6-chloro-3′- |
| | methyl-2-biphenylyl)-4-hydroxybutyl]acetamide |
| I-58 | N-{4-(6-chloro-3′-methyl-2-biphenylyl)-4-[1-({2-fluoro-4- |
| | [(methylamino)methyl]phenyl}carbonyl)-3-piperidinyl]-4-hydroxybutyl}acetamide |
| I-59 | N-{4-(6-chloro-3′-methyl-2-biphenylyl)-4-hydroxy-4-[1-({4-[2,2,2-trifluoro-1- |
| | hydroxy-1-(trifluoromethyl)ethyl]phenyl}carbonyl)-3-piperidinyl]butyl}acetamide |
| I-60 | 4-{[3-(1-(6-chloro-3′-ethyl-2-biphenylyl)-1-hydroxy-4- |
| | {[(methyloxy)carbonyl]amino}butyl)-1-piperidinyl]carbonyl}benzoic acid |
| I-61 | methyl {4-(6-chloro-3′-methyl-2-biphenylyl)-4-hydroxy-4-[1-({4- |
| | [(methylsulfonyl)methyl]phenyl}carbonyl)-3-piperidinyl]butyl}carbamate |
| I-62 | (4-{[(3R)-3-((1S)-1-(6-chloro-3′-ethyl-2-biphenylyl)-1-hydroxy-4- |
| | {[(methyloxy)carbonyl]amino}butyl)-1-piperidinyl]carbonyl}phenyl)-N,N,N- |
| | trimethylmethanaminium |
| I-63 | methyl [4-hydroxy-4-[1-({4-[(methylamino)methyl]phenyl}carbonyl)-3- |
| | piperidinyl]-4-(2′,4,6-trifluoro-5′-methyl-2-biphenylyl)butyl]carbamate |
| I-64 | methyl [4-(1-{[4-(2-aminoethyl)phenyl]carbonyl}-3-piperidinyl)-4-(6-chloro-3′- |
| | methyl-2-biphenylyl)-4-hydroxybutyl]carbamate |
| I-65 | methyl [4-[1-({2-fluoro-4-[(methylamino)methyl]phenyl}carbonyl)-3- |
| | piperidinyl]-4-hydroxy-4-(2′,4,6-trifluoro-5′-methyl-2-biphenylyl)butyl]carbamate |
| I-66 | methyl [4-[1-({4-[(2-aminoethyl)oxy]phenyl}carbonyl)-3-piperidinyl]-4-(6- |
| | chloro-3′-methyl-2-biphenylyl)-4-hydroxybutyl]carbamate |
| I-67 | methyl [4-(1-{[4-(aminomethyl)phenyl]carbonyl}-3-piperidinyl)-4-(6-chloro-3′- |
| | methyl-2-biphenylyl)-4-hydroxybutyl]carbamate |
| I-68 | methyl [4-(1-{[3-(aminomethyl)phenyl]acetyl}-3-piperidinyl)-4-(6-chloro-3′- |
| | methyl-2-biphenylyl)-4-hydroxybutyl]carbamate |
| I-69 | methyl {4-(6-fluoro-3′-methyl-2-biphenylyl)-4-hydroxy-4-[1-({4- |
| | [(methylamino)methyl]phenyl}carbonyl)-3-piperidinyl]butyl}carbamate |
| I-70 | N-{4-(6-fluoro-3′-methyl-2-biphenylyl)-4-hydroxy-4-[1-({4- |
| | [(methylamino)methyl]phenyl}carbonyl)-3-piperidinyl]butyl}-2-hydroxyacetamide |
| I-71 | 2-chloro-N-{4-(6-chloro-3′-ethyl-2-biphenylyl)-4-hydroxy-4-[1-({4- |
| | [(methylamino)methyl]phenyl}carbonyl)-3-piperidinyl]butyl}acetamide |
| I-72 | methyl {4-(6-chloro-3′-ethyl-2-biphenylyl)-4-[1-({4- |
| | [(cyclohexylamino)methyl]phenyl}carbonyl)-3-piperidinyl]-4- |
| | hydroxybutyl}carbamate |
| I-73 | methyl {4-(6-chloro-3′-ethyl-2-biphenylyl)-4-hydroxy-4-[1-({4-[(tetrahydro-2H- |
| | pyran-4-ylamino)methyl]phenyl}carbonyl)-3-piperidinyl]butyl}carbamate |
| I-74 | methyl [4-[1-({2-bromo-4-[(methylamino)methyl]phenyl}carbonyl)-3- |
| | piperidinyl]-4-(6-chloro-3′-ethyl-2-biphenylyl)-4-hydroxybutyl]carbamate |
| I-75 | methyl {4-(6-chloro-3′-ethyl-2-biphenylyl)-4-hydroxy-4-[1-({5- |
| | [(methylamino)methyl]-2-biphenylyl}carbonyl)-3-piperidinyl]butyl}carbamate |
| I-76 | methyl {4-(6-chloro-3′-ethyl-2-biphenylyl)-4-hydroxy-4-[1-({4- |
| | [(methylamino)methyl]-1-naphthalenyl}carbonyl)-3-piperidinyl]butyl}carbamate |
| I-77 | methyl [4-[1-({2-butyl-4-[(methylamino)methyl]phenyl}carbonyl)-3- |
| | piperidinyl]-4-(6-chloro-3′-ethyl-2-biphenylyl)-4-hydroxybutyl]carbamate |
| I-78 | methyl {4-(6-chloro-3′-ethyl-2-biphenylyl)-4-[1-({2-ethyl-4- |
| | [(methylamino)methyl]phenyl}carbonyl)-3-piperidinyl]-4-hydroxybutyl}carbamate |
| I-79 | methyl {4-(6-chloro-3′-ethyl-2-biphenylyl)-4-hydroxy-4-[1-({2-methyl-4- |
| | [(methylamino)methyl]phenyl}carbonyl)-3-piperidinyl]butyl}carbamate |
| I-80 | methyl [4-(6-chloro-3′-ethyl-2-biphenylyl)-4-hydroxy-4-(1-{[4- |
| | [(methylamino)methyl]-2-(2-methylpropyl)phenyl]carbonyl}-3- |
| | piperidinyl)butyl]carbamate |
| I-81 | methyl {4-(6-chloro-3′-ethyl-2-biphenylyl)-4-[1-({2-[2-(1,3-dioxolan-2- |
| | yl)ethyl]-4-[(methylamino)methyl]phenyl}carbonyl)-3-piperidinyl]-4- |
| | hydroxybutyl}carbamate |
| I-82 | methyl {4-(6-chloro-3′-ethyl-2-biphenylyl)-4-[1-({2-(2-cyanoethyl)-4- |
| | [(methylamino)methyl]phenyl}carbonyl)-3-piperidinyl]-4-hydroxybutyl}carbamate |
| I-83 | methyl {4-(6-chloro-3′-ethyl-2-biphenylyl)-4-hydroxy-4-[1-({4- |
| | [(methylamino)methyl]-2-[2-(1H-tetrazol-5-yl)ethyl]phenyl}carbonyl)-3- |
| | piperidinyl]butyl}carbamate |
| I-84 | methyl [4-[1-({2-(3-amino-3-oxopropyl)-4- |
| | [(methylamino)methyl]phenyl}carbonyl)-3-piperidinyl]-4-(6-chloro-3′-ethyl-2- |
| | biphenylyl)-4-hydroxybutyl]carbamate |
| I-85 | methyl {4-(6-chloro-3′-ethyl-2-biphenylyl)-4-[1-({2-cyano-4- |
| | [(methylamino)methyl]phenyl}carbonyl)-3-piperidinyl]-4-hydroxybutyl}carbamate |
| I-86 | methyl [4-[1-({2-(aminocarbonyl)-4-[(methylamino)methyl]phenyl}carbonyl)-3- |
| | piperidinyl]-4-(6-chloro-3′-ethyl-2-biphenylyl)-4-hydroxybutyl]carbamate |
| I-87 | methyl {4-[6-fluoro-3′-methyl-5-(methyloxy)-2-biphenylyl]-4-hydroxy-4-[1-({4- |
| | [(methylamino)methyl]phenyl}carbonyl)-3-piperidinyl]butyl}carbamate |
| I-88 | methyl [4-(6-chloro-3′-ethyl-2-biphenylyl)-4-hydroxy-4-(1-{[4- |
| | [(methylamino)methyl]-2-(methyloxy)phenyl]carbonyl}-3- |
| | piperidinyl)butyl]carbamate |
| I-89 | 2-{[3-(1-(6-chloro-3′-ethyl-2-biphenylyl)-1-hydroxy-4- |
| | {[(methyloxy)carbonyl]amino}butyl)-1-piperidinyl]carbonyl}-5- |
| | [(methylamino)methyl]benzoic acid |
| I-90 | methyl [4-[1-({2-(2-amino-2-oxoethyl)-4- |
| | [(methylamino)methyl]phenyl}carbonyl)-3-piperidinyl]-4-(6-chloro-3′-ethyl-2- |
| | biphenylyl)-4-hydroxybutyl]carbamate |
| I-91 | methyl {4-(3′-ethyl-6-fluoro-2-biphenylyl)-4-hydroxy-4-[4-({4- |
| | [(methylamino)methyl]phenyl}carbonyl)-2-morpholinyl]butyl}carbamate |
| I-92 | 1-(4-{[4-(aminomethyl)phenyl]carbonyl}-2-morpholinyl)-1-[2-chloro-3-(3- |
| | ethylphenyl)-4-pyridinyl]-5-(methyloxy)-1-pentanol |
| I-93 | 1-(4-{[4-(aminomethyl)phenyl]carbonyl}-2-morpholinyl)-1-[2-(1-benzothien-3- |
| | yl)-3-chlorophenyl]-5-(methyloxy)-1-pentanol |
| I-94 | methyl {4-[3-chloro-2-(3-quinolinyl)phenyl]-4-hydroxy-4-[1-({4- |
| | [(methylamino)methyl]phenyl}carbonyl)-3-piperidinyl]butyl}carbamate |
| I-95 | 1-[4-chloro-3-(3-ethylphenyl)-2-pyridinyl]-1-[4-({4- |
| | [(methylamino)methyl]phenyl}carbonyl)-2-morpholinyl]-5-(methyloxy)-1-pentanol |
| I-96 | methyl {4-{3-chloro-2-[4-(1-methylethyl)-2-quinazolinyl]phenyl}-4-hydroxy-4- |
| | [1-({4-[(methylamino)methyl]phenyl}carbonyl)-3-piperidinyl]butyl}carbamate |
| I-97 | methyl {4-[2-chloro-3-(3-ethylphenyl)-4-pyridinyl]-4-hydroxy-4-[1-({4- |
| | [(methylamino)methyl]phenyl}carbonyl)-3-piperidinyl]butyl}carbamate |
| I-98 | methyl {4-(6-chloro-3′-methyl-2-biphenylyl)-4-hydroxy-4-[4-({4- |
| | [(methylamino)methyl]phenyl}carbonyl)-2-morpholinyl]butyl}carbamate |
| I-99 | 1-(1-{[4-(aminomethyl)phenyl]carbonyl}-3-piperidinyl)-1-{3-chloro-2-[(3- |
| | methylphenyl)oxy]phenyl}-5-(methyloxy)-1-pentanol |
| I-100 | methyl (4-(1-{[4-(aminomethyl)phenyl]carbonyl}-3-piperidinyl)-4-{3-chloro-2- |
| | [(3-methylphenyl)oxy]phenyl}-4-hydroxybutyl)carbamate |
| I-101 | methyl (4-(1-{[4-(aminomethyl)phenyl]carbonyl}-3-piperidinyl)-4-{2-[(2,6- |
| | dimethylphenyl)oxy]phenyl}-4-hydroxybutyl)carbamate |
| I-102 | methyl {4-{3-chloro-2-[8-(1-methylethyl)-2-quinolinyl]phenyl}-4-hydroxy-4-[1- |
| | ({4-[(methylamino)methyl]phenyl}carbonyl)-3-piperidinyl]butyl}carbamate |
| I-103 | methyl [4-(6-chloro-3′-ethyl-2-biphenylyl)-4-hydroxy-4-(1-{[4- |
| | (hydroxymethyl)phenyl]carbonyl}-3-piperidinyl)butyl]carbamate |
| I-104 | methyl (4-(6-chloro-3′-ethyl-2-biphenylyl)-4-hydroxy-4-{1-[(4-{[(2- |
| | hydroxyethyl)amino]methyl}phenyl)carbonyl]-3-piperidinyl}butyl)carbamate |
| I-105 | methyl (4-(6-chloro-3′-ethyl-2-biphenylyl)-4-hydroxy-4-{1-[(4-{[(3- |
| | hydroxypropyl)amino]methyl}phenyl)carbonyl]-3-piperidinyl}butyl)carbamate |
| I-106 | methyl [4-(6-chloro-3′-ethyl-2-biphenylyl)-4-hydroxy-4-(1-{[4-({[2- |
| | (methyloxy)ethyl]amino}methyl)phenyl]carbonyl}-3-piperidinyl)butyl]carbamate |
| I-107 | methyl [4-(6-chloro-3′-ethyl-2-biphenylyl)-4-(1-{[4-({[2- |
| | (ethyloxy)ethyl]amino}methyl)phenyl]carbonyl}-3-piperidinyl)-4- |
| | hydroxybutyl]carbamate |
| I-108 | methyl [4-(6-chloro-3′-ethyl-2-biphenylyl)-4-hydroxy-4-(1-{[4-({[1-methyl-2- |
| | (methyloxy)ethyl]amino}methyl)phenyl]carbonyl}-3-piperidinyl)butyl]carbamate |
| I-109 | methyl {4-(6-chloro-3′-ethyl-2-biphenylyl)-4-hydroxy-4-[1-({4-[({2-[(1- |
| | methylethyl)oxy]ethyl}amino)methyl]phenyl}carbonyl)-3- |
| | piperidinyl]butyl}carbamate |
| I-110 | methyl (4-(6-chloro-3′-ethyl-2-biphenylyl)-4-hydroxy-4-{1-[(4-{[(tetrahydro-2- |
| | furanylmethyl)amino]methyl}phenyl)carbonyl]-3-piperidinyl}butyl)carbamate |
| I-111 | methyl [2-({(6-chloro-3′-ethyl-2-biphenylyl)[1-({4- |
| | [(methylamino)methyl]phenyl}carbonyl)-3-piperidinyl]methyl}oxy)ethyl]carbamate |
| I-112 | methyl [2-({(6-chloro-3′-ethyl-2-biphenylyl)[4-({4- |
| | [(methylamino)methyl]phenyl}carbonyl)-2- |
| | morpholinyl]methyl}oxy)ethyl]carbamate |
| I-113 | methyl [4-(6-chloro-3′-ethyl-2-biphenylyl)-4-hydroxy-4-(1-{[4-({[2- |
| | (methylthio)ethyl]amino}methyl)phenyl]carbonyl}-3-piperidinyl)butyl]carbamate |
| I-114 | methyl [4-(6-chloro-3′-ethyl-2-biphenylyl)-4-hydroxy-4-(1-{[4-({[2- |
| | (methylsulfonyl)ethyl]amino}methyl)phenyl]carbonyl}-3- |
| | piperidinyl)butyl]carbamate |
| I-115 | methyl {4-(6-fluoro-3′-methyl-2-biphenylyl)-4-hydroxy-4-[1-({4- |
| | [(methylamino)methyl]phenyl}sulfonyl)-3-piperidinyl]butyl}carbamate |
| I-116 | methyl [4-{1-[(4-{[(2-cyanoethyl)amino]methyl}phenyl)carbonyl]-3- |
| | piperidinyl}-4-(6-fluoro-3′-methyl-2-biphenylyl)-4-hydroxybutyl]carbamate |
| I-117 | N-[(4-{[3-(1-(6-fluoro-3′-methyl-2-biphenylyl)-1-hydroxy-4- |
| | {[(methyloxy)carbonyl]amino}butyl)-1-piperidinyl]carbonyl}phenyl)methyl]-β- |
| | alanine |
| I-118 | methyl [4-[1-({4-[amino(imino)methyl]phenyl}carbonyl)-3-piperidinyl]-4-(6- |
| | fluoro-3′-methyl-2-biphenylyl)-4-hydroxybutyl]carbamate |
| I-119 | N-[(4-{[3-(1-(6-fluoro-3′-methyl-2-biphenylyl)-1-hydroxy-4- |
| | {[(methyloxy)carbonyl]amino}butyl)-1-piperidinyl]carbonyl}phenyl)methyl]glycine |
| I-120 | methyl [4-(1-{[4-(aminocarbonyl)phenyl]carbonyl}-3-piperidinyl)-4-(6-fluoro- |
| | 3′-methyl-2-biphenylyl)-4-hydroxybutyl]carbamate |
| I-121 | methyl [4-{1-[(4-{[(3-amino-3-oxopropyl)amino]methyl}phenyl)carbonyl]-3- |
| | piperidinyl}-4-(6-fluoro-3′-methyl-2-biphenylyl)-4-hydroxybutyl] |
| I-122 | methyl [4-(6-fluoro-3′-methyl-2-biphenylyl)-4-hydroxy-4-(1-{[4-({[2-(1H- |
| | tetrazol-5-yl)ethyl]amino}methyl)phenyl]carbonyl}-3-piperidinyl)butyl]carbamate |
| I-123 | methyl (4-(6-fluoro-3′-methyl-2-biphenylyl)-4-hydroxy-4-{1-[(4-{[(4- |
| | hydroxybutyl)amino]methyl}phenyl)carbonyl]-3-piperidinyl}butyl)carbamate |
| I-124 | methyl [4-(1-{[4-({[2-(dimethylamino)ethyl]amino}methyl)phenyl]carbonyl}-3- |
| | piperidinyl)-4-(6-fluoro-3′-methyl-2-biphenylyl)-4-hydroxybutyl]carbamate |
| I-125 | methyl [4-(1-{[4-({[3-(dimethylamino)propyl]amino}methyl)phenyl]carbonyl}- |
| | 3-piperidinyl)-4-(6-fluoro-3′-methyl-2-biphenylyl)-4-hydroxybutyl]carbamate |
| I-126 | methyl [4-(1-{[4-({[amino(imino)methyl]amino}methyl)phenyl]carbonyl}-3- |
| | piperidinyl)-4-(6-fluoro-3′-methyl-2-biphenylyl)-4-hydroxybutyl]carbamate |
| I-127 | methyl [4-{1-[(4-{[(cyanomethyl)amino]methyl}phenyl)carbonyl]-3- |
| | piperidinyl}-4-(6-fluoro-3′-methyl-2-biphenylyl)-4-hydroxybutyl]carbamate |
| I-128 | methyl {4-[3-chloro-2-(8-methyl-2-quinolinyl)phenyl]-4-hydroxy-4-[1-({4- |
| | [(methylamino)methyl]phenyl}carbonyl)-3-piperidinyl]butyl}carbamate |
| I-130 | methyl {4-(1-{[4-(aminomethyl)-2-fluorophenyl]carbonyl}-3-piperidinyl)-4-[3- |
| | chloro-2-(3-quinolinyl)phenyl]-4-hydroxybutyl}carbamate |
| I-131 | methyl {4-[3-fluoro-2-(3-quinolinyl)phenyl]-4-hydroxy-4-[1-({4- |
| | [(methylamino)methyl]phenyl}carbonyl)-3-piperidinyl]butyl}carbamate |
| I-132 | methyl {4-{2-chloro-3-[3-(1-methylethyl)phenyl]-4-pyridinyl}-4-hydroxy-4-[1- |
| | ({4-[(methylamino)methyl]phenyl}carbonyl)-3-piperidinyl]butyl}carbamate |
| I-133 | methyl {4-[3-chloro-2-(5-methyl-2-furanyl)phenyl]-4-hydroxy-4-[1-({4- |
| | [(methylamino)methyl]phenyl}carbonyl)-3-piperidinyl]butyl}carbamate |
| I-134 | 1-{2-chloro-3-[3-(1-methylethyl)phenyl]-4-pyridinyl}-1-[4-({4- |
| | [(methylamino)methyl]phenyl}carbonyl)-2-morpholinyl]-5-(methyloxy)-1-pentanol |
| I-135 | methyl {4-[5-chloro-4-(3-ethylphenyl)-3-pyridinyl]-4-hydroxy-4-[1-({4- |
| | [(methylamino)methyl]phenyl}carbonyl)-3-piperidinyl]butyl}carbamate |
| I-136 | methyl {4-{5-chloro-4-[3-(1-methylethyl)phenyl]-3-pyridinyl}-4-hydroxy-4-[1- |
| | ({4-[(methylamino)methyl]phenyl}carbonyl)-3-piperidinyl]butyl}carbamate |
| I-137 | methyl [4-{1-[(4-{[(4-amino-4-oxobutyl)amino]methyl}phenyl)carbonyl]-3- |
| | piperidinyl}-4-(6-fluoro-3′-methyl-2-biphenylyl)-4-hydroxybutyl]carbamate |
| I-138 | methyl {4-(6-fluoro-3′-methyl-2-biphenylyl)-4-hydroxy-4-[1-({4-[({2- |
| | [(methylsulfonyl)amino]ethyl}amino)methyl]phenyl}carbonyl)-3- |
| | piperidinyl]butyl}carbamate |
| I-139 | methyl {4-(6-fluoro-3′-methyl-2-biphenylyl)-4-hydroxy-4-[1-({4-[({2- |
| | [(methylsulfonyl)amino]ethyl}amino)methyl]phenyl}carbonyl)-3- |
| | piperidinyl]butyl}carbamate |
| I-140 | methyl [4-{1-[(4-{[(2-amino-2-oxoethyl)amino]methyl}phenyl)carbonyl]-3- |
| | piperidinyl}-4-(6-fluoro-3′-methyl-2-biphenylyl)-4-hydroxybutyl]carbamate |
| I-141 | methyl (4-(6-fluoro-3′-methyl-2-biphenylyl)-4-hydroxy-4-{1-[(4-{[(1H-tetrazol- |
| | 5-ylmethyl)amino]methyl}phenyl)carbonyl]-3-piperidinyl}butyl)carbamate |
| I-142 | 1-(6-chloro-3′-ethyl-2-biphenylyl)-1-[1-({4- |
| | [(methylamino)methyl]phenyl}carbonyl)-3-piperidinyl]-1,6-hexanediol |
| I-143 | N-{4-(1-{[4-(aminomethyl)phenyl]carbonyl}-3-piperidinyl)-4-[6-fluoro-3′- |
| | (methyloxy)-2-biphenylyl]-4-hydroxybutyl}-2-hydroxyacetamide |
| I-144 | N-{4-[6-fluoro-3′-(methyloxy)-2-biphenylyl]-4-hydroxy-4-[1-({4- |
| | [(methylamino)methyl]phenyl}carbonyl)-3-piperidinyl]butyl}-2-hydroxyacetamide |
| I-145 | N-{4-(1-{[4-(aminomethyl)-2-fluorophenyl]carbonyl}-3-piperidinyl)-4-[6- |
| | fluoro-3′-(methyloxy)-2-biphenylyl]-4-hydroxybutyl}-2-hydroxyacetamide |
| I-146 | N-{4-(1-{[4-(aminomethyl)phenyl]carbonyl}-3-piperidinyl)-4-[6-chloro-3′- |
| | (methyloxy)-2-biphenylyl]-4-hydroxybutyl}-2-hydroxyacetamide |
| I-147 | N-{4-[6-chloro-3′-(methyloxy)-2-biphenylyl]-4-hydroxy-4-[1-({4- |
| | [(methylamino)methyl]phenyl}carbonyl)-3-piperidinyl]butyl}-2-hydroxyacetamide |
| I-148 | N-{4-(1-{[4-(aminomethyl)-2-fluorophenyl]carbonyl}-3-piperidinyl)-4-[6- |
| | chloro-3′-(methyloxy)-2-biphenylyl]-4-hydroxybutyl}-2-hydroxyacetamide |
| I-149 | methyl {4-(6-chloro-3′-fluoro-5′-methyl-2-biphenylyl)-4-[1-({2-fluoro-4- |
| | [(methylamino)methyl]phenyl}carbonyl)-3-piperidinyl]-4-hydroxybutyl}carbamate |
| I-150 | methyl {4-(6-chloro-3′-fluoro-5′-methyl-2-biphenylyl)-4-hydroxy-4-[1-({4- |
| | [(methylamino)methyl]phenyl}carbonyl)-3-piperidinyl]butyl}carbamate |
| I-151 | methyl {4-(3′,6-difluoro-5′-methyl-2-biphenylyl)-4-[1-({2-fluoro-4- |
| | [(methylamino)methyl]phenyl}carbonyl)-3-piperidinyl]-4-hydroxybutyl}carbamate |
| I-152 | methyl {4-[3-chloro-2-(2,3-dihydro-1-benzofuran-6-yl)phenyl]-4-[1-({2-fluoro- |
| | 4-[(methylamino)methyl]phenyl}carbonyl)-3-piperidinyl]-4-hydroxybutyl}carbamate |
| I-153 | methyl {4-[3-chloro-2-(2,3-dihydro-1-benzofuran-6-yl)phenyl]-4-hydroxy-4-[1- |
| | ({4-[(methylamino)methyl]phenyl}carbonyl)-3-piperidinyl]butyl}carbamate |
| I-154 | methyl {4-[6-chloro-2′-(methyloxy)-5′-(trifluoromethyl)-2-biphenylyl]-4-[1-({2- |
| | fluoro-4-[(methylamino)methyl]phenyl}carbonyl)-3-piperidinyl]-4- |
| | hydroxybutyl}carbamate |
| I-155 | methyl {4-[6-chloro-2′-(methyloxy)-5′-(trifluoromethyl)-2-biphenylyl]-4- |
| | hydroxy-4-[1-({4-[(methylamino)methyl]phenyl}carbonyl)-3- |
| | piperidinyl]butyl}carbamate |
| I-156 | methyl (4-(1-{[4-(aminomethyl)phenyl]carbonyl}-3-piperidinyl)-4-{3-chloro-2- |
| | [(2-ethylphenyl)oxy]phenyl}-4-hydroxybutyl)carbamate |
| I-157 | methyl (4-(1-{[4-(aminomethyl)phenyl]carbonyl}-3-piperidinyl)-4-{3-chloro-2- |
| | [(2-methylphenyl)oxy]phenyl}-4-hydroxybutyl)carbamate |
| I-158 | methyl {4-(3′-ethyl-6-fluoro-2-biphenylyl)-4-hydroxy-4-[1-({4- |
| | [(methylamino)methyl]phenyl}carbonyl)-3-piperidinyl]butyl}carbamate |
| I-159 | N-{4-(2′,6-difluoro-5′-methyl-2-biphenylyl)-4-hydroxy-4-[1-({4- |
| | [(methylamino)methyl]phenyl}carbonyl)-3-piperidinyl]butyl}acetamide |
| I-160 | N-{4-(6-fluoro-3′-methyl-2-biphenylyl)-4-hydroxy-4-[1-({4- |
| | [(methylamino)methyl]phenyl}carbonyl)-3-piperidinyl]butyl}acetamide |
| I-161 | methyl {4-(3′-ethyl-6-fluoro-2-biphenylyl)-4-[1-({2-fluoro-4- |
| | [(methylamino)methyl]phenyl}carbonyl)-3-piperidinyl]-4-hydroxybutyl}carbamate |
| I-162 | N-{4-(3′-ethyl-6-fluoro-2-biphenylyl)-4-hydroxy-4-[1-({4- |
| | [(methylamino)methyl]phenyl}carbonyl)-3-piperidinyl]butyl}acetamide |
| I-163 | methyl [4-[1-({2-fluoro-4-[(methylamino)methyl]phenyl}carbonyl)-3- |
| | piperidinyl]-4-(6-fluoro-3′-methyl-2-biphenylyl)-4-hydroxybutyl]carbamate |
| I-164 | N-{4-(2′,6-difluoro-5′-methyl-2-biphenylyl)-4-[1-({2-fluoro-4- |
| | [(methylamino)methyl]phenyl}carbonyl)-3-piperidinyl]-4-hydroxybutyl}-2- |
| | hydroxyacetamide |
| I-165 | methyl {4-(2′,6-difluoro-5′-methyl-2-biphenylyl)-4-[1-({2-fluoro-4- |
| | [(methylamino)methyl]phenyl}carbonyl)-3-piperidinyl]-4-hydroxybutyl}carbamate |
| I-166 | N-{4-(2′,6-difluoro-5′-methyl-2-biphenylyl)-4-hydroxy-4-[1-({4- |
| | [(methylamino)methyl]phenyl}carbonyl)-3-piperidinyl]butyl}-2,2,2- |
| | trifluoroacetamide |
| I-167 | methyl {4-[6-fluoro-3′-(1-methylethyl)-2-biphenylyl]-4-hydroxy-4-[1-({4- |
| | [(methylamino)methyl]phenyl}carbonyl)-3-piperidinyl]butyl}carbamate |
| I-168 | 1-(1-{[4-(aminomethyl)phenyl]carbonyl}-3-piperidinyl)-1-{3-fluoro-2-[(3- |
| | methylphenyl)oxy]phenyl}-5-(methyloxy)-1-pentanol |
| I-169 | methyl (4-(1-{[4-(aminomethyl)phenyl]carbonyl}-3-piperidinyl)-4-{3-chloro-2- |
| | [(3-ethylphenyl)oxy]phenyl}-4-hydroxybutyl)carbamate |
| I-170 | methyl (4-(1-{[4-(aminomethyl)phenyl]carbonyl}-3-piperidinyl)-4-{3-fluoro-2- |
| | [(3-methylphenyl)oxy]phenyl}-4-hydroxybutyl)carbamate |
| I-171 | methyl {4-{3-chloro-2-[(3-ethylphenyl)oxy]phenyl}-4-hydroxy-4-[1-({4- |
| | [(methylamino)methyl]phenyl}carbonyl)-3-piperidinyl]butyl}carbamate |
| I-172 | 1-{3-chloro-2-[(3-methylphenyl)methyl]phenyl}-1-[1-({4- |
| | [(methylamino)methyl]phenyl}carbonyl)-3-piperidinyl]-5-(methyloxy)-1-pentanol |
| I-173 | methyl {4-{3-chloro-2-[(3-methylphenyl)methyl]phenyl}-4-hydroxy-4-[1-({4- |
| | [(methylamino)methyl]phenyl}carbonyl)-3-piperidinyl]butyl}carbamate |
| I-174 | methyl {4-{3-chloro-2-[(3-methylphenyl)methyl]phenyl}-4-[1-({2-fluoro-4- |
| | [(methylamino)methyl]phenyl}carbonyl)-3-piperidinyl]-4-hydroxybutyl}carbamate |

or a diastereomer, enantiomer or salt thereof.

It will also be appreciated by those skilled in the art that each enantiomer and diastereomer of the compounds of this invention will likely demonstrate a different level of effectiveness of inhibiting the action of aspartic proteases, particularly renin. It will be further appreciated that for the most active compounds, all enantiomers and/or diastereomers may demonstrate some level of activity, but that for compounds with lower activity, certain enantiomers and/or diastereomers may demonstrate such low levels of activity as to be considered inactive. It is understood that the following represent the preferred relative and absolute configuration of the compounds of the invention. It will be appreciated that each of the different enantiomeric and/or diastereomeric forms of the compounds of this invention, including the stereoisomeric forms depicted below, may be separately obtained using conventional procedures (e.g. stereospecific synthesis or resolution via chiral chromatography, crystallization, etc.).

The following are compounds of the invention:

| Cpd. | | |
|---|---|---|
| No. | Structure | Name |
| I-1a | 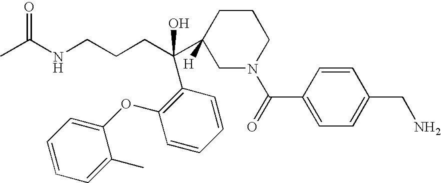 | N-((S)-4-((R)-1-(4- (aminomethyl)benzoyl)piperidin-3-yl)-4- hydroxy-4-(2-(o-tolyloxy)phenyl)butyl) acetamide |
| I-2a | 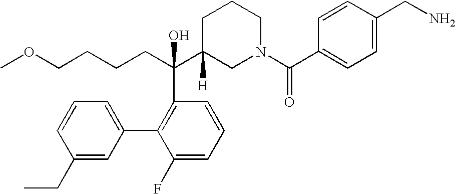 | (4-(aminomethyl)phenyl)((R)-3-((S)-1-(3′- ethyl-6-fluorobiphenyl-2-yl)-1-hydroxy-5- methoxypentyl)piperidin-1-yl)methanone |
| I-3a | 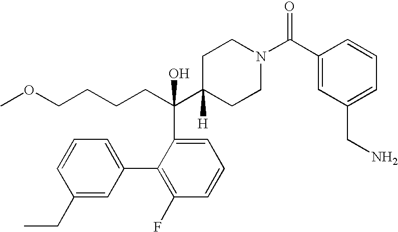 | (1S)-1-((3R)-1-{[3- (aminomethyl)phenyl]carbonyl}-3-piperidinyl)- 1-(3′-ethyl-6-fluoro-2-biphenylyl)-5- (methyloxy)-1-pentanol |
| I-4a | 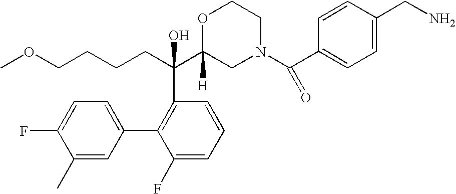 | (4-(aminomethyl)phenyl)((R)-2-((R)-1- (4′,6-difluoro-3′-methylbiphenyl-2-yl)-1- hydroxy-5-methoxypentyl)morpholino) methanone |
| I-5a | 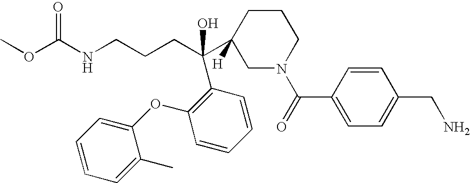 | methyl (S)-4-((R)-1-(4- (aminomethyl)benzoyl)piperidin-3-yl)-4- hydroxy-4-(2-(o-tolyloxy)phenyl)butyl carbamate |
| I-6a | 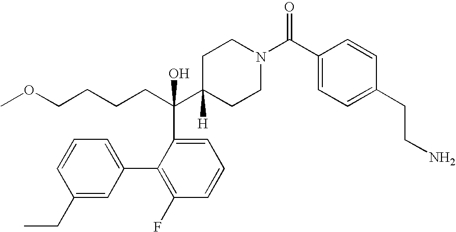 | (1S)-1-(1-{[4-(2-aminoethyl)phenyl] carbonyl}-4-piperidinyl)-1-(3′-ethyl-6-fluoro-2- biphenylyl)-5-(methyloxy)-1-pentanol |
| I-7a | 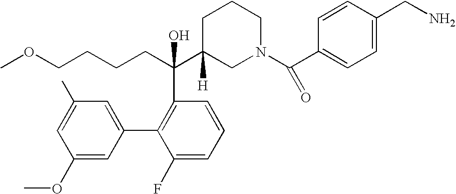 | (4-(aminomethyl)phenyl)((R)-3-((S)-1-(6- fluoro-3′-methoxy-5′-methylbiphenyl-2-yl)-1- hydroxy-5-methoxypentyl)piperidin-1- yl)methanone |
| I-8a | 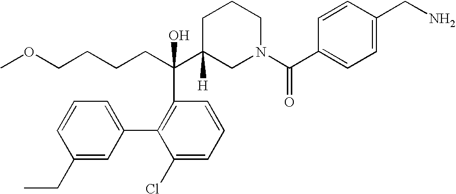 | (4-(aminomethyl)phenyl)((R)-3-((S)-1-(6- chloro-3′-ethylbiphenyl-2-yl)-1-hydroxy-5- methoxypentyl)piperidin-1-yl)methanone |
| I-9a | 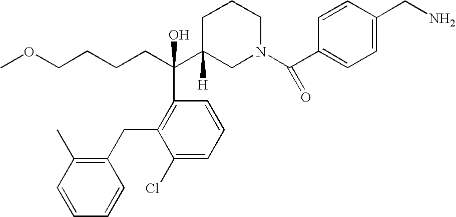 | (4-(aminomethyl)phenyl)((R)-3-((S)-1-(3- chloro-2-(2-methylbenzyl)phenyl)-1-hydroxy-5- methoxypentyl)piperidin-1-yl)methanone |
| I-10a | 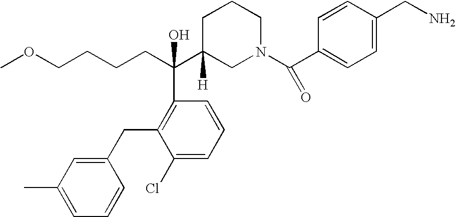 | (4-(aminomethyl)phenyl)((R)-3-((S)-1-(3- chloro-2-(3-methylbenzyl)phenyl)-1-hydroxy-5- methoxypentyl)piperidin-1-yl)methanone |
| I-11a | 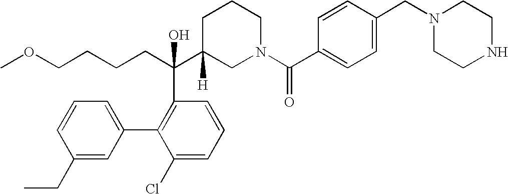 | ((R)-3-((S)-1-(6-chloro-3′-ethylbiphenyl-2- yl)-1-hydroxy-5-methoxypentyl)piperidin-1- yl)(4-(piperazin-1-ylmethyl)phenyl)methanone |
| I-12a | 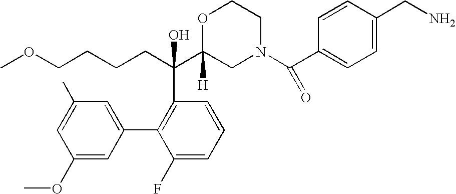 | (4-(aminomethyl)phenyl)((R)-2-((R)-1-(6- fluoro-3′-methoxy-5′-methylbiphenyl-2-yl)-1- hydroxy-5- methoxypentyl)morpholino)methanone |
| I-13a | 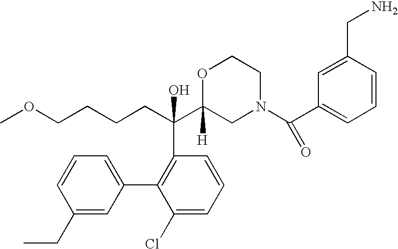 | (3-(aminomethyl)phenyl)((R)-2-((R)-1-(6- chloro-3′-ethylbiphenyl-2-yl)-1-hydroxy-5- methoxypentyl)morpholino)methanone |
| I-14a | 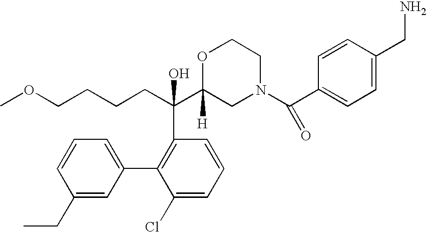 | (4-(aminomethyl)phenyl)((R)-2-((R)-1-(6- chloro-3′-ethylbiphenyl-2-yl)-1-hydroxy-5- methoxypentyl)morpholino)methanone |
| I-15a | 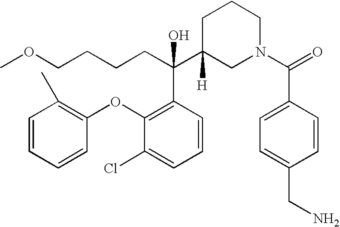 | (4-(aminomethyl)phenyl)((R)-3-((S)-1-(3- chloro-2-(o-tolyloxy)phenyl)-1-hydroxy-5- methoxypentyl)piperidin-1-yl)methanone |
| I-16a | 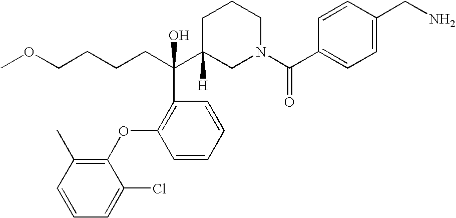 | (4-(aminomethyl)phenyl)((R)-3-((S)-1-(2- (2-chloro-6-methylphenoxy)phenyl)-1-hydroxy- 5-methoxypentyl)piperidin-1-yl)methanone |
| I-17a | 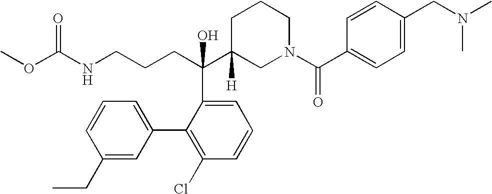 | methyl (S)-4-(6-chloro-3′-ethylbiphenyl-2- yl)-4-((R)-1-(4-((dimethylamino)methyl) benzoyl)piperidin-3-yl)-4-hydroxybutyl carbamate |
| I-18a | 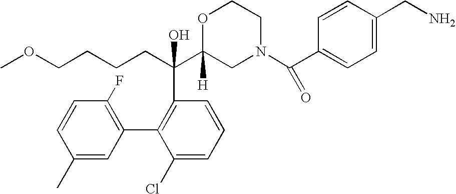 | (4-(aminomethyl)phenyl)((2R)-2-((1R)-1- (6-chloro-2′-fluoro-5′-methylbiphenyl-2-yl)-1- hydroxy-5-methoxypentyl)morpholino) methanone |
| I-19a | 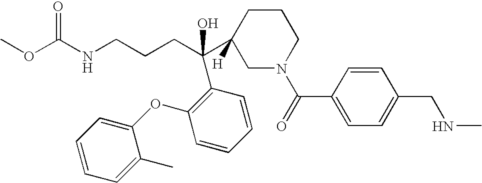 | methyl (S)-4-hydroxy-4-((R)-1-(4- ((methylamino)methyl)benzoyl)piperidin-3-yl)- 4-(2-(o-tolyloxy)phenyl)butylcarbamate |
| I-20a | 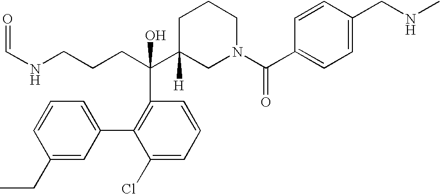 | N-((S)-4-(6-chloro-3′-ethylbiphenyl-2-yl)- 4-hydroxy-4-((R)-1-(4-((methylamino)methyl) benzoyl)piperidin-3-yl)butyl)formamide |
| I-21a | 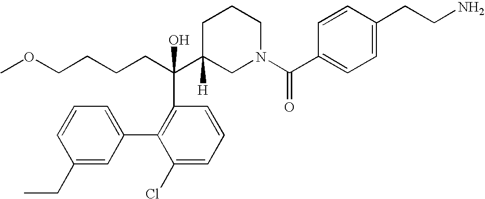 | (4-(2-aminoethyl)phenyl)((R)-3-((S)-1-(6- chloro-3′-ethylbiphenyl-2-yl)-1-hydroxy-5- methoxypentyl)piperidin-1-yl)methanone |
| I-22a | 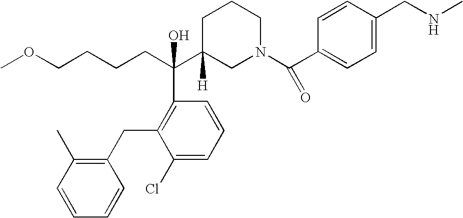 | ((R)-3-((S)-1-(3-chloro-2-(2- methylbenzyl)phenyl)-1-hydroxy-5- methoxypentyl)piperidin-1-yl)(4- ((methylamino)methyl)phenyl)methanone |
| I-23a | 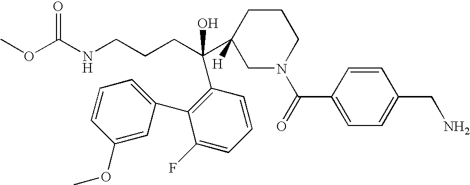 | methyl (S)-4-((R)-1-(4- (aminomethyl)benzoyl)piperidin-3-yl)-4-(6- fluoro-3′-methoxybiphenyl-2-yl)-4- hydroxybutylcarbamate |
| I-24a | 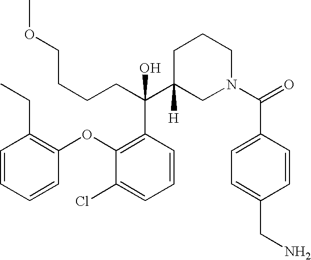 | (4-(aminomethyl)phenyl)((R)-3-((S)-1-(3- chloro-2-(2-ethylphenoxy)phenyl)-1-hydroxy- 5-methoxypentyl)piperidin-1-yl)methanone |
| I-25a | 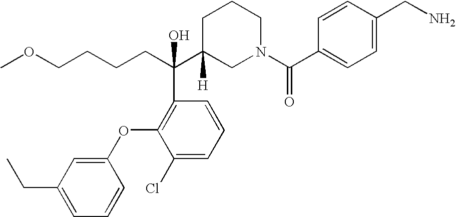 | (4-(aminomethyl)phenyl)((R)-3-((S)-1-(3- chloro-2-(3-ethylphenoxy)phenyl)-1-hydroxy- 5-methoxypentyl)piperidin-1-yl)methanone |
| I-26a | 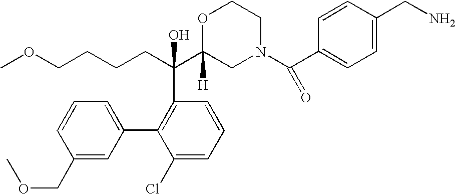 | (4-(aminomethyl)phenyl)((R)-2-((R)-1-(6- chloro-3′-(methoxymethyl)biphenyl-2-yl)-1- hydroxy-5-methoxypentyl)morpholino) methanone |
| I-27a | 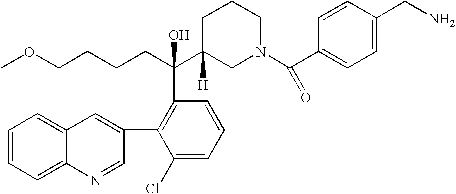 | (4-(aminomethyl)phenyl)((R)-3-((S)-1-(3- chloro-2-(quinolin-3-yl)phenyl)-1-hydroxy-5- methoxypentyl)piperidin-1-yl)methanone |
| I-28a | 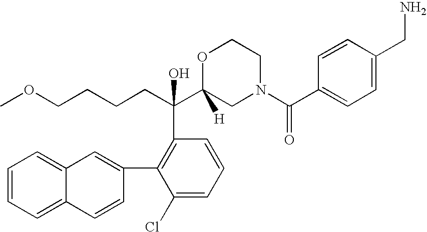 | (4-(aminomethyl)phenyl)((R)-2-((R)-1-(3- chloro-2-(naphthalen-2-yl)phenyl)-1-hydroxy- 5-methoxypentyl)morpholino)methanone |
| I-29a | 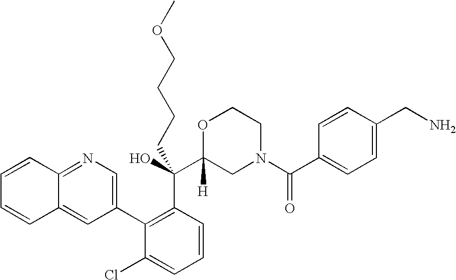 | (4-(aminomethyl)phenyl)((R)-2-((R)-1-(3- chloro-2-(quinolin-3-yl)phenyl)-1-hydroxy-5- methoxypentyl)morpholino)methanone |
| I-29b | 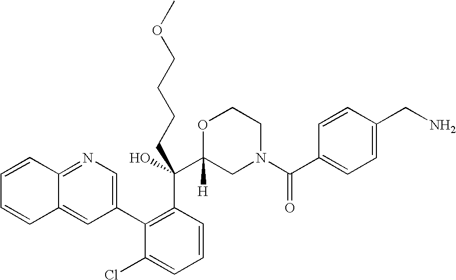 | (4-(aminomethyl)phenyl)((R)-2-((S)-1-(3- chloro-2-(quinolin-3-yl)phenyl)-1-hydroxy-5- methoxypentyl)morpholino)methanone |
| I-30a | 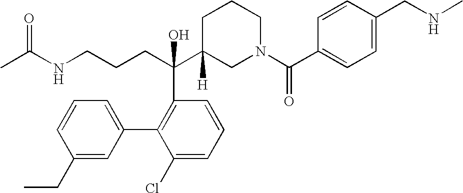 | N-((S)-4-(6-chloro-3′-ethylbiphenyl-2-yl)- 4-hydroxy-4-((R)-1-(4- ((methylamino)methyl)benzoyl)piperidin-3- yl)butyl)acetamide |
| I-31a | 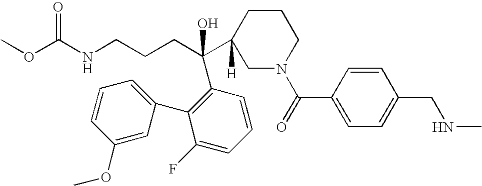 | methyl (S)-4-(6-fluoro-3′- methoxybiphenyl-2-yl)-4-hydroxy-4-((R)-1-(4- ((methylamino)methyl)benzoyl)piperidin-3- yl)butylcarbamate |
| I-32a | 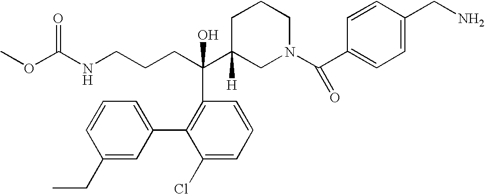 | methyl (S)-4-((R)-1-(4- (aminomethyl)benzoyl)piperidin-3-yl)-4-(6- chloro-3′-ethylbiphenyl-2-yl)-4- hydroxybutylcarbamate |
| I-32b | 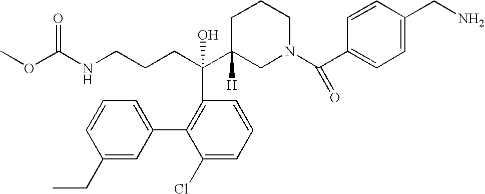 | methyl (R)-4-((R)-1-(4- (aminomethyl)benzoyl)piperidin-3-yl)-4-(6- chloro-3′-ethylbiphenyl-2-yl)-4- hydroxybutylcarbamate |
| I-33a | 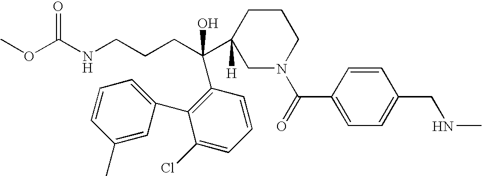 | methyl (S)-4-(6-chloro-3′-methylbiphenyl- 2-yl)-4-hydroxy-4-((R)-1-(4-((methylamino) methyl)benzoyl)piperidin-3-yl)butylcarbamate |
| I-34a | 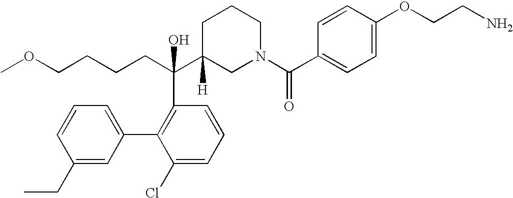 | (4-(2-aminoethoxy)phenyl)((R)-3-((S)-1- (6-chloro-3′-ethylbiphenyl-2-yl)-1-hydroxy-5- methoxypentyl)piperidin-1-yl)methanone |
| I-35a | 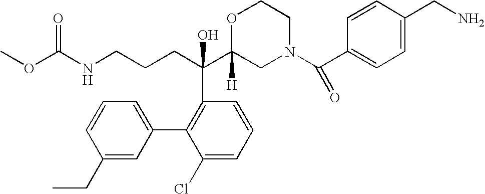 | methyl (R)-4-((R)-4-(4-(aminomethyl) benzoyl)morpholin-2-yl)-4-(6-chloro-3′- ethylbiphenyl-2-yl)-4-hydroxybutylcarbamate |
| I-36a | 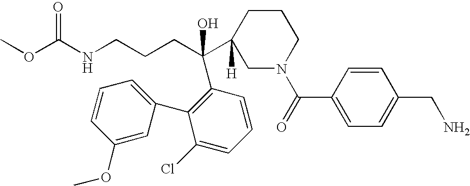 | methyl (S)-4-((R)-1-(4-(aminomethyl) benzoyl)piperidin-3-yl)-4-(6-chloro-3′- methoxybiphenyl-2-yl)-4- hydroxybutylcarbamate |
| I-37a | 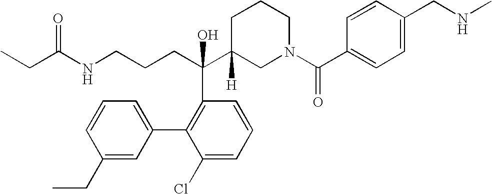 | N-((S)-4-(6-chloro-3′-ethylbiphenyl-2-yl)- 4-hydroxy-4-((R)-1-(4-((methylamino)methyl) benzoyl)piperidin-3-yl)butyl)propionamide |
| I-38a | 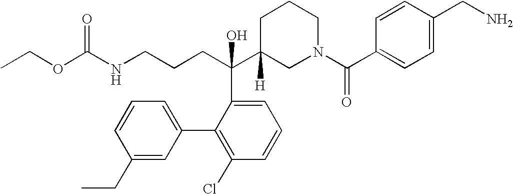 | ethyl (S)-4-((R)-1-(4-(aminomethyl) benzoyl)piperidin-3-yl)-4-(6-chloro-3′- ethylbiphenyl-2-yl)-4-hydroxybutylcarbamate |
| I-39a | 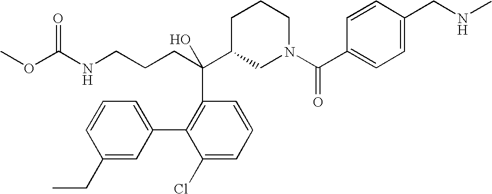 | methyl {(4S)-4-(6-chloro-3′-ethyl-2- biphenylyl)-4-hydroxy-4-[(3R)-1-({4- [(methylamino)methyl]phenyl}carbonyl)-3- piperidinyl]butyl}carbamate |
| I-39b | 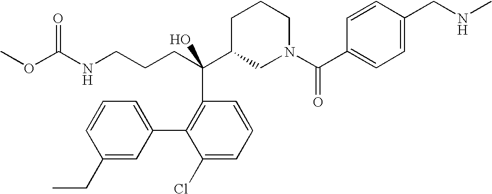 | methyl (S)-4-(6-chloro-3′-ethylbiphenyl-2- yl)-4-hydroxy-4-((R)-1-(4- ((methylamino)methyl)benzoyl)piperidin-3- yl)butylcarbamate |
| I-40a | 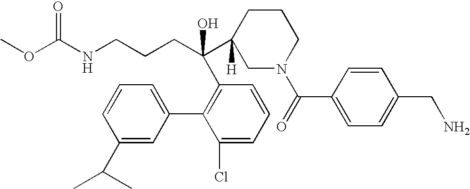 | methyl (S)-4-((R)-1-(4- (aminomethyl)benzoyl)piperidin-3-yl)-4-(6- chloro-3′-isopropylbiphenyl-2-yl)-4- hydroxybutylcarbamate |
| I-41a | 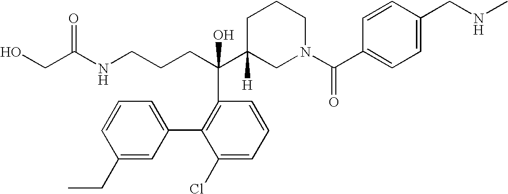 | N-((S)-4-(6-chloro-3′-ethylbiphenyl-2-yl)- 4-hydroxy-4-((R)-1-(4- ((methylamino)methyl)benzoyl)piperidin-3- yl)butyl)-2-hydroxyacetamide |
| I-42a | 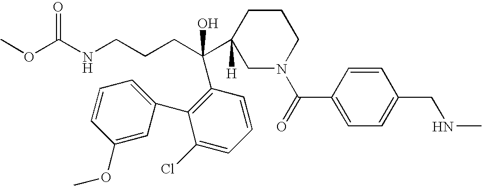 | methyl (S)-4-(6-chloro-3′- methoxybiphenyl-2-yl)-4-hydroxy-4-((R)-1-(4- ((methylamino)methyl)benzoyl)piperidin-3- yl)butylcarbamate |
| I-43a | 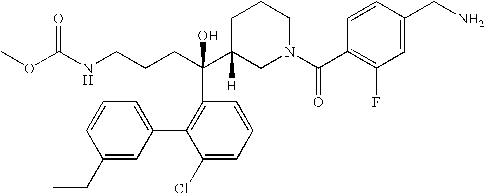 | methyl (S)-4-((R)-1-(4-(aminomethyl)-2- fluorobenzoyl)piperidin-3-yl)-4-(6-chloro-3′- ethylbiphenyl-2-yl)-4-hydroxybutylcarbamate |
| I-44a | 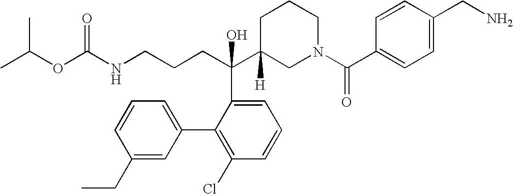 | isopropyl (S)-4-((R)-1-(4- (aminomethyl)benzoyl)piperidin-3-yl)-4-(6- chloro-3′-ethylbiphenyl-2-yl)-4- hydroxybutylcarbamate |
| I-45a | 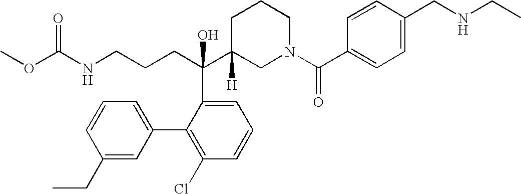 | methyl (S)-4-(6-chloro-3′-ethylbiphenyl-2- yl)-4-((R)-1-(4-((ethylamino) methyl)benzoyl)piperidin-3-yl)-4- hydroxybutylcarbamate |
| I-46a | 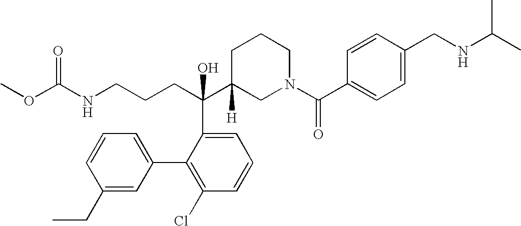 | methyl (S)-4-(6-chloro-3′-ethylbiphenyl-2- yl)-4-hydroxy-4-((R)-1-(4-((isopropylamino) methyl)benzoyl)piperidin-3-yl)butylcarbamate |
| I-47a | 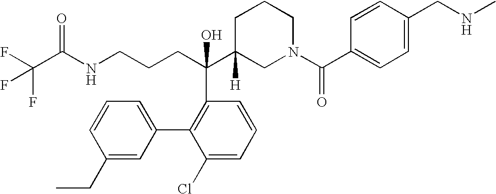 | N-((S)-4-(6-chloro-3′-ethylbiphenyl-2-yl)- 4-hydroxy-4-((R)-1-(4-((methylamino) methyl)benzoyl)piperidin-3-yl)butyl)-2,2,2- trifluoroacetamide |
| I-48 | 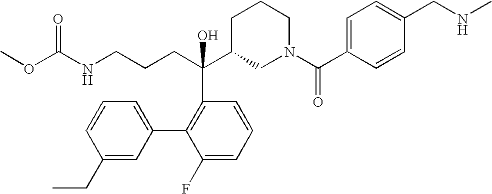 | methyl {(4S)-4-(3′-ethyl-6-fluoro-2- biphenylyl)-4-hydroxy-4-[(3R)-1-({4- [(methylamino)methyl]phenyl}carbonyl)-3- piperidinyl]butyl}carbamate |
| I-49 | 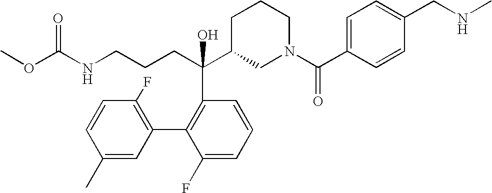 | methyl {(4S)-4-(2′,6-difluoro-5′-methyl-2- biphenylyl)-4-hydroxy-4-[(3R)-1-({4- [(methylamino)methyl]phenyl}carbonyl)-3- piperidinyl]butyl}carbamate |
| I-50 | 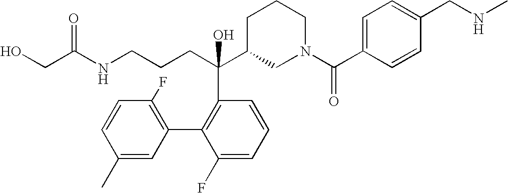 | N-{(4S)-4-(2′,6-difluoro-5′-methyl-2- biphenylyl)-4-hydroxy-4-[(3R)-1-({4- [(methylamino)methyl]phenyl}carbonyl)-3- piperidinyl]butyl}-2-hydroxyacetamide |
| I-51a | 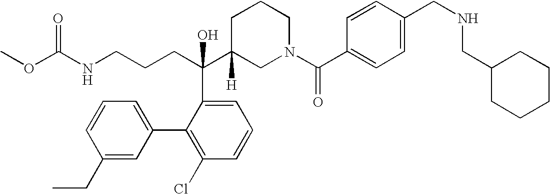 | methyl (S)-4-(6-chloro-3′-ethylbiphenyl-2- yl)-4-((R)-1-(4-((cyclohexylmethylamino) methyl)benzoyl)piperidin-3-yl)-4- hydroxybutylcarbamate |
| I-52a | 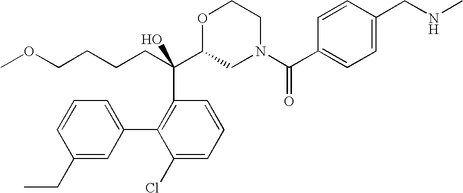 | (1R)-1-(6-chloro-3′-ethyl-2-biphenylyl)-1- [(2R)-4-({4-[(methylamino)methyl]phenyl} carbonyl)-2-morpholinyl]-5-(methyloxy)-1- pentanol |
| I-53a | 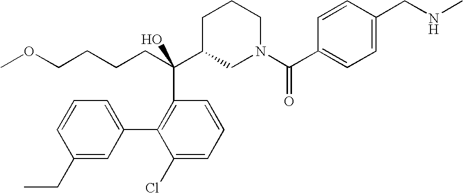 | (1S)-1-(6-chloro-3′-ethyl-2-biphenylyl)-1- [(3R)-1-({4-[(methylamino)methyl]phenyl} carbonyl)-3-piperidinyl]-5-(methyloxy)-1- pentanol |
| I-54a | 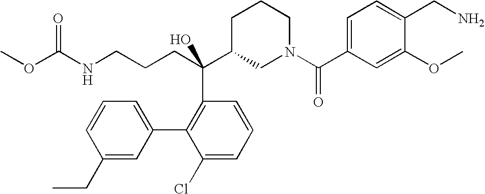 | methyl [(4S)-4-((3R)-1-{[4-(aminomethyl)- 3-(methyloxy)phenyl]carbonyl}-3-piperidinyl)- 4-(6-chloro-3′-ethyl-2-biphenylyl)-4- hydroxybutyl]carbamate |
| I-55a | 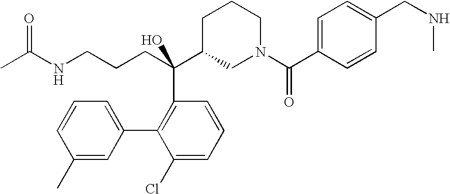 | N-{(4S)-4-(6-chloro-3′-methyl-2- biphenylyl)-4-hydroxy-4-[(3R)-1-({4- [(methylamino)methyl]phenyl}carbonyl)-3- piperidinyl]butyl}acetamide |
| I-56a | 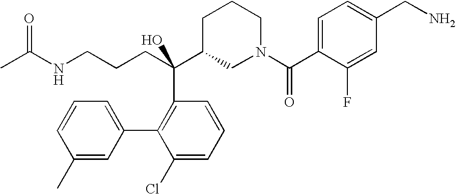 | N-[(4S)-4-((3R)-1-{[4-(aminomethyl)-2- fluorophenyl]carbonyl}-3-piperidinyl)-4-(6- chloro-3′-methyl-2-biphenylyl)-4- hydroxybutyl]acetamide |
| I-57a | 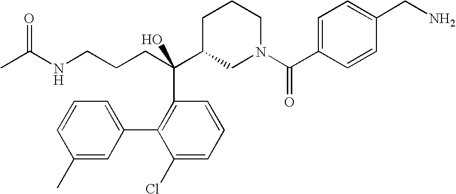 | N-[(4S)-4-((3R)-1-{[4-(aminomethyl) phenyl]carbonyl}-3-piperidinyl)-4-(6-chloro-3′- methyl-2-biphenylyl)-4- hydroxybutyl]acetamide |
| I-58a | 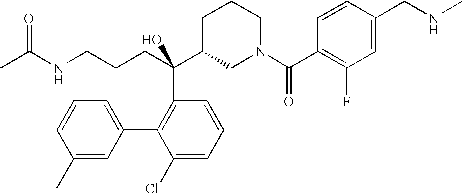 | N-{(4S)-4-(6-chloro-3′-methyl-2- biphenylyl)-4-[(3R)-1-({2-fluoro-4- [(methylamino)methyl]phenyl}carbonyl)-3- piperidinyl]-4-hydroxybutyl}acetamide |
| I-59a | 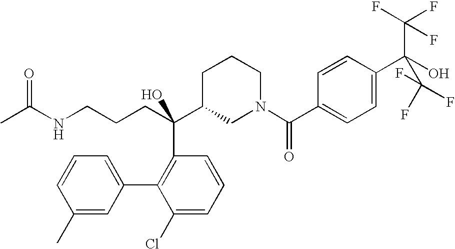 | N-{(4S)-4-(6-chloro-3′-methyl-2- biphenylyl)-4-hydroxy-4-[(3R)-1-({4-[2,2,2- trifluoro-1-hydroxy-1-(trifluoromethyl) ethyl]phenyl}carbonyl)-3- piperidinyl]butyl}acetamide |
| I-60a | 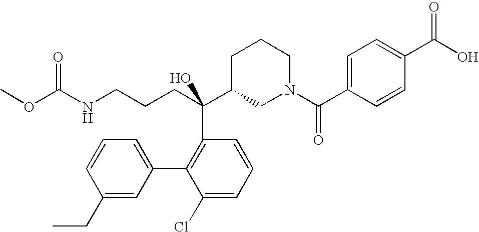 | 4-{[(3R)-3-((1S)-1-(6-chloro-3′-ethyl-2- biphenylyl)-1-hydroxy-4-{[(methyloxy) carbonyl]amino}butyl)-1-piperidinyl] carbonyl}benzoic acid |
| I-61a | 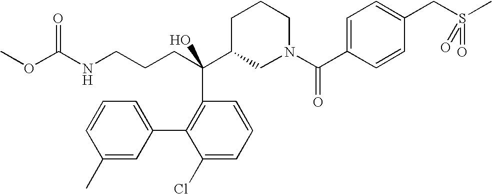 | methyl {(4S)-4-(6-chloro-3′-methyl-2- biphenylyl)-4-hydroxy-4-[(3R)-1-({4- [(methylsulfonyl)methyl]phenyl}carbonyl)-3- piperidinyl]butyl}carbamate |
| I-62a | 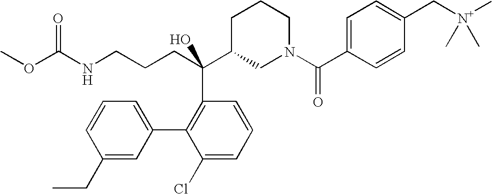 | (4-{[(3R)-3-((1S)-1-(6-chloro-3′-ethyl-2- biphenylyl)-1-hydroxy-4- {[(methyloxy)carbonyl]amino}butyl)-1- piperidinyl]carbonyl}phenyl)-N,N,N- trimethylmethanaminium |
| I-63a | 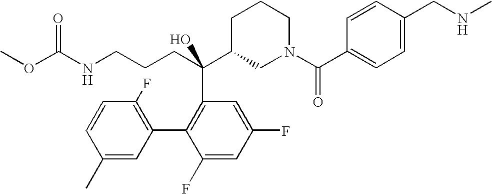 | methyl [(4S)-4-hydroxy-4-[(3R)-1-({4- [(methylamino)methyl]phenyl}carbonyl)-3- piperidinyl]-4-(2′,4,6-trifluoro-5′-methyl-2- biphenylyl)butyl]carbamate |
| I-64a | 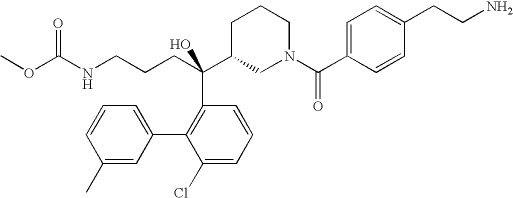 | methyl [(4S)-4-((3R)-1-{[4-(2- aminoethyl)phenyl]carbonyl}-3-piperidinyl)-4- (6-chloro-3′-methyl-2-biphenylyl)-4- hydroxybutyl]carbamate |
| I-65a | 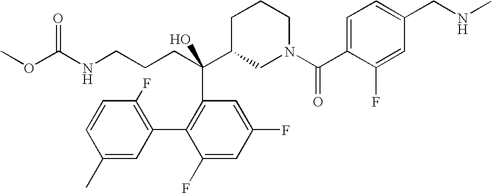 | methyl [(4S)-4-[(3R)-1-({2-fluoro-4- [(methylamino)methyl]phenyl}carbonyl)-3- piperidinyl]-4-hydroxy-4-(2′,4,6-trifluoro-5′- methyl-2-biphenylyl)butyl]carbamate |
| I-66a | 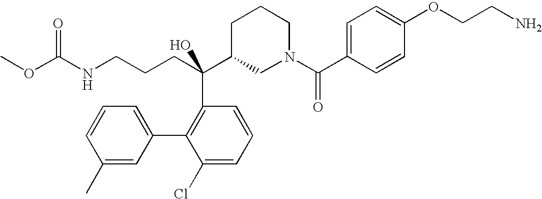 | methyl [(4S)-4-[(3R)-1-({4-[(2- aminoethyl)oxy]phenyl}carbonyl)-3- piperidinyl]-4-(6-chloro-3′-methyl-2- biphenylyl)-4-hydroxybutyl]carbamate |
| I-67a | 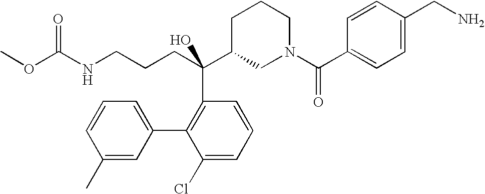 | methyl [(4S)-4-((3R)-1-{[4- (aminomethyl)phenyl]carbonyl}-3-piperidinyl)- 4-(6-chloro-3′-methyl-2-biphenylyl)-4- hydroxybutyl]carbamate |
| I-68a | 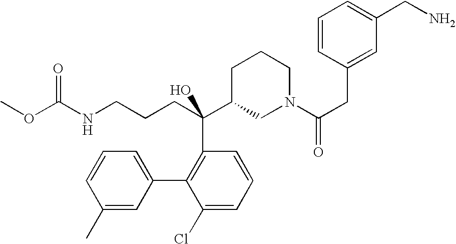 | methyl [(4S)-4-((3R)-1-{[3- (aminomethyl)phenyl]acetyl}-3-piperidinyl)-4- (6-chloro-3′-methyl-2-biphenylyl)-4- hydroxybutyl]carbamate |
| I-69a | 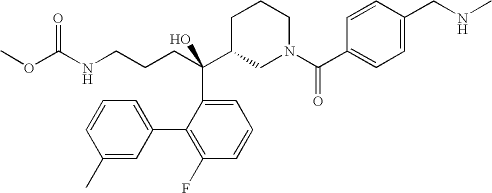 | methyl {(4S)-4-(6-fluoro-3′-methyl-2- biphenylyl)-4-hydroxy-4-[(3R)-1-({4- [(methylamino)methyl]phenyl}carbonyl)-3- piperidinyl]butyl}carbamate |
| I-70a | 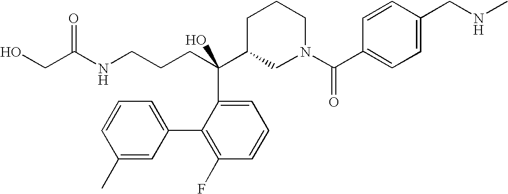 | N-{(4S)-4-(6-fluoro-3′-methyl-2- biphenylyl)-4-hydroxy-4-[(3R)-1-({4- [(methylamino)methyl]phenyl}carbonyl)-3- piperidinyl]butyl}-2-hydroxyacetamide |
| I-71a | 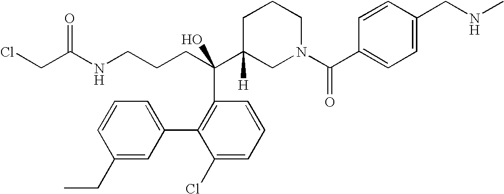 | 2-chloro-N-{(4S)-4-(6-chloro-3′-ethyl-2- biphenylyl)-4-hydroxy-4-[(3R)-1-({4- [(methylamino)methyl]phenyl}carbonyl)-3- piperidinyl]butyl}acetamide |
| I-72a | 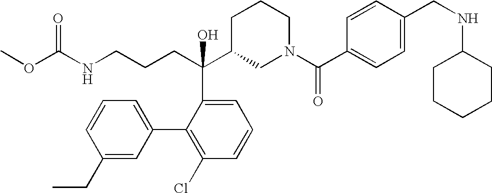 | methyl {(4S)-4-(6-chloro-3′-ethyl-2- biphenylyl)-4-[(3R)-1-({4- [(cyclohexylamino)methyl]phenyl}carbonyl)-3- piperidinyl]-4-hydroxybutyl}carbamate |
| I-73a | 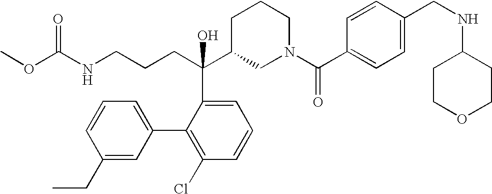 | methyl {(4S)-4-(6-chloro-3′-ethyl-2- biphenylyl)-4-hydroxy-4-[(3R)-1-({4- [(tetrahydro-2H-pyran-4- ylamino)methyl]phenyl}carbonyl)-3- piperidinyl]butyl}carbamate |
| I-74a | 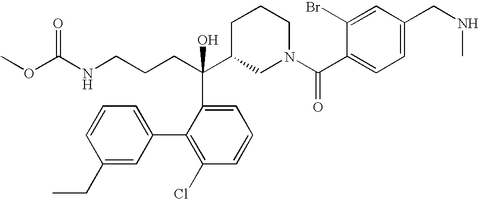 | methyl [(4S)-4-[(3R)-1-({2-bromo-4- [(methylamino)methyl]phenyl}carbonyl)-3- piperidinyl]-4-(6-chloro-3′-ethyl-2-biphenylyl)- 4-hydroxybutyl]carbamate |
| I-75a | 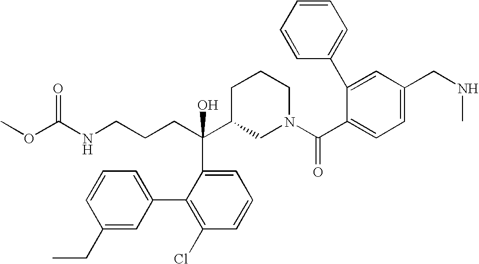 | methyl {(4S)-4-(6-chloro-3′-ethyl-2- biphenylyl)-4-hydroxy-4-[(3R)-1-({5- [(methylamino)methyl]-2- biphenylyl}carbonyl)-3- piperidinyl]butyl}carbamate |
| I-76a | 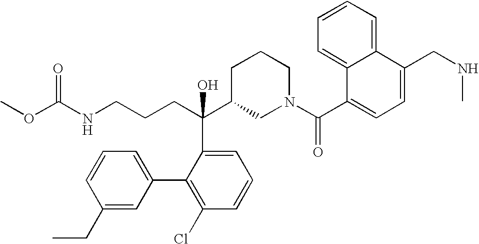 | methyl {(4S)-4-(6-chloro-3′-ethyl-2- biphenylyl)-4-hydroxy-4-[(3R)-1-({4- [(methylamino)methyl]-1- naphthalenyl}carbonyl)-3- piperidinyl]butyl}carbamate |
| I-77a | 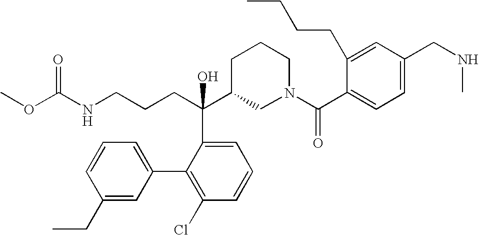 | methyl [(4S)-4-[(3R)-1-({2-butyl-4- [(methylamino)methyl]phenyl}carbonyl)-3- piperidinyl]-4-(6-chloro-3′-ethyl-2-biphenylyl)- 4-hydroxybutyl]carbamate |
| I-78a | 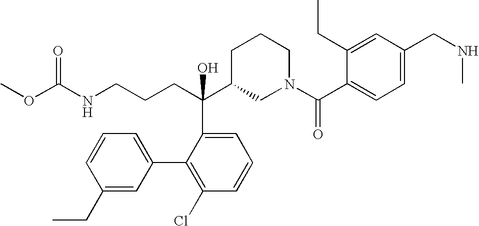 | methyl {(4S)-4-(6-chloro-3′-ethyl-2- biphenylyl)-4-[(3R)-1-({2-ethyl-4- [(methylamino)methyl]phenyl}carbonyl)-3- piperidinyl]-4-hydroxybutyl}carbamate |
| I-79a | 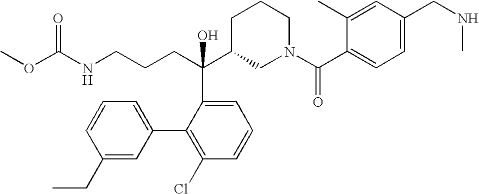 | methyl {(4S)-4-(6-chloro-3′-ethyl-2- biphenylyl)-4-hydroxy-4-[(3R)-1-({2-methyl-4- [(methylamino)methyl]phenyl}carbonyl)-3- piperidinyl]butyl}carbamate |
| I-80a | 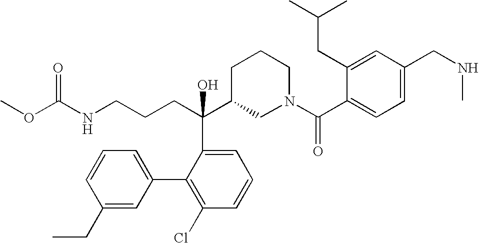 | methyl [(4S)-4-(6-chloro-3′-ethyl-2- biphenylyl)-4-hydroxy-4-((3R)-1-{[4- [(methylamino)methyl]-2-(2- methylpropyl)phenyl]carbonyl}-3- piperidinyl)butyl]carbamate |
| I-81a | 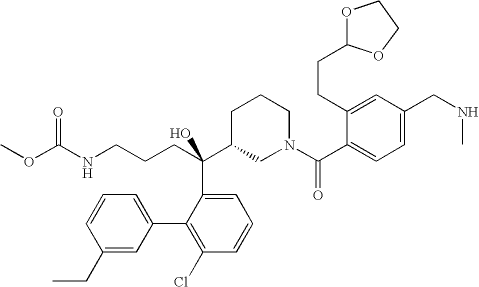 | methyl {(4S)-4-(6-chloro-3′-ethyl-2- biphenylyl)-4-[(3R)-1-({2-[2-(1,3-dioxolan-2- yl)ethyl]-4- [(methylamino)methyl]phenyl}carbonyl)-3- piperidinyl]-4-hydroxybutyl}carbamate |
| I-82a | 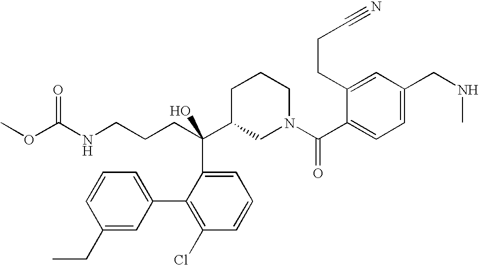 | methyl {(4S)-4-(6-chloro-3′-ethyl-2- biphenylyl)-4-[(3R)-1-({2-(2-cyanoethyl)-4- [(methylamino)methyl]phenyl}carbonyl)-3- piperidinyl]-4-hydroxybutyl}carbamate |
| I-83a | 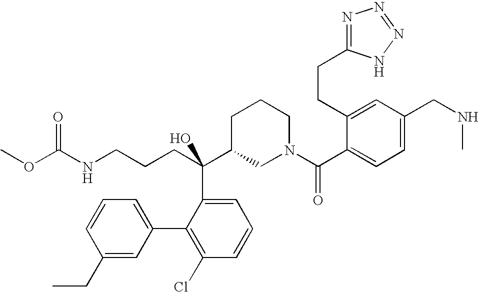 | methyl {(4S)-4-(6-chloro-3′-ethyl-2- biphenylyl)-4-hydroxy-4-[(3R)-1-({4- [(methylamino)methyl]-2-[2-(1H-tetrazol-5- yl)ethyl]phenyl}carbonyl)-3- piperidinyl]butyl}carbamate |
| I-84a | 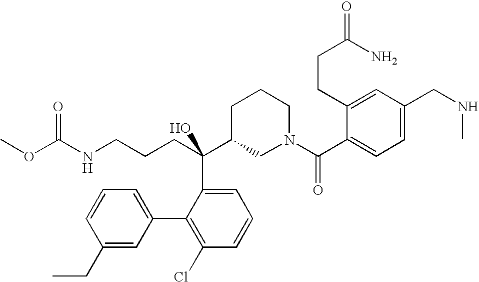 | methyl [(4S)-4-[(3R)-1-({2-(3-amino-3- oxopropyl)-4-[(methylamino) methyl]phenyl}carbonyl)-3-piperidinyl]-4-(6- chloro-3′-ethyl-2-biphenylyl)-4- hydroxybutyl]carbamate |
| I-85a | 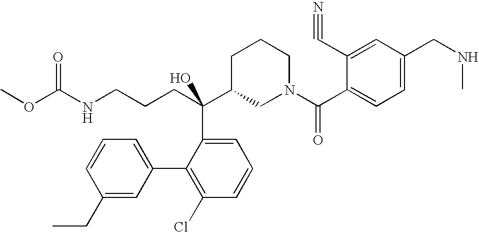 | methyl {(4S)-4-(6-chloro-3′-ethyl-2- biphenylyl)-4-[(3R)-1-({2-cyano-4- [(methylamino)methyl]phenyl}carbonyl)-3- piperidinyl]-4-hydroxybutyl}carbamate |
| I-86a | 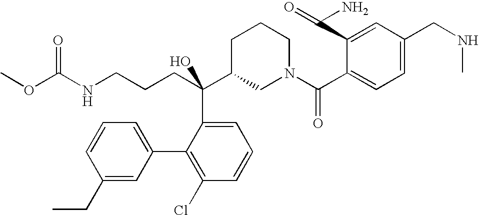 | methyl [(4S)-4-[(3R)-1-({2- (aminocarbonyl)-4-[(methylamino) methyl]phenyl}carbonyl)-3-piperidinyl]-4-(6- chloro-3′-ethyl-2-biphenylyl)-4- hydroxybutyl]carbamate |
| I-87a | 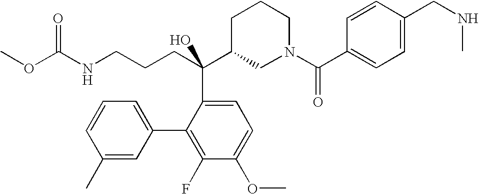 | methyl {(4S)-4-[6-fluoro-3′-methyl-5- (methyloxy)-2-biphenylyl]-4-hydroxy-4-[(3R)- 1-({4-[(methylamino)methyl]phenyl}carbonyl)- 3-piperidinyl]butyl}carbamate |
| I-88a | 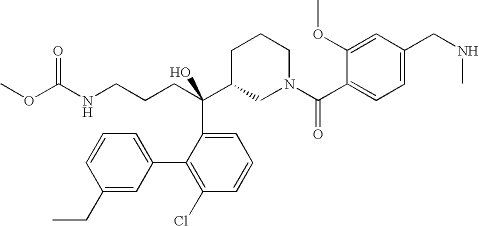 | methyl [(4S)-4-(6-chloro-3′-ethyl-2- biphenylyl)-4-hydroxy-4-((3R)-1-{[4- [(methylamino)methyl]-2- (methyloxy)phenyl]carbonyl}-3- piperidinyl)butyl]carbamate |
| I-89a | 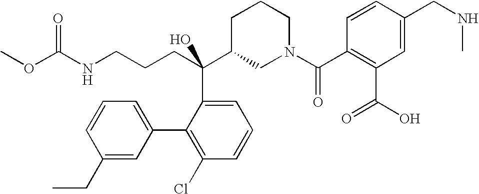 | 2-{[(3R)-3-((1S)-1-(6-chloro-3′-ethyl-2- biphenylyl)-1-hydroxy-4- {[(methyloxy)carbonyl]amino}butyl)-1- piperidinyl]carbonyl}-5- [(methylamino)methyl]benzoic acid |
| I-90a | 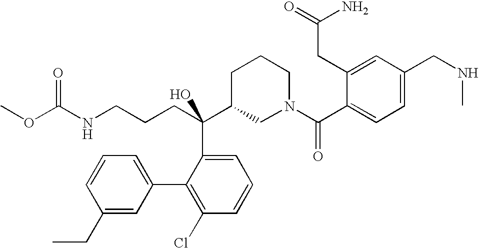 | methyl [(4S)-4-[(3R)-1-({2-(2-amino-2- oxoethyl)-4-[(methylamino) methyl]phenyl}carbonyl)-3-piperidinyl]-4-(6- chloro-3′-ethyl-2-biphenylyl)-4- hydroxybutyl]carbamate |
| I-91a | 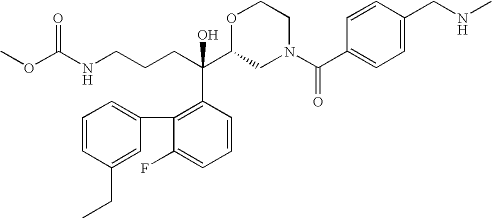 | methyl {(4R)-4-(3′-ethyl-6-fluoro-2- biphenylyl)-4-hydroxy-4-[(2R)-4-({4- [(methylamino)methyl]phenyl}carbonyl)-2- morpholinyl]butyl}carbamate |
| I-92a | 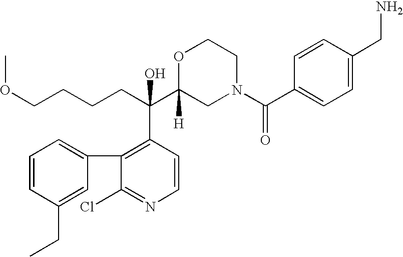 | (1R)-1-((2R)-4-{[4- (aminomethyl)phenyl]carbonyl}-2- morpholinyl)-1-[2-chloro-3-(3-ethylphenyl)-4- pyridinyl]-5-(methyloxy)-1-pentanol |
| I-93a | 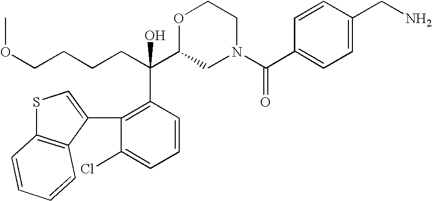 | (1R)-1-((2R)-4-{[4- (aminomethyl)phenyl]carbonyl}-2- morpholinyl)-1-[2-(1-benzothien-3-yl)-3- chlorophenyl]-5-(methyloxy)-1-pentanol |
| I-94a | 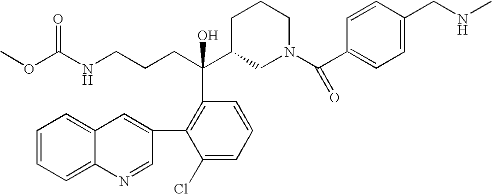 | methyl {(4S)-4-[3-chloro-2-(3- quinolinyl)phenyl]-4-hydroxy-4-[(3R)-1-({4- [(methylamino)methyl]phenyl}carbonyl)-3- piperidinyl]butyl}carbamate |
| I-95a | 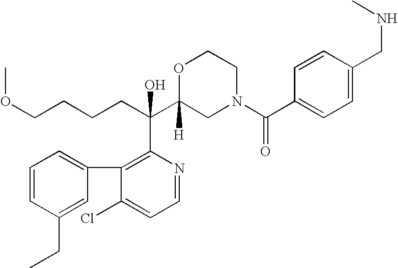 | (1R)-1-[4-chloro-3-(3-ethylphenyl)-2- pyridinyl]-1-[(2R)-4-({4-[(methylamino) methyl]phenyl}carbonyl)-2-morpholinyl]-5- (methyloxy)-1-pentanol |
| I-96a | 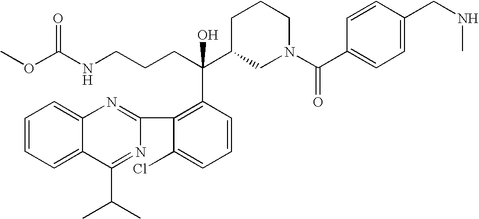 | methyl {(4S)-4-{3-chloro-2-[4-(1- methylethyl)-2-quinazolinyl]phenyl}-4- hydroxy-4-[(3R)-1-({4-[(methylamino) methyl]phenyl}carbonyl)-3-piperidinyl]butyl} carbamate |
| I-97a | 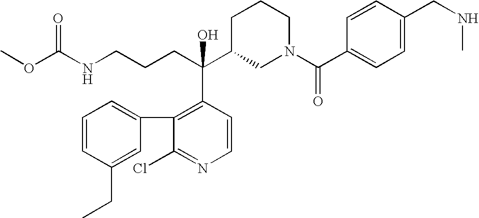 | methyl {(4S)-4-[2-chloro-3-(3- ethylphenyl)-4-pyridinyl]-4-hydroxy-4-[(3R)-1- ({4-[(methylamino)methyl]phenyl}carbonyl)-3- piperidinyl]butyl}carbamate |
| I-98a | 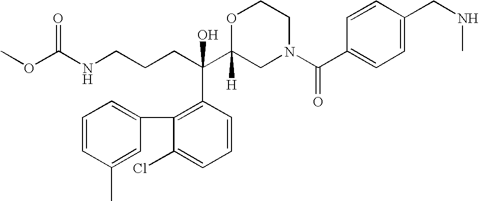 | methyl {(4R)-4-(6-chloro-3′-methyl-2- biphenylyl)-4-hydroxy-4-[(2R)-4-({4- [(methylamino)methyl]phenyl}carbonyl)-2- morpholinyl]butyl}carbamate |
| I-99a | 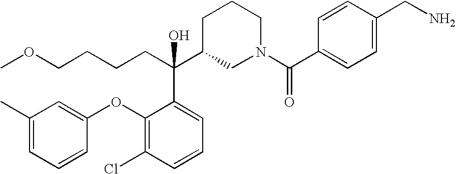 | (1S)-1-((3R)-1-{[4- (aminomethyl)phenyl]carbonyl}-3-piperidinyl)- 1-{3-chloro-2-[(3-methylphenyl)oxy]phenyl}- 5-(methyloxy)-1-pentanol |
| I-100a | 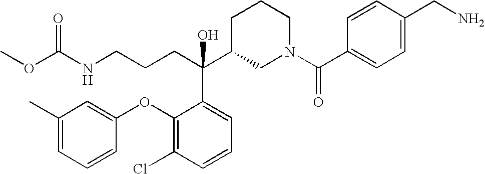 | methyl ((4S)-4-((3R)-1-{[4- (aminomethyl)phenyl]carbonyl}-3-piperidinyl)- 4-{3-chloro-2-[(3-methylphenyl)oxy]phenyl}- 4-hydroxybutyl)carbamate |
| I-101a | 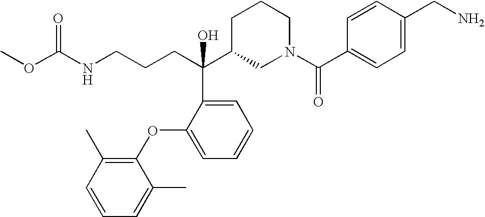 | methyl ((4S)-4-((3R)-1-{[4- (aminomethyl)phenyl]carbonyl}-3-piperidinyl)- 4-{2-[(2,6-dimethylphenyl)oxy]phenyl}-4- hydroxybutyl)carbamate |
| I-102a | 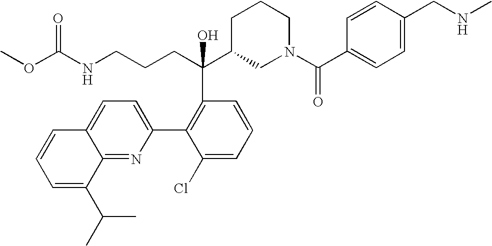 | methyl {(4S)-4-{3-chloro-2-[8-(1- methylethyl)-2-quinolinyl]phenyl}-4-hydroxy- 4-[(3R)-1-({4-[(methylamino) methyl]phenyl}carbonyl)-3- piperidinyl]butyl}carbamate |
| I-103a | 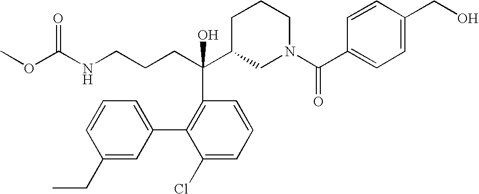 | methyl [(4S)-4-(6-chloro-3′-ethyl-2- biphenylyl)-4-hydroxy-4-((3R)-1-{[4- (hydroxymethyl)phenyl]carbonyl}-3- piperidinyl)butyl]carbamate |
| I-104a | 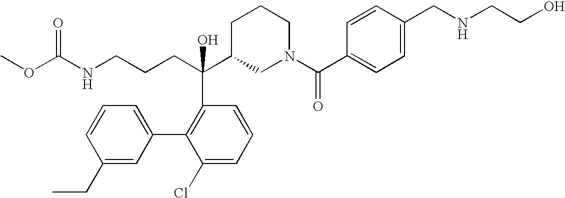 | methyl ((4S)-4-(6-chloro-3′-ethyl-2- biphenylyl)-4-hydroxy-4-{(3R)-1-[(4-{[(2- hydroxyethyl)amino]methyl}phenyl)carbonyl]- 3-piperidinyl}butyl)carbamate |
| I-105a | 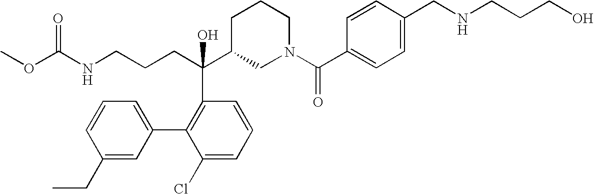 | methyl ((4S)-4-(6-chloro-3′-ethyl-2- biphenylyl)-4-hydroxy-4-{(3R)-1-[(4-{[(3- hydroxypropyl)amino]methyl}phenyl)carbonyl]- 3-piperidinyl}butyl)carbamate |
| I-106a | 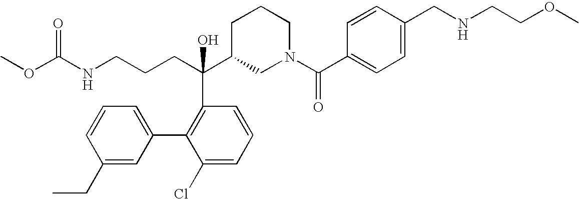 | methyl [(4S)-4-(6-chloro-3′-ethyl-2- biphenylyl)-4-hydroxy-4-((3R)-1-{[4-({[2- (methyloxy)ethyl]amino}methyl)phenyl]carbon- yl}-3-piperidinyl)butyl]carbamate |
| I-107a | 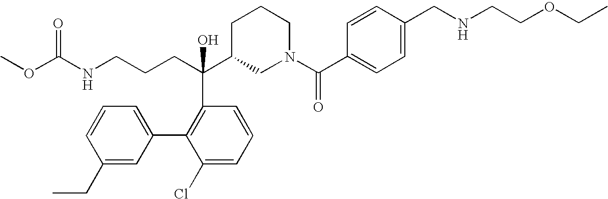 | methyl [(4S)-4-(6-chloro-3′-ethyl-2- biphenylyl)-4-((3R)-1-{[4-({[2-(ethyloxy) ethyl]amino}methyl)phenyl]carbonyl}-3- piperidinyl)-4-hydroxybutyl]carbamate |
| I-108a | 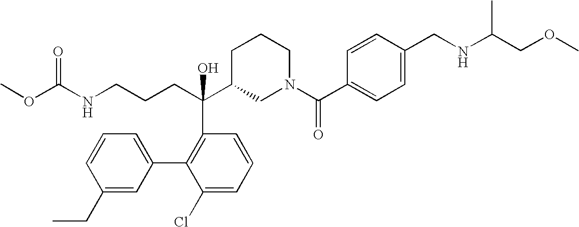 | methyl [(4S)-4-(6-chloro-3′-ethyl-2- biphenylyl)-4-hydroxy-4-((3R)-1-{[4-({[1- methyl-2-(methyloxy)ethyl]amino}methyl) phenyl]carbonyl}-3-piperidinyl)butyl] carbamate |
| I-109a | 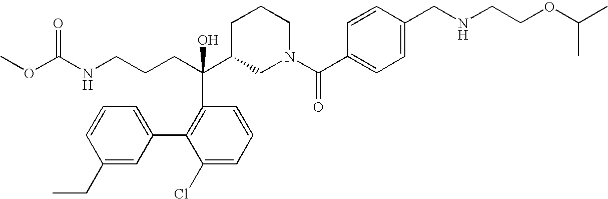 | methyl {(4S)-4-(6-chloro-3′-ethyl-2- biphenylyl)-4-hydroxy-4-[(3R)-1-({4-[({2-[(1- methylethyl)oxy]ethyl}amino)methyl]phenyl} carbonyl)-3-piperidinyl]butyl}carbamate |
| I-110a | 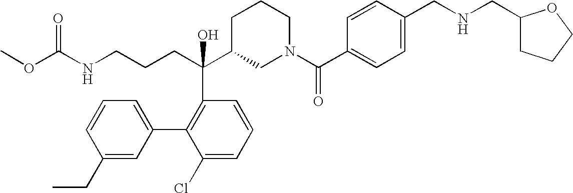 | methyl ((4S)-4-(6-chloro-3′-ethyl-2- biphenylyl)-4-hydroxy-4-{(3R)-1-[(4- {[(tetrahydro-2-furanylmethyl) amino]methyl}phenyl)carbonyl]-3- piperidinyl}butyl)carbamate |
| I-111a | 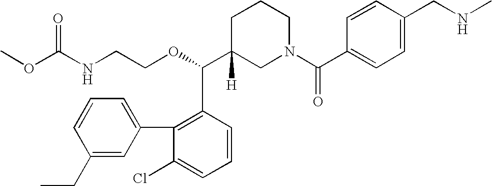 | methyl [2-({(R)-(6-chloro-3′-ethyl-2- biphenylyl)[(3R)-1-({4-[(methylamino)methyl] phenyl}carbonyl)-3-piperidinyl]methyl}oxy) ethyl]carbamate |
| I-112a | 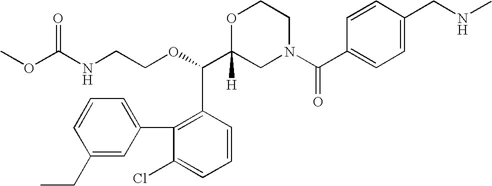 | methyl [2-({(S)-(6-chloro-3′-ethyl-2- biphenylyl)[(2R)-4-({4-[(methylamino) methyl]phenyl}carbonyl)-2-morpholinyl] methyl}oxy)ethyl]carbamate |
| I-113a | 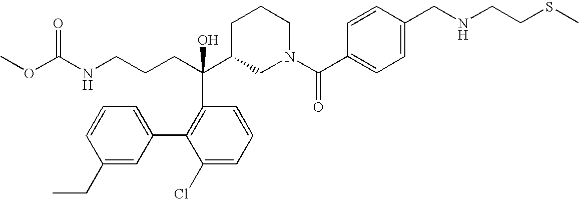 | methyl [(4S)-4-(6-chloro-3′-ethyl-2- biphenylyl)-4-hydroxy-4-((3R)-1-{[4-({[2- (methylthio)ethyl]amino}methyl)phenyl] carbonyl}-3-piperidinyl)butyl]carbamate |
| I-114a | 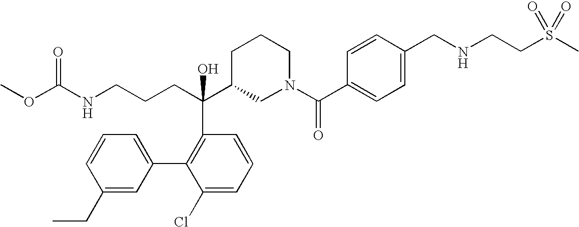 | methyl [(4S)-4-(6-chloro-3′-ethyl-2- biphenylyl)-4-hydroxy-4-((3R)-1-{[4-({[2- (methylsulfonyl)ethyl]amino}methyl)phenyl] carbonyl}-3-piperidinyl)butyl]carbamate |
| I-115a | 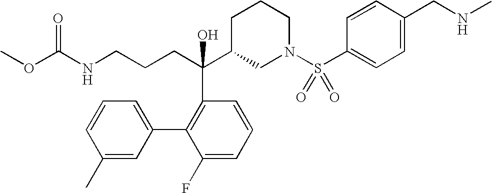 | methyl {(4S)-4-(6-fluoro-3′-methyl-2- biphenylyl)-4-hydroxy-4-[(3R)-1-({4- [(methylamino)methyl]phenyl}sulfonyl)-3- piperidinyl]butyl}carbamate |
| I-116a | 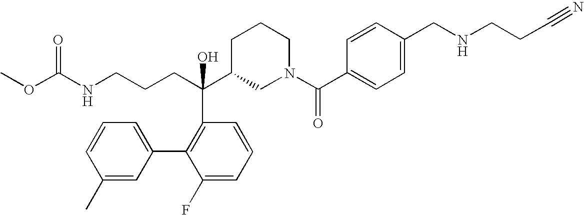 | methyl [(4S)-4-{(3R)-1-[(4-{[(2- cyanoethyl)amino]methyl}phenyl)carbonyl]-3- piperidinyl}-4-(6-fluoro-3′-methyl-2- biphenylyl)-4-hydroxybutyl]carbamate |
| I-117a | 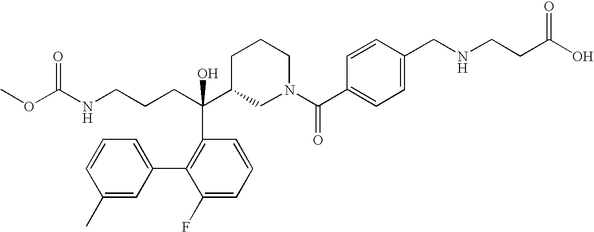 | N-[(4-{[(3R)-3-((1S)-1-(6-fluoro-3′-methyl- 2-biphenylyl)-1-hydroxy-4- {[(methyloxy)carbonyl]amino}butyl)-1- piperidinyl]carbonyl}phenyl)methyl]-□-alanine |
| I-118a | 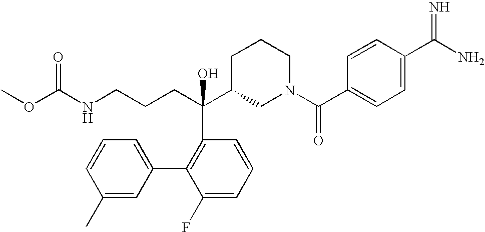 | methyl [(4S)-4-[(3R)-1-({4- [amino(imino)methyl]phenyl}carbonyl)-3- piperidinyl]-4-(6-fluoro-3′-methyl-2- biphenylyl)-4-hydroxybutyl]carbamate |
| I-119a | 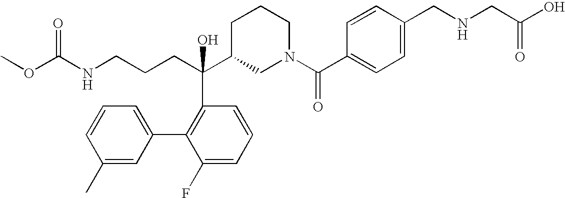 | N-[(4-{[(3R)-3-((S)-1-(6-fluoro-3′-methyl- 2-biphenylyl)-1-hydroxy-4- {[(methyloxy)carbonyl]amino}butyl)-1- piperidinyl]carbonyl}phenyl)methyl]glycine |
| I-120a | 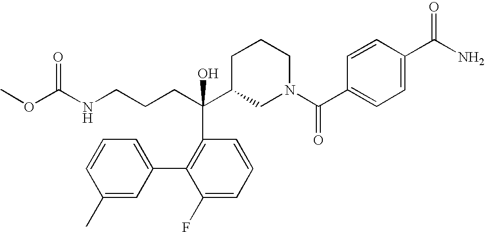 | methyl [(4S)-4-((3R)-1-{[4- (aminocarbonyl)phenyl]carbonyl}-3- piperidinyl)-4-(6-fluoro-3′-methyl-2- biphenylyl)-4-hydroxybutyl]carbamate |
| I-121a | 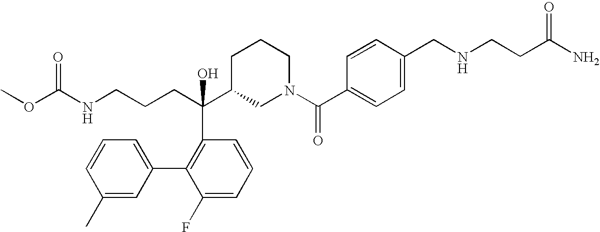 | methyl [(4S)-4-{(3R)-1-[(4-{[(3-amino-3- oxopropyl)amino]methyl}phenyl)carbonyl]-3- piperidinyl}-4-(6-fluoro-3′-methyl-2- biphenylyl)-4-hydroxybutyl]carbamate |
| I-122a | 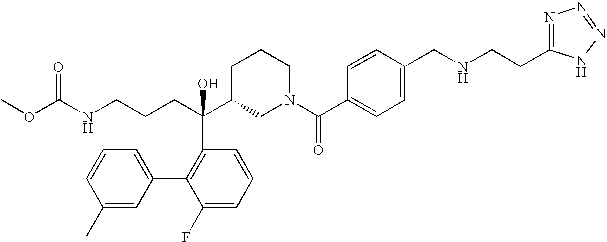 | methyl [(4S)-4-(6-fluoro-3′-methyl-2- biphenylyl)-4-hydroxy-4-((3R)-1-{[4-({[2-(1H- tetrazol-5-yl)ethyl]amino}methyl) phenyl]carbonyl}-3-piperidinyl)butyl] carbamate |
| I-123a | 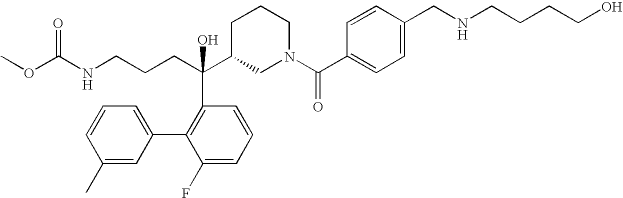 | methyl ((4S)-4-(6-fluoro-3′-methyl-2- biphenylyl)-4-hydroxy-4-{(3R)-1-[(4-{[(4- hydroxybutyl)amino]methyl}phenyl)carbonyl]- 3-piperidinyl}butyl)carbamate |
| I-124a | 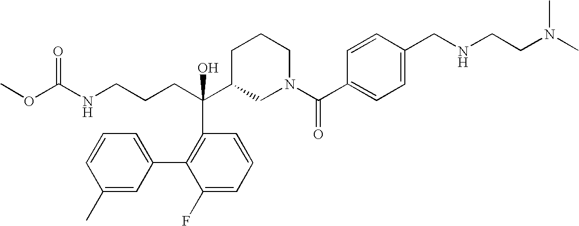 | methyl [(4S)-4-((3R)-1-{[4-({[2- (dimethylamino)ethyl]amino}methyl)phenyl] carbonyl}-3-piperidinyl)-4-(6-fluoro-3′-methyl-2- biphenylyl)-4-hydroxybutyl]carbamate |
| I-125a | 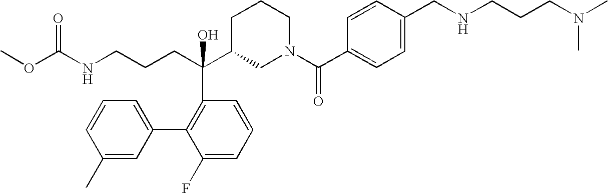 | methyl [(4S)-4-((3R)-1-{[4-({[3- (dimethylamino)propyl]amino}methyl)phenyl] carbonyl}-3-piperidinyl)-4-(6-fluoro-3′-methyl- 2-biphenylyl)-4-hydroxybutyl]carbamate |
| I-126a | 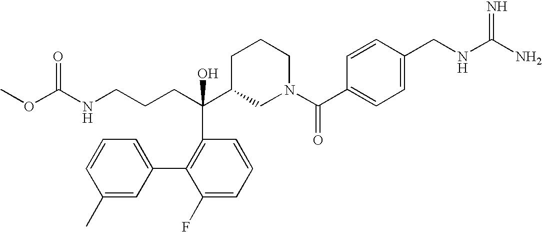 | methyl [(4S)-4-((3R)-1-{[4- ({[amino(imino)methyl]amino}methyl)phenyl] carbonyl}-3-piperidinyl)-4-(6-fluoro-3′-methyl- 2-biphenylyl)-4-hydroxybutyl]carbamate |
| I-127a | 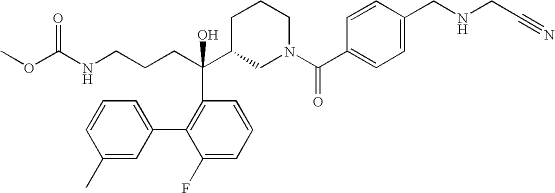 | methyl [(4S)-4-{(3R)-1-[(4- {[(cyanomethyl)amino]methyl}phenyl)carbonyl]- 3-piperidinyl}-4-(6-fluoro-3′-methyl-2- biphenylyl)-4-hydroxybutyl]carbamate |
| I-128a | 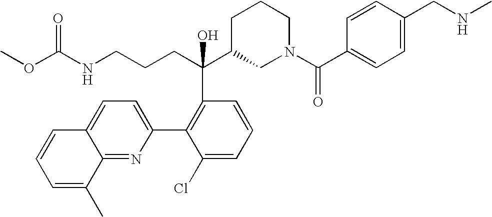 | methyl {(4S)-4-[3-chloro-2-(8-methyl-2- quinolinyl)phenyl]-4-hydroxy-4-[(3R)-1-({4- [(methylamino)methyl]phenyl}carbonyl)-3- piperidinyl]butyl}carbamate |
| I-130a | 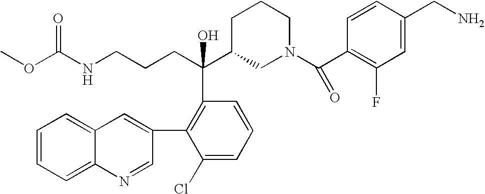 | methyl {(4S)-4-((3R)-1-{[4- (aminomethyl)-2-fluorophenyl]carbonyl}-3- piperidinyl)-4-[3-chloro-2-(3- quinolinyl)phenyl]-4-hydroxybutyl}carbamate |
| I-131a | 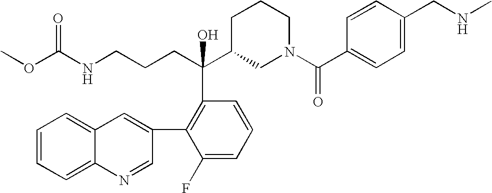 | methyl {(4S)-4-[3-fluoro-2-(3- quinolinyl)phenyl]-4-hydroxy-4-[(3R)-1-({4- [(methylamino)methyl]phenyl}carbonyl)-3- piperidinyl]butyl}carbamate |
| I-132a | 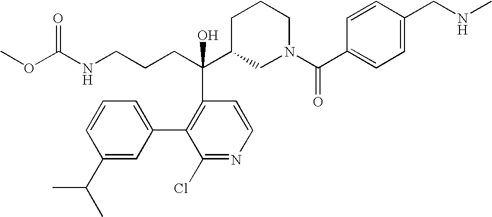 | methyl {(4S)-4-{2-chloro-3-[3-(1- methylethyl)phenyl]-4-pyridinyl}-4-hydroxy-4- [(3R)-1-({4- [(methylamino)methyl]phenyl}carbonyl)-3- piperidinyl]butyl}carbamate |
| I-133a | 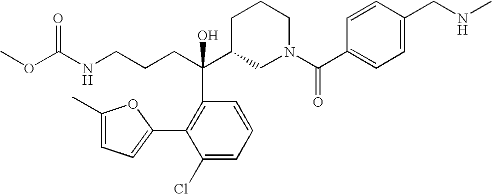 | methyl {(4S)-4-[3-chloro-2-(5-methyl-2- furanyl)phenyl]-4-hydroxy-4-[(3R)-1-({4- [(methylamino)methyl]phenyl}carbonyl)-3- piperidinyl]butyl}carbamate |
| I-134a | 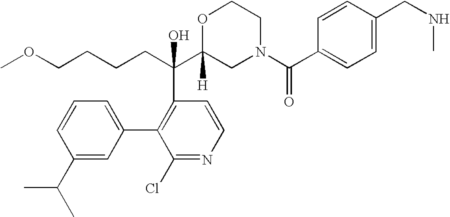 | (1R)-1-{2-chloro-3-[3-(1- methylethyl)phenyl]-4-pyridinyl}-1-[(2R)-4- ({4-[(methylamino)methyl]phenyl}carbonyl)-2- morpholinyl]-5-(methyloxy)-1-pentanol |
| I-135a | 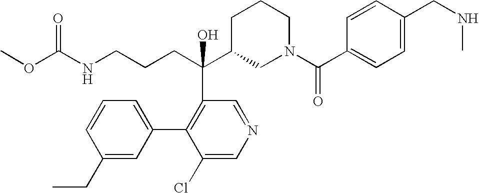 | methyl {(4S)-4-[5-chloro-4-(3- ethylphenyl)-3-pyridinyl]-4-hydroxy-4-[(3R)-1- ({4-[(methylamino)methyl]phenyl}carbonyl)-3- piperidinyl]butyl}carbamate |
| I-136a | 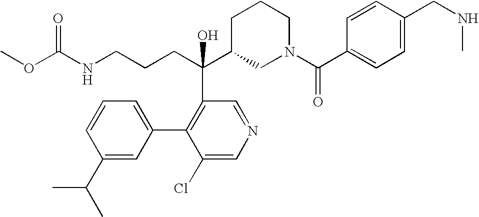 | methyl {(4S)-4-{5-chloro-4-[3-(1- methylethyl)phenyl]-3-pyridinyl}-4-hydroxy-4- [(3R)-1-({4-[(methylamino)methyl] phenyl}carbonyl)-3-piperidinyl]butyl} carbamate |
| I-137a | 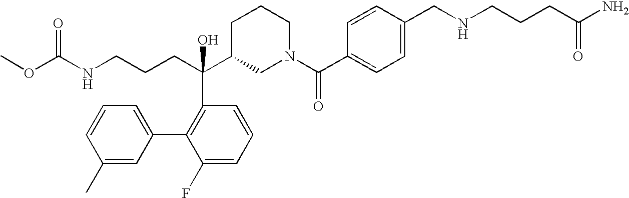 | methyl [(4S)-4-{(3R)-1-[(4-{[(4-amino-4- oxobutyl)amino]methyl}phenyl)carbonyl]-3- piperidinyl}-4-(6-fluoro-3′-methyl-2- biphenylyl)-4-hydroxybutyl]carbamate |
| I-138a | 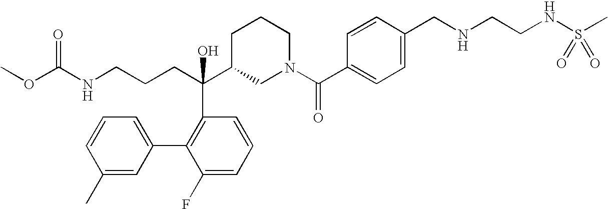 | methyl {(4S)-4-(6-fluoro-3′-methyl-2- biphenylyl)-4-hydroxy-4-[(3R)-1-({4-[({2- [(methylsulfonyl)amino]ethyl}amino)methyl] phenyl}carbonyl)-3-piperidinyl]butyl}carbamate |
| I-139a | 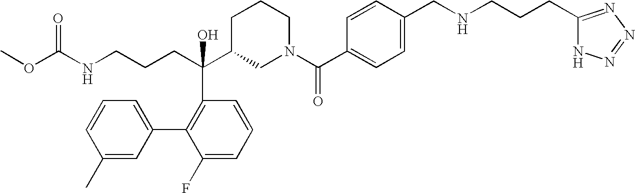 | methyl [(4S)-4-(6-fluoro-3′-methyl-2- biphenylyl)-4-hydroxy-4-((3R)-1-{[4-({[3-(1H- tetrazol-5- yl)propyl]amino}methyl)phenyl]carbonyl}-3- piperidinyl)butyl]carbamate |
| I-140a | 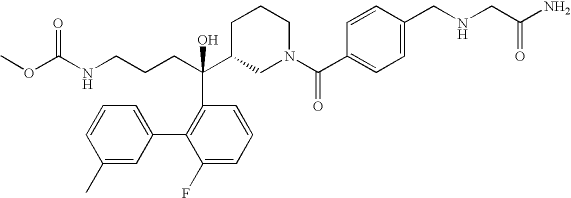 | methyl [(4S)-4-{(3R)-1-[(4-{[(2-amino-2- oxoethyl)amino]methyl}phenyl)carbonyl]-3- piperidinyl}-4-(6-fluoro-3′-methyl-2- biphenylyl)-4-hydroxybutyl]carbamate |
| I-141a | 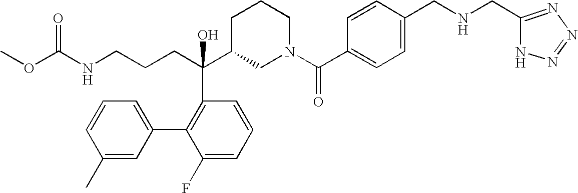 | methyl ((4S)-4-(6-fluoro-3′-methyl-2- biphenylyl)-4-hydroxy-4-{(3R)-1-[(4-{[(1H- tetrazol-5-ylmethyl)amino]methyl} phenyl)carbonyl]-3-piperidinyl}butyl) carbamate |
| I-142a | 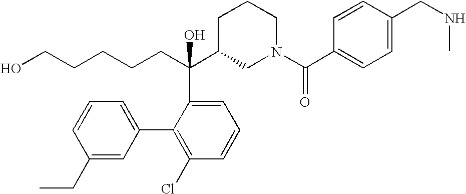 | (1S)-1-(6-chloro-3′-ethyl-2-biphenylyl)-1- [(3R)-1-({4-[(methylamino)methyl]phenyl} carbonyl)-3-piperidinyl]-1,6-hexanediol |
| I-143a | 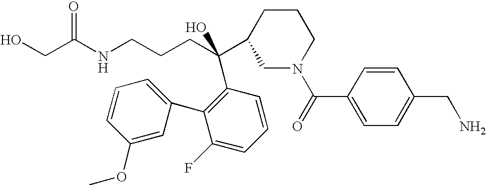 | N-{(4S)-4-((3R)-1-{[4-(aminomethyl) phenyl]carbonyl}-3-piperidinyl)-4-[6-fluoro-3′- (methyloxy)-2-biphenylyl]-4-hydroxybutyl}-2- hydroxyacetamide |
| I-144a | 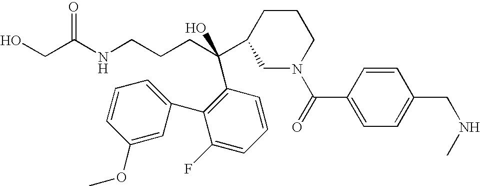 | N-{(4S)-4-[6-fluoro-3′-(methyloxy)-2- biphenylyl]-4-hydroxy-4-[(3R)-1-({4- [(methylamino)methyl]phenyl}carbonyl)-3- piperidinyl]butyl}-2-hydroxyacetamide |
| I-145a | 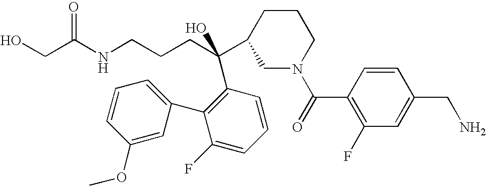 | N-{(4S)-4-((3R)-1-{[4-(aminomethyl)-2- fluorophenyl]carbonyl}-3-piperidinyl)-4-[6- fluoro-3′-(methyloxy)-2-biphenylyl]-4- hydroxybutyl}-2-hydroxyacetamide |
| I-146a | 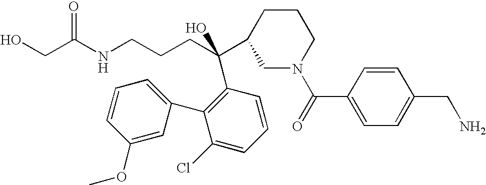 | N-{(4S)-4-((3R)-1-{[4- (aminomethyl)phenyl]carbonyl}-3-piperidinyl)- 4-[6-chloro-3′-(methyloxy)-2-biphenylyl]-4- hydroxybutyl}-2-hydroxyacetamide |
| I-147a | 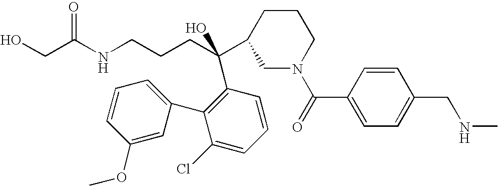 | N-{(4S)-4-[6-chloro-3′-(methyloxy)-2- biphenylyl]-4-hydroxy-4-[(3R)-1-({4- [(methylamino)methyl]phenyl}carbonyl)-3- piperidinyl]butyl}-2-hydroxyacetamide |
| I-148a | 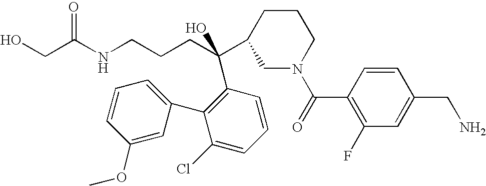 | N-{(4S)-4-((3R)-1-{[4-(aminomethyl)-2- fluorophenyl]carbonyl}-3-piperidinyl)-4-[6- chloro-3′-(methyloxy)-2-biphenylyl]-4- hydroxybutyl}-2-hydroxyacetamide |
| I-149a | 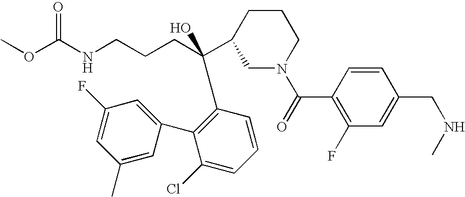 | methyl {(4S)-4-(6-chloro-3′-fluoro-5′- methyl-2-biphenylyl)-4-[(3R)-1-({2-fluoro-4- [(methylamino)methyl]phenyl}carbonyl)-3- piperidinyl]-4-hydroxybutyl}carbamate |
| I-150a | 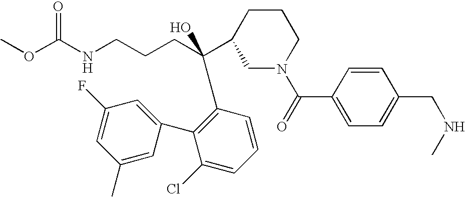 | methyl {(4S)-4-(6-chloro-3′-fluoro-5′- methyl-2-biphenylyl)-4-hydroxy-4-[(3R)-1-({4- [(methylamino)methyl]phenyl}carbonyl)-3- piperidinyl]butyl}carbamate |
| I-151a | 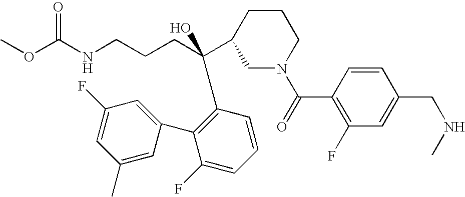 | methyl {(4S)-4-(3′,6-difluoro-5′-methyl-2- biphenylyl)-4-[(3R)-1-({2-fluoro-4- [(methylamino)methyl]phenyl}carbonyl)-3- piperidinyl]-4-hydroxybutyl}carbamate |
| I-152a | 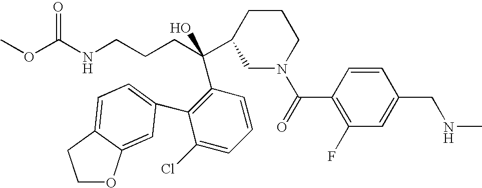 | methyl {(4S)-4-[3-chloro-2-(2,3-dihydro-1- benzofuran-6-yl)phenyl]-4-[(3R)-1-({2-fluoro- 4-[(methylamino)methyl]phenyl}carbonyl)-3- piperidinyl]-4-hydroxybutyl}carbamate |
| I-153a | 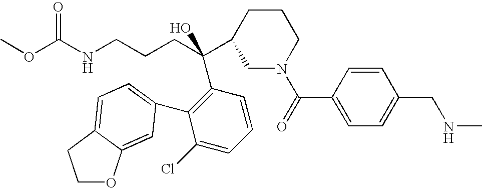 | methyl {(4S)-4-[3-chloro-2-(2,3-dihydro-1- benzofuran-6-yl)phenyl]-4-hydroxy-4-[(3R)-1- ({4-[(methylamino)methyl]phenyl}carbonyl)-3- piperidinyl]butyl}carbamate |
| I-154a | 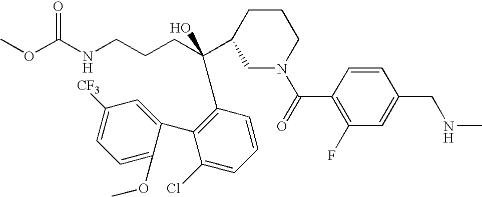 | methyl {(4S)-4-[6-chloro-2′-(methyloxy)- 5′-(trifluoromethyl)-2-biphenylyl]-4-[(3R)-1- ({2-fluoro-4- [(methylamino)methyl]phenyl}carbonyl)-3- piperidinyl]-4-hydroxybutyl}carbamate |
| I-155a | 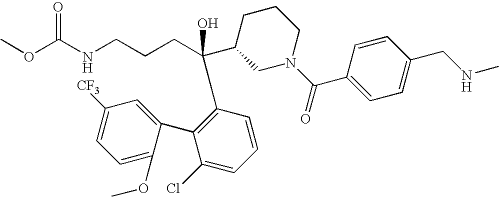 | methyl {(4S)-4-[6-chloro-2′-(methyloxy)- 5′-(trifluoromethyl)-2-biphenylyl]-4-hydroxy-4- [(3R)-1-({4- [(methylamino)methyl]phenyl}carbonyl)-3- piperidinyl]butyl}carbamate |
| I-156a | 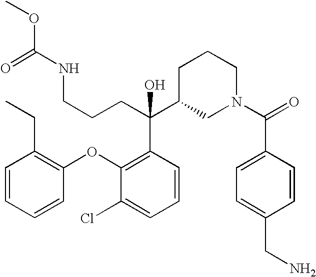 | methyl ((4S)-4-((3R)-1-{[4- (aminomethyl)phenyl]carbonyl}-3-piperidinyl)- 4-{3-chloro-2-[(2-ethylphenyl)oxy]phenyl}-4- hydroxybutyl)carbamate |
| I-157a | 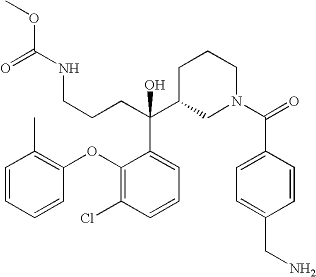 | methyl ((4S)-4-((3R)-1-{[4- (aminomethyl)phenyl]carbonyl}-3-piperidinyl)- 4-{3-chloro-2-[(2-methylphenyl)oxy]phenyl}- 4-hydroxybutyl)carbamate |
| I-158a | 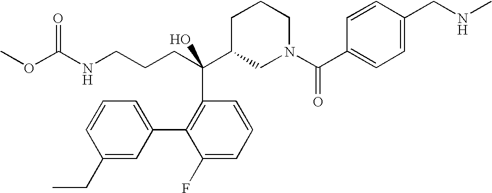 | methyl {(4S)-4-(3′-ethyl-6-fluoro-2- biphenylyl)-4-hydroxy-4-[(3R)-1-({4- [(methylamino)methyl]phenyl}carbonyl)-3- piperidinyl]butyl}carbamate |
| I-159a | 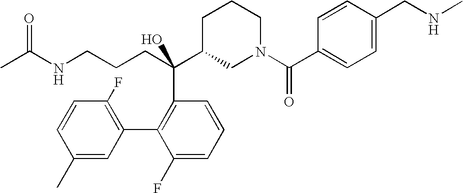 | N-{(4S)-4-(2′,6-difluoro-5′-methyl-2- biphenylyl)-4-hydroxy-4-[(3R)-1-({4- [(methylamino)methyl]phenyl}carbonyl)-3- piperidinyl]butyl}acetamide |
| I-160a | 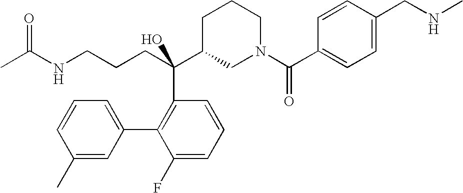 | N-{(4S)-4-(6-fluoro-3′-methyl-2- biphenylyl)-4-hydroxy-4-[(3R)-1-({4- [(methylamino)methyl]phenyl}carbonyl)-3- piperidinyl]butyl}acetamide |
| I-161a | 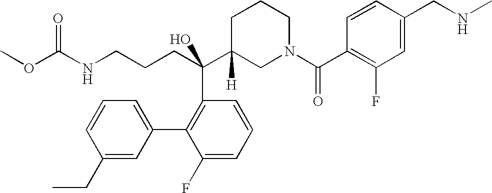 | methyl {(4S)-4-(3′-ethyl-6-fluoro-2- biphenylyl)-4-[(3R)-1-({2-fluoro-4- [(methylamino)methyl]phenyl}carbonyl)-3- piperidinyl]-4-hydroxybutyl}carbamate |
| I-162a | 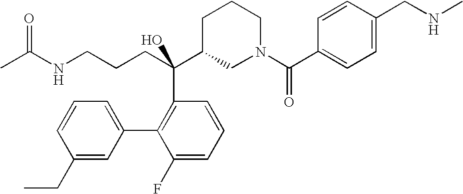 | N-{(4S)-4-(3′-ethyl-6-fluoro-2-biphenylyl)- 4-hydroxy-4-[(3R)-1-({4- [(methylamino)methyl]phenyl}carbonyl)-3- piperidinyl]butyl}acetamide |
| I-163a | 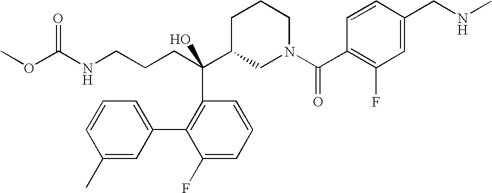 | methyl [(4S)-4-[(3R)-1-({2-fluoro-4- [(methylamino)methyl]phenyl}carbonyl)-3- piperidinyl]-4-(6-fluoro-3′-methyl-2- biphenylyl)-4-hydroxybutyl]carbamate |
| I-164a | 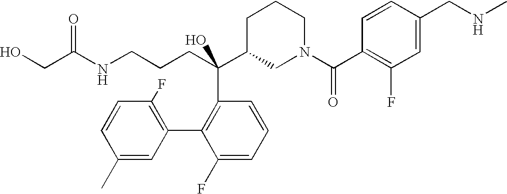 | N-{(4S)-4-(2′,6-difluoro-5′-methyl-2- biphenylyl)-4-[(3R)-1-({2-fluoro-4- [(methylamino)methyl]phenyl}carbonyl)-3- piperidinyl]-4-hydroxybutyl}-2- hydroxyacetamide |
| I-165a | 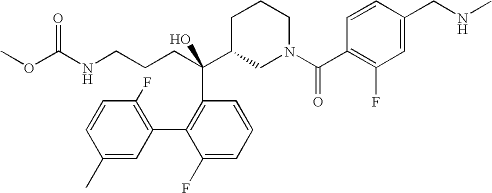 | methyl {(4S)-4-(2′,6-difluoro-5′-methyl-2- biphenylyl)-4-[(3R)-1-({2-fluoro-4- [(methylamino)methyl]phenyl}carbonyl)-3- piperidinyl]-4-hydroxybutyl}carbamate |
| I-166a | 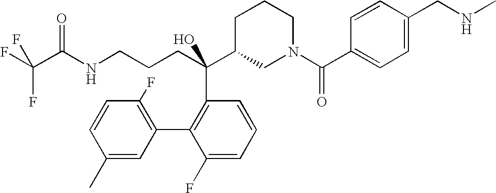 | N-{(4S)-4-(2′,6-difluoro-5′-methyl-2- biphenylyl)-4-hydroxy-4-[(3R)-1-({4- [(methylamino)methyl]phenyl}carbonyl)-3- piperidinyl]butyl}-2,2,2-trifluoroacetamide |
| I-167a | 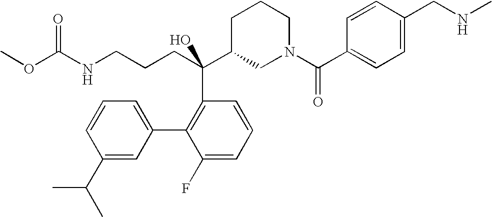 | methyl {(4S)-4-[6-fluoro-3′-(1- methylethyl)-2-biphenylyl]-4-hydroxy-4-[(3R)- 1-({4-[(methylamino)methyl]phenyl}carbonyl)- 3-piperidinyl]butyl}carbamate |
| I-168a | 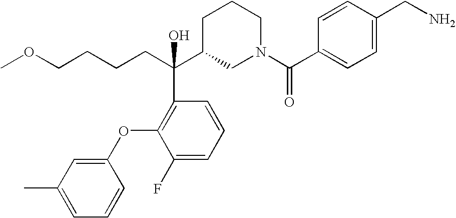 | (1S)-1-((3R)-1-{[4- (aminomethyl)phenyl]carbonyl}-3-piperidinyl)- 1-{3-fluoro-2-[(3-methylphenyl)oxy]phenyl}-5- (methyloxy)-1-pentanol |
| I-169a | 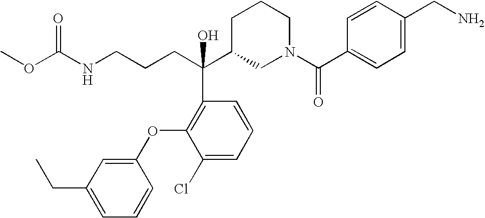 | methyl ((4S)-4-((3R)-1-{[4- (aminomethyl)phenyl]carbonyl}-3- piperidinyl)-4-{3-chloro-2-[(3- ethylphenyl)oxy]phenyl}-4-hydroxybutyl) carbamate |
| I-170a | 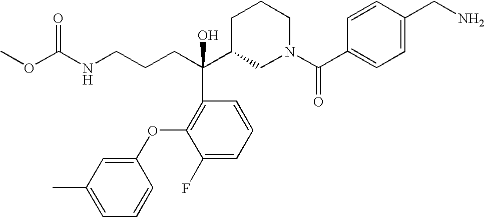 | methyl ((4S)-4-((3R)-1-{[4- (aminomethyl)phenyl]carbonyl}-3-piperidinyl)- 4-{3-fluoro-2-[(3-methylphenyl)oxy]phenyl}-4- hydroxybutyl)carbamate |
| I-171a | 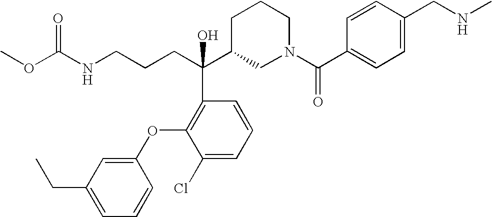 | methyl {(4S)-4-{3-chloro-2-[(3- ethylphenyl)oxy]phenyl}-4-hydroxy-4-[(3R)-1- ({4-[(methylamino)methyl]phenyl}carbonyl)-3- piperidinyl]butyl}carbamate |
| I-172a | 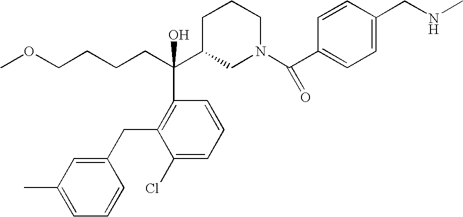 | (1S)-1-{3-chloro-2-[(3- methylphenyl)methyl]phenyl}-1-[(3R)-1-({4- [(methylamino)methyl]phenyl}carbonyl)-3- piperidinyl]-5-(methyloxy)-1-pentanol |
| I-173a | 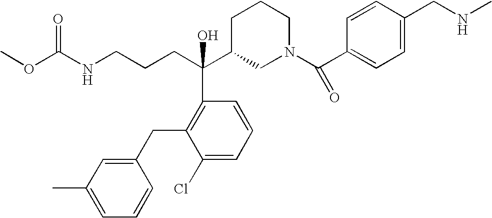 | methyl {(4S)-4-{3-chloro-2-[(3- methylphenyl)methyl]phenyl}-4-hydroxy-4- [(3R)-1-({4-[(methylamino)methyl]phenyl} carbonyl)-3-piperidinyl]butyl}carbamate |
| I-174a | 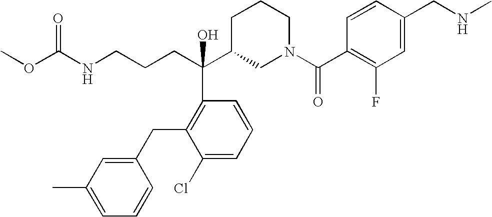 | methyl {(4S)-4-{3-chloro-2-[(3- methylphenyl)methyl]phenyl}-4-[(3R)-1-({2- fluoro-4-[(methylamino)methyl]phenyl} carbonyl)-3-piperidinyl]-4-ydroxybutyl} carbamate |
| | and the salts thereof. | |

The following Compound Nos. represent preferred compounds of this invention: I-52, I-53, I-54, I-55, I-56, I-57, I-58, I-64, I-70, I-75, I-77, I-80, I-81, I-83, I-84, I-89, I-96, I-97, I-100a, I-115a, I-116a, I-117a, I-119a, I-121a, I-122a, I-124a, I-125a, I-130a, I-132a, I-133a, I-139a, I-140a, I-141a, I-142a, I-144a, I-147a, I-148a, I-153a, I-154a, I-163a, and I-170a, or a salt thereof. The following Compound Nos. represent the more preferred compounds of this invention: I-62a, I-63a, I-65a, I-67a, I-69a, I-71a, I-72a, I-73a, I-74a, I-78a, I-79a, I-82a, I-85a, I-86a, I-88a, I-91a, I-90a, I-94a, I-98a, I-102a, I-104a, I-105a, I-106a, I-107a, I-108a, I-109a, I-110a, I-113a, I-114a, I-118a, I-123a, I-128a, I-131a, I-135a, I-136a, I-137a, I-138a, I-149a, I-150a, I-151a, I-152a, I-155a, I-156a, I-157a, I-158a, I-159a, I-160a, I-161a, I-162a, I-164a, I-165a, I-166a, I-167a, I-169a, and I-171a, or a salt thereof.

The compounds of the invention (Compound # 1-174) exhibit 50% renin inhibition (as determined using the method of Example 22) at concentrations of from approximately 5000 nM to approximately 0.01 nM. Preferred compounds of the invention exhibit 50% inhibition at concentrations of from approximately 50 nM to approximately 0.01 nM. More preferred compounds of the invention exhibit 50% inhibition at concentrations of from approximately 5 nM to approximately 0.01 nM. When any variable (e.g., aryl, heterocyclyl, R¹, R², etc.) occurs more than once in a compound, its definition on each occurrence is independent of any other occurrence.

“Alkyl” means a saturated aliphatic branched or straight-chain mono- or di-valent hydrocarbon radical having the specified number of carbon atoms. Thus, “(C₁-C₈)alkyl” means a radical having from 1-8 carbon atoms in a linear or branched arrangement. “(C₁-C₆)alkyl” includes methyl, ethyl, propyl, butyl, pentyl, and hexyl.

“Cycloalkyl” means a saturated aliphatic cyclic hydrocarbon radical having the specified number of carbon atoms. Thus, (C₃-C₇)cycloalkyl means a radical having from 3-8 carbon atoms arranged in a ring. (C₃-C₇)cycloalkyl includes cyclopropyl, cyclobutyl, cyclopentyl, cyclohexyl, and cycloheptyl.

Haloalkyl and halocycloalkyl include mono, poly, and perhaloalkyl groups where the halogens are independently selected from fluorine, chlorine, and bromine.

“Heterocyclyl” means a heteroaryl or a saturated heterocyclic ring group.

Saturated heterocyclic rings are 4-, 5-, 6-, and 7-membered heterocyclic rings containing 1 to 4 heteroatoms independently selected from N, O, and S, and include pyrrolidine, piperidine, tetrahydrofuran, tetrahydropyran, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine 1,1-dioxide, tetrahydro-2H-1,2-thiazine 1,1-dioxide, and isothiazolidine 1,1-dioxide. Oxo substituted saturated heterocyclic rings include tetrahydrothiophene 1-oxide, tetrahydrothiophene 1,1-dioxide, thiomorpholine 1-oxide, thiomorpholine 1,1-dioxide, tetrahydro-2H-1,2-thiazine 1,1-dioxide, and isothiazolidine 1,1-dioxide, pyrrolidin-2-one, piperidin-2-one, piperazin-2-one, and morpholin-2-one.

“Heteroaryl” means a monovalent heteroaromatic monocyclic and polycyclic ring radical. Heteroaryl rings are 5- and 6-membered aromatic heterocyclic rings containing 1 to 4 heteroatoms independently selected from N, O, and S, and include furan, thiophene, pyrrole, imidazole, pyrazole, oxazole, isoxazole, thiazole, isothiazole, 1,2,3-triazole, 1,2,4-triazole, 1,3,4-oxadiazole, 1,2,5-thiadiazole, 1,2,5-thiadiazole 1-oxide, 1,2,5-thiadiazole 1,1-dioxide, 1,3,4-thiadiazole, pyridine, pyridine-N-oxide, pyrazine, pyrimidine, pyridazine, 1,2,4-triazine, 1,3,5-triazine, and tetrazole. Bicyclic heteroaryl rings are bicyclo[4.4.0] and bicyclo[4.3.0] fused ring systems containing 1 to 4 heteroatoms independently selected from N, O, and S, and include indolizine, indole, isoindole, benzo[b]furan, benzo[b]thiophene, indazole, benzimidazole, benzthiazole, purine, 4H-quinolizine, quinoline, isoquinoline, cinnoline, phthalazine, quinazoline, quinoxaline, 1,8-naphthyridine, and pteridine.

“Alkoxy” means an alkyl radical attached through an oxygen linking atom. “(C₁-C₄)-alkoxy” includes methoxy, ethoxy, propoxy, and butoxy.

“Aromatic” means an unsaturated cycloalkyl ring system.

“Aryl” means an aromatic monocyclic, or polycyclic ring system. Aryl systems include phenyl, naphthalenyl, fluorenyl, indenyl, azulenyl, and anthracenyl.

“Arylheterocyclyl” means a phenyl group fused to a partially saturated 5-6 membered heterocyclic ring with 1-2 heteroatoms independently selected from N, S, and O, including dihydrobenzofuranyl, dihydrobenzothienyl, benzodioxolyl, dihydrochromenyl, dihydrobenzodioxinyl, dihydrobenzoxazinyl, tetrahydroquinolinyl, tetrahydroisoquinolinyl, and tetrahydroquinoxalinyl.

“Hetero” refers to the replacement of at least one carbon atom member in a ring system with at least one heteroatom selected from N, S, and O. A hetero ring may have 1, 2, 3, or 4 carbon atom members replaced by a heteroatom.

“Unsaturated ring” means a ring containing one or more double bonds and include cyclopentene, cyclohexene, cyclopheptene, cyclohexadiene, benzene, pyrroline, pyrazole, 4,5-dihydro-1H-imidazole, imidazole, 1,2,3,4-tetrahydropyridine, 1,2,3,6-tetrahydropyridine, pyridine and pyrimidine.

### Enantiomers, Diastereomers, and Salts

Certain compounds of Formula I may exist in various stereoisomeric or tautomeric forms. The invention encompasses all such forms of the compounds described herein, including an enantiomer or diastereomer thereof. The invention also encompasses active compounds in the form of essentially pure enantiomers, racemic mixtures, and tautomers, including forms those not depicted structurally.

The compounds of the invention may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

The compounds of the invention include pharmaceutically acceptable anionic salt forms, wherein the anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate, and triethiodide salts.

The anionic salt form of a compound of the invention includes the acetate, bromide, camsylate, chloride, edisylate, fumarate, hydrobromide, hydrochloride, iodide, isethionate, lactate, mesylate, maleate, napsylate, salicylate, sulfate, and tosylate salts.

When a disclosed compound or its pharmaceutically acceptable salt is named or depicted by structure, it is to be understood that solvates or hydrates of the compound or its pharmaceutically acceptable salts are also included. “Solvates” refer to crystalline forms wherein solvent molecules are incorporated into the crystal lattice during crystallization. Solvate may include water or nonaqueous solvents such as ethanol, isopropanol, DMSO, acetic acid, ethanolamine, and EtOAc. Solvates, wherein water is the solvent molecule incorporated into the crystal lattice, are typically referred to as “hydrates”. Hydrates include stoichiometric hydrates as well as compositions containing variable amounts of water.

When a disclosed compound or its pharmaceutically acceptable salt is named or depicted by structure, it is to be understood that the compound, including solvates thereof, may exist in crystalline forms, non-crystalline forms or a mixture thereof. The compound or its pharmaceutically acceptable salts or solvates may also exhibit polymorphism (i.e. the capacity to occur in different crystalline forms). These different crystalline forms are typically known as “polymorphs.” It is to be understood that when named or depicted by structure, the disclosed compound and its pharmaceutically acceptable salts, solvates or hydrates also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in solidifying the compound. For example, changes in temperature, pressure, or solvent may result in different polymorphs. In addition, one polymorph may spontaneously convert to another polymorph under certain conditions.

It may be necessary and/or desirable during synthesis to protect sensitive or reactive groups on any of the molecules concerned. Representative conventional protecting groups are described in T. W. Greene and P. G. M. Wuts “Protective Groups in Organic Synthesis” John Wiley & Sons, Inc., New York 1999. Protecting groups may be added and removed using methods well known in the art.

The invention also includes various isomers and mixtures thereof. “Isomer” refers to compounds that have the same composition and molecular weight but differ in physical and/or chemical properties. The structural difference may be in constitution (geometric isomers) or in the ability to rotate the plane of polarized light (stereoisomers).

Certain of the disclosed aspartic protease inhibitors may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. The symbol “*” in a structural formula represents the presence of a chiral carbon center. “R” and “S” represent the configuration of substituents around one or more chiral carbon atoms. Thus, “R*” and “S*” denote the relative configurations of substituents around one or more chiral carbon atoms. When a chiral center is not defined as R or S, a mixture of both configurations is present.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on opposite sides of the carbon-carbon double bond) or Z (substituents are oriented on the same side) configuration.

Atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. A mixture of “cis” and “trans” species is designated “cis/trans”.

The point at which a group or moiety is attached to the remainder of the compound or another group or moiety can be indicated by “ [symbol] ” which represents [symbol] [symbol] or “-”.

“R,” “S,” “S*,” “R*,” “E,” “Z,” “cis,” and “trans,” indicate configurations relative to the core molecule.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a disclosed compound is named or depicted by structure without indicating the stereochemistry, and the inhibitor has at least one chiral center, it is to be understood that the name or structure encompasses one enantiomer of inhibitor free from the corresponding optical isomer, a racemic mixture of the inhibitor and mixtures enriched in one enantiomer relative to its corresponding optical isomer.

When a disclosed aspartic protease inhibitor is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The compounds of the invention are useful for ameliorating or treating disorders or diseases in which decreasing the levels of aspartic protease products is effective in treating the disease state or in treating infections in which the infectious agent depends upon the activity of an aspartic protease. In hypertension elevated levels of angiotensin I, the product of renin catalyzed cleavage of angiotensinogen are present. Thus, the compounds of the invention can be used in the treatment of hypertension, heart failure such as (acute and chronic) congestive heart failure; left ventricular dysfunction; cardiac hypertrophy; cardiac fibrosis; cardiomyopathy (e.g., diabetic cardiac myopathy and post-infarction cardiac myopathy); supraventricular and ventricular arrhythmias; arial fibrillation; atrial flutter; detrimental vascular remodeling; myocardial infarction and its sequelae; atherosclerosis; angina (whether unstable or stable); renal failure conditions, such as diabetic nephropathy; glomerulonephritis; renal fibrosis; scleroderma; glomerular sclerosis; microvascular complications, for example, diabetic retinopathy; renal vascular hypertension; vasculopathy; neuropathy; complications resulting from diabetes, including nephropathy, vasculopathy, retinopathy and neuropathy, diseases of the coronary vessels, proteinuria, albumenuria, post-surgical hypertension, metabolic syndrome, obesity, restenosis following angioplasty, eye diseases and associated abnormalities including raised intra-ocular pressure, glaucoma, retinopathy, abnormal vascular growth and remodelling, angiogenesis-related disorders, such as neovascular age related macular degeneration; hyperaldosteronism, anxiety states, and cognitive disorders (Fisher N. D.; Hollenberg N. K. Expert Opin. Investig. Drugs. 2001, 10, 417-26).

Elevated levels of βamyloid, the product of the activity of the well-characterized aspartic protease β-secretase (BACE) activity on amyloid precursor protein, are widely believed to be responsible for the development and progression of amyloid plaques in the brains of Alzheimer's disease patients. The secreted aspartic proteases of Candida albicans are associated with its pathogenic virulence (Naglik, J. R.; Challacombe, S. J.; Hube, B. Microbiology and Molecular Biology Reviews 2003, 67, 400-428). The viruses HIV and HTLV depend on their respective aspartic proteases for viral maturation. Plasmodium falciparum uses plasmepsins I and II to degrade hemoglobin.

A pharmaceutical composition of the invention may, alternatively or in addition to a compound of Formula I, comprise a pharmaceutically acceptable salt of a compound of Formula I or a prodrug or pharmaceutically active metabolite of such a compound or salt and one or more pharmaceutically acceptable carriers therefor.

The compositions of the invention are aspartic protease inhibitors. Said compositions contain compounds having a mean inhibition constant (IC₅₀) against aspartic proteases of between about 5,000 nM to about 0.01 nM; preferably between about 50 nM to about 0.01 nM; and more preferably between about 5 nM to about 0.01 nM.

The compositions of the invention reduce blood pressure. Said compositions include compounds having an IC₅₀ for renin of between about 5,000 nM to about 0.01 nM; preferably between about 50 nM to about 0.01 nM; and more preferably between about 5 nM to about 0.01 nM.

The invention includes a therapeutic method for treating or ameliorating an aspartic protease mediated disorder in a subject in need thereof comprising administering to a subject in need thereof an effective amount of a compound of Formula I, or the enantiomers, diastereomers, or salts thereof or composition thereof.

Administration methods include administering an effective amount (i.e., a therapeutically effective amount) of a compound or composition of the invention at different times during the course of therapy or concurrently in a combination form. The methods of the invention include all known therapeutic treatment regimens.

“Prodrug” means a pharmaceutically acceptable form of an effective derivative of a compound (or a salt thereof) of the invention, wherein the prodrug may be: 1) a relatively active precursor which converts in vivo to a compound of the invention; 2) a relatively inactive precursor which converts in vivo to a compound of the invention; or 3) a relatively less active component of the compound that contributes to therapeutic activity after becoming available in vivo (i.e., as a metabolite). See “Design of Prodrugs”, ed. H. Bundgaard, Elsevier, 1985.

“Metabolite” means a pharmaceutically acceptable form of a metabolic derivative of a compound (or a salt thereof) of the invention, wherein the derivative is an active compound that contributes to therapeutic activity after becoming available in vivo.

“Effective amount” means that amount of active compound agent that elicits the desired biological response in a subject. Such response includes alleviation of the symptoms of the disease or disorder being treated. The effective amount of a compound of the invention in such a therapeutic method is from about 10 mg/kg/day to about 0.01 mg/kg/day, preferably from about 0.5 mg/kg/day to 5 mg/kg/day.

The invention includes the use of a compound of the invention for the preparation of a composition for treating or ameliorating an aspartic protease mediated chronic disorder or disease or infection in a subject in need thereof, wherein the composition comprises a mixture one or more compounds of the invention and an optional pharmaceutically acceptable carrier.

“Pharmaceutically acceptable carrier” means compounds and compositions that are of sufficient purity and quality for use in the formulation of a composition of the invention and that, when appropriately administered to an animal or human, do not produce an adverse reaction.

“Aspartic protease mediated disorder or disease” includes disorders or diseases associated with the elevated expression or overexpression of aspartic proteases and conditions that accompany such diseases.

An embodiment of the invention includes administering a renin inhibiting compound of this invention or composition thereof in a combination therapy (U.S. Pat. No. 5,821,232, U.S. Pat. No. 6,716,875, U.S. Pat. No. 5,663,188, Fossa, A. A.; DePasquale, M. J.; Ringer, L. J.; Winslow, R. L. “Synergistic effect on reduction in blood pressure with coadministration of a renin inhibitor or an angiotensin-converting enzyme inhibitor with an angiotensin II receptor antagonist” Drug Development Research 1994, 33(4), 422-8) with one or more additional agents for the treatment of hypertension including α-blockers, β-blockers, calcium channel blockers, diuretics, natriuretics, saluretics, centrally acting antihypertensives, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist. α-Blockers include doxazocin, prazosin, tamsulosin, and terazosin. β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts. Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts. A diuretic is, for example, a thiazide derivative selected from amiloride, chlorothiazide, hydrochlorothiazide, methylchlorothiazide, and chlorothalidon. ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril. Dual ACE/NEP inhibitors are, for example, omapatrilat, fasidotril, and fasidotrilat. Preferred ARBs include candesartan, eprosartan, irbesartan, losartan, olmesartan, tasosartan, telmisartan, and valsartan. Preferred aldosterone synthase inhibitors are anastrozole, fadrozole, and exemestane. Preferred aldosterone-receptor antagonists are spironolactone and eplerenone. A preferred endothelin antagonist is, for example, bosentan, enrasentan, atrasentan, darusentan, sitaxentan, and tezosentan and their pharmaceutically acceptable salts.

An embodiment of the invention includes administering an HIV protease inhibiting compound of this invention or composition thereof in a combination therapy with one or more additional agents for the treatment of AIDS including reverse transcriptase inhibitors, non-nucleoside reverse transcriptase inhibitors, other HIV protease inhibitors, HIV integrase inhibitors, attachment and fusion inhibitors, antisense drugs and immune stimulators. Preferred reverse transcriptase inhibitors are zidovudine, didanosine, zalcitabine, stavudine, lamivudine, abacavir, tenofovir, and emtricitabine. Preferred non-nucleoside reverse transcriptase inhibitors are nevirapine, delaviridine, and efavirenz. Preferred HIV protease inhibitors are saquinavir, ritonavir, indinavir, nelfinavir, amprenavir, lopinavir, atazanavir, and fosamprenavir. Preferred HIV integrase inhibitors are L-870,810 and S-1360. A preferred attachment and fusion inhibitor is enfuvirtide.

An embodiment of the invention includes administering β-secretase inhibiting compound of this invention or composition thereof in a combination therapy with one or more additional agents for the treatment of Alzheimer's disease including tacrine, donepezil, rivastigmine, galantamine, and memantine.

An embodiment of the invention includes administering a plasmepsin inhibiting compound of this invention or composition thereof in a combination therapy with one or more additional agents for the treatment of malaria including artemisinin, chloroquine, halofantrine, hydroxychloroquine, mefloquine, primaquine, pyrimethamine, quinine, sulfadoxine.

Combination therapy includes co-administration of the compound of the invention and said other agent, sequential administration of the compound and the other agent, administration of a composition containing the compound and the other agent, or simultaneous administration of separate compositions containing of the compound and the other agent.

The invention further includes the process for making the composition comprising mixing one or more of the present compounds and an optional pharmaceutically acceptable carrier; and includes those compositions resulting from such a process, which process includes conventional pharmaceutical techniques.

The compositions of the invention include ocular, oral, nasal, transdermal, topical with or without occlusion, intravenous (both bolus and infusion), and injection (intraperitoneally, subcutaneously, intramuscularly, intratumorally, or parenterally). The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The compositions of the invention may be administered in a form suitable for once-weekly or once-monthly administration. For example, an insoluble salt of the active compound may be adapted to provide a depot preparation for intramuscular injection (e.g., a decanoate salt) or to provide a solution for ophthalmic administration.

The dosage form containing the composition of the invention contains a therapeutically effective amount of the active ingredient necessary to provide a therapeutic effect. The composition may contain from about 5,000 mg to about 0.5 mg (preferably, from about 1,000 mg to about 0.5 mg) of a compound of the invention or salt form thereof and may be constituted into any form suitable for the selected mode of administration. The composition may be administered about 1 to about 5 times per day. Daily administration or post-periodic dosing may be employed.

For oral administration, the composition is preferably in the form of a tablet or capsule containing, e.g., 500 to 0.5 milligrams of the active compound. Dosages will vary depending on factors associated with the particular patient being treated (e.g., age, weight, diet, and time of administration), the severity of the condition being treated, the compound being employed, the mode of administration, and the strength of the preparation.

The oral composition is preferably formulated as a homogeneous composition, wherein the active ingredient is dispersed evenly throughout the mixture, which may be readily subdivided into dosage units containing equal amounts of a compound of the invention. Preferably, the compositions are prepared by mixing a compound of the invention (or pharmaceutically acceptable salt thereof) with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binder agents include starch, gelatin, natural sugars (e.g., glucose and beta-lactose), corn sweeteners and natural and synthetic gums (e.g., acacia and tragacanth). Disintegrating agents include starch, methyl cellulose, agar, and bentonite.

Tablets and capsules represent an advantageous oral dosage unit form. Tablets may be sugarcoated or filmcoated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

Compounds of the invention may also be administered via a slow release composition; wherein the composition includes a compound of the invention and a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain an active agent(s) and which slowly degrade/dissolve in a suitable environment (e.g., aqueous, acidic, basic and the like) to release the agent. Such particles degrade/dissolve in body fluids to release the active compound(s) therein. The particles are preferably nanoparticles (e.g., in the range of about 1 to 500 nm in diameter, preferably about 50-200 nm in diameter, and most preferably about 100 nm in diameter). In a process for preparing a slow release composition, a slow release carrier and a compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

The compound of Formula I may be incorporated for administration orally or by injection in a liquid form such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The compounds may be administered parenterally via injection. A parenteral formulation may consist of the active ingredient dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the active ingredient in the liquid carrier whereby the final dosage unit contains from 0.005 to 10% by weight of the active ingredient. Other additives include preservatives, isotonizers, solubilizers, stabilizers, and pain-soothing agents. Injectable suspensions may also be prepared, in which case appropriate liquid carriers, suspending agents and the like may be employed.

Compounds of the invention may be administered intranasally using a suitable intranasal vehicle.

Compounds of the invention may also be administered topically using a suitable topical transdermal vehicle or a transdermal patch.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the compound of the invention, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration n the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4 to 8.

In the discussion below R, R¹, R², R³, X, Y, A, Q, E, and G are defined as described above for compounds of Formula I. In cases where the synthetic intermediates and final products of Formula I described below contain potentially reactive functional groups, for example amino, hydroxyl, thiol and carboxylic acid groups, that may interfere with the desired reaction, it may be advantageous to employ protected forms of the intermediate. Methods for the selection, introduction and subsequent removal of protecting groups are well known to those skilled in the art. (T. W. Greene and P. G. M. Wuts “Protective Groups in Organic Synthesis” John Wiley & Sons, Inc., New York 1999). In the discussion below all intermediates are assumed to be protected when necessary and protection/deprotection are generally not described.

In the first process of the invention, a compound of Formula I, in which a nitrogen atom that is part of A is attached to Q, is prepared by reaction of an amine of Formula II and an intermediate of Formula III:

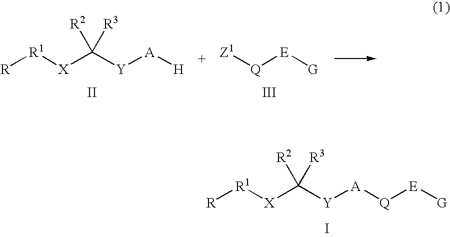

wherein Z¹ in III is a leaving group such as halide, alkanesulfonate, haloalkanesulfonate, carboxylate, arylsulfonate, aryloxy, heteroaryloxy, azole, azolium salt, alkoxy, alkylthio, or arylthio.

Intermediates of formula II wherein H is attached to a nitrogen atom that is part of A are prepared from intermediates of Formula IV:

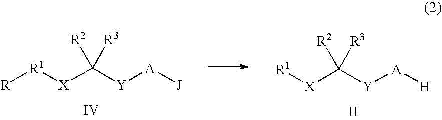

wherein J is an amine protecting group, including carbamate, amide, and sulfonamide protecting groups known in the art (T. W. Greene and P. G. M. Wuts “Protective Groups in Organic Synthesis” John Wiley & Sons, Inc., New York 1999).

Intermediates of Formula IV wherein R³═OH are prepared from ketone intermediates of formula V by addition of an organometallic reagent of formula VI, where M is for example Li, MgCl, MgBr, or MgI, to the carbonyl group of V:

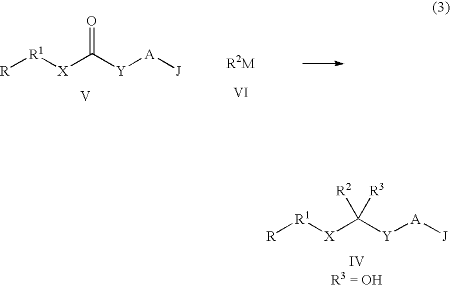

Intermediates of Formula IV wherein R³═H and R² is a group attached by an ether linkage are prepared from alcohol intermediates of formula VII by reaction with an alkylating agent under basic conditions.

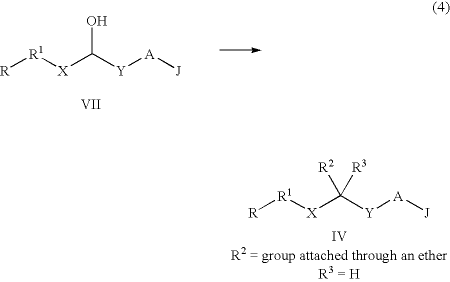

Alcohol intermediates of formula VII are prepared by reduction of ketone intermediates of formula V:

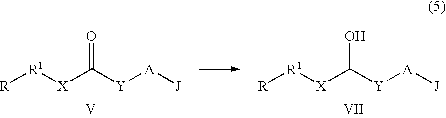

or by addition of an organometallic reagent of formula VIII, wherein M is, for example Li, MgCl, MgBr, or MgI, to an aldehyde of Formula IX:

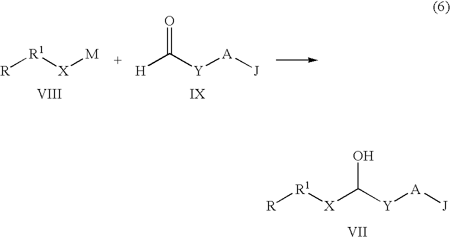

Ketone intermediates of formula V are prepared by the addition of an organometallic reagent of formula VIII, wherein M is, for example Li, MgCl, MgBr, MgI, to a carboxylic acid derivative of formula X wherein Z² is an alkoxy, dialkylamino group, or an N-alkoxy-N-alkylamino group:

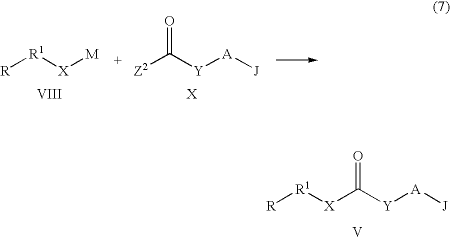

Intermediates of Formula III, wherein Q is Q1 attached to a carbon atom of E and Z¹ is alkanesulfonate, haloalkanesulfonate, carboxylate, arylsulfonate, or represents an active ester are prepared by activation of carboxylic acids of Formula XV:

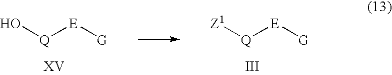

Reagents used to effect carboxylic activation are well known in the literature and include thionyl chloride and oxalyl chloride used to prepare acid chlorides, alkanesulfonyl chlorides used to prepare mixed anhydrides, alkyl chloroformates used to prepare mixed anhydrides, and carbodiimides used to prepare active esters. Intermediates of formula III are often prepared and used in situ without isolation.

In the first process of the invention, a compound of Formula Ia, in which A¹ is a nitrogen atom is prepared by reaction of an amine of Formula IIa and an intermediate of Formula IIa:

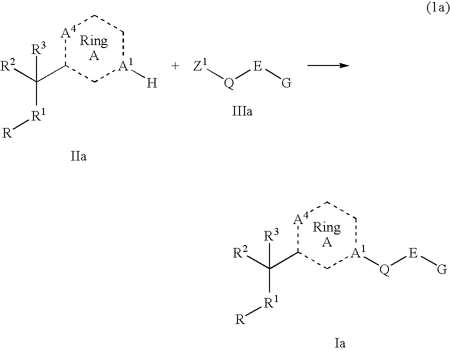

wherein Z¹ in IIIa is a leaving group such as halide, alkanesulfonate, haloalkanesulfonate, carboxylate, arylsulfonate, aryloxy, heteroaryloxy, azole, azolium salt, alkoxy, alkylthio, or arylthio.

Intermediates of formula IIa in which A¹ is a nitrogen atom are prepared from intermediates of Formula IVa:

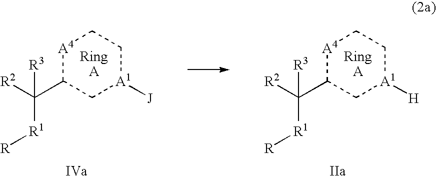

wherein J is an amine protecting group, including carbamate, amide and sulfonamide protecting groups known in the art (T. W. Greene and P. G. M. Wuts “Protective Groups in Organic Synthesis” John Wiley & Sons, Inc., New York 1999).

Intermediates of Formula IVa wherein R³═OH are prepared from ketone intermediates of formula Va by addition of an organometallic reagent of formula VIa, where M is for example Li, MgCl, MgBr, or MgI, to the carbonyl group of Va:

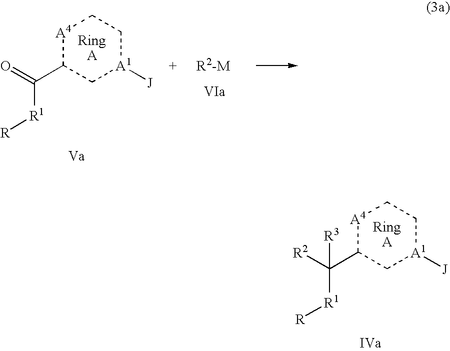

Intermediates of Formula IVa wherein R³═H and R² is a group attached by an ether linkage are prepared from alcohol intermediates of formula VIIa by reaction with an alkylating agent under basic conditions.

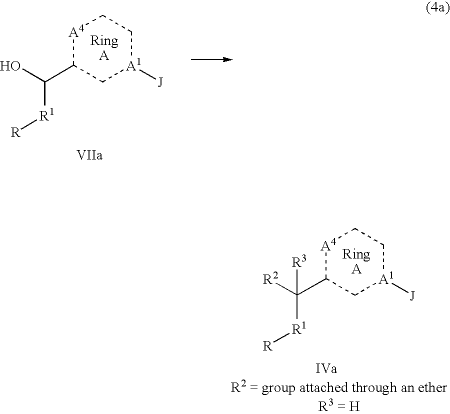

Alcohol intermediates of formula VIIa are prepared by reduction of ketone intermediates of formula Va using reagents known in the art (Handbook of Reagents for Organic Synthesis: Oxidizing and Reducing Reagents Ed. S. D. Burke and R. L. Danheiser, John Wiley & Sons, New York, 1999):

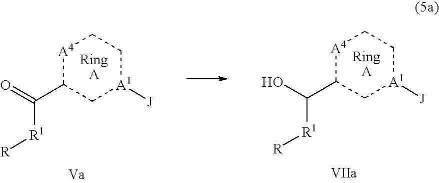

or by addition of an organometallic reagent of formula VIIIa, wherein M is, for example Li, MgCl, MgBr, or MgI, to an aldehyde of Formula IXa:

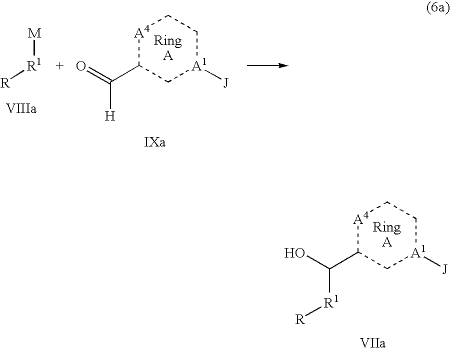

Ketone intermediates of formula Va are prepared by the addition of an organometallic reagent of formula VIIIa, wherein M is Li, MgCl, MgBr, MgI, to a carboxylic acid derivative of formula Xa wherein Z² is an alkoxy, dialkylamino group, or an N-alkoxy-N-alkylamino group:

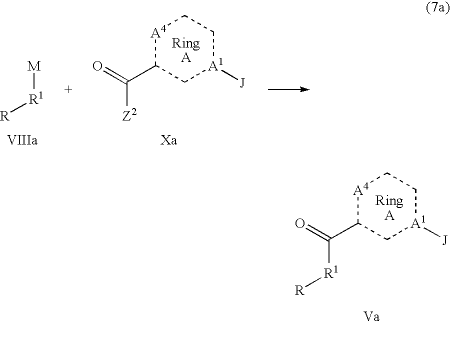

Intermediates of formula Va are also prepared by oxidation of alcohol intermediates of formula VIIa using reagents known in the art (Handbook of Reagents for Organic Synthesis: Oxidizing and Reducing Reagents Ed. S. D. Burke and R. L. Danheiser, John Wiley & Sons, New York, 1999):

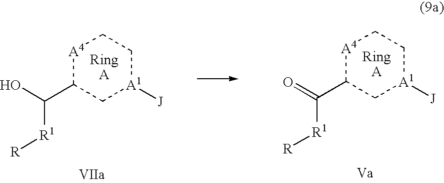

Intermediates of Formula Ma, wherein Q is Q1 attached to a carbon atom of E and Z¹ is alkanesulfonate, haloalkanesulfonate, carboxylate, arylsulfonate, or represents an active ester are prepared by activation of carboxylic acids of Formula XVa:

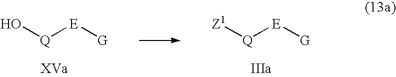

Reagents used to effect carboxylic activation are well known in the literature and include thionyl chloride and oxalyl chloride used to prepare acid chlorides, alkanesulfonyl chlorides used to prepare mixed anhydrides, alkyl chloroformates used to prepare mixed anhydrides, and carbodiimides used to prepare active esters. Intermediates of formula IIIa are often prepared and used in situ without isolation.

The invention is further defined by reference to the examples, which are intended to be illustrative and not limiting.

Representative compounds of the invention can be synthesized in accordance with the general synthetic schemes described above and are illustrated in the examples that follow. The methods for preparing the various starting materials used in the schemes and examples are well within the knowledge of persons skilled in the art. During the course of preparing aryl 3-piperidinyl ketones, as described in the following protocols (e.g. Preparations 5-7, 13 and 15), racemization of the stereocenter adjacent to the carbonyl group can occur and was specifically observed during the preparation of (R)-tert-butyl 3-(6-chloro-3′-ethylbiphenylcarbonyl)piperidine-1-carboxylate. In this case, the racemic product was detected when the reaction mixture was allowed to stir at room temperature for prolonged times (e.g. overnight) but was not observed when the ketone forming reaction was quenched at −78° C. (by addition of aqueous ammonium chloride). When racemization does occur, the resulting stereoisomers may be resolved using conventional methods well known to those skilled in the art. Accordingly, it will be appreciated by those skilled in the art, that in the following Experimental section, any identification of a specific stereoisomer (e.g., assignment of configuration of a chiral center) in a final or intermediate product compound name or structure is to be understood to represent the intended relative or absolute configuration of that chiral center, but not necessarily the only stereoisomer obtained.

The following abbreviations have the indicated meanings

| Abbreviation | Meaning |
|---|---|
| aq | aqueous |
| Boc | tert-butoxy carbonyl or t-butoxy carbonyl |
| (Boc)₂O | di-tert-butyl dicarbonate |
| brine | saturated aqueous NaCl |
| CH₂Cl₂ | methylene chloride |
| CH₃CN | acetonitrile |
| or MeCN | |
| Cpd | compound |
| d | day |
| DBU | 1,8-diazabicyclo[5.4.0]undec-7-ene |
| DIEA | N,N-diisopropylethylamine |
| DMAP | 4-(dimethylamino)pyridine |
| DMF | N,N-dimethylformamide |
| DMPU | 1,3-dimethyl-3,4,5,6-tetrahydro-2(1H)-pyrimidinone |
| EDC•HCl | 1-[3-(dimethylamino)propyl]-3-ethylcarbodiimide |
| | hydrochloride |
| equiv | equivalents |
| Et | ethyl |
| Et₂O | ethyl ether |
| EtOAc | ethyl acetate |
| Fmoc | 1-[[(9H-fluoren-9-ylmethoxy)carbonyl]oxy]- |
| Fmoc-OSu | 1-[[(9H-fluoren-9-ylmethoxy)carbonyl]oxy]-2,5- |
| | pyrrolidinedione |
| h, hr | hour |
| HOBt | 1-hydroxybenzotriazole |
| HATU | 2-(7-Aza-1H-benzotriazole-1-yl)-1,1,3,3- |
| | tetramethyluronium hexafluorophosphate |
| HBTU | 2-(1H-Benzotriazole-1-yl)-1,1,3,3-tetramethyluronium |
| | hexafluorophosphate |
| KHMDS | potassium hexamethyldisilazane |
| LAH or | lithium aluminum hydride |
| LiAlH₄ | |
| LC-MS | liquid chromatography-mass spectroscopy |
| LHMDS | lithium hexamethyldisilazane |
| Me | methyl |
| MeCN | acetonitrile |
| MeOH | methanol |
| MsCl | methanesulfonyl chloride |
| min | minute |
| MS | mass spectrum |
| NaH | sodium hydride |
| NaHCO₃ | sodium bicarbonate |
| NaN₃ | sodium azide |
| NaOH | sodium hydroxide |
| Na₂SO₄ | sodium sulfate |
| NMP | N-methylpyrrolidinone |
| P₄-t-Bu | 1-tert-butyl-4,4,4-tris(dimethylamino)-2,2- |
| | bis[tris(dimethylamino)-phosphoranylidenamino]-2Λ⁵, |
| | 4Λ⁵-catenadi(phosphazene) |
| Pd₂(dba)₃ | tris(dibenzylideneacetone)dipalladium(0) |
| Ph | phenyl |
| rt | room temperature |
| satd | saturated |
| SOCl₂ | thionyl chloride |
| TBAF | tetrabutylammonium fluoride |
| TEA | triethylamine or Et₃N |
| TEAF | tetraethylammonium fluoride |
| TEMPO | 2,2,6,6-tetramethyl-1-piperidinyloxy, free radical |
| Teoc | 1-[2-(trimethylsilyl)ethoxycarbonyloxy]- |
| Teoc-OSu | 1-[2-(trimethylsilyl)ethoxycarbonyloxy]pyrrolidin-2,5-dione |
| TFA | trifluoroacetic acid |
| THF | tetrahydrofuran |
| TMSCl | chlorotrimethylsilane or trimethylsilyl chloride |
| t_{R} | retention time |

### LC-MS Methods

Method 1 [Instrument 1]: Analytical LC-MS was conducted on an Agilent 1100 Series LC/MSD SL or VL using electrospray positive [ES+ve to give MH⁺] equipped with a Sunfire C₁₈ 5.0 μm column (3.050 mm×50 3.0 mm, i.d.), eluting with 0.05% TFA in water (solvent A) and 0.05% TFA in acetonitrile (solvent B), using the following elution gradient 10%-99% (solvent B) over 3.0 min and holding at 99% for 1.0 min at a flow rate of 1.0 ml/min.

Method 2 [Instrument 2]: Analytical LC-MS was conducted on an PE Sciex API 150 single quadrupole mass spectrometer using electrospray positive [ES+ve to give MH+] equipped with a Aquasil C18 5 μm column (1 mm×40 mm), eluting with 0.02% TFA in water (solvent A) and 0.018% TFA in acetonitrile (solvent B), using the following elution gradient 4.5%-90% (solvent B) over 3.2 min and holding at 90% for 0.4 min at a flow rate of 0.3 ml/min.

Method 3: Analytical LC-MS was conducted on an Agilent 1200 Series LC/MSD VL using electrospray positive [ES+ve to give MH⁺] equipped with a YMC C₁₈ 5.0 μm column (2.0 mm×50, 2.0 mm, i.d.), eluting with 0.0375% TFA in water (solvent A) and 0.01875% TFA in acetonitrile (solvent B), using the following elution gradient 10%-80% (solvent B) over 2.0 min and holding at 80% for 0.5 min at a flow rate of 1.0 ml/min.

### Chiral HPLC Method

Column: Chiralpak AD-H, 0.46 cm×25 cm

### Solvent A: 0.025% Diethylamine in Hexane

### Solvent B: Isopropanol

Flow rate: 1 mL/min.
40 min. run

### Gradient:

| Time (min) | A (%) | B (%) |
|---|---|---|
| 0 | 95 | 5 |
| 40 | 90 | 10 |

The following procedures describe preparation of intermediates used in the synthesis of compounds of Formula I

### Preparation 1

### Weinreb Amide

### (R)-tert-butyl 3-(N-methoxy-N-methylcarbamoyl)piperidine-1-carboxylate

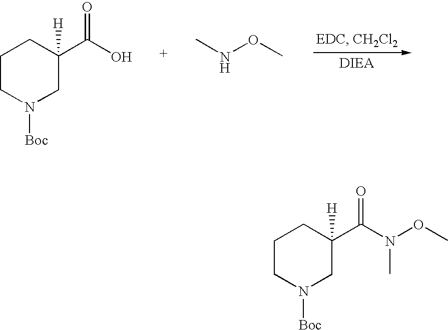

(R)-1-(tert-butoxy carbonyl)piperidine-3-carboxylic acid (25 g, 0.11 mol, 1.0 equiv), N,O-dimethylhydroxylamine hydrochloride, (10.5 g, 0.14 mol, 1.25 equiv), EDC.HCl (26.3 g, 0.14 mol, 1.25 equiv) and DIEA (48 mL, 0.28 mol, 2.5 equiv) were dissolved in CH₂Cl₂ (400 mL) and stirred overnight at rt. The reaction mixture was diluted with EtOAc, washed with 5% aq HCl (2×150 mL), satd aq NaHCO₃ (150 mL), brine (100 mL), and dried over Na₂SO₄. Concentration afforded (R)-tert-butyl 3-(N-methoxy-N-methylcarbamoyl)-piperidine-1-carboxylate (24.42 g, 82%) as a clear oil.

### Preparation 2

### Halodiphenyl Ethers from Halophenols and Benzeneboronic Acids

### 1-(3-Fluorophenoxy)-2-bromobenzene

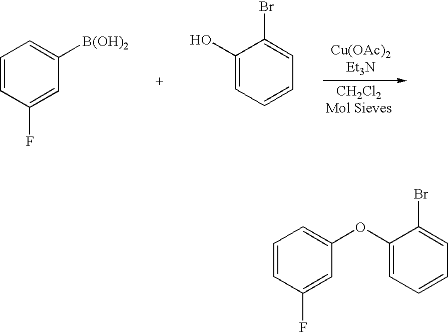

To a stirring solution of 3-fluorophenylboronic acid (2.10 g, 15 mmol), 2-bromophenol (1.77 g, 10 mmol) and Cu(OAc)₂ (0.93 g, 5 mmol) in anhydrous CH₂Cl₂ (25 mL) is added activated 4 Å molecular sieves (˜0.1 g), followed by anhydrous Et₃N (3.5 mL, 25 mmol). The resulting dark green solution is stirred at rt for 48 h. The mixture is evaporated under reduced pressure and the residue washed several times with Et₂O (˜150 mL). The Et₂O solution is washed with satd aq NH₄Cl and 1 N aq HCl. The organic layer is evaporated and the crude product is purified by flash column chromatography to give 1-(3-fluorophenoxy)-2-bromobenzene (1.28 g, 48%) as clear oil.

The following halodiphenyl ethers were prepared following the procedure described above.

| Halodiphenyl ether | Phenol | Boronic Acid |
|---|---|---|
| 1-bromo-3-chloro-2-[(2- | 2-bromo-6-chlorophenol | 2-methylphenylboronic acid |
| methylphenyl)oxy]benzene | | |
| 1-bromo-3-chloro-2-[(2- | 2-bromo-6-chlorophenol | 2-ethylphenylboronic acid |
| ethylphenyl)oxy]benzene | | |
| 1-bromo-3-chloro-2-[(3- | 2-bromo-6-chlorophenol | 3-ethylphenylboronic acid |
| ethylphenyl)oxy]benzene | | |
| 1-bromo-2-[(3-methylphenyl)oxy]- | 2-bromo-6-fluorophenol | (3-methylphenyl)boronic acid |
| 3-fluorobenzene | | |
| 1-bromo-3-chloro-2-[(3- | 2-bromo-6-chlorophenol | (3-methylphenyl)boronic acid |
| methylphenyl)oxy]benzene | | |

### Preparation 3

### Halodiphenyl Ethers from Phenoxyanilines

### 1-(O-tolyloxy)-2-iodobenzene

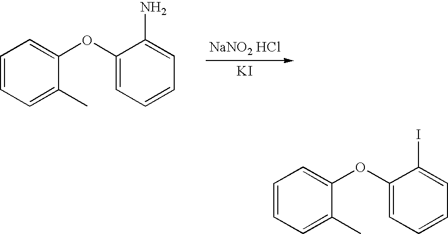

To a solution of 2-(o-tolyloxy)aniline (40 g, 0.2 mol) in 1N aq HCl (400 mL, 0.4 mol, 2 equiv) cooled to 0° C. was added dropwise a solution of NaNO₂ (18 g, 0.26 mol, 1.3 equiv) in water (520 ml). The mixture was stirred for 1 h at 0° C. and a solution of KI (83 g, 0.5 mol, 2.5 equiv) in water (500 mL) was added dropwise with vigorous stirring. After 0.5 h the mixture was warmed to 90-100° C. for 1 h, cooled to rt and washed with satd NaHSO₃ until the aqueous layer became clear. The mixture was extracted with EtOAc (3×200 mL) and the combined organic layers were washed with aq Na₂S₂O₄ and dried over Na₂SO₄. After evaporation of the solvent, the solution was passed through a short silica gel column to afford 1-(o-tolyloxy)-2-iodobenzene (40.0 g, 65%).

### Preparation 4

### Halodiphenyl Ethers from Phenols and Fluoronitrobenzenes

### 1-(2-Iodophenoxy)-2-chlorobenzene

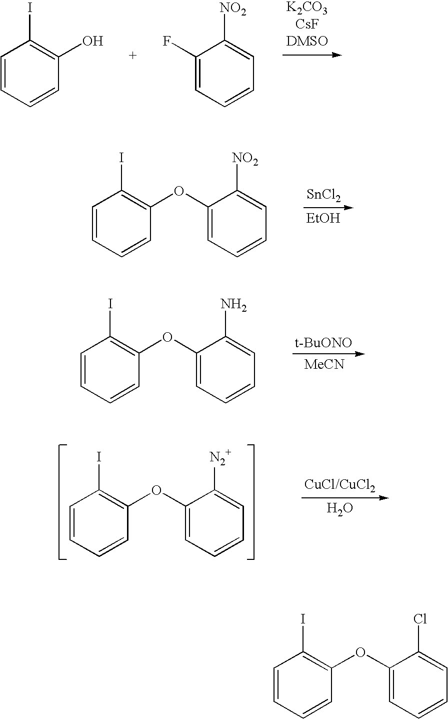

### Step 1. 1-(2-Iodophenoxy)-2-nitrobenzene

To a solution of 2-iodophenol (11.82 g, 52.7 mmol) and 1-fluoro-2-nitrobenzene (5.0 g, 35.1 mmol) in DMSO (50 mL) is added K₂CO₃ (14.5 g, 105.3 mmol), followed by CsF (8.0 g, 52.7 mmol). The resulting suspension is stirred at 50° C. until no starting material remains (˜5 h), cooled to rt and partitioned between water (50 mL) and CH₂Cl₂ (50 mL). The water layer is separated and extracted with CH₂Cl₂ (2×10 mL). The combined organic layers are washed with 1 aq N NaOH (10 mL) and brine, and dried over Na₂SO₄. Solvent is removed under vacuum to give 1-(2-iodophenoxy)-2-nitrobenzene (11.2 g, 93%) as an oil, which can be used for the next step without purification.

### Step 2. 2-(2-Iodophenoxy)benzenamine

A solution of 1-(2-iodophenoxy)-2-nitrobenzene (9.60 g, 28.1 mmol) and SnCl.2H₂O (13.0 g, 56.0 mmol) in ethanol (25 mL) and water (5 mL) is refluxed until no starting material remains (˜1 h). The ethanol is removed in vacuo and the aq layer is basified to pH>10 and extracted with CH₂Cl₂ (4×10 mL). The combined organic layers are dried over Na₂SO₄, and the solvent is removed to give a crude 2-(2-Iodophenoxy)benzenamine (8.57 g, 98%), which can be used for the next step without purification.

### Step 3. 1-(2-Iodophenoxy)-2-chlorobenzene

A solution of crude 2-(2-iodophenoxy)benzenamine (8.57 g, 27.6 mmol) in MeCN (60 mL) is cooled to 0° C. and treated with HBF₄ (54 wt % in Et₂O, 4.93 mL, 35.9 mmol). The reaction mixture is stirred at 0° C. for 5 min and t-BuONO (4.10 g, 35.9 mmol) is added dropwise. The resulting mixture is stirred at 0° C. for 10 min, cooled to −20° C., and added to a solution of CuCl (41 g, 414.1 mmol) and CuCl₂ (70 g, 414.1 mmol) in water (500 mL) at 0° C. The mixture is stirred vigorously at 25° C. for 2 h, and partitioned between EtOAc and water. The water layer is extracted with EtOAc (3×10 mL) and the combined organic layers are washed with brine, dried over Na₂SO₄ and concentrated under vacuum. Flash column chromatography gives 1-(2-iodophenoxy)-2-chlorobenzene (5.35 g, 58%).

The following halodiphenyl ethers were prepared following the procedures described above using the starting materials and reagents indicated:

| | Phenol in | |
|---|---|---|
| Halopdiphenyl ether | Step 1 | Halide in Step 3 |
| 2-[(2-bromophenyl)oxy]-1-chloro-3- | 2-chloro-6- | CuBr/CuBr₂ |
| methylbenzene | methylphenol | |
| 1-bromo-3-chloro-2-[(2- | 2-methylphenol | CuBr/CuBr₂ |
| methylphenyl)oxy]benzene | | |

### Preparation 5

### Piperidines from Weinreb Amides and Halodiphenylethers

### (S)-1-(2-(3-Fluorophenoxy)phenyl)-5-methoxy-1-((R)-piperidin-3-yl)pentan-1-ol

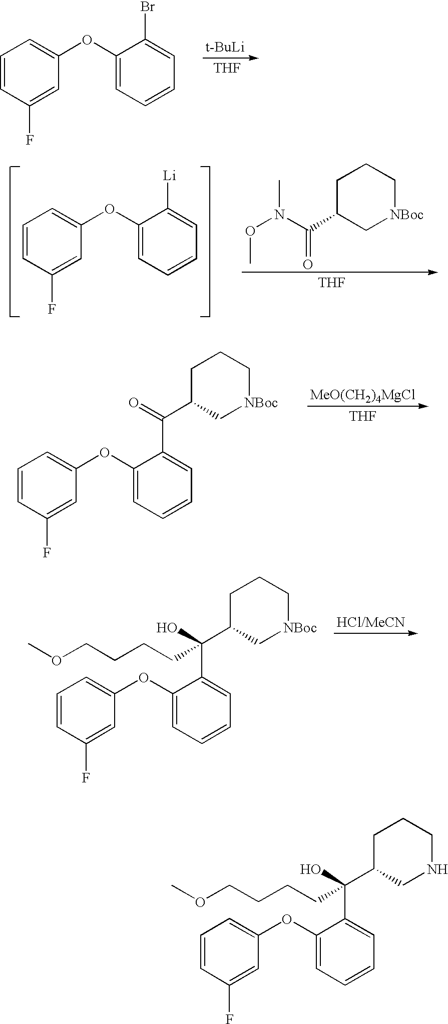

### Step 1. 2-(3-Fluorophenoxy)phenyllithium

To a stirred solution of 1-(3-fluorophenoxy)-2-bromobenzene (1.27 g, 4.75 mmol) in THF (10 mL) at −70° C. is added 1.7 M t-BuLi in pentane (5.6 mL, 9.50 mmol) dropwise to keep the temperature below −70° C. The resulting solution is stirred at −70° C. for 30 min, and this can be used for the next step directly.

### Step 2. (3R)-1-(tert-butoxycarbonyl)-3-((3-fluorophenoxy)benzoyl)piperidine

To a solution of (R)-tert-butyl 3-(N-methoxy-N-methylcarbamoyl)piperidine-1-carboxylate (0.65 g, 2.37 mmol) in THF (4 mL) at −20° C. is added dropwise the solution of 2-(3-fluorophenoxy)phenyllithium that is prepared in Step 2 above. After the addition is complete, the resulting solution is allowed to warm to rt slowly, and left at rt for 1 h. The reaction is quenched with 1N HCl (˜6 mL) and extracted with Et₂O (4×10 mL). The combined organic layers are washed with satd aq NaHCO₃ and brine, and dried over Na₂SO₄. Removal of the solvent left the crude ketone (1.49 g, quantitative), which is used for next step without further purification.

### Step 3. (3R)-tert-butyl 3-(1-(2-(3-fluorophenoxy)phenyl)-1-hydroxy-5-methoxypentyl)piperidine-1-carboxylate

To a solution of (3R)-1-(tert-butoxycarbonyl)-3-((3-fluorophenoxy)benzoyl)piperidine (0.95 g, 2.37 mmol) in THF (3 mL) at −20° C. is added 1.45 M 4-methoxybutyl magnesium chloride in THF (3.3 mL, 4.76 mmol) dropwise. The resulting solution is warmed to rt slowly, and the completion of reaction is confirmed by LC-MS (˜20 min). The reaction is quenched with satd aq NH₄Cl (4 mL) and extracted with Et₂O (4×5 mL). The combined organic layers are washed with water and brine, and the solvent is removed in vacuo to give a crude product which is purified by flash column chromatography to afford (3R)-tert-butyl 3-(1-(2-(3-fluorophenoxy)phenyl)-1-hydroxy-5-methoxypentyl)piperidine-1-carboxylate (0.50 g, 43%).

### Step 4. 1-(2-(3-Fluorophenoxy)phenyl)-5-methoxy-1-((R)-piperidin-3-yl)pentan-1-ol

To a solution of (3R)-tert-butyl 3-(1-(2-(3-fluorophenoxy)phenyl)-1-hydroxy-5-methoxypentyl)piperidine-1-carboxylate (0.50 g, 1.03 mmol) in MeCN (60 mL) is added 2 N aq HCl (60 mL) slowly at rt. The resulting solution is stirred at rt overnight, then basified to pH=10 with 10 N aq NaOH. The mixture is evaporated under reduced pressure to remove MeCN. The aq layer is extracted with CH₂Cl₂ (4×10 mL), and the combined organic layers are washed with brine and dried over Na₂SO₄. The solvent is removed under vacuum to give (S)-1-(2-(3-fluorophenoxy)phenyl)-5-methoxy-1-((R)-piperidin-3-yl)pentan-1-ol (0.40 g, quantitative) as a free amine.

The following piperidines prepared using the above procedures using the halodiphenyl ethers listed below in Step 1.

| Piperidine | Halodiphenyl ether |
|---|---|
| 1-(3-chloro-2-(o-tolyloxy)phenyl)-5- | 1-bromo-3-chloro-2-(o- |
| methoxy-1-((R)-piperidin-3-yl)pentan-1-ol | tolyloxy)benzene |
| 1-(2-(2-chloro-6-methylphenoxy)phenyl)-5- | 2-(2-bromophenoxy)-1- |
| methoxy-1-((R)-piperidin-3-yl)pentan-1-ol | chloro-3-methylbenzene |
| 1-(3-chloro-2-(2-ethylphenoxy)phenyl)-5- | 1-bromo-3-chloro-2-(2- |
| methoxy-1-((R)-piperidin-3-yl)pentan-1-ol | ethylphenoxy)benzene |
| 1-(3-chloro-2-(3-ethylphenoxy)phenyl)-5- | 1-bromo-3-chloro-2-(3- |
| methoxy-1-((R)-piperidin-3-yl)pentan-1-ol | ethylphenoxy)benzene |
| 1-{3-chloro-2-[(3-methylphenyl)oxy]- | 1-bromo-3-chloro-2-[(3- |
| phenyl}-5-(methyloxy)-1-[(3R)-3- | methylphenyl)oxy]benzene |
| piperidinyl]-1-pentanol | |
| 1-{3-fluoro-2-[(3-methylphenyl)oxy]- | 1-bromo-3-fluoro-2-[(3- |
| phenyl}-5-(methyloxy)-1-[(3R)-3- | methylphenyl)oxy]benzene |
| piperidinyl]-1-pentanol | |

### Preparation 6

### Alternate Piperidines from Weinreb Amides And Halodiphenylethers

### Methyl {4-{3-fluoro-2-[(3-methylphenyl)oxy]phenyl}-4-hydroxy-4-[(3R)-3-piperidinyl]butyl}carbamate

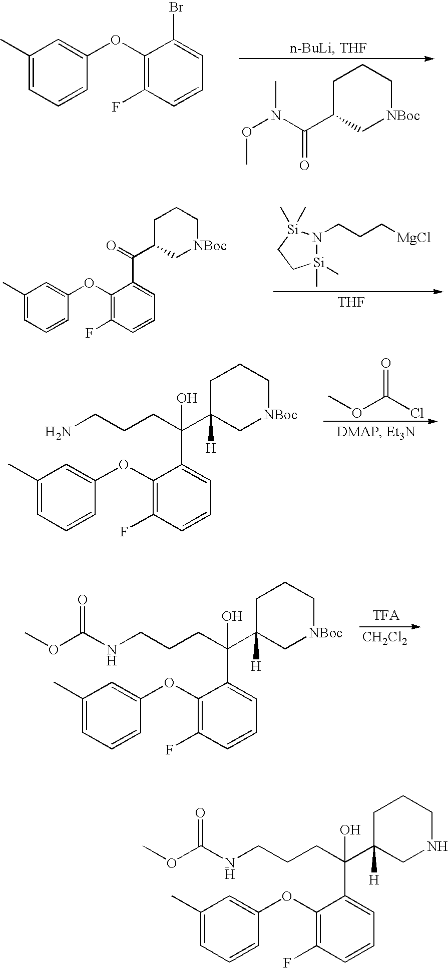

### Step 1. 1,1-dimethylethyl (3R)-3-({3-fluoro-2-[(3-methylphenyl)oxy]phenyl}carbonyl)-1-piperidinecarboxylate

To a solution of 1-bromo-3-fluoro-2-[(3-methylphenyl)oxy]benzene (3.27 g, 11.7 mmol) in THF at −78° C., was added n-BuLi (2.5 M, 5.5 mL, 13.8 mmol). The resulting solution was stirred at −78° C. for 1 h. A solution of 1,1-dimethylethyl (3R)-3-{[methyl(methyloxy)amino]carbonyl}-1-piperidinecarboxylate (2.89 g, 10.6 mmol) in THF was then added dropwise and the resulting mixture warmed to room temperature for 2 h before it was quenched with saturated NH₄Cl. The organic layer was separated and aqueous layer extracted with ethyl acetate. Combined organic layers are washed with brine, concentrated in vacuo to give crude 1,1-dimethylethyl (3R)-3-({3-fluoro-2-[(3-methylphenyl)oxy]phenyl}carbonyl)-1-piperidinecarboxylate (5.1 g) which was used in the next reaction without further purification.

### Step 2. 1,1-dimethylethyl (3R)-3-(4-amino-1-{3-fluoro-2-[(3-methylphenyl)oxy]phenyl}-1-hydroxybutyl)-1-piperidinecarboxylate

To a solution of 1,1-dimethylethyl (3R)-3-({3-fluoro-2-[(3-methylphenyl)oxy]phenyl}carbonyl)-1-piperidinecarboxylate (5 g, 12.1 mmol) in THF at −78° C. was added dropwise a solution of (3-(2,2,5,5-tetramethyl-1,2,5-azadisilolidin-1-yl)propyl)magnesium chloride (1.45 M, 10.5 mL, 36.5 mmol). After the addition was complete, the resulting solution was allowed to warm to rt slowly, and left at rt for 1 h. The reaction was quenched with saturated NH₄Cl and extracted with ethyl acetate. The combined organic layers were washed with brine, dried over Na₂SO₄, filtered and concentrated in vacuo to give crude 1,1-dimethylethyl (3R)-3-(4-amino-1-{3-fluoro-2-[(3-methylphenyl)oxy]phenyl}-1-hydroxybutyl)-1-piperidinecarboxylate (6.6 g) which was used in the next step without further purification. MS (E/Z): 473.1 (M+H⁺)

### Step 3. 1,1-dimethylethyl (3R)-3-(1-{3-fluoro-2-[(3-methylphenyl)oxy]phenyl}-1-hydroxy-4-{[(methyloxy)carbonyl]amino}butyl)-1-piperidinecarboxylate

To a solution of 1,1-dimethylethyl (3R)-3-(4-amino-1-{3-fluoro-2-[(3-methylphenyl)oxy]phenyl}-1-hydroxybutyl)-1-piperidinecarboxylate (5.93 g, 13.2 mmol) and DMAP (0.81 g, 0.6 mmol) in CH₂Cl₂ was added Et₃N (4.0 g, 39.6 mmol). The resulting mixture was cooled to 5° C. and methyl chloroformate (6.2 g, 66 mmol) added and the mixture maintained at 5° C. for 2 h. The reaction was quenched with water and extracted with CH₂Cl₂. The combined organic layers were washed with 10% citric acid and brine, dried over Na₂SO₄, filtered and concentrated in vacuo to give a crude product which was purified by flash column chromatography to afford 1,1-dimethylethyl (3R)-3-(1-{3-fluoro-2-[(3-methylphenyl)oxy]phenyl}-1-hydroxy-4-{[(methyloxy)carbonyl]amino}butyl)-1-piperidinecarboxylate (3.2 g, 48%). MS (E/Z): 531.1 (M+H⁺)

### Step 4. Methyl {4-{3-fluoro-2-[(3-methylphenyl)oxy]phenyl}-4-hydroxy-4-[(3R)-3-piperidinyl]butyl}carbamate

To a solution of 1,1-dimethylethyl (3R)-3-(1-{3-fluoro-2-[(3-methylphenyl)oxy]phenyl}-1-hydroxy-4-{[(methyloxy)carbonyl]amino}butyl)-1-piperidinecarboxylate (3.19 g, 6.0 mmol) in CH₂Cl₂ (31.9 mL) was added TFA (31.9 mL) slowly at rt. The resulting mixture was stirred at rt for 15 min then neutralized to pH=7 with aqueous NaHCO₃ and extracted with CH₂Cl₂. The combined extracts were washed with brine, dried over Na₂SO₄, filtered and concentrated in vacuo to give methyl {4-{3-fluoro-2-[(3-methylphenyl)oxy]phenyl}-4-hydroxy-4-[(3R)-3-piperidinyl]butyl}carbamate (2.9 g) which was used in the next step without further purification. MS (E/Z): 431.1 (M+H⁺)

The following piperidines prepared using the above procedures using the halodiphenyl ethers listed below in Step 1.

| Piperidine | Halodiphenyl ether |
|---|---|
| methyl {4-{3-chloro-2-[(3- | 1-bromo-3-chloro-2-[(3- |
| methylphenyl)oxy]phenyl}-4-hydroxy-4- | methylphenyl)oxy]benzene |
| [(3R)-3-piperidinyl]butyl}carbamate | |
| methyl {4-{2-[(2,6- | 2-[(2-bromophenyl)oxy]-1,3- |
| dimethylphenyl)oxy]phenyl}-4-hydroxy-4- | dimethylbenzene |
| [(3R)-3-piperidinyl]butyl}carbamate | |
| methyl {4-{3-chloro-2-[(2- | 1-bromo-3-chloro-2-[(2- |
| methylphenyl)oxy]phenyl}-4-hydroxy-4- | methylphenyl)oxy]benzene |
| [(3R)-3-piperidinyl]butyl}carbamate | |

### Preparation 7

### Piperidines from Weinreb Amides and Bromobiaryls

### 1-(2′-chloro-2-biphenylyl)-5-(methyloxy)-1-[(3R)-3-piperidinyl]-1-pentanol

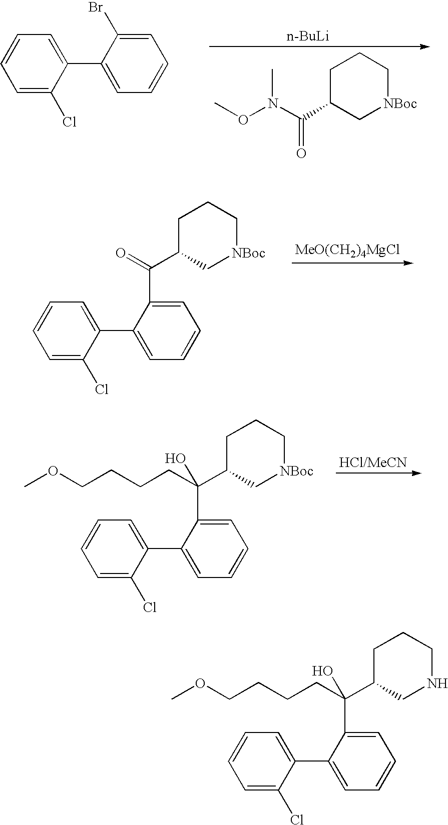

### Step 1. (3R)-1-(tert-butoxycarbonyl)-3-((2-(2-chlorophenyl))benzoyl)piperidine

To a solution of 2′-bromo-2-chloro-biphenyl (5.34 g, 20 mmol) in anhydrous THF (50 mL) cooled to −78° C. was added dropwise a solution of 1.6 M n-BuLi in hexane (12.5 mL, 20 mmol). The reaction mixture was stirred at −78° C. for 1 h and a solution of (R)-tert-butyl 3-(N-methoxy-N-methylcarbamoyl)-piperidine-1-carboxylate (5.44 g, 20 mmol) in anhydrous THF (50 mL) was added. The mixture was allowed to warm to rt and stirred overnight. The mixture was quenched with saturated aqueous NH₄Cl (100 mL) and extracted with EtOAc (3×75 mL). The combined organic layers were dried over Na₂SO₄ and concentrated to give the crude product, which was purified by flash column chromatography to afford (3R)-1-(tert-butoxycarbonyl)-3-((2-(2-chlorophenyl))benzoyl)piperidine (4.43 g, 55%).

### Step 2. 1,1-dimethylethyl (3R)-3-[1-(2′-chloro-2-biphenylyl)-1-hydroxy-5-(methyloxy)pentyl]-1-piperidinecarboxylate

A 250 mL three-necked flask is charged with magnesium turning (2.88 g, 0.12 mol) and a small crystal of iodine. The flask was evacuated and refilled with N₂. A solution of 1-chloro-4-methoxybutane (15 g, 0.12 mol) in THF (60 ml) was added dropwise to the above mixture. After heating under reflux for 2 h most of magnesium was consumed and the Grignard solution was cooled to rt. A 250 mL three-necked flask was charged with (3R)-1-(tert-butoxycarbonyl)-3-((2-(2-chlorophenyl))benzoyl)piperidine (4.43 g, 11 mmol) and THF (50 mL), evacuated and refilled with N₂. The mixture was cooled in a dry ice-acetone bath and the Grignard reagent added dropwise. The mixture was allowed to warm slowly to rt and stirred overnight. The mixture was quenched with saturated aqueous NH₄Cl (100 mL) and extracted with EtOAc. The combined organic layers were dried over Na₂SO₄ and concentrated to give the crude product which was purified by flash column chromatography to afford pure 1,1-dimethylethyl (3R)-3-[1-(2′-chloro-2-biphenylyl)-1-hydroxy-5-(methyloxy)pentyl]-1-piperidinecarboxylate (2.5 g, 47%).

### Step 3. 1-(2′-chloro-2-biphenylyl)-5-(methyloxy)-1-[(3R)-3-piperidinyl]-1-pentanol

The Boc protecting group was removed using the protocol described in Preparation 5 Step 4.

The following piperidines were prepared using procedures analogous to those described above substituting the bromobiphenyls indicated in Step 1:

| Piperidine | Bromobiphenyl |
|---|---|
| 1-(6-fluoro-3′-methoxy-5′-methylbiphenyl-2-yl)-5- | 2-bromo-6-fluoro-3′-methoxy-5′- |
| methoxy-1-((R)-piperidin-3-yl)pentan-1-ol | methylbiphenyl |
| 1-(6-chloro-3′-ethylbiphenyl-2-yl)-5-methoxy-1-((R)- | 2-bromo-6-chloro-3′-ethylbiphenyl |
| piperidin-3-yl)pentan-1-ol | |
| 1-(3-chloro-2-(2-methylbenzyl)phenyl)-5- | 1-bromo-3-chloro-2-(2- |
| methoxy-1-((R)-piperidin-3-yl)pentan-1-ol | methylbenzyl)benzene |
| 1-(3′-ethyl-6-fluorobiphenyl-2-yl)-5-methoxy-1- | 2-bromo-3′-ethyl-6-fluorobiphenyl |
| ((R)-piperidin-3-yl)pentan-1-ol | |
| 1-(3-chloro-2-(quinolin-3-yl)phenyl)-5-methoxy- | 3-(2-bromo-6- |
| 1-((R)-piperidin-3-yl)pentan-1-ol | chlorophenyl)quinoline |
| 1-{3-chloro-2-[(3-methylphenyl)methyl]phenyl}- | 1-bromo-3-chloro-2-[(3- |
| 5-(methyloxy)-1-[(3R)-3-piperidinyl]-1-pentanol | methylphenyl)methyl]benzene |

### Preparation 8

### Biaryl Syntheses

### a) 6-Bromo-2-fluoro-3′-methylbiphenyl

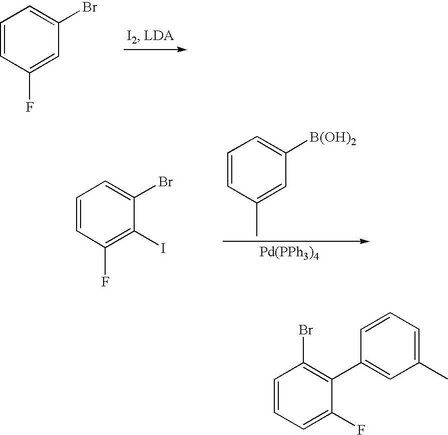

### Step 1. 1-Bromo-3-fluoro-2-iodobenzene

To a solution of diisopropylamine (76 mL, 0.4 mol) in dry THF (664 mL) and n-hexane (220 mL) is added 2.5 M n-BuLi (160 mL. 0.4 mol) dropwise at −78° C. during a period of 1 h. The mixture is stirred for 1 h at −78° C. Then a solution of 1-bromo-3-fluoro-benzene (69 g, 0.4 mol) in dry THF (300 mL) at −78° C. is added to the above mixture dropwise. After stirring for an additional 1 h at −78° C., the mixture is added a solution of iodine (101 g, 0.4 mol) in dry THF (400 mL) dropwise at −78° C. The temperature is raised from −78° C. to rt during 2 h. After stirring for 18 h at rt, the mixture is concentrated in vacuo to give crude product (120 g) which is distilled under reduced pressure to afford 1-bromo-3-fluoro-2-iodobenzene (110 g). ¹H NMR (400 MHz, DMSO): 7.24-7.19 (t, 1H), 7.38-7.32 (m, 1H), 7.55-7.53 (d, 1H).

### Step 2. 6-Bromo-2-fluoro-3′-methylbiphenyl

Pd(Ph₃P)₄ in a 500-mL round-bottom flask under N₂ atmosphere is treated sequentially with a solution of 1-bromo-3-fluoro-2-iodo-benzene (30 g, 0.1 mol) in toluene (250 mL), a solution of 2N aq Na₂CO₃ (200 mL) and 3-methyl phenylboronic acid in ethanol (62 mL). This mixture is heated at reflux under N₂ for 12 h, then cooled to rt. The mixture is partitioned between water and EtOAc. The combined organic layers are washed with brine, dried over MgSO₄, evaporated and purified by column chromatography to give 6-bromo-2-fluoro-3′-methyl-biphenyl (12 g). ¹H NMR (400 MHz, CD₃OD): 7.03 (m, 2H), 7.48-7.04 (m, 4H), 7.50 (d, 1H).

### b) 6-Bromo-2-chloro-3′-methyl-biphenyl

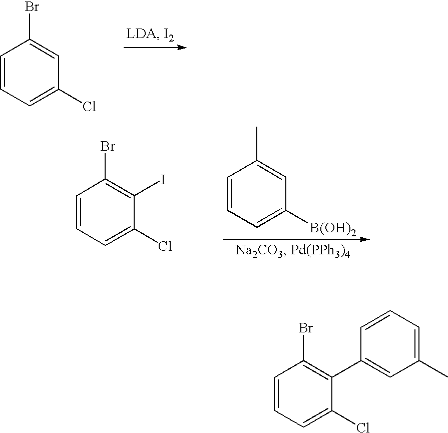

### Step 1. 1-bromo-3-chloro-2-iodobenzene

To a solution of diisopropylamine (76 mL, 0.4 mol) in anhydrous THF (664 mL) and n-hexane (220 mL) was added 2.5 M n-BuLi (160 mL, 0.4 mol) dropwise at −78° C. over 1 h. The mixture was stirred for 1 h at −78° C. and a solution of 1-bromo-3-chlorobenzene (76 g, 0.4 mol) in anhydrous THF (300 mL) was added dropwise at −78° C. After stirring for an additional 1 h at the same temperature, a solution of iodine (101 g, 0.4 mol) in anhydrous THF (400 mL) was added dropwise at −78° C. The temperature was raised from −78° C. to rt during 2 h. After stirring for 18 h at rt, the mixture was concentrated in vacuo to give the crude product (120 g) which was distilled under reduced pressure to give 1-bromo-3-fluoro-2-iodobenzene (115 g, 91%). ¹H NMR (400 MHz, CDCl₃): 7.12-7.18 (t, 1H), 7.35-7.41 (dd, 1H), 7.49-7.54 (dd, 1H); MS (E/Z): 317 (M+H⁺)

### Step 2. 6-bromo-2-chloro-3′-methyl-biphenyl

A 500-mL round-bottom flask under N₂ atmosphere was charged sequentially with Pd(Ph₃P)₄, 1-bromo-3-fluoro-2-iodobenzene (10 g, 0.032 mol) in toluene (80 mL), 2N aqueous sodium carbonate (55 mL) and 3-methylphenylboronic acid (5.16 g, 0.032 mol) dissolved in ethanol (40 mL). This mixture was heated at reflux under N₂ for 12 h and cooled to rt. The mixture was partitioned between water and EtOAc. The combined organic layers were washed with brine, dried over MgSO₄, and concentrated. The residue was purified by column chromatography to give 6-bromo-2-chloro-3′-methyl-biphenyl (6 g, 67%). ¹H NMR (400 MHz, CD₃OD): 6.90-7.00 (t, 2H), 7.14-7.24 (m, 2H), 7.26-7.33 (t, 1H), 7.44-7.50 (d, 1H), 7.58-7.62 (d, 1H); MS (E/Z): 281 (M+H⁺)

The following biaryls were prepared from aryl halides and the boronic acids indicated using the procedures described in Preparations 7a Step 2 and 7b Step 2:

| Biaryl | Aryl halide | Boronic acid |
|---|---|---|
| 2′-bromo-6′-chloro-2-fluoro- | 1-bromo-3-chloro- | 2-fluoro-5-methylphenylboronic |
| 5-methylbiphenyl | 2-iodobenzene | acid |
| 2-bromo-6-chloro-3′- | 1-bromo-3-chloro- | 3-methylphenylboronic acid |
| methylbiphenyl | 2-iodobenzene | |
| 2-bromo-6-chloro-3′- | 1-bromo-3-chloro- | 3-ethylphenylboronic acid |
| ethylbiphenyl | 2-iodobenzene | |
| 2-bromo-3′-ethyl-6- | 1-bromo-3-fluoro- | 3-ethylphenylboronic acid |
| fluorobiphenyl | 2-iodobenzene | |
| 2-bromo-6-chloro-3′- | 1-bromo-3-chloro- | 3-isopropylphenylboronic acid |
| isopropylbiphenyl | 2-iodobenzene | |
| 2-bromo-4′,6-difluoro-3′- | 1-bromo-3-fluoro- | (4-fluoro-3-methylphenyl)boronic |
| methylbiphenyl | 2-iodobenzene | acid |
| 2-bromo-6-chloro-3′- | 1-bromo-3-chloro- | [3-(methoxy)phenyl]boronic acid |
| (methoxy)biphenyl | 2-iodobenzene | |
| 2-bromo-6-fluoro-3′- | 1-bromo-3-fluoro- | [3-(methoxy)phenyl]boronic acid |
| (methoxy)biphenyl | 2-iodobenzene | |
| 2-bromo-6-fluoro-3′- | 1-bromo-3-fluoro- | [3-methyl-5- |
| methyl-5′- | 2-iodobenzene | (methoxy)phenyl]boronic acid |
| (methoxy)biphenyl | | |
| 3-(2-bromo-6- | 1-bromo-3-chloro- | 3-quinolinylboronic acid |
| chlorophenyl)quinoline | 2-iodobenzene | |
| 2-(2-bromo-6- | 1-bromo-3-chloro- | 2-naphthalenylboronic acid |
| chlorophenyl)naphthalene | 2-iodobenzene | |
| 2-bromo-6-chloro-3′- | 1-bromo-3-chloro- | {3- |
| [(methoxy)methyl]biphenyl | 2-iodobenzene | [(methoxy)methyl]phenyl}boronic |
| | | acid |
| 2′-bromo-2,6′-difluoro-5- | 1-bromo-3-fluoro- | 2-fluoro-5-methylphenylboronic |
| methylbiphenyl | 2-iodobenzene | acid |
| 2-bromo-2′,4,6-trifluoro-5′- | 1-bromo-3,5- | (2-fluoro-5-methylphenyl)boronic |
| methylbiphenyl | difluoro-2- | acid |
| | iodobenzene | |
| 2-bromo-6-fluoro-3′- | 1-bromo-3-fluoro- | (3-methylphenyl)boronic acid |
| methylbiphenyl | 2-iodobenzene | |
| 4-bromo-2-chloro-3-(3- | 4-bromo-2-chloro- | (3-ethylphenyl)boronic acid |
| ethylphenyl)pyridine | 3-iodopyridine | |
| 3-(2-bromo-6- | 1-bromo-3-chloro- | 1-benzothien-2-ylboronic acid |
| chlorophenyl)-1- | 2-iodobenzene | |
| benzothiophene | | |
| 2-(2-bromo-6- | 1-bromo-3-chloro- | (5-methyl-2-furanyl)boronic acid |
| chlorophenyl)-5- | 2-iodobenzene | |
| methylfuran | | |
| 3-(2-bromo-6- | 1-bromo-3-fluoro- | 3-quinolinylboronic acid |
| fluorophenyl)quinoline | 2-iodobenzene | |
| 2′-bromo-6′-chloro-2- | 1-bromo-3-chloro- | [2-(methyloxy)-5- |
| (methyloxy)-5- | 2-iodobenzene | (trifluoromethyl)phenyl]boronic |
| (trifluoromethyl)biphenyl | | acid |
| 2-bromo-3′,6-difluoro-5′- | 1-bromo-3-fluoro- | 3-fluoro-5-methylphenylboronic |
| methylbiphenyl | 2-iodobenzene | acid |
| 2-bromo-6-chloro-3′-fluoro- | 1-bromo-3-chloro- | 3-fluoro-5-methylphenylboronic |
| 5′-methylbiphenyl | 2-iodobenzene | acid |
| 6-(2-bromo-6- | 1-bromo-3-chloro- | 2,3-dihydrobenzofuran-6- |
| chlorophenyl)-2,3- | 2-iodobenzene | ylboronic acid |
| dihydrobenzofuran | | |
| 2-bromo-6-fluoro-3′-(1- | 1-bromo-3-fluoro- | 3-isopropylphenylboronic acid |
| methylethyl)biphenyl | 2-iodobenzene | |
| 4-bromo-2-chloro-3-(3- | 4-bromo-2-chloro- | (3-ethylphenyl)boronic acid |
| ethylphenyl)pyridine | 3-iodopyridine | |
| 4-bromo-2-chloro-3-[3-(1- | 4-bromo-2-chloro- | [3-(1-methylethyl)phenyl]boronic |
| methylethyl)phenyl]pyridine | 3-iodopyridine | acid |

### c) 6-bromo-2-fluoro-3′-methyl-3-(methyloxy)biphenyl

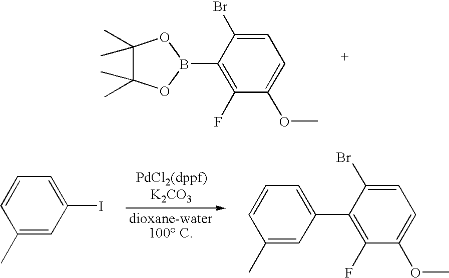

### Step 1. 6-Bromo-2-fluoro-3′-methyl-3-(methyloxy)biphenyl

To a deoxygenated mixture of m-iodotoluene (4.80 g, 22 mmol), 6-bromo-2-fluoro-3-methoxyphenylboronic acid pinacol ester (7.94 g, 24 mmol) and potassium carbonate (12.14 g, 88 mmol) in dioxane (250 ml) and water (100 ml) was added Pd(dppf)Cl₂ (1.80 g, 2.2 mmol). The resulting mixture was then heated to 100° C. for 18 h. The catalyst was removed by filtration and the filtrate concentrated in vacuo. The residue was then dissolved in EtOAc and washed with brine, dried and concentrated to give the crude product as a dark brown oil. Purification by column chromatography (80 g silica gel 60, 230-400 mesh, 3% then 5% CH₂Cl₂ in hexane as eluent) afforded 6-bromo-2-fluoro-3′-methyl-3-(methyloxy)biphenyl (2.37 g, 36%) as a clear oil. MS (E/Z): 295.3 (M+H⁺)

### d) 2-(2-bromo-6-chlorophenyl)-8-(1-methylethyl)quinoline

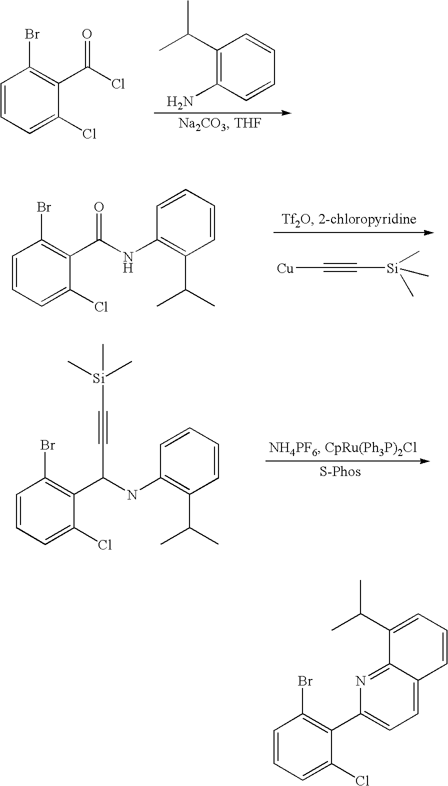

### Step 1. 2-bromo-6-chloro-N-[2-(1-methylethyl)phenyl]benzamide

To a solution of 2-bromo-6-chlorobenzoyl chloride (39.3 g, 149.1 mmol) and Na₂CO₃ (31.4 g, 318.3 mmol) in THF (232 mL) and water (23 mL) was added [2-(1-methylethyl)phenyl]amine (22.2 g, 164.1 mmol). The resulting mixture was stirred at room temperature for 1 h. The mixture was extracted with ethyl acetate and the pH adjusted to 2. The organic layers were then washed with Na₂CO₃, brine, dried over Na₂SO₄, filtered and concentrated to give 2-bromo-6-chloro-N-[2-(1-methylethyl)phenyl]benzamide (35.5 g) which was used in the subsequent step without further purification. MS (E/Z): 353.9 (M+H⁺)

### Step 2. N-[(1)-1-(2-bromo-6-chlorophenyl)-3-(trimethylsilyl)-2-propyn-1-ylidene]-2-(1-methylethyl)aniline

To a stirred solution of 2-bromo-6-chloro-N-[2-(1-methylethyl)phenyl]benzamide (50 g, 137.8 mmol) and 2-chloropyridine (50.5 mL, 554.9 mmol) in CH₂Cl₂ (300 mL) under argon at −78° C. was added Tf₂O (28.2 mL, 167.6 mmol). After 5 min, the reaction mixture was warmed to 0° C. and maintained at that temperature for 20 min before recooling to −78° C. [(Trimethylsilyl)ethynyl]copper (730 mL, 383.4 mmol) was then added via cannula as a solution in THF. The resulting mixture was maintained at −78° C. for 5 min before warming to 0° C. After 10 min the crude mixture was filtered through Celite and the filtrate concentrated in vacuo. The crude material was purified via column chromatography to give N-[(1)-1-(2-bromo-6-chlorophenyl)-3-(trimethylsilyl)-2-propyn-1-ylidene]-2-(1-methylethyl)aniline (9.58 g, 16%). MS (E/Z): 433.1 (M+H⁺)

### Step 3. 2-(2-bromo-6-chlorophenyl)-8-(1-methylethyl)quinoline

To a mixture of ammonium hexafluorophosphate purum (3.7 g, 22.2 mmol) and CpRu(Ph₃P)₂Cl (1.64 g, 2.3 mmol) in toluene (110 mL) was added N-[(1)-1-(2-bromo-6-chlorophenyl)-3-(trimethylsilyl)-2-propyn-1-ylidene]-2-(1-methylethyl)aniline (9.1 g, 21.0 mmol). The resulting mixture was then heated to 115° C. for 19 h. The reaction was then cooled to rt, diluted with CH₂Cl₂ and the solvent removed in vacuo. The residue was then purified via column chromatography to afford 2-(2-bromo-6-chlorophenyl)-8-(1-methylethyl)quinoline (3.7 g, 49%). MS (E/Z): 360.0 (M+H⁺)

The following biaryls were prepared from the aniline indicated using the procedures described in Preparations 8d, Steps 1-3:

| Biaryl | Aniline |
|---|---|
| 2-(2-bromo-6-chlorophenyl)-8- | 2-methyl aniline |
| methylquinoline | |

### e) 2-(2-bromo-6-chlorophenol)-4-(1-methylethyl)quinazoline

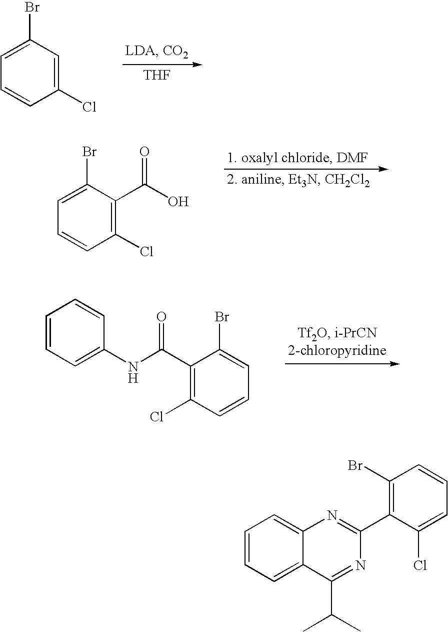

### Step 1. 2-bromo-6-chlorobenzoic acid

To a stirred solution of n-BuLi (90.0 mmol, 36 ml of a 2.5 M solution in hexanes) in 160 mL of dry THF at −78° C., was added dropwise diisopropylamine (12.4 ml, 90 mmol) in 20 mL of dry THF. The resulting solution was stirred for 0.5 h at −78° C. A solution of 1-bromo-3-chlorobenzene (14.3 g, 75.0 mmol) in 20 ml of dry THF was added and the resulting mixture was stirred for an additional hour −78° C. Then dry ice (CO₂) was added in small portions (large gas evolution) and after 20 min the solution was quenched with 100 mL of 2N HCl. The mixture was extracted with ethyl acetate (1000 ml) and the crude 2-bromo-6-chlorobenzoic acid (white solid) was triturated with Et₂O and used in the next step without other purification. MS (E/Z): 234.9 (M+H⁺)

### Step 2. 2-bromo-6-chloro-N-phenylbenzamide

To a stirred solution of 2-bromo-6-chlorobenzoic acid (3.15 g, 13.4 mmol) in 20.0 mL of dry methylene chloride, were added DMF (catalytic amount) and oxalyl chloride (1.45 mL, 16.1 mmol) dropwise. The resulting solution was stirred for 2 h at room temperature. The solvent was removed in vacuo and the crude dissolved in 20.0 mL of dry DCM. Triethylamine (3.7 mL, 26.8 mmol) and aniline (1.78 mL, 18.7 mmol) were added and the resulting mixture was stirred over night at room temperature. HPLC/MS showed that the reaction was completed at this time. The reaction mixture was quenched with 0.6N HCl and extracted with methylene chloride. The organic layer was then dried, filtered and concentrated to afford 2-bromo-6-chloro-N-phenylbenzamide, which was used in the next step without further purification. MS (E/Z): 309.9 (M+H⁺)

### Step 3. 2-(2-bromo-6-chlorophenyl)-4-(1-methylethyl)quinazoline

To a stirred solution of 2-bromo-6-chloro-N-phenylbenzamide (930 mg, 3.0 mmol) and 2-chloropyridine (406 μl, 3.6 mmol) in 10 ml of dry methylene chloride was added at −78° C. followed by Tf₂O (1015 μl, 3.6 mmol). The solution was stirred at −78° C. and then was warmed to 0° C. and i-PrCN (354 μl, 3.6 mmol) added. The resulting solution was stirred overnight 70° C. in a microwave vial. The HPLC/MS showed product as well as starting material. The reaction mixture was quenched with 0.6N HCl and extracted with methylene chloride. The organic layer was then dried, filtered, and concentrated to afford the crude material. Column chromatography then gave 2-(2-bromo-6-chlorophenyl)-4-(1-methylethyl)quinazoline (0.418 g, 40%). MS (E/Z): 361.0 (M+H⁺)

### f.) 3-Bromo-5-chloro-4-[3-(1-methylethyl)phenyl]pyridine

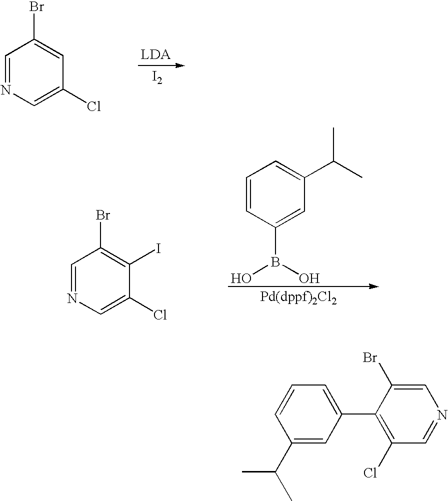

### Step 1: 3-bromo-5-chloro-4-iodopyridine

To a −78° C. solution of diisopropylamine (3.7 mL, 26 mmol) in anhydrous THF (50 mL) was added n-BuLi (10.4 mL, 2.5 M hexanes, 26 mmol). After stirring for 30 minutes, a solution of 3-bromo-5-chloropyridine (5.0 g, 26 mmol) in THF (10 mL) was added dropwise. After stirring for an additional 1 hour at −78° C., a solution of iodine (7.9 g, 31.2 mmol) in THF (25 mL) was added. The reaction was slowly allowed to warm to room temperature and continued to stir overnight. The reaction was quenched with water (25 ml) and a saturated sodium thiosulfate solution (25 mL). The phases were separated. The organic layer was washed with Na₂S₂O₃ solution (25 mL). The aqueous phase was back extracted with EtOAc (3×25 mL). The combined organic extracts were washed with brine, dried over MgSO₄, filtered and concentrated to give 7 g of a brown solid. The crude product was triturated with Et₂O twice and isolated 3-bromo-5-chloro-4-iodopyridine (3.3 g, 40% yield) as a brown solid (powder).

### Step 2: 3-bromo-5-chloro-4-(3-isopropylphenyl)pyridine

3-Bromo-5-chloro-4-iodopyridine (1.6 g, 5.0 mmol), (3-isopropylphenyl)boronic acid (0.99 g, 6.0 mmol), Na₂CO₃ (1.1 g, 10 mmol) and Pd(PPh₃)₂Cl₂ (0.50 g, 0.50 mmol) were added to a flask with 1,4-dioxane/water (2:1) (15 mL). The reaction mixture was immersed into a preheated oil bath (85° C.) and stirred overnight (18 hours). The reaction mixture was allowed to cool to room temperature and then diluted with EtOAc and water. The phases were separated and the aqueous phase was extracted with EtOAc (2×). The combined organics were washed with brine, dried over MgSO₄, filtered and concentrated to give 2 g of a brown oil. The crude residue was purified by flash chromatography on silica gel and isolated 0.78 g (50% yield) of 3-bromo-5-chloro-4-(3-isopropylphenyl)pyridine as a clear oil.

### g.) 2-bromo-4-chloro-3-(3-ethylphenyl)pyridine

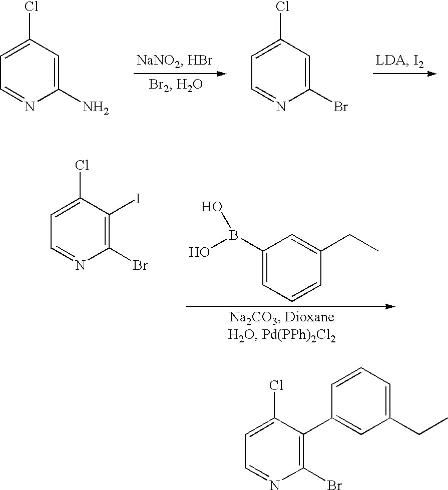

### Step 1. 2-bromo-4-chloropyridine

To an aqueous solution of 48% strength hydrobromic acid (82 mL) at 0° C. was added 4-chloro-2-pyridinamine (8.9 g, 69.2 mmol) followed by addition of bromine (33.4 g, 209 mmol) over 10 min. The resulting mixture was cooled to −10° C. and a solution of NaNO₂ (10.65 g, 154 mmol) in H₂O (20 mL) was poured in over a period of 30 min. The mixture was warmed at room temperature and stirred overnight. The mixture was recooled to 0° C. and NaOH (35%) added until the pH>10. The mixture was then extracted with ethyl acetate. The organic layer was then dried, filtered, and concentrated in vacuo. The product was purified via column chromatography (0-20% ethyl acetate/hexane) to afford 2-bromo-4-chloropyridine (12.1 g, 92%).

### Step 2. 2-bromo-4-chloro-3-iodopyridine

To a stirred solution of diisopropylamine (8.24 mL, 60.0 mmol) in THF (100 mL) at −78° C. was added n-BuLi (24.0 mL, 60.0 mmol) and the solution was stirred at this temperature for 30 min. Then, a solution of 2-bromo-4-chloropyridine (12.1 g, 60.0 mmol) dissolved in THF (100 mL) was added dropwise. The resulting mixture was stirred for 1 h at −78° C. Then I₂ (21.0 g, 66.0 mmol) was added in three portions. The solution was warmed to room temperature and stirred overnight. The mixture was diluted with ethyl acetate and washed with water. The organic layer was then dried, filtered, and concentrated in vacuo. The crude material was purified via column chromatography to give 2-bromo-4-chloro-3-iodopyridine (7.3 g, 38%). MS (E/Z): 317.8 (M+H⁺).

### Step 3. 2-bromo-4-chloro-3-(3-ethylphenyl)pyridine

To a solution of 2-bromo-4-chloro-3-iodopyridine (3.17 g, 10 mmol) in dioxane (20 mL) and water (10 mL) was added (3-ethylphenyl)boronic acid (1.9 g, 13.0 mmol) followed by Pd(Ph)₂Cl₂ (0.350 g, 0.5 mmol). The resulting mixture was then heated at 80° C. overnight. In the morning, the reaction mixture was diluted with ethyl acetate, washed with water then brine, dried, filtered and concentrated in vacuo.

The crude material was then purified via column chromatography to afford 2-bromo-4-chloro-3-(3-ethylphenyl)pyridine (1.5 g, 50%).

### Preparation 9

### Morpholine Synthesis

### (R)-1-(6-Fluoro-3′-methylbiphenyl-2-yl)-5-methoxy-1-((R)-morpholin-2-yl)pentan-1-ol

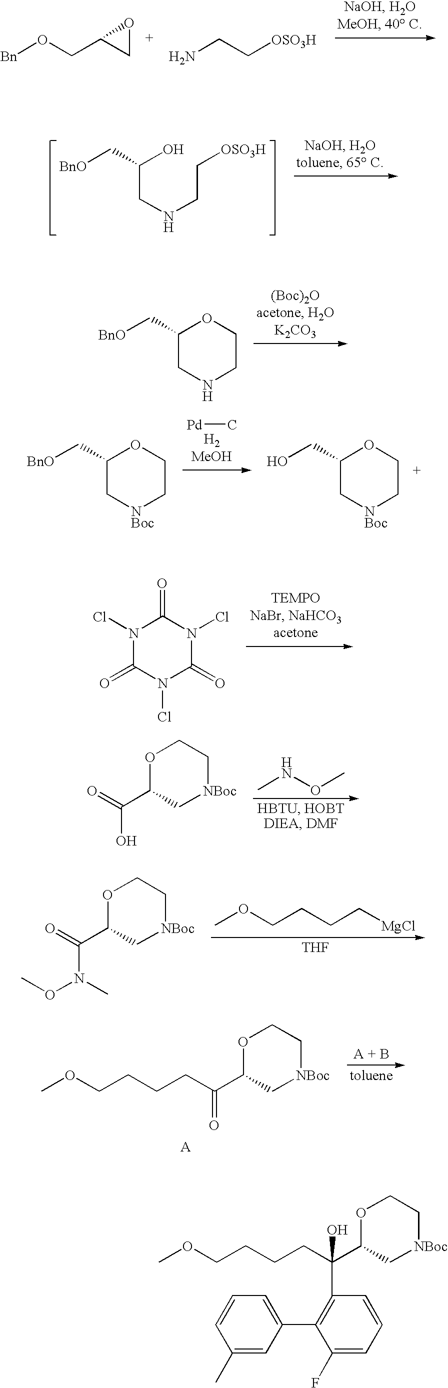

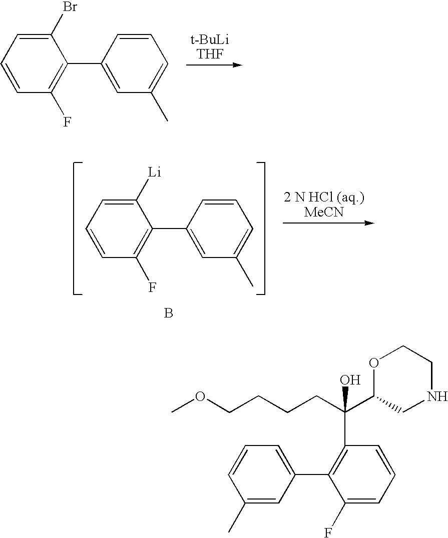

### Step 1. (R)-2-(Benzyloxymethyl)morpholine

To a stirred mixture of (R)-2-(benzyloxymethyl)oxirane (10.0 g, 60.9 mmol) and NaOH (19.49 g, 487.2 mmol) in H₂O (46 mL) and MeOH (18 mL), there was added 2-aminoethyl hydrogen sulfate (36.8 g, 255.8 mmol) in portions. After addition was complete, the reaction mixture was stirred at 40° C. for 2 h. After cooling, the mixture was treated with NaOH (15.0 g, 375.0 mmol), followed by toluene (70 mL), and stirred at 65° C. overnight. The mixture was cooled, diluted with toluene (27 mL) and H₂O (92 mL). The toluene layer was separated and the aqueous layer was extracted with CH₂Cl₂ (2×50 mL). The combined organic layers were concentrated to give crude (R)-2-(benzyloxymethyl)morpholine (˜14 g), which was used without purification. MS m/z 208 (M+H⁺).

### Step 2. (R)-tert-Butyl 2-(benzyloxymethyl)morpholine-4-carboxylate

To a solution of crude (R)-2-(benzyloxymethyl)morpholine (˜14 g) in acetone (100 mL) and H₂O (30 mL) at 0° C., there was added K₂CO₃ (25.2 g, 182.7 mmol), followed by (Boc)₂O (14.6 g, 67.0 mmol). The resulting solution was warmed to rt, and stirred until no starting material remained (˜30 min). Acetone was removed under vacuum, and the aqueous solution was extracted with CH₂Cl₂ (4×10 mL). The combined organic layers were washed with H₂O (10 mL) and the solvent was removed. The residue was purified by flash column chromatography to give (R)-tert-butyl 2-(benzyloxymethyl)morpholine-4-carboxylate (8.33 g, 44% over 2 steps). ¹H NMR (400 MHz, CDCl₃): 7.34 (m, 5H), 4.56 (s, 2H), 3.88 (d, 2H), 3.82 (br, 1H), 3.40 (m, 1H), 3.48 (m, 3H), 2.94 (m, 1H), 2.76 (m, 1H), 1.44 (s, 9H); MS m/z 330 (M+Na⁺)

### Step 3. (R)-tert-Butyl 2-(hydroxymethyl)morpholine-4-carboxylate

To a solution of (R)-tert-butyl 2-(benzyloxymethyl)morpholine-4-carboxylate (8.33 g, 27.1 mmol) in EtOH was added Pd—C (wet, 3.6 g), and the resulting mixture was stirred at rt under a H₂ balloon overnight. After filtration, the solvent was removed under vacuum, and the residue was purified by flash column chromatography to give (R)-tert-butyl 2-(hydroxymethyl)morpholine-4-carboxylate (5.84 g, 99%) as a clear oil. ¹H NMR (400 MHz, CDCl₃): 3.88 (d, 2H), 3.82 (br, 1 H), 3.64 (d, 1H), 3.56 (m, 3H), 2.94 (m, 1H), 2.76 (m, 1H), 1.90 (br, 1H), 1.44 (s, 9H); MS m/z 218 (M+H⁺).

### Step 4. (R)-4-(tert-Butoxycarbonyl)morpholine-2-carboxylic acid

Satd aq NaHCO₃ (15 mL) was added to a solution of (R)-tert-butyl 2-(hydroxymethyl)-morpholine-4-carboxylate (1.09 g, 5.0 mmol) in acetone (50 mL), stirred and maintained at 0° C. Solid NaBr (0.1 g, 1 mmol) and TEMPO (0.015 g, 0.1 mmol) were added. Trichloroisocyanuric acid (2.32 g, 10.0 mmol) was then added slowly within 20 min at 0° C. After addition, the mixture was warmed to rt and stirred overnight. 2-Propanol (3 mL) was added, and the resulting solution was stirred at rt for 30 min, filtered through a pad of Celite, concentrated under vacuum, and treated with satd aq Na₂CO₃ (15 mL). The aqueous solution was washed with EtOAc (5 mL), acidified with 6 N HCl, and extracted with EtOAc (5×10 mL). The combined organic layers were dried over Na₂SO₄ and the solvent was removed to give (R)-4-(tert-butoxycarbonyl)morpholine-2-carboxylic acid (1.07 g, 92%) as a white solid. ¹H NMR (400 MHz, CDCl₃): 4.20 (br, 1H), 4.12 (d, 1H), 4.02 (d, 1H), 3.84 (m, 1H), 3.62 (m, 1H), 3.04 (m, 2H), 1.44 (s, 9H); MS m/z 232 (M+H⁺).

### Step 5. (R)-tert-Butyl 2-(methoxy(methyl)carbamoyl)morpholine-4-carboxylate

To a solution of (R)-4-(tert-butoxycarbonyl)morpholine-2-carboxylic acid (1.05 g, 4.54 mmol) in DMF (10 mL) at 0° C., was added DIEA (3.9 mL, 22.7 mmol), followed by HBTU (1.89 g, 4.99 mmol) and HOBt (0.67 g, 4.99 mmol). MeONHMe.HCl (0.48 g, 4.92 mmol) was added and the resulting solution was warmed to rt and stirred until no starting material remained (˜2 h). The mixture was diluted with H₂O (10 mL) and extracted with EtOAc (4×10 mL). The combined organic layers were washed with 1 N aq HCl (10 mL), 1 N aq NaOH (3×10 mL), water (2×10 mL) and brine (10 mL), and dried over Na₂SO₄. The solvent was removed under vacuum to give (R)-tert-butyl 2-(methoxy(methyl)carbamoyl)morpholine-4-carboxylate (1.40 g, quant.), which was used without further purification. ¹H NMR (400 MHz, CDCl₃): 4.36 (br, 1H), 4.08 (m, 1H), 4.00 (d, 1H), 3.84 (m, 1H), 3.76 (s, 3H), 3.58 (m, 1H), 3.20 (s, 3H), 3.04 (m, 2H), 1.44 (s, 9H); MS m/z 297 (M+Na⁺).

### Step 6. (R)-tert-Butyl 2-(5-methoxypentanoyl)morpholine-4-carboxylate

To a stirred solution of (R)-tert-butyl 2-(methoxy(methyl)carbamoyl)morpholine-4-carboxylate (1.37 g, 5.0 mmol) in THF (10 mL) at −20° C., there was added 1.47 M 4-methoxybutylmagnesium chloride in THF (10.2 mL, 15.0 mmol) dropwise to keep the temperature below −20° C. After addition, the resulting solution was warmed to rt and quenched with 1 N aq HCl (10 mL). The organic layer was separated, and the aqueous layer was extracted with ether (3×5 mL). Combined organic layers were washed with satd aq NaHCO₃ (10 mL) and brine (5 mL) and dried over Na₂SO₄. Removal of the solvent under vacuum gave (R)-tert-butyl 2-(5-methoxypentanoyl)morpholine-4-carboxylate (1.41 g, 93%), which was used without purification. MS m/s 324 (M+Na⁺).

### Step 7. (R)-tert-Butyl 2-((R)-1-(6-fluoro-3′-methylbiphenyl-2-yl)-1-hydroxy-5-methoxypentyl)-morpholine-4-carboxylate

To a solution of 2-bromo-6-fluoro-3′-methylbiphenyl (1.90 g, 7.17 mmol) in ether (8 mL) at −78° C., is added t-BuLi in pentane (1.70 M, 8.43 mL, 14.33 mmol) dropwise to keep the temperature below −70° C. The resulting solution is stirred at −78° C.

To a solution of (R)-tert-butyl 2-(5-methoxypentanoyl)morpholine-4-carboxylate (0.68 g, 2.26 mmol) in toluene (8 mL) at −20° C. there is added the above lithium reagent dropwise to keep the solution temperature below −20° C. After addition, the resulting mixture is warmed to rt slowly, and quenched with saturated NH₄Cl (8 mL). The organic layer is separated, and aqueous layer is extracted with ether (3×5 mL). Combined organic layers are washed with water (10 mL), concentrated, and the residue is purified by flash column chromatography to give (R)-tert-butyl 2-((R)-1-(6-fluoro-3′-methylbiphenyl-2-yl)-1-hydroxy-5-methoxypentyl)-morpholine-4-carboxylate (0.48 g, 44%) as a foam. ¹H NMR (400 MHz, CDCl₃): 7.40 (m, 1H), 7.32 (m, 2H), 7.20 (d, 1H), 7.04 (m, 3H), 3.84 (m, 1H), 3.78 (m, 2H), 3.40-3.24 (ms, 7H), 2.82 (s, 3H), 1.70-1.20 (m, 5H), 1.44 (s, 9H), 0.94 (m, 1H); MS m/z 510 (M+Na⁺).

### Step 8. (R)-1-(6-Fluoro-3′-methylbiphenyl-2-yl)-5-methoxy-1-((R)-morpholin-2-yl)-pentan-1-ol

To a solution of (R)-tert-butyl 2-((R)-1-(6-fluoro-3′-methylbiphenyl-2-yl)-1-hydroxy-5-methoxypentyl)morpholine-4-carboxylate (0.46 g, 0.96 mmol) in acetonitrile (50 mL) is added 2 N aq HCl (50 mL). The resulting solution is stirred at rt overnight and basified with 10 N aq NaOH to pH 10. Acetonitrile is removed under vacuum, and the aqueous residue is extracted with CH₂Cl₂ (4×5 mL). The combined organic layers are washed with brine (5 mL), dried over Na₂SO₄, and concentrated to give (R)-1-(6-fluoro-3′-methylbiphenyl-2-yl)-5-methoxy-1-((R)-morpholin-2-yl)pentan-1-ol (0.38, quant.). MS m/z 388 (M+H⁺).

The following morpholines were prepared using procedures analogous to those described above:
(R)-1-(6-chloro-3′-ethylbiphenyl-2-yl)-5-methoxy-1-((R)-morpholin-2-yl)pentan-1-ol using 2-bromo-6-chloro-3′-ethylbiphenyl in Step 7.
(R)-1-(4′,6-difluoro-3′-methylbiphenyl-2-yl)-5-methoxy-1-((R)-morpholin-2-yl)pentan-1-ol using 2-bromo-4′,6-difluoro-3′-methylbiphenyl in Step 7.
(R)-1-(6-fluoro-3′-methoxy-5′-methylbiphenyl-2-yl)-5-methoxy-1-((R)-morpholin-2-yl)pentan-1-ol using 2-bromo-6-fluoro-3′-methoxy-5′-methylbiphenyl in Step 7.
(1R)-1-(6-chloro-2′-fluoro-5′-methylbiphenyl-2-yl)-5-methoxy-1-((R)-morpholin-2-yl)pentan-1-ol using 2′-bromo-6′-chloro-2-fluoro-5-methylbiphenyl in Step 7.
(R)-1-(3-chloro-2-(naphthalen-2-yl)phenyl)-5-methoxy-1-((R)-morpholin-2-yl)pentan-1-ol using 2-(2-bromo-6-chlorophenyl)naphthalene in Step 7.
(R)-1-(3-chloro-2-(quinolin-3-yl)phenyl)-5-methoxy-1-((R)-morpholin-2-yl)pentan-1-ol using 3-(2-bromo-6-chlorophenyl)quinoline in Step 7.
(R)-1-(6-chloro-3′-(methoxymethyl)biphenyl-2-yl)-5-methoxy-1-((R)-morpholin-2-yl)pentan-1-ol using 2-bromo-6-chloro-3′-(methoxymethyl)biphenyl in Step 7.
(1R)-1-[2-chloro-3-(3-ethylphenyl)-4-pyridinyl]-5-(methyloxy)-1-[(2R)-2-morpholinyl]-1-pentanol using 4-bromo-2-chloro-3-(3-ethylphenyl)pyridine in Step 7.
1-[2-(1-benzothien-3-yl)-3-chlorophenyl]-5-(methyloxy)-1-[(2R)-2-morpholinyl]-1-pentanol using 3-(2-bromo-6-chlorophenyl)-1-benzothiophene in Step 7.
(1R)-1-[4-chloro-3-(3-ethylphenyl)-2-pyridinyl]-5-(methyloxy)-1-[(2R)-2-morpholinyl]-1-pentanol using 2-bromo-4-chloro-3-(3-ethylphenyl)pyridine in Step 7.
(1R)-1-{2-chloro-3-[3-(1-methylethyl)phenyl]-4-pyridinyl}-5-(methyloxy)-1-[(2R)-2-morpholinyl]-1-pentanol using 4-bromo-2-chloro-3-[3-(1-methylethyl)phenyl]pyridine in Step 7.

### Preparation 10

### Methyl {(4R)-4-(3′-ethyl-6-fluoro-2-biphenylyl)-4-hydroxy-4-[(2R)-2-morpholinyl]butyl}carbamate

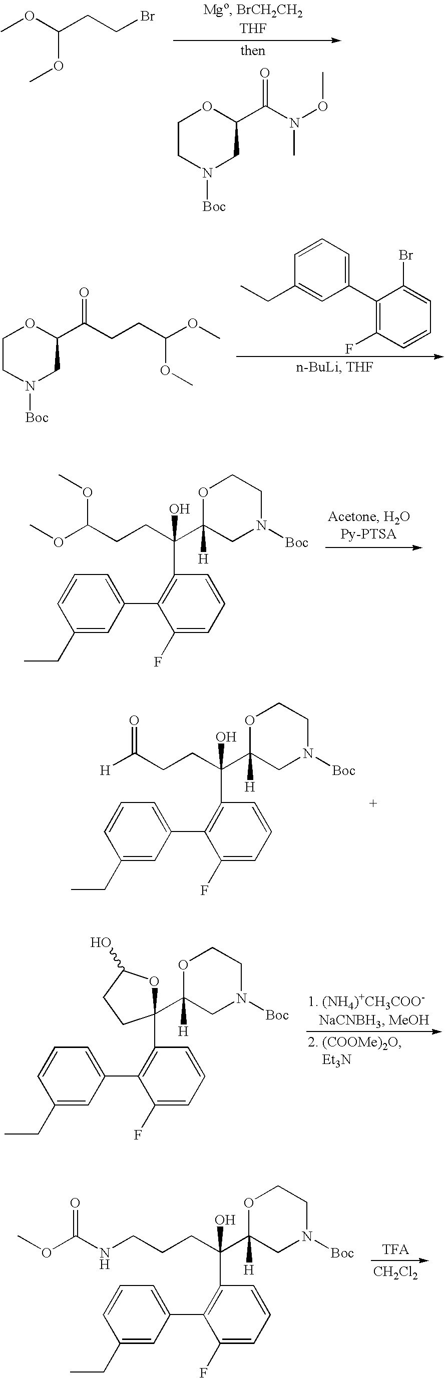

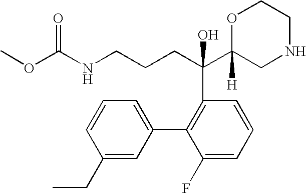

### Step 1. 1,1-dimethylethyl (2R)-2-[4,4-bis(methyloxy)butanoyl]-4-morpholinecarboxylate

To a stirred solution of Mg^{o} (960 mg, 40.0 mmol) in 35.0 mL of dry THF, was added at reflux 0.3 mL of BrCH₂CH₂Br (0.3 mL, 3.5 mmol) and 3-bromo-1,1-bis(methyloxy)propane (6.45 g, 35.0 mmol). The resulting mixture was then heated at reflux for 1 h. Then the solution was cooled to room temperature and added to a solution of 1,1-dimethylethyl (2R)-2-{[methyl(methyloxy)amino]carbonyl}-4-morpholinecarboxylate (5.46 g, 20.0 mmol) dissolved in 20.0 mL of dry THF at −30° C. The mixture was warmed to room temperature and stirred overnight. The mixture was quenched with NH₄Cl and extracted with ethyl acetate. The organic layer was then dried, filtered, and concentrated in vacuo. The crude material was then purified via column chromatography to afford 1,1-dimethylethyl (2R)-2-[4,4-bis(methyloxy)butanoyl]-4-morpholinecarboxylate (5.06 g, 81%).

### Step 2. 1,1-dimethylethyl (2R)-2-[(1R)-1-(3′-ethyl-6-fluoro-2-biphenylyl)-1-hydroxy-4,4-bis(methyloxy)butyl]-4-morpholinecarboxylate

To a stirred solution of 2-bromo-3′-ethyl-6-fluorobiphenyl (6.6 g, 23.6 mmol) in 15.0 mL of dry THF at −78° C., was added n-BuLi (10 mL, 25 mmol). The resulting mixture was stirred for 30 min at −78° C. Then, a solution of 1,1-dimethylethyl (2R)-2-[4,4-bis(methyloxy)butanoyl]-4-morpholinecarboxylate (5.0 g, 15.7 mmol) dissolved in 15.0 ml of dry THF was added. The resulting solution was warmed to room temperature over 3 h and quenched with 0.5N HCl. The mixture was then extracted with ethyl acetate. The organic layer was then dried, filtered, and concentrated in vacuo. The crude residue was purified via column chromatography to give 1,1-dimethylethyl (2R)-2-[(1R)-1-(3′-ethyl-6-fluoro-2-biphenylyl)-1-hydroxy-4,4-bis(methyloxy)butyl]-4-morpholinecarboxylate (6.7 g, 82%).

### Step 3. Mixture of 1,1-dimethylethyl (2R)-2-[(1R)-1-(3′-ethyl-6-fluoro-2-biphenylyl)-1-hydroxy-4-oxobutyl]-4-morpholinecarboxylate and 1,1-dimethylethyl (2R)-2-[(2R)-2-(3′-ethyl-6-fluoro-2-biphenylyl)-5-hydroxytetrahydro-2-furanyl]-4-morpholinecarboxylate

To a microwave vial containing 1,1-dimethylethyl (2R)-2-[(1R)-1-(3′-ethyl-6-fluoro-2-biphenylyl)-1-hydroxy-4,4-bis(methyloxy)butyl]-4-morpholinecarboxylate (180 mg, 0.35 mmol) dissolved in acetone (2 mL) and H₂O (0.8 mL), a catalytic amount of pyridinium p-toluenesulfonate (22 mg, 0.088 mmol) was added and the mixture was stirred under microwave irradiation for 30 min 100° C. The mixture was quenched with NaHCO₃ and then extracted with ethyl acetate. The organic layer was then dried, filtered, and concentrated in vacuo. The crude mixture of 1,1-dimethylethyl (2R)-2-[(1R)-1-(3′-ethyl-6-fluoro-2-biphenylyl)-1-hydroxy-4-oxobutyl]-4-morpholinecarboxylate and 1,1-dimethylethyl (2R)-2-[(2R)-2-(3′-ethyl-6-fluoro-2-biphenylyl)-5-hydroxytetrahydro-2-furanyl]-4-morpholinecarboxylate was used in the next step without other purification.

### Step 4. 1,1-dimethylethyl (2R)-2-((1R)-1-(3′-ethyl-6-fluoro-2-biphenylyl)-1-hydroxy-4-{[(methyloxy)carbonyl]amino}butyl)-4-morpholinecarboxylate

To a microwave vial containing the mixture of 1,1-dimethylethyl (2R)-2-[(1R)-1-(3′-ethyl-6-fluoro-2-biphenylyl)-1-hydroxy-4-oxobutyl]-4-morpholinecarboxylate and 1,1-dimethylethyl (2R)-2-[(2R)-2-(3′-ethyl-6-fluoro-2-biphenylyl)-5-hydroxytetrahydro-2-furanyl]-4-morpholinecarboxylate (500 mg, 1.1 mmol) dissolved in dry MeOH (8 mL), was added (NH₄)⁺CH₃COO⁻ (3.0 g) was followed by NaCNBH₃ (135 mg, 2.2 mmol). The mixture was then stirred for 30 min at 100° C. under microwave irradiation. The solvent was removed in vacuo and the residue redissolved in methylene chloride and washed with NaHCO₃. The organic layer was then dried, filtered, and concentrated in vacuo. The crude material was then purified via SCX (10 g) column. The amine was then dissolved in methylene chloride (8.0 mL) and Et₃N (0.300 g, 3.0 mmol) and (COOMe)₂O (0.328 g, 2.0 mmol) added. The resulting mixture was stirred for 20 min at room temperature. The reaction was diluted with methylene chloride and washed with NaHCO₃. The organic layer was then dried, filtered, and concentrated in vacuo. The crude material was then purified by flash chromatography to afford 1,1-dimethylethyl (2R)-2-((1R)-1-(3′-ethyl-6-fluoro-2-biphenylyl)-1-hydroxy-4-{[(methyloxy)carbonyl]amino}butyl)-4-morpholinecarboxylate (0.135 g, 23%). MS (E/Z): 431.2 (M-Boc+H⁺)

### Step 5. Methyl {(4R)-4-(3′-ethyl-6-fluoro-2-biphenylyl)-4-hydroxy-4-[(2R)-2-morpholinyl]butyl}carbamate

To a solution of 1,1-dimethylethyl (2R)-2-41R)-1-(3′-ethyl-6-fluoro-2-biphenylyl)-1-hydroxy-4-{[(methyloxy)carbonyl]amino}butyl)-4-morpholinecarboxylate (135 mg, 0.25 mmol) in methylene chloride (4 mL) at 0° C., was added TFA (1.0 mL, 25% v/v). The solution was stirred for 1.5 h at room temperature before the solvent was removed in vacuo and the crude material filtered through an SCX column (5 g) to afford methyl {(4R)-4-(3′-ethyl-6-fluoro-2-biphenylyl)-4-hydroxy-4-[(2R)-2-morpholinyl]butyl}carbamate (0.100 g, >99%).

The following morpholines were prepared using procedures analogous to those described above:
Methyl {(4R)-4-(6-chloro-3′-methyl-2-biphenylyl)-4-hydroxy-4-[(2R)-2-morpholinyl]butyl}carbamate using 2-bromo-6-chloro-3′-methylbiphenyl in Step 2.

### Preparation 11

### methyl 1,2,3-oxathiazolidine-3-carboxylate 2,2-dioxide

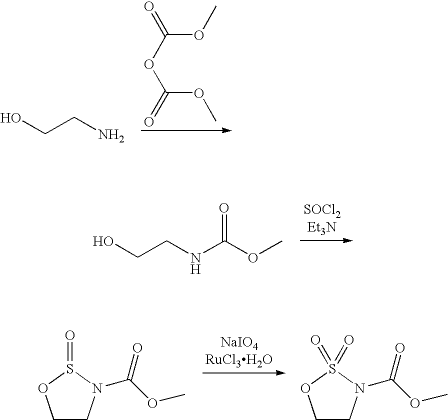

### Step 1. Methyl (2-hydroxyethyl)carbamate

To a stirred solution of 2-aminoethanol (6.11 g, 100 mmol) in dry dichloromethane (120 mL) at room temperature was added dropwise a solution of dimethyl dicarbonate (14.1 g, 105 mmol) in 20 mL. The resulting mixture was stirred for 5 hours before the solvent was removed in vacuo to afford methyl (2-hydroxyethyl)carbamate (12.1 g) as a colorless oil. ¹H NMR (400 MHz, CDCl₃): 2.21 (broad, 1H), 3.37 (q, 2H), 3.70-3.73[s (3H)+q (2H)], 5.20 (broad, 1H).

### Step 2. Methyl 1,2,3-oxathiazolidine-3-carboxylate 2-oxide

To a stirred suspension of methyl (2-hydroxyethyl)carbamate (100 mmol, 12.1 g) in dry dichloromethane (700 mL) at −78° C. was added triethylamine (30.4 g, 42 ml, 300 mmol) followed by thionyl chloride (17.9 g, 11 mL, 150 mmol). The resulting yellow suspension was stirred at −78° C. for 3 hours before it was quenched with methanol (3,2 g, 4 ml, 100 mmol) and warmed to room temperature. The reaction mixture was filtered and the Filtrate concentrated to remove all the dichloromethane before being re-dissolved in 900 ml Et₂O, filtered and concentrated again. The resulting crude was purified by passing through a 15 cm silica plug eluted with 30% ethyl acetate in hexane to afford methyl 1,2,3-oxathiazolidine-3-carboxylate 2-oxide (7.905 g, 48%) as a yellowish oil. ¹H NMR (400 MHz, CDCl₃): 3.64 (m, 1H), 3.88 (s, 3H), 3.97 (m, 1H), 4.77 (m, 1H), 5.03 (m, 1H).

### Step 3. Methyl 1,2,3-oxathiazolidine-3-carboxylate 2,2-dioxide

To a stirred solution of methyl 1,2,3-oxathiazolidine-3-carboxylate 2-oxide (7.905 g, 47.9 mmol) in acetonitrile (45 mL) at 0° C. was added RuCl₃.H₂O (54 mg, 0.24 mmol) followed by NaIO₄ (15.4 g, 71.8 mmol) and water (45 mL). The resulting mixture was allowed to warm to room temperature and stir for two hours before it was filtered. The filtrate was concentrated in vacuo and then redistributed in 800 mL MTBE and filtered again. The resulting solution was washed with water (50 mL), brine (2×100 mL), dried over Na₂SO₄, filtered and concentrated in vacuo to provide methyl 1,2,3-oxathiazolidine-3-carboxylate 2,2-dioxide (6.5 g, 75%) as an off-white solid. ¹H NMR (400 MHz, CDCl₃): 3.95 (s, 3H), 4.14 (t, 2H), 4.69 (t, 2H).

### Preparation 12

### methyl [2-({(S)-(6-chloro-3′-ethyl-2-biphenylyl)[(2R)-2-morpholinyl]methyl}oxy)ethyl]carbamate

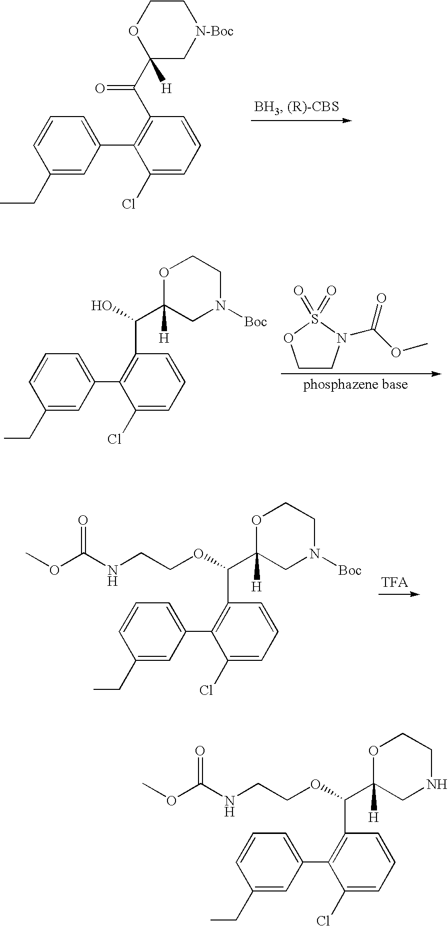

### Step 1. (1,1-dimethylethyl (2R)-2-[(S)-(6-chloro-3′-ethyl-2-biphenylyl)(hydroxy)methyl]-4-morpholinecarboxylate

To a stirred solution of 1,1-dimethylethyl (2R)-2-[(6-chloro-3′-ethyl-2-biphenylyl)carbonyl]-4-morpholinecarboxylate (2.87 g, 6.7 mmol) in TBME (75 mL) under argon at room temperature was added drop-wise borane-methyl sulfide complex (2M in toluene, 4.5 mL, 9 mmol) and (R)-2-methyl-CBS-oxazaborolidine (1 M in toluene, 0.7 mL, 0.7 mmol). The resulting solution was heated at 40° C. for 4 hours at which time TLC analysis showed complete consumption of the ketone. The reaction was quenched with 2 mL of water added slowly and then partitioned between 700 mL Et₂O and 150 mL brine. The organic layer was washed with brine (1×50 mL), dried over Na₂SO₄, filtered, and concentrated under reduced pressure. The crude was purified by flash chromatography (ISCO, 120 g column, EtOAc Hexane, 0-25%) to provide (1,1-dimethylethyl (2R)-2-[(S)-(6-chloro-3′-ethyl-2-biphenylyl)(hydroxy)methyl]-4-morpholinecarboxylate (1.392 g, 48%, the less polar diastereomer).

### Step 2. 1,1-dimethylethyl (2R)-2-{(S)-(6-chloro-3′-ethyl-2-biphenylyl)[(2-{[(methyloxy)carbonyl]amino}ethyl)oxy]methyl}-4-morpholinecarboxylate

To a stirred solution of (1,1-dimethylethyl (2R)-2-[(S)-(6-chloro-3′-ethyl-2-biphenylyl)(hydroxy)methyl]-4-morpholinecarboxylate (0.432 g, 1 mmol) in 6 mL of dry DMF at room temperature was added phosphazene base P₄-t-Bu (1.0M in n-hexane, 2 ml, 2 mmol). The resulting mixture was stirred for 10 minutes under argon before a solution of methyl 1,2,3-oxathiazolidine-3-carboxylate 2,2-dioxide (0.362 g, 2 mmol) in 2 mL dry DMF was added. The resulting solution was stirred at room temperature overnight (18 hours). The crude reaction mixture was purified via HPLC (Gilson, C₁₈ column, 5 um, 50×100 mm), CH₃CN/water (w/0.1% TFA) 65-95%) to provide 1,1-dimethylethyl (2R)-2-{(S)-(6-chloro-3′-ethyl-2-biphenylyl)[(2-{[(methyloxy) carbonyl]amino}ethyl)oxy]methyl}-4-morpholinecarboxylate as colorless oil. MS (E/Z): 533.4 (M+H⁺).

### Step 3. Methyl [2-({(S)-(6-chloro-3′-ethyl-2-biphenylyl)[(2R)-2-morpholinyl]methyl}oxy)ethyl]carbamate

To a stirred solution of 1,1-dimethylethyl (2R)-2-{(S)-(6-chloro-3′-ethyl-2-biphenylyl) [(2-{[(methyloxy)carbonyl]amino}ethyl)oxy]methyl}-4-morpholinecarboxylate in DCM (4 mL) at room temperature was added TFA (4 mL). The resulting mixture was stirred at room temperature for 1.5 h. The crude was concentrated under reduced pressure and then partitioned between 450 mL DCM and 50 mL saturated Na₂CO₃ solution. The organic layer was washed with brine, dried over Na₂SO₄, filtered and concentrated to provide methyl [2-({(S)-(6-chloro-3′-ethyl-2-biphenylyl)[(2R)-2-morpholinyl]methyl}oxy)ethyl]carbamate (407 mg, 68% for steps 2 and 3). MS (E/Z): 433.0 (M+H⁺).

### Preparation 13

### Methyl {4-(6-chloro-3′-methyl-2-biphenylyl)-4-hydroxy-4-[(3R)-3-piperidinyl]butyl}carbamate

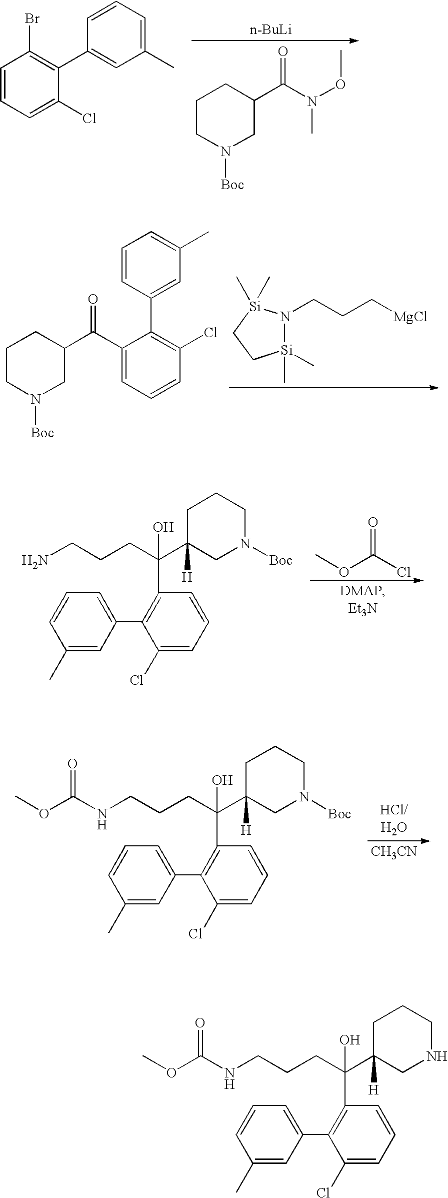

### Step 1. (R)-tert-butyl 3-(6-chloro-3′-methylbiphenylcarbonyl)piperidine-1-carboxylate

To a solution of 6-bromo-2-fluoro-3′-methylbiphenyl (2 g, 7.14 mmol) in anhydrous THF (30 mL) cooled to −78° C. was added dropwise a solution of 1.6 M of n-BuLi in hexane (4.46 mL). The reaction mixture was stirred at −78° C. for 1 h and a solution of (R)-tert-butyl 3-(methoxy(methyl)carbamoyl)piperidine-1-carboxylate (1.94 g, 7.14 mmol) in anhydrous THF (20 mL) was added. The mixture was allowed to warm to rt and stirred overnight. The mixture was quenched with satd aq NH₄Cl (40 mL) and extracted with EtOAc (40 mL). The combined organic layers were dried over Na₂SO₄ and concentrated to give crude product, which was purified by flash column chromatography to afford (R)-tert-butyl 3-(6-chloro-3′-methylbiphenylcarbonyl)piperidine-1-carboxylate (1 g, 34%). ¹H NMR (400 MHz, CD₃OD): 0.80-1.20 (m, 8H), 1.30 (s, 1H), 1.40 (s, 1H), 1.40-1.60 (m, 2H), 2.00-2.18 (s, 1H), 2.30-2.40 (s, 3H), 2.60-2.80 (m, 2H), 3.50-3.80 (m, 2H), 7.00-7.15 (s, 2H), 7.20-7.30 (d, 1H), 7.30-7.40 (t, 2H), 7.39-7.48 (t, 1H), 7.60-7.70 (d, 1H); MS (E/Z): 414 (M+H⁺)

### Step 2. 1,1-dimethylethyl (3R)-3-[4-amino-1-(6-chloro-3′-methyl-2-biphenylyl)-1-hydroxybutyl]-1-piperidinecarboxylate

To a solution of (R)-tert-butyl 3-(6-chloro-3′-methylbiphenylcarbonyl)piperidine-1-carboxylate (800 mg, 1.94 mmol) in anhydrous THF (15 mL) cooled to −78° C. was added dropwise a solution of 2 M (3-(2,2,5,5-tetramethyl-1,2,5-azadisilolidin-1-yl)propyl)magnesium chloride in THF (0.968 mL, 1.94 mmol). After addition, the reaction mixture was allowed to warm slowly to rt while stirring overnight. The mixture was quenched with satd aq NH₄Cl (15 mL) and extracted with CH₂Cl₂ (3×). The combined organic layers were dried over Na₂SO₄ and concentrated to give crude 1,1-dimethylethyl (3R)-3-[4-amino-1-(6-chloro-3′-methyl-2-biphenylyl)-1-hydroxybutyl]-1-piperidinecarboxylate (900 mg), which was used in the next step without further purification.

### Step 3. 1,1-dimethylethyl (3R)-3-(1-(6-chloro-3′-methyl-2-biphenylyl)-1-hydroxy-4-{[(methyloxy)carbonyl]amino}butyl)-1-piperidinecarboxylate

To a solution of (1,1-dimethylethyl (3R)-3-[4-amino-1-(6-chloro-3′-methyl-2-biphenylyl)-1-hydroxybutyl]-1-piperidinecarboxylate (800 mg, 1.69 mmol) in anhydrous CH₂Cl₂ (15 mL) were added 4-dimethylaminopyridine (1.24 g, 10.17 mmol) and Et₃N (2.35 mL, 16.95 mmol). The mixture was cooled with an ice bath and methyl chloroformate (0.65 mL, 8.47 mmol) in CH₂Cl₂ (5 mL) was added. The reaction mixture was allowed to warm slowly to rt while stirring overnight. The solvent was removed in vacuo and the residue was purified by column chromatography to afford 1,1-dimethylethyl (3R)-3-(1-(6-chloro-3′-methyl-2-biphenylyl)-1-hydroxy-4-{[(methyloxy)carbonyl]amino}butyl)-1-piperidinecarboxylate (700 mg, 78%). ¹H NMR (400 MHz, CD₃OD): 1.00-1.70 (m, 17H), 2.30-2.50 (d, 3H), 2.50-2.70 (s, 1H), 2.90-2.31 (m, 2H), 3.50-3.52 (m, 3H), 3.80-4.20 (m, 2H), 6.0-7.15 (m, 3H), 7.15-7.40 (m, 3H), 7.50-7.70 (m, 1H); MS (E/Z): 531 (M+H⁺)

### Step 4. Methyl {4-(6-chloro-3′-methyl-2-biphenylyl)-4-hydroxy-4-[(3R)-3-piperidinyl]butyl}carbamate

To a solution of 1,1-dimethylethyl (3R)-3-(1-(6-chloro-3′-methyl-2-biphenylyl)-1-hydroxy-4-{[(methyloxy)carbonyl]amino}butyl)-1-piperidinecarboxylate (600 mg, 1.13 mg) in CH₃CN (18 mL) was added 2N aq HCl (15 mL) and the reaction mixture was vigorously stirred overnight at rt. The solvents were removed in vacuo to give methyl methyl {4-(6-chloro-3′-methyl-2-biphenylyl)-4-hydroxy-4-[(3R)-3-piperidinyl]butyl}carbamate as its hydrochloride salt (500 mg, 95.8%). ¹H NMR (400 MHz, CD₃OD): 1.00-1.20 (m, 1H), 1.30-1.80 (m, 8H), 1.80-2.00 (m, 2H), 2.40-2.50 (d, 3H), 2.75-2.90 (t, 1H), 2.90-3.05 (m, 3H), 3.05-3.12 (t, 1H), 3.20-3.30 (m, 1H), 3.30-3.40 (m, 1H), 3.60-3.70 (d, 4H), 6.90-6.98 (d, 1H), 7.00-7.12 (m, 1H), 7.25-7.50 (m, 4H), 7.75-7.85 (d, 1H); MS (E/Z): 431 (M+H⁺)

The following piperidines were prepared using procedures analogous to those described above:
Methyl 4-(6-chloro-3′-methylbiphenyl-2-yl)-4-hydroxy-4-((R)-piperidin-3-yl)butylcarbamate using 6-bromo-2-chloro-3′-methylbiphenyl in Step 1.
N-(4-hydroxy-4-((R)-piperidin-3-yl)-4-(2-(o-tolyloxy)phenyl)butyl)acetamide using 1-bromo-2-(o-tolyloxy)benzene in Step 1 and acetyl chloride in place of methyl chloroformate in Step 3.
Methyl 4-hydroxy-4-((R)-piperidin-3-yl)-4-(2-(o-tolyloxy)phenyl)butylcarbamate using 1-bromo-2-(o-tolyloxy)benzene in Step 1.
Methyl 4-(3′-ethyl-6-fluorobiphenyl-2-yl)-4-hydroxy-4-((R)-piperidin-3-yl)butylcarbamate using 2-bromo-3′-ethyl-6-fluorobiphenyl in Step 1.
Methyl 4-(6-fluoro-3′-methoxybiphenyl-2-yl)-4-hydroxy-4-((R)-piperidin-3-yl)butylcarbamate using 2-bromo-6-fluoro-3′-methoxybiphenyl in Step 1.
Methyl 4-(6-chloro-3′-isopropylbiphenyl-2-yl)-4-hydroxy-4-((R)-piperidin-3-yl)butylcarbamate using 2-bromo-6-chloro-3′-isopropylbiphenyl in Step 1.
Methyl 4-(6-chloro-3′-methoxybiphenyl-2-yl)-4-hydroxy-4-((R)-piperidin-3-yl)butylcarbamate using 2-bromo-6-chloro-3′-methoxybiphenyl in Step 1.
Methyl 4-(2′,6-difluoro-5′-methylbiphenyl-2-yl)-4-hydroxy-4-((R)-piperidin-3-yl)butylcarbamate using 2′-bromo-2,6′-difluoro-5-methylbiphenyl in Step 1.
Methyl {4-(6-chloro-3′-ethyl-2-biphenylyl)-4-hydroxy-4-[(3R)-3-piperidinyl]butyl}carbamate using 2-bromo-6-chloro-3′-ethylbiphenyl in Step 1.
Methyl [4-hydroxy-4-[(3R)-3-piperidinyl]-4-(2′,4,6-trifluoro-5′-methyl-2-biphenylyl)butyl]carbamate using 2-bromo-2′,4,6-trifluoro-5′-methylbiphenyl in Step 1.
Methyl {4-(6-fluoro-3′-methyl-2-biphenylyl)-4-hydroxy-4-[(3R)-3-piperidinyl]butyl}carbamate using 2-bromo-6-fluoro-3′-methylbiphenyl in Step 1.
Methyl {4-[6-fluoro-3′-methyl-5-(methyloxy)-2-biphenyl]-4-hydroxy-4-[(3R)-3-piperidinyl]butyl}carbamate using 6-bromo-2-fluoro-3′-methyl-3-(methyloxy)biphenyl in Step 1.
Methyl {4-[3-chloro-2-(3-quinolinyl)phenyl]-4-hydroxy-4-[(3R)-3-piperidinyl]butyl}carbamate using 3-(2-bromo-6-chlorophenyl)quinoline in Step 1.
Methyl {4-{3-chloro-2-[4-(1-methylethyl)-2-quinazolinyl]phenyl}-4-hydroxy-4-[(3R)-3-piperidinyl]butyl}carbamate using 2-(2-bromo-6-chlorophenyl)-4-(1-methylethyl)quinazoline in Step 1.
Methyl {4-[2-chloro-3-(3-ethylphenyl)-4-pyridinyl]-4-hydroxy-4-[(3R)-3-piperidinyl]butyl}carbamate using 4-bromo-2-chloro-3-(3-ethylphenyl)pyridine in Step 1.
Methyl {4-{3-chloro-2-[8-(1-methylethyl)-2-quinolinyl]phenyl}-4-hydroxy-4-[(3R)-3-piperidinyl]butyl}carbamate using 2-(2-bromo-6-chlorophenyl)-8-(1-methylethyl)quinoline in Step 1.
Methyl {4-[3-fluoro-2-(3-quinolinyl)phenyl]-4-hydroxy-4-[(3R)-3-piperidinyl]butyl}carbamate using 3-(2-bromo-6-fluorophenyl)quinoline in Step 1.
Methyl {4-[3-chloro-2-(5-methyl-2-furanyl)phenyl]-4-hydroxy-4-[(3R)-3-piperidinyl]butyl}carbamate using 2-(2-bromo-6-chlorophenyl)-5-methylfuran in Step 1.
Methyl {4-(6-chloro-3′-fluoro-5′-methyl-2-biphenylyl)-4-hydroxy-4-[(3R)-3-piperidinyl]butyl}carbamate using 2-bromo-6-chloro-3′-fluoro-5′-methylbiphenyl in Step 1.
Methyl {4-(3′,6-difluoro-5′-methyl-2-biphenylyl)-4-hydroxy-4-[(3R)-3-piperidinyl]butyl}carbamate using 2-bromo-3′,6-difluoro-5′-methylbiphenyl in Step 1.
Methyl {4-[3-chloro-2-(2,3-dihydro-1-benzofuran-6-yl)phenyl]-4-hydroxy-4-[(3R)-3-piperidinyl]butyl}carbamate using 6-(2-bromo-6-chlorophenyl)-2,3-dihydro-1-benzofuran in Step 1.
Methyl {4-[6-chloro-2′-(methyloxy)-5′-(trifluoromethyl)-2-biphenyl)-1]-4-hydroxy-4-[(3R)-3-piperidinyl]butyl}carbamate using 2′-bromo-6′-chloro-2-(methyloxy)-5-(trifluoromethyl)biphenyl in Step 1.
Methyl {4-(2′,6-difluoro-5′-methyl-2-biphenylyl)-4-hydroxy-4-[(3R)-3-piperidinyl]butyl}carbamate using 2′-bromo-2,6′-difluoro-5-methylbiphenyl in Step 1.
Methyl {4-[6-fluoro-3′-(1-methylethyl)-2-biphenyl]-4-hydroxy-4-[(3R)-3-piperidinyl]butyl}carbamate using 2-bromo-6-fluoro-3′-(1-methylethyl)biphenyl in Step 1.
Methyl {4-{3-chloro-2-[(3-methylphenyl)methyl]phenyl}-4-hydroxy-4-[(3R)-3-piperidinyl]butyl}carbamate using 1-bromo-3-chloro-2-[(3-methylphenyl)methyl]benzene in Step 1.

### Preparation 14

### 1,1-dimethylethyl (3R)-3-418)-1-(6-chloro-3′-ethyl-2-biphenylyl)-1-hydroxy-4-{[(methyloxy)carbonyl]amino}butyl)-1-piperidinecarboxylate

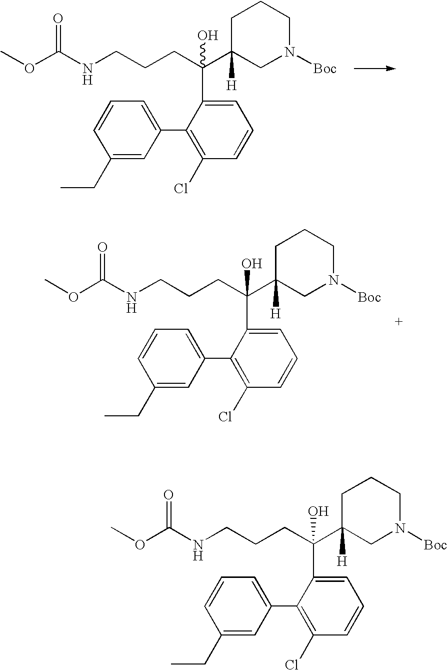

The two diastereomers of 1,1-dimethylethyl (3R)-3-(1-(6-chloro-3′-ethyl-2-biphenylyl)-1-hydroxy-4-{[(methyloxy)carbonyl]amino}butyl)-1-piperidinecarboxylate (400 mg, 1:1 ratio) were separated via reverse phase HPLC. The sample was dissolved in 1 mL DMSO and 2 mL MeOH was loaded onto the reverse phase HPLC (100×50 mm Gemini 10 μM column (C18 110R) connected directly to a 100×50 mm Sunfire column (C18 OBD), 60:40 acetonitrile/water containing 0.1% TFA, 50 minute separation time). The early fractions were combined, neutralized with NH₄OH, and concentrated. The remaining aqueous mixture was extracted with CH₂Cl₂. The combined organic layers were dried over Na₂SO₄, filtered, and concentrated. A total of 4.4 g of a 1,1-dimethylethyl (3R)-3-(1-(6-chloro-3′-ethyl-2-biphenylyl)-1-hydroxy-4-{[(methyloxy)carbonyl]amino}butyl)-1-piperidinecarboxylate (1:1 diastereomeric mixture) was separated by repeating this method to provide the desired diastereomer 1,1-dimethylethyl (3R)-3-((1S)-1-(6-chloro-3′-ethyl-2-biphenylyl)-1-hydroxy-4-{[(methyloxy)carbonyl]amino}butyl)-1-piperidinecarboxylate (1.5 g, 34% yield).

### Preparation 15

### Methyl {4-{5-chloro-4-[3-(1-methylethyl)phenyl]-3-pyridinyl}-4-hydroxy-4-[(3R)-3-piperidinyl]butyl}carbamate

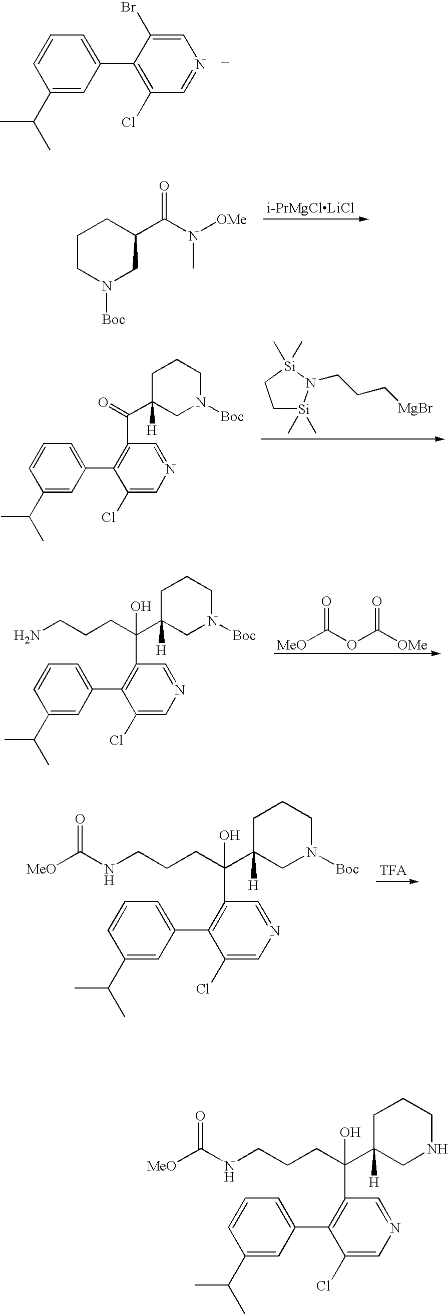

### Step 1: 1,1-dimethylethyl (3R)-3-({5-chloro-4-[3-(1-methylethyl)phenyl]-3-pyridinyl}carbonyl)-1-piperidinecarboxylate

To a cold (0° C.) solution of 3-bromo-5-chloro-4-(3-isopropylphenyl)pyridine (0.78 g, 2.51 mmol) in THF (1.5 mL) was added a i-PrMgCl.LiCl solution (2.6 mL, 1.0 M in THF, 2.6 mmol). After stirring for 45 minutes at 0° C., a solution 1,1-dimethylethyl (3R)-3-{[methyl(methyloxy)amino]carbonyl}-1-piperidinecarboxylate (0.53 g, 1.93 mmol) in THF (1.5 mL) was added. The reaction was stirred at 0° C. for 2 hours and then room temperature for 22 hours. The reaction was quenched with a sat. NH₄Cl solution (3 mL). EtOAc (10 mL) and water (2 mL) were added and then the phases separated. The organic phase was washed with brine, dried over MgSO₄, filtered and concentrated to give 1.3 g of a dark brown oil. The crude ketone was used without purification in the subsequent reaction.

### Step 2: 1,1-dimethylethyl (3R)-3-(4-amino-1-{5-chloro-4-[3-(1-methylethyl)phenyl]-3-pyridinyl}-1-hydroxybutyl)-1-piperidinecarboxylate

To a solution of 1,1-dimethylethyl (3R)-3-({5-chloro-4-[3-(1-methylethyl)phenyl]-3-pyridinyl}carbonyl)-1-piperidinecarboxylate (1.93 mmol) in THF (3 mL) at −50° C. was added a hot solution of the alkyl Grignard (9 mL, 0.72 M THF, 5.80 mmol) quickly. The reaction was slowly allowed to warm to room temperature and stirred overnight. The reaction was quenched with a sat. NH₄Cl solution (3 mL). EtOAc and water were added and then the phases were separated. The organic phase was washed with brine, dried over MgSO₄, filtered and concentrated to give 1.6 g of a dark brown oil. The crude 1,1-dimethylethyl (3R)-3-(4-amino-1-{5-chloro-4-[3-(1-methylethyl)phenyl]-3-pyridinyl}-1-hydroxybutyl)-1-piperidinecarboxylate was used without further purification in the subsequent reaction.

### Step 3. 1,1-dimethylethyl (3R)-3-(1-{5-chloro-4-[3-(1-methylethyl)phenyl]-3-pyridinyl}-1-hydroxy-4-{[(methyloxy)carbonyl]amino}butyl)-1-piperidinecarboxylate

To a cold (0° C.) solution of the 1,1-dimethylethyl (3R)-3-(4-amino-1-{5-chloro-4-[3-(1-methylethyl)phenyl]-3-pyridinyl}-1-hydroxybutyl)-1-piperidinecarboxylate (1.93 mmol) in CH₂Cl₂ were added i-Pr₂NEt (1.3 mL, 7.72 mmol) and then dimethyldicarbonate (0.62 mL, 5.79 mmol). After stirring for 1.5 hour at 0° C., the reaction was quenched with a sat. NH₄Cl solution (2 mL). The reaction was diluted with CH₂Cl₂ and phases separated. The aqueous phase was extracted with CH₂Cl₂. The combined organic extracts were washed with brine, dried over MgSO₄, filtered and concentrated to give 2 g of a dark brown oil. The crude residue was purified by flash chromatography on silica gel to give 1,1-dimethylethyl (3R)-3-(1-{5-chloro-4-[3-(1-methylethyl)phenyl]-3-pyridinyl}-1-hydroxy-4-{[(methyloxy)carbonyl]amino}butyl)-1-piperidinecarboxylate (350 mg, 33% yield over 3 steps) as a dark yellow solid.

### Step 4. Methyl {4-{5-chloro-4-[3-(1-methylethyl)phenyl]-3-pyridinyl}-4-hydroxy-4-[(3R)-3-piperidinyl]butyl}carbamate

To a solution of 1,1-dimethylethyl (3R)-3-(1-{5-chloro-4-[3-(1-methylethyl)phenyl]-3-pyridinyl}-1-hydroxy-4-{[(methyloxy)carbonyl]amino}butyl)-1-piperidinecarboxylate (0.35 g, 0.62 mmol) in CH₂Cl₂ (5 mL) was added trifluoroacetic acid (1 mL). After stirring for 2 hours at room temperature, the reaction was concentrated to give a yellow oil. The crude residue was purified with a 5 gm Strata SCX ion exchange resin eluting with 0.6 M NH₃ in MeOH to afford methyl {4-{5-chloro-4-[3-(1-methylethyl)phenyl]-3-pyridinyl}-4-hydroxy-4-[(3R)-3-piperidinyl]butyl}carbamate (278 mg, 97% yield) as a yellow oil.

The following piperidines were prepared using procedures analogous to those described above:
Methyl {4-[5-chloro-4-(3-ethylphenyl)-3-pyridinyl]-4-hydroxy-4-[(3R)-3-piperidinyl]butyl}carbamate

### Preparation 16

### Methyl [2-({(R)-(6-chloro-3′-ethyl-2-biphenylyl)[(3R)-3-piperidinyl]methyl}oxy)ethyl]carbamate

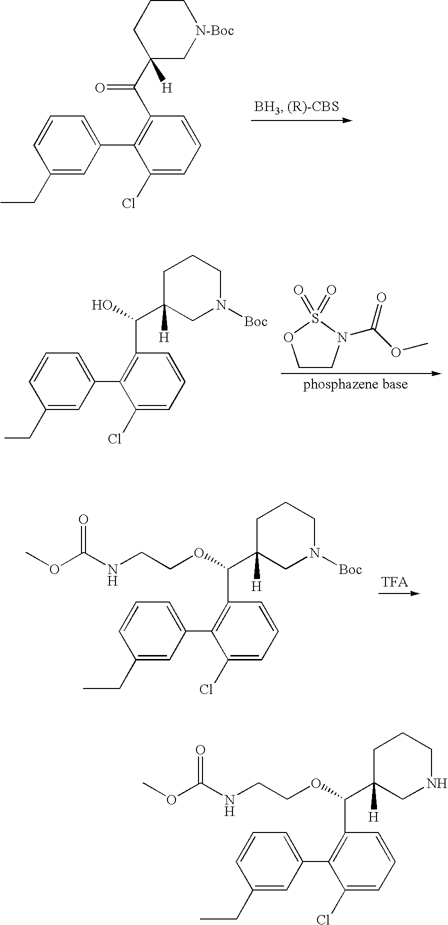

### Step 1. 1,1-dimethylethyl (3R)-3-[(R)-(6-chloro-3′-ethyl-2-biphenylyl)(hydroxy)methyl]-1-piperidinecarboxylate

To a stirred solution of 1,1-dimethylethyl (3R)-3-[(6-chloro-3′-ethyl-2-biphenylyl)carbonyl]-1-piperidinecarboxylate (1.6 g, 3.74 mmol) in TBME (60 mL) under argon at room temperature was added drop-wise simultaneously borane-methyl sulfide complex (2M in toluene, 2.5 ml, 5 mmol) and (R)-2-methyl-CBS-oxazaborolidine (1 M in toluene, 0.4 ml, 0.4 mmol). The resulting solution was heated at 40° C. for 3 h at which time TLC showed that the reaction was complete. The reaction was quenched with 1 mL water added slowly and then partitioned between 600 mL Et₂O and 50 mL water. The organic layer was washed with brine (1×50 mL), dried over Na₂SO₄, filtered, and concentrated under reduced pressure. The crude was purified by flash chromatography (ISCO, 120 g column, 0-30% ethyl acetate/hexane) to provide 1,1-dimethylethyl (3R)-3-[(R)-(6-chloro-3′-ethyl-2-biphenylyl)(hydroxy)methyl]-1-piperidinecarboxylate (1.2 g, 74.6%, less polar diastereomer). MS (E/Z): 430.4 (M+H⁺)

### Step 2. 1,1-dimethylethyl (3R)-3-{(R)-(6-chloro-3′-ethyl-2-biphenylyl)[(2-{[(methyloxy)carbonyl]amino}ethyl)oxy]methyl}-1-piperidinecarboxylate

To a stirred solution of 1,1-dimethylethyl (3R)-3-[(R)-(6-chloro-3′-ethyl-2-biphenylyl)(hydroxy)methyl]-1-piperidinecarboxylate (0.354 g, 0.82 mmol) in 8 mL of dry DMF at room temperature was added phosphazene base P₄-t-Bu (1.0M in n-hexane, 1.64 mL, 1.64 mmol). The resulting mixture was stirred for 10 minutes under argon before a solution of methyl 1,2,3-oxathiazolidine-3-carboxylate 2,2-dioxide (0.298 g, 1.64 mmol) in 2 ml dry DMF was added. The resulting solution was stirred at room temperature overnight (18 hours) before it was quenched with 10 mL saturated NH₄Cl solution. The product was extracted with 300 mL EtOAc and the organic layer was washed with saturated NH₄Cl solution (3×50 mL), HCl (2M, 3×50 mL), brine, and dried over Na₂SO₄, filtered and concentrated in vacuo to provide 1,1-dimethylethyl (3R)-3-{(R)-(6-chloro-3′-ethyl-2-biphenylyl)[(2-{[(methyloxy) carbonyl]amino}ethyl)oxy]methyl}-1-piperidinecarboxylate (452 mg), which was directly used in the next step without further purification. MS (E/Z): 531.4 (M+H⁺)

### Step 3. Methyl [2-({(R)-(6-chloro-3′-ethyl-2-biphenylyl)[(3R)-3-piperidinyl]methyl}oxy)ethyl]carbamate

To a solution of 1,1-dimethylethyl (3R)-3-{(R)-(6-chloro-3′-ethyl-2-biphenylyl)[(2-{[(methyloxy)carbonyl]amino}ethyl)oxy]methyl}-1-piperidinecarboxylate (0.452 g, 0.82 mmol) in DCM (14 mL) at room temperature was added TFA (4 mL). The resulting solution was stirred for 1.5 h. At this time the solvent was removed under reduced pressure and the crude material partitioned between 200 mL DCM and 50 mL 5% Na₂CO₃ solution. The organic layer was washed with water (50 mL), brine, dried over Na₂SO₄, filtered, and concentrated under reduced pressure to provide methyl [2-({(R)-(6-chloro-3′-ethyl-2-biphenylyl)[(3R)-3-piperidinyl]methyl}oxy)ethyl]carbamate (0.35 g, 99%). MS (E/Z): 431.5 (M+H⁺).

### Preparation 17

### N-{4-(6-chloro-3′-ethyl-2-biphenylyl)-4-hydroxy-4-[(3R)-3-piperidinyl]butyl}acetamide

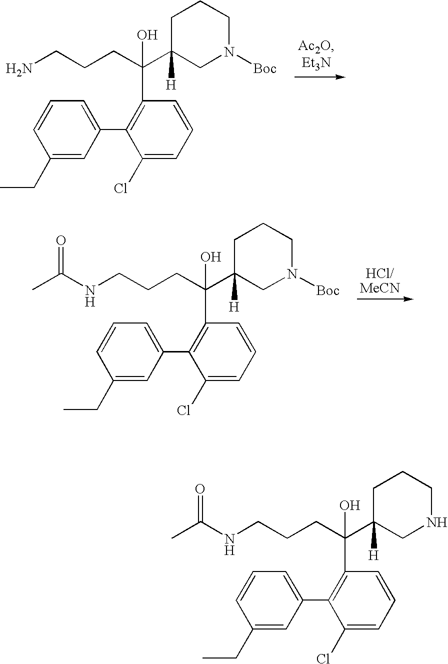

### Step 1. 1,1-dimethylethyl (3R)-3-[4-(acetylamino)-1-(6-chloro-3′-ethyl-2-biphenylyl)-1-hydroxybutyl]-1-piperidinecarboxylate

A solution of 1,1-dimethylethyl (3R)-3-[4-amino-1-(6-chloro-3′-ethyl-2-biphenylyl)-1-hydroxybutyl]-1-piperidinecarboxylate (75 mg, 0.14 mmol) and Et₃N (0.6 mL, 4.3 mmol) in 2 mL of CH₂Cl₂ at 0° C. was treated with a solution of acetic anhydride (0.047 mL, 0.5 mmol) in 2 mL of CH₂Cl₂ and stirred for 2 h. The mixture was concentrated under reduced pressure and subjected to flash chromatography to provide 1,1-dimethylethyl (3R)-3-[4-(acetylamino)-1-(6-chloro-3′-ethyl-2-biphenylyl)-1-hydroxybutyl]-1-piperidinecarboxylate as a colorless oil (53 mg, 73%). MS (m/z) 529.2 (M+H⁺).

### Step 2. N-{4-(6-chloro-3′-ethyl-2-biphenylyl)-4-hydroxy-4-[(3R)-3-piperidinyl]butyl}acetamide

A solution of 1,1-dimethylethyl (3R)-3-[4-(acetylamino)-1-(6-chloro-3′-ethyl-2-biphenylyl)-1-hydroxybutyl]-1-piperidinecarboxylate (50 mg, 0.095 mmol) in 3 mL of CH₃CN at 25° C. was treated with 3 mL of aqueous 2N HCl. After 24 h, the mixture was concentrated under reduced pressure to provide N-{4-(6-chloro-3′-ethyl-2-biphenylyl)-4-hydroxy-4-[(3R)-3-piperidinyl]butyl}acetamide as a white solid (48 mg, quantitative). MS (m/z) 429.2 (M+H⁺).

The following piperidines were prepared following procedures analogous to those described above by substituting the indicated reagent for acetic anhydride in Step 1:

| Structure | Name | Reagent Used in Step 1 |
|---|---|---|
| 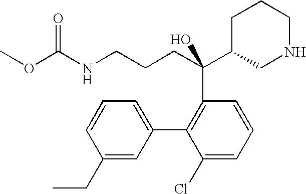 | methyl {4-(6-chloro-3′-ethyl-2- biphenylyl)-4-hydroxy-4-[(3R)-3- piperidinyl]butyl}carbamate | methyl chloroformate |
| 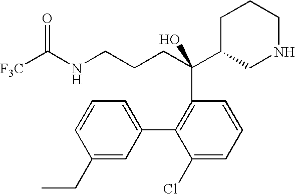 | N-{4-(6-chloro-3′-ethyl-2-biphenylyl)-4- hydroxy-4-[(3R)-3-piperidinyl]butyl}- 2,2,2-trifluoroacetamide | trifluoroacetic acid |
| 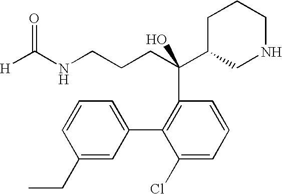 | {4-(6-chloro-3′-ethyl-2-biphenylyl)-4- hydroxy-4-[(3R)-3- piperidinyl]butyl}formamide | methyl formate |
| 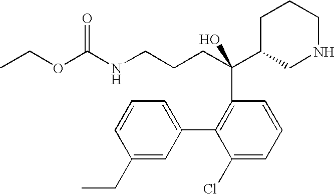 | ethyl {4-(6-chloro-3′-ethyl-2-biphenylyl)- 4-hydroxy-4-[(3R)-3- piperidinyl]butyl}carbamate | ethyl chloroformate |
| 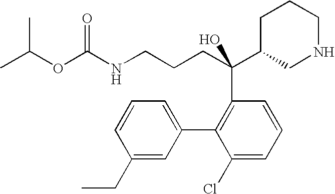 | 1-methylethyl {4-(6-chloro-3′-ethyl-2- biphenylyl)-4-hydroxy-4-[(3R)-3- piperidinyl]butyl}carbamate | isopropyl chloroformate |
| 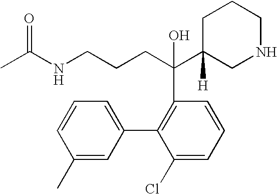 | N-{4-(6-chloro-3′-methyl-2-biphenylyl)-4- hydroxy-4-[(3R)-3- piperidinyl]butyl}acetamide | acetic anhydride |
| 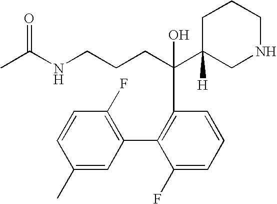 | N-{4-(2′,6-difluoro-5′-methyl-2- biphenylyl)-4-hydroxy-4-[(3R)-3- piperidinyl]butyl}acetamide | acetic anhydride |
| 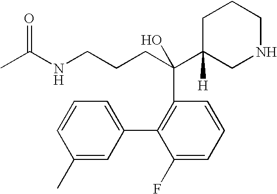 | N-{4-(6-fluoro-3′-methyl-2-biphenylyl)-4- hydroxy-4-[(3R)-3- piperidinyl]butyl}acetamide | acetic anhydride |

### Preparation 18

### N-{4-(6-chloro-3′-ethyl-2-biphenylyl)-4-hydroxy-4-[(3R)-3-piperidinyl]butyl}-2-hydroxyacetamide

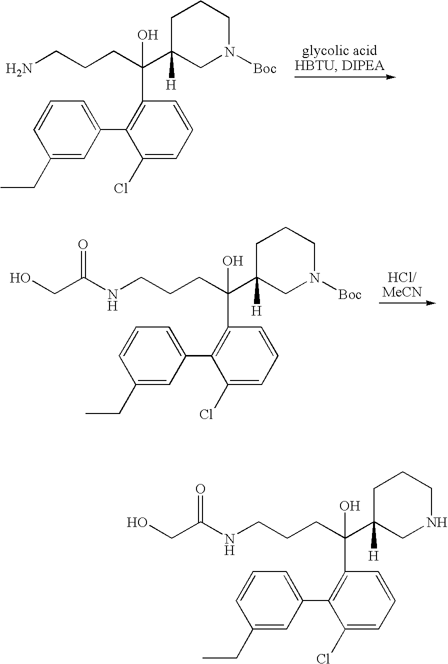

### Step 1. 1,1-dimethylethyl (3R)-3-{1-(6-chloro-3′-ethyl-2-biphenylyl)-1-hydroxy-4-[hydroxyacetyl)amino]butyl}-1-piperidinecarboxylate

A solution of 1,1-dimethylethyl (3R)-3-[4-amino-1-(6-chloro-3′-ethyl-2-biphenylyl)-1-hydroxybutyl]-1-piperidinecarboxylate (75 mg, 0.14 mmol) in 0.5 mL of DMF at 25° C. was treated with glycolic acid (13 mg, 0.17 mmol), i-Pr₂NEt (0.122 mL, 0.7 mmol), and HBTU (64 mg, 0.17 mmol). After 24 h, H₂O was added and the mixture was extracted with EtOAc. The organic extracts were washed (1N aq HCl, 1N aq NaOH, H₂O, brine), dried (Na₂SO₄), concentrated under reduced pressure, and subjected to flash chromatography to provide 1,1-dimethylethyl (3R)-3-{1-(6-chloro-3′-ethyl-2-biphenylyl)-1-hydroxy-4-[(hydroxyacetyl)amino]butyl}-1-piperidinecarboxylate as a colorless oil (39 mg, 51%). MS (m/z) 567.2 (M+Na⁺).

### Step 2. N-{4-(6-chloro-3′-ethyl-2-biphenylyl)-4-hydroxy-4-[(3R)-3-piperidinyl]butyl}-2-hydroxyacetamide

A solution of 1,1-dimethylethyl (3R)-3-{1-(6-chloro-3′-ethyl-2-biphenylyl)-1-hydroxy-4-[(hydroxyacetyl)amino]butyl}-1-piperidinecarboxylate (45 mg, 0.08 mmol) in 3 mL of CH₃CN at 25° C. was treated with 3 mL of aq 2N HCl. After 24 h, the mixture was concentrated under reduced pressure to provide N-{4-(6-chloro-3′-ethyl-2-biphenylyl)-4-hydroxy-4-[(3R)-3-piperidinyl]butyl}-2-hydroxyacetamide as a white solid (41 mg, quantitative). MS (m/z) 445.2 (M+H⁺).

The following piperidines were prepared following procedures analogous to those described above using the appropriate piperidine and the indicated acid in place of glycolic acid in Step 1:

| Structure | Name | Acid Used in Step 1 |
|---|---|---|
| 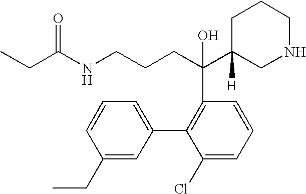 | N-{4-(6-chloro-3′-ethyl-2- biphenylyl)-4-hydroxy-4-[(3R)-3- piperidinyl]butyl}propanamide | propionic acid |
| 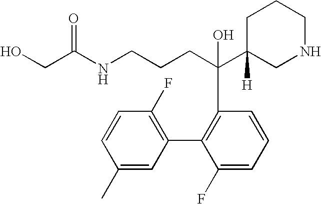 | N-(4-(2′,6-difluoro-5′- methylbiphenyl-2-yl)-4-hydroxy-4- ((R)-piperidin-3-yl)butyl)-2- hydroxyacetamide | glycolic acid |
| 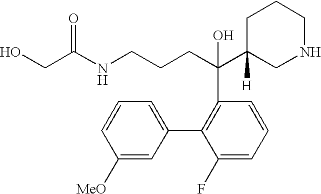 | N-{4-[6-fluoro-3′-(methyloxy)-2- biphenylyl]-4-hydroxy-3-[(3R)-3- piperidinyl]butyl}-2- hydroxyacetamide | glycolic acid |
| 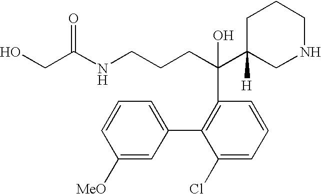 | N-{4-[6-chloro-3′-(methyloxy)-2- biphenylyl]-4-hydroxy-4-[(3R)-3- piperidinyl]butyl}-2- hydroxyacetamide | glycolic acid |
| 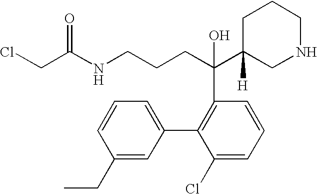 | 2-chloro-N-{(4S)-4-(6-chloro-3′- ethyl-2-biphenylyl)-4-hydroxy-4- [(3R)-3-piperidinyl]butyl}acetamide | chloroacetic acid |

### Preparation 19

### 1-bromo-3-chloro-2-[(3-methylphenyl)methyl]benzene

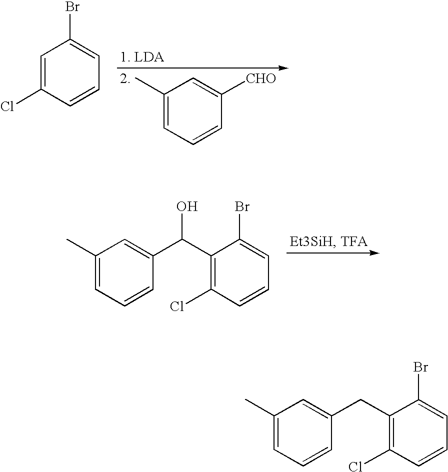

### Step 1. (2-bromo-6-chlorophenyl)(m-tolyl)methanol

To a −78° C. solution of diisopropylamine (9.9 mL, 70 mmol) in anhydrous THF (80 mL) was added dropwise a n-BuLi solution (31.5 mL, 50 mmol, 1.6M hexanes). The reaction was stirred for 20 min at −78° C. and 1-chloro-3-bromobenzene (5.9 mL, 50 mmol) was added. After stirring for 30 min at −78° C., m-tolualdehyde (5.9 mL, 50 mmol) was added. The reaction was gradually allowed to warm to rt and then stirred overnight. The reaction was quenched with the addition of water and then extracted with EtOAc. The organic extracts were dried over MgSO₄, filtered and concentrated. The crude residue was purified by flash chromatography on silica gel (ISCO Combiflash, 120 gm column, Hexane/EtOAc 0→10%) and isolated 10.7 g of (2-bromo-6-chlorophenyl)(m-tolyl)methanol.

### Step 2. 1-bromo-3-chloro-2-[(3-methylphenyl)methyl]benzene

(2-bromo-6-chlorophenyl)(m-tolyl)methanol (10.7 g, 34.4 mmol) was dissolved in CH₂Cl₂ (50 mL) and then Et₃SiH (22 mL, 138 mmol) and trifluoroacetic acid (10.6 mL, 138 mmol) were added. After stirring at rt overnight, the reaction was concentrated to remove solvent. The crude residue was purified by flash chromatography on silica gel (ISCO Combiflash, 120 gm column, Hexane/EtOAc 0→10%) and isolated 8.7 g of 1-bromo-3-chloro-2-[(3-methylphenyl)methyl]benzene as a white solid.

1-bromo-3-chloro-2-[(2-methylphenyl)methyl]benzene was prepared using procedures analogous to those described above using o-tolualdehyde in Step 1.

### Preparation 20

### 3-methoxy-5-methylphenylboronic acid

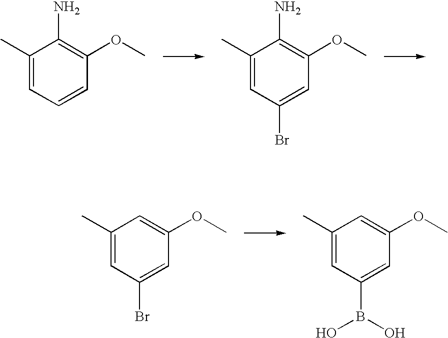

### Step 1. 4-bromo-2-methoxy-6-methylaniline

2-methoxy-6-methylaniline (24.2 g, 182 mmol) was dissolved in MeOH (81 mL) and acetic acid (27 mL) and a solution of bromine (28 g, 182 mmol) in acetic acid (81 mL) was added dropwise. The reaction was allowed to stand at rt for 2 h and concentrated to remove solvents. The crude product was recrystallized from hexanes to give 36 g of 4-bromo-2-methoxy-6-methylaniline as a brown solid.

### Step 2. 1-bromo-3-methoxy-5-methylbenzene

To a cold (0° C.) solution of 4-bromo-2-methoxy-6-methylaniline (36 g, 167 mmol) in a mixture of acetic acid (280 mL), water (120 mL) and concentrated HCl (32 mL) was added dropwise a solution of NaNO₂ (13.8 g, 200 mmol) in water (40 mL). The reaction mixture was stirred for 30 min at 0° C. and 50% aq H₃PO₂ (320 mL) was added. After stirring for 8 h at 0° C., the reaction mixture was allowed to stand at rt for 48 h. The reaction mixture was extracted with EtOAc/Et₂O. The crude residue was purified by flash chromatography on silica gel (ISCO Combiflash, 330 g column, 100% hexane) to afford 27.5 g of 1-bromo-3-methoxy-5-methylbenzene as a colorless oil.

### Step 3. 3-methoxy-5-methylphenylboronic acid

To a −78° C. solution of 1-bromo-3-methoxy-5-methylbenzene (10 g, 49.8 mmol) in anhydrous THF (200 mL) was added dropwise a n-BuLi solution (37.3 mL, 59.7 mmol, 1.6 M Hexane). After stirring for 30 min at −78° C., trimethyl borate (13.9 mL, 124.3 mmol) was added. The resulting mixture was stirred at −78° C. for 30 min and then warmed to rt and stirred for an additional 60 min. The reaction mixture was poured into an ice/H₂O mixture and acidified with 2N HCl to pH=3. The aqueous solution was extracted with Et₂O. The combined organic extracts were dried over Na₂SO₄, filtered and concentrated in vacuo. The crude residue (13 g) was washed with hexanes. The precipitate was collected and recrystallized from hexanes to give 6.5 g (79%) of 3-methoxy-5-methylphenylboronic acid as a white solid.

### Preparation 21

### 4-((tert-butoxycarbonylamino)methyl)-2-fluorobenzoic acid

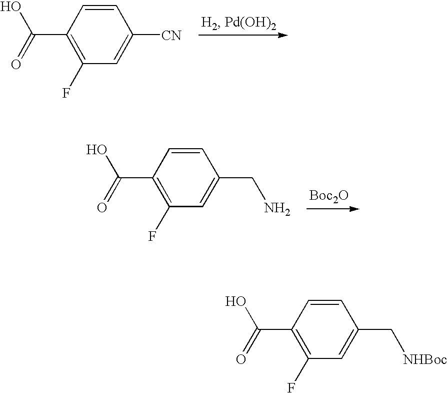

### Step 1. 4-(aminomethyl)-2-fluorobenzoic acid

A solution of 4-cyano-2-fluorobenzoic acid (1.0 g, 6.06 mmol) in 20 mL of MeOH at 25° C. was treated with of 20% Pd(OH)₂/C (300 mg, wet) and stirred overnight under an atmosphere of hydrogen. The reaction mixture was filtered and concentrated under reduced pressure to provide 4-(aminomethyl)-2-fluorobenzoic acid (1.0 g, quantitative).

### Step 2. 4-((tert-butoxycarbonylamino)methyl)-2-fluorobenzoic acid

A solution of 4-(aminomethyl)-2-fluorobenzoic acid (1.0 g, 6.0 mmol) in 50 mL of THF at 25° C. was treated with 50 mL of 1N aq NaOH and Boc₂O (1.5 g, 6.9 mmol) and the mixture was stirred overnight before being diluted with the addition of 25 mL of water and 10 mL of brine, acidified slowly to pH 3 using 1N aq HCl, and extracted with EtOAc (3×20 ml). The combined organic extracts were dried (Na₂SO₄) and concentrated under reduced pressure to provide 4-((tert-butoxycarbonylamino)methyl)-2-fluorobenzoic acid.

The following benzoic acids were prepared following procedures analogous to those described above by using the indicated starting material and catalyst in Step 1:

| Structure | Name | Starting Material | Catalyst |
|---|---|---|---|
| 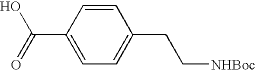 | 4-[2-({[(1,1- dimethylethyl)oxy]carbonyl} amino)ethyl]benzoic acid | 4-(2-aminoethyl)benzoic acid (Step 1 omitted) | Step 1 Omitted |

### Preparation 22

### 4-((tert-butoxycarbonyl(methyl)amino)methyl)benzoic acid

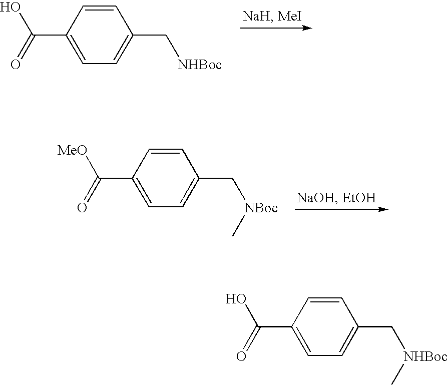

Step 1. Methyl 4-((tert-butoxycarbonyl(methyl)amino)methyl)benzoate

A solution of 4-((tert-butoxycarbonylamino)methyl)benzoic acid (1.01 g, 4.0 mmol) in 10 mL of DMF at 0° C. was treated with NaH (60% in oil, 400 mg, 10 mmol) and warmed to 25° C. After 10 min, methyl iodide (3 mL) was added and the mixture was stirred at 25° C. for 16 h before being concentrated under reduced pressure. The residue was treated with water (20 mL) and extracted with EtOAc (3×20 mL). The combined organic extracts were washed (brine), dried (Na₂SO₄), concentrated, and subjected to flash chromatography to provide methyl 4-((tert-butoxycarbonyl(methyl)amino)methyl)benzoate as a clear oil (849 mg, 76%). MS (m/z) 280.3 (M+H⁺).

### Step 2. 4-((tert-butoxycarbonyl(methyl)amino)methyl)benzoic acid

A solution of methyl 4-((tert-butoxycarbonyl(methyl)amino)methyl)benzoate (300 mg, 1.08 mmol) in EtOH (10 ml) at 25° C. was treated with aqueous 1N NaOH (2.16 mL, 2. 16 mmol) and the mixture was stirred for 16 h before being extracted with EtOAc (2×5 mL). The aqueous layer was acidified by the addition of aqueous 1N HCl and then extracted with EtOAc (3×10 ml). The combined organic extracts were washed (brine), dried (Na₂SO₄), and concentrated to provide 4-((tert-butoxycarbonyl(methyl)amino)methyl)benzoic acid as a white solid (215 mg, 75%). MS (m/z) 266.1 (M+H⁺).

The following benzoic acids were prepared following procedures analogous to those described above by using the indicated starting material and alkylating agent in Step 1:

| | | | Alkylating |
|---|---|---|---|
| Structure | Name | Starting Material | Agent |
| 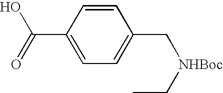 | 4-{[{[(1,1-dimethylethyl) oxy]carbonyl} (ethyl)amino] methyl}benzoic acid | 4-[({[(1,1-dimethyl ethyl)oxy]carbonyl} amino)methyl]benzoic acid | Ethyl iodide |

### Preparation 23

### 4-((tert-butoxycarbonyl(isopropyl)amino)methyl)benzoic acid

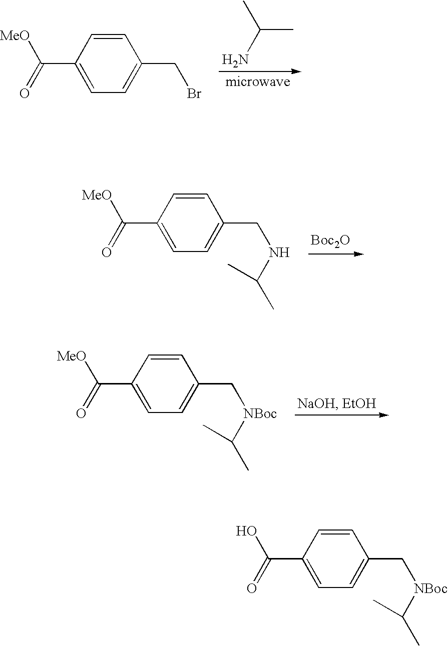

### Step 1. Methyl 4-((isopropylamino)methyl)benzoate

A solution of methyl 4-(bromomethyl)benzoate (1.15 g, 5 mmol) and isopropyl amine (25 mL, 2M in THF, 50 mmol) was heated under microwave irradiation at 100° C. for 10 min before being concentrated under reduced pressure and partitioned between EtOAc and aqueous 1N NaOH. The organic layer was washed (brine), dried (MgSO₄), and concentrated under reduced pressure to provide methyl 4-((isopropylamino)methyl)benzoate as an amber oil (860 mg, 89%). MS (m/z) 208.1 (M+H⁺).

### Step 2. methyl 4-((tert-butoxycarbonyl)isopropyl)amino)methyl)benzoate

A solution of methyl 4-((isopropylamino)methyl)benzoate (1.02 g, 4.92 mmol) in THF (20 ml) at 25° C. was treated with saturated aqueous NaHCO₃ (15 ml) and (Boc)₂O (1.13 g, 5.17 mmol) and stirred for 16 h. The reaction mixture was diluted with EtOAc and the organic phase was separated, washed (H₂O, brine), dried (Na₂SO₄), concentrated under reduced pressure, and subjected to flash chromatography to provide methyl 4-((tert-butoxycarbonyl(isopropyl)amino)methyl)benzoate as a clear oil (1.47 g, 97%). MS (m/z) 308.3 (M+H⁺).

### Step 3. 4-((tert-butoxycarbonyl)isopropyl)amino)methyl)benzoic acid

A solution of methyl 4-((tert-butoxycarbonyl(isopropyl)amino)methyl)benzoate (860 mg, 4.1 mmol) in EtOH (40 mL) at 25° C. was treated with aqueous 1N NaOH (8.2 mL, 8.2 mmol) and the mixture was stirred for 16 h before being extracted with EtOAc (2×20 mL). The aqueous layer was acidified by the addition of aqueous 1N HCl and then extracted with EtOAc (3×40 mL). The combined organic extracts were washed (brine), dried (Na₂SO₄), and concentrated to provide 4-((tert-butoxycarbonyl(isopropyl)amino)methyl)benzoic acid as a white solid (625 mg, 75%). MS (m/z) 238 (M+H⁺-t-Bu).

The following benzoic acids were prepared following procedures analogous to those described above by using the indicated amine in place of isopropylamine in Step 1:

| Structure | Name | Amine | Note |
|---|---|---|---|
| 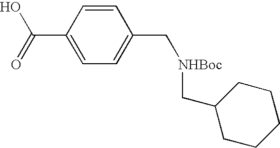 | 4-[((cyclohexylmethyl) {[(1,1dimethylethyl)oxy] carbonyl}amino)ethyl] benzoic acid | cyclohexylmethylamine | |
| 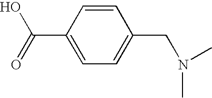 | 4-[(dimethylamino)methyl] benzoic acid | dimethylamine | Step 2 omitted |
| 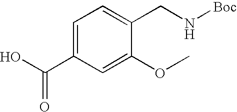 | 4-[({[(1,1- dimethylethyl)oxy]carbonyl} amino)methyl]-3- (methyloxy)benzoic acid | phthalamide, hydrozine | |
| 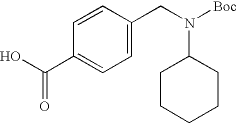 | 4-[(cyclohexyl{[(1,1- dimethylethyl)oxy]carbonyl} amino)methyl]benzoic acid | cyclohexylamine | |
| 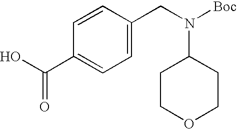 | 4-{[{[(1,1-dimethylethyl) oxy]carbonly}(tetrahydro- 2H-pyran-4-yl)amino] methyl}benzoic acid | tetrahydro-2H-pyran-4- amine | Substituting K₂CO₃ in refluxing EtOH in for microwave Step 1. |

### Preparation 24

### 4-[(methylsulfonyl)methyl]benzoic acid

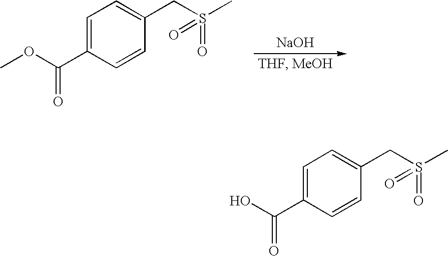

### Step 1. 4-[(methylsulfonyl)methyl]benzoic acid

To a solution of methyl 4-[(methylsulfonyl)methyl]benzoate (2 g, 8.76 mmol) in THF (15 mL) and MeOH (15 mL), 2N NaOH (10 mL) was added. The resulting mixture was stirred overnight. The reaction was quenched with 2N HCl and extracted with ethyl acetate. The combined organics were then dried, filtered and concentrated to afford 4-[(methylsulfonyl)methyl]benzoic acid which was used in the next reaction without further purification. MS (m/z) 237.0 (M+Na⁺).

### Preparation 25

### a.) 2-bromo-4-{[{[(1,1-dimethylethyl)oxy]carbonyl}(methyl)amino]carbonyl}benzoic acid

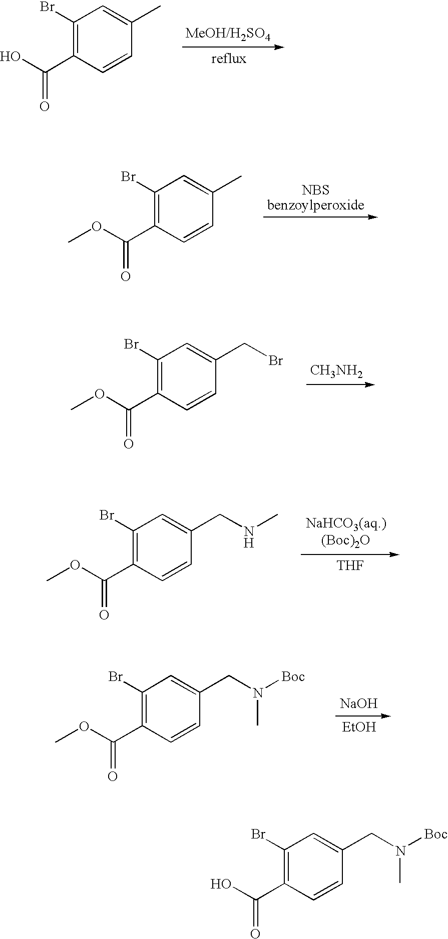

### Step 1. Methyl 2-bromo-4-methylbenzoate

Concentrated sulfuric acid (4 mL) was added to a solution of 2-Bromo-4-methyl benzoic acid (10.0 g, 46.5 mmol) in dry methanol (200 ml). The reaction mixture was heated to reflux for 18 h. After cooling, the reaction mix was concentrated and the residual was partitioned between water and ethyl acetate. The layers were separated and the aqueous was extracted with EtOAc (3×100 ml). The combined organic extracts were washed with saturated NaHCO₃ (2×50 ml), brine, dried over MgSO₄, filtered, and concentrated to give methyl 2-bromo-4-methylbenzoate (10.50 g, 99%) as a pale amber oil. MS (m/z) 229.2 (M+H⁺).

### Step 2. Methyl 2-bromo-4-(bromomethyl)benzoate

NBS (8.53 g, 48 mmol) was added to a solution of methyl 2-bromo-4-methylbenzoate (10.4 g, 45.6 mmol) in CCl₄ (400 ml), followed by benzyol peroxide (441 mg, 1.82 mmol). The reaction mixture was then heated to reflux and after 2 h, the reaction mix became colorless. The mixture was allowed to cool to room temperature and before it was filtered. The filtrate was concentrated to give 12 g of crude material which was purified by column chromatography (440 g silica gel 60, 230-400 mesh, 10 then 20% CH₂Cl₂/hexane) to give methyl 2-bromo-4-(bromomethyl)benzoate (5.07 g, 36%) as a white solid. MS (m/z) 307.3 (M+H⁺).

### Step 3. Methyl 2-bromo-4-[(methylamino)methyl]benzoate

A mixture of methyl 2-bromo-4-(bromomethyl)benzoate (5.06 g, 16.5 mmol), methyl amine (2M in THF, 16.5 ml, 33 mmol) and K₂CO₃ (2.28 g, 16.5 mmol) in THF (40 ml) was heated to reflux for 16 h. The reaction mixture was filtered and the filtrate was concentrated to afford 5.2 g as an light amber oil which was purified by column chromatography (200 g silica gel 60, 230-400 mesh, 0-2% MeOH/CH₂Cl₂) to give methyl 2-bromo-4-[(methylamino)methyl]benzoate (3.41 g, 80%) as a clear oil. MS (ES): MS (m/z) 258.3 (M+H⁺).

### Step 4: Methyl 2-bromo-4-{[{[(1,1-dimethylethyl)oxy]carbonyl}(methyl)amino]methyl}benzoate

Saturated aqueous NaHCO₃ (15 mL) was added to a solution of methyl 2-bromo-4-[(methylamino)methyl]benzoate (1.29 g, 5 mmol) in THF (15 mL) followed by (Boc)₂O (1.14 g, 5.25 mmol). The mixture was stirred vigorously for 16 h. The reaction mixture was diluted with EtOAc and the organic phase was separated, washed with water, brine, dried and concentrated to afford methyl 2-bromo-4-{[{[(1,1-dimethylethyl)oxy]carbonyl}(methyl)amino]methyl}benzoate (1.78 g, >99%) as a clear oil which in the next reaction without further purification. MS (ES): MS (m/z) 357.8 (M+H⁺).

### Step 5: 2-bromo-4-{[{[(1,1-dimethylethyl)oxy]carbonyl}(methyl)amino]methyl}benzoic acid

NaOH (1N, 2 mL) was added to a solution of methyl 2-bromo-4-{[{[(1,1-dimethylethyl)oxy]carbonyl}(methyl)amino]methyl}benzoate (357 mg, 1 mmol) in EtOH (10 mL). The resulting mixture was stirred at rt for 16 h. The reaction mixture was then concentrated and the residue was taken into water (10 mL) and washed with EtOAc (2×5 mL). The aqueous layer was acidified with HCl (1M) and the product was extracted with ethyl acetate (3×10 mL). The combined organic extracts were washed with brine, dried over MgSO₄, filtered and concentrated to give 2-bromo-4-{[{[(1,1-dimethylethyl)oxy]carbonyl}(methyl)amino]methyl}benzoic acid (253 mg, 73%) as a clear oil. MS (m/z) 344.2 (M+H⁺).

The following benzoic acids/esters were prepared following procedures analogous to those described above by using the indicated starting material in place of 2-Bromo-4-methyl benzoic acid in Step 1:

| Benzoic | | | |
|---|---|---|---|
| Acid/Ester | Name | Starting Material | Notes |
| 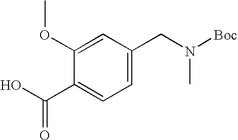 | 4-{[{[(1,1-dimethylethyl) oxy]carbonyl}(methyl)amino] methyl}-2- (methyloxy)benzoic acid | 4-methyl-2- (methyloxy)benzoic acid | |
| 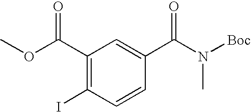 | methyl 5-{[{[(1,1- dimethylethyl)oxy]carbonyl} (methyl)amino]methyl}- 2-iodobenzoate | 2-iodo-5- methylbenzoic acid | Step 5 omitted. |

### b.) 4-{[{[(1,1-dimethylethyl)oxy]carbonyl}(methyl)amino]methyl}-2-[(methyloxy)carbonyl]benzoic acid

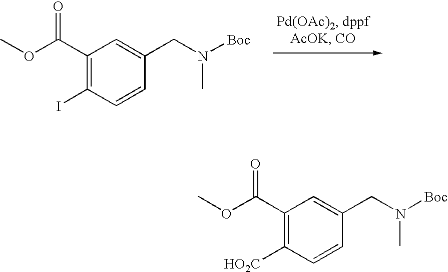

### Step 1. 4-{[{[(1,1-dimethylethyl)oxy]carbonyl}(methyl)amino]methyl}-2-[(methyloxy)carbonyl]benzoic acid

A mixture of methyl 5-{[{[(1,1-dimethylethyl)oxy]carbonyl}(methyl)amino]methyl}-2-iodobenzoate (422 mg, 1.05 mmol), potassium acetate (412 mg, 4.2 mmol), palladium acetate (12 mg, 0.052 mmol) and dppf (116 mg, 0.21 mmol) in DMSO (6 mL) was purged with carbon monoxide for 10 min then stirred under a carbon monoxide balloon at 65° C. for 5 h. At this time, HPLC as well as LCMC showed that the starting material had been consumed. The reaction mixture was diluted with water (6 mL), acidified with 1N HCl and extracted by EtOAc (2×15 mL). The combined organic extracts were washed with water, brine, dried, filtered and concentrated to afford 816 mg of crude material as a dark brown oil. Purification by column chromatography (32 g silica gel 60, 230-400 mesh, 1-2% MeOH containing 5% HOAc in CH₂Cl₂) provided 4-{[{[(1,1-dimethylethyl)oxy]carbonyl}(methyl)amino]methyl}-2-[(methyloxy)carbonyl]benzoic acid (173 mg, 51%) as an amber oil. MS (m/z) 324.5 (M+H⁺).

### c.) 4-{[{[1,1-dimethylethyl)oxy]carbonyl}(methyl)amino]methyl}-1-naphthalenecarboxylic acid

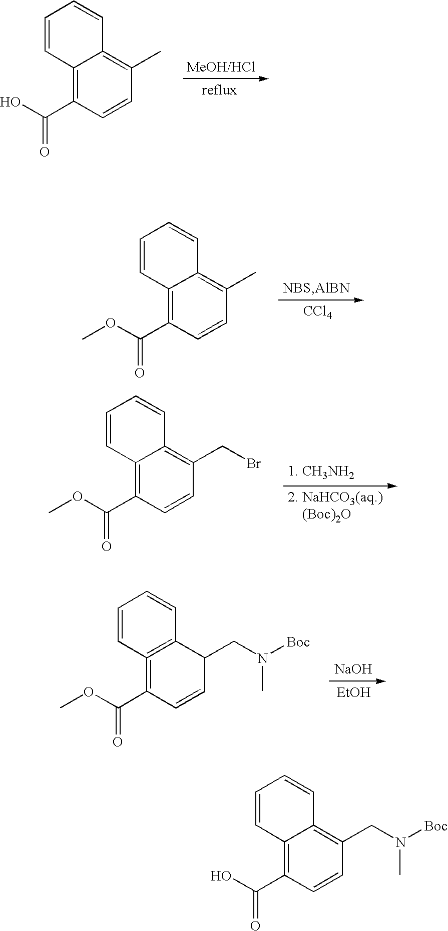

### Step 1. Methyl 4-methyl-1-naphthalenecarboxylate

HCl was bubbled through a solution of 4-methyl-1-naphthoic acid (3.72 g, 20 mmol) in dry MeOH (200 ml) at 0° C. for 5 min. The resulting mixture was then refluxed for 16 h. Solvent was removed in vacuo and the residue dissolved in EtOAc and washed with NaOH (1N, 2×20 ml). The organic layer was then washed with water, brine, dried over MgSO₄, filtered, and concentrated to afford methyl 4-methyl-1-naphthalenecarboxylate (3.79 g, 94.5%) as an off white solid.

### Step 2. Methyl 4-(bromomethyl)-1-naphthalenecarboxylate

NBS (3.53 g, 18.9 mmol) was added to a solution of methyl 4-methyl-1-naphthalenecarboxylate (3.78 g, 18.9 mmol) in CCl₄ (200 ml), followed by AIBN (310 mg, 1.89 mmol). The reaction mixture was then heated at 70° C. for 6 h. After cooling, the reaction mixture was filtered, the filtrate concentrated, and the residue dissolved in chloroform and washed with 1N NaOH, and then brine, dried, filtered and concentrated to give 4.97 g of crude material as a pale yellow solid. Purification via column chromatography (200 g silica gel 60, 230-400 mesh, 10-20% CH₂Cl₂/hexane) gave methyl 4-(bromomethyl)-1-naphthalenecarboxylate (3.04 g, 58%) as a white powder.

### Step 3. Methyl 4-{[{[(1,1-dimethylethyl)oxy]carbonyl}(methyl)amino]methyl}-1-naphthalenecarboxylate

A mixture methyl 4-(bromomethyl)-1-naphthalenecarboxylate (1.45 g, 5.2 mmol) and methyl amine (2 M in THF, 24 ml, 48 mmol) was heated under microwave irradiation at 100° C. for 10 min. The reaction mixture was concentrated and the residual was partitioned between EtOAc and 1N NaOH. The organic layer was washed with saturated brine, dried over MgSO₄, filtered, and concentrated in vacuo to give an amber oil which dissolved THF (20 mL) treated with saturated aqueous NaHCO₃ (15 mL) and (Boc)₂O (1.25 g, 5.72 mmol). The mixture was stirred vigorously for 16 h. The reaction mixture was then diluted with EtOAc and the organic phase separated, washed with water, brine, dried and concentrated to give 1.6 g of crude material as an amber oil. The product was purified via column chromatography (64 g silica gel 60, 230-400 mesh, 5% EtOAc/hexane) to give methyl 4-{[{[(1,1-dimethylethyl)oxy]carbonyl}(methyl)amino]methyl}-1-naphthalenecarboxylate (1.53 g, 89% over 2 steps) as a clear oil. MS (m/z) 330.4 (M+H⁺).

### Step 4. 4-{[{[(1,1-dimethylethyl)oxy]carbonyl}(methyl)amino]methyl}-1-naphthalenecarboxylic acid

NaOH (1N, 2 mL) was added to a solution of give methyl 4-{[{[(1,1-dimethylethyl)oxy]carbonyl}(methyl)amino]methyl}-1-naphthalene carboxylate (1.51 g, 4.6 mmol) in EtOH (30 mL). The resulting mixture was stirred at rt for 20 h. At this time, LCMS as well as HPLC indicated the reaction was completed. The reaction was extracted with ethyl acetate (2×10 mL), the aqueous was acidified with HCl (1M) and then extracted with ethyl acetate (3×10 mL). The combined extracts were washed with brine, dried over Na₂SO₄, filtered, and concentrated in vacuo to provide 4-{[{[(1,1-dimethylethyl)oxy]carbonyl}(methyl)amino]methyl}-1-naphthalenecarboxylic acid (1.30 g, 90%) as a clear oil, which was used in subsequent reactions without further purification. MS (m/z) 316.3 (M+H⁺).

### Preparation 26

### a.) 5-{[{[(1,1-dimethylethyl)oxy]carbonyl}(methyl)amino]methyl}-2-biphenylcarboxylic acid

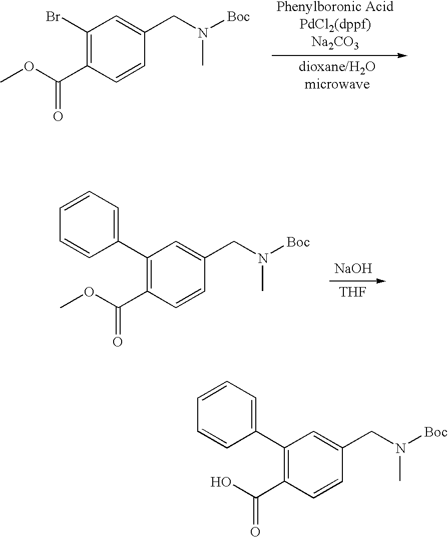

### Step 1. Methyl 5-{[{[(1,1-dimethylethyl)oxy]carbonyl}(methyl)amino]methyl}-2-biphenylcarboxylate

A mixture of methyl 2-bromo-4-{[{[(1,1-dimethylethyl)oxy]carbonyl}(methyl)amino]methyl}benzoate (480 mg, 1.35 mmol), phenyl boronic acid (247 mg, 2.03 mmol) and sodium carbonate (1N, 5.4 ml, 5.4 mmol) in dioxane (13.6 ml)) was deoxygenated, then Pd(dppf)Cl₂ (60 mg) was added. The resulting mixture was heated under microwave irradiation at 140° C. for 10 min. The catalyst was removed by filtration, the filtrate concentrated, and the residue dissolved in EtOAc and washed with brine, dried and concentrated to give 891 mg of crude material as a dark brown solid which was purified by column chromatography (35 g silica gel 60, 230-400 mesh, 10% EtOAc in hexane as eluent) to give methyl 5-{[{[(1,1-dimethylethyl)oxy]carbonyl}(methyl)amino]methyl}-2-biphenylcarboxylate (344 mg, 72%) as a white solid. MS (ES): MS (m/z) 356.4 (M+H⁺).

### Step 2. 5-{[{[(1,1-dimethylethyl)oxy]carbonyl}(methyl)amino]methyl}-2-biphenylcarboxylic acid

NaOH (1N, 2 ml) was added to a solution of methyl 5-{[{[(1,1-dimethylethyl)oxy]carbonyl}(methyl)amino]methyl}-2biphenylcarboxylate (355 mg, 1 mmol) in EtOH (10 mL). The resulting mixture was stirred at rt overnight. HPLC as well as LCMS indicated only a trace of the desired product had formed at this point. The reaction was heated to reflux and monitored by HPLC. After 40 h, LCMS as well as HPLC indicated the reaction was complete. The reaction was extracted with ethyl acetate (2×5 mL) and the aqueous solution acidified with HCl (1M) and then extracted with ethyl acetate (3×10 mL), washed with brine, dried over Na₂SO₄, filtered, and concentrated to afford 5-{[{[(1,1-dimethylethyl)oxy]carbonyl}(methyl)amino]methyl}-2-biphenylcarboxylic acid (270 mg, 79%) as a clear oil. MS (m/z) 342.3 (M+H⁺).

### b.) 2-butyl-4-{[{[(1,1-dimethylethyl)oxy]carbonyl}(methyl)amino]methyl}benzoic acid

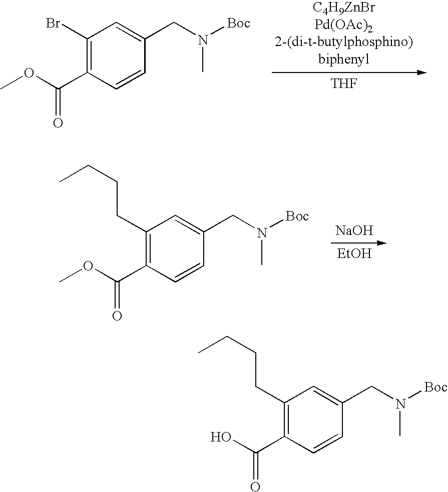

### Step 1. Methyl 2-butyl-4-{[{[(1,1-dimethylethyl)oxy]carbonyl}(methyl)amino]methyl}benzoate

Methyl 2-bromo-4-{[{[(1,1-dimethylethyl)oxy]carbonyl}(methyl)amino]methyl}benzoate (714 mg, 2 mmol) was added to a deoxygenated solution of palladium acetate (90 mg, 0.4 mmol) and 2-(di-t-butylphosphino)biphenyl (60 mg, 0.2 mmol) in dry THF (10 mL) in a flame dried flask, followed by butyl zinc bromide (0.5 M in THF, 12 mL, 6 mmol). The resulting dark mixture was stirred at rt under for 18 h. The catalyst was removed by filtration through a pad of Celite and the filtrate was partitioned between EtOAc and water. The aqueous layer was extracted with EtOAc (3×10 mL) and the combined extracts were washed with brine, dried, filtered and concentrated to give 700 mg of crude material as a dark brown oil. Purification via column chromatography (28 g silica gel 60, 230-400 mesh, 3% EtOAc/hexane) gave methyl 2-butyl-4-{[{[(1,1-dimethylethyl)oxy]carbonyl}(methyl)amino]methyl}benzoate (388 mg, 58%) as a clear oil. MS (m/z) 336.4 (M+H⁺).

### Step 2. 2-butyl-4-{[{[(1,1-dimethylethyl)oxy]carbonyl}(methyl)amino]methyl}benzoic acid

NaOH (1N, 2 mL) was added to a solution of methyl 2-butyl-4-{[{[(1,1-dimethylethyl)oxy]carbonyl}(methyl)amino]methyl}benzoate (368 mg, 1.1 mmol) in EtOH (10 mL). The resulting mixture was stirred at rt over the weekend. LCMS as well as HPLC analysis taken after this time indicated the reaction was complete. The reaction mixture was extracted with ethyl acetate (2×5 mL) and the aqueous was acidified by HCl (1M) and then extracted with ethyl acetate (3×10 mL). The organic extracts were washed with brine, dried over Na₂SO₄, filtered and concentrated to afford 2-butyl-4-{[{[(1,1-dimethylethyl)oxy]carbonyl}(methyl)amino]methyl}benzoic acid (170 mg, 48%) as a clear oil. MS (m/z) 322.4 (M+H⁺).

The following benzoic acids were prepared following procedures analogous to those described above by using the indicated alkyl zinc species in place of butylzinc bromide in Step 1:

| Benzoic Acid | Name | Alkyl Zinc |
|---|---|---|
| 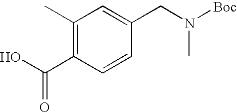 | 4-{[{[(1,1- dimethylethyl)oxy]carbonyl}(methyl) amino]methyl}-2-methylbenzoic acid | Methylzinc bromide |
| 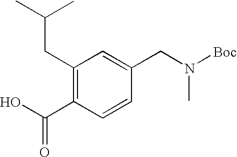 | 4-{[{[(1,1- dimethylethyl)oxy]carbonyl}(methyl) amino]methyl}-2-(2- methylpropyl)benzoic acid | Isobutylzinc bromide |
| 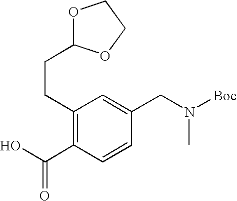 | 4-{[{[(1,1- dimethylethyl)oxy]carbonyl}(methyl) amino]methyl}-2-[2-(1,3-dioxolan-2- yl)ethyl]benzoic acid | 2-[(1,3-Dioxol-2- yl)ethyl]zinc bromide |
| 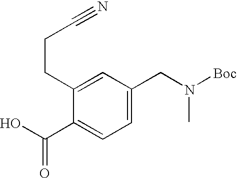 | 2-(2-cyanomethyl)-4-{[{[(1,1- dimethylethyl)oxy]carbonyl}(methyl) amino]methyl}benzoic acid | 2-Cyanoethylzinc bromide |

### c.) 4-{[{[(1,1-dimethylethyl)oxy]carbonyl}(methyl)amino]methyl}-2-ethylbenzoic acid

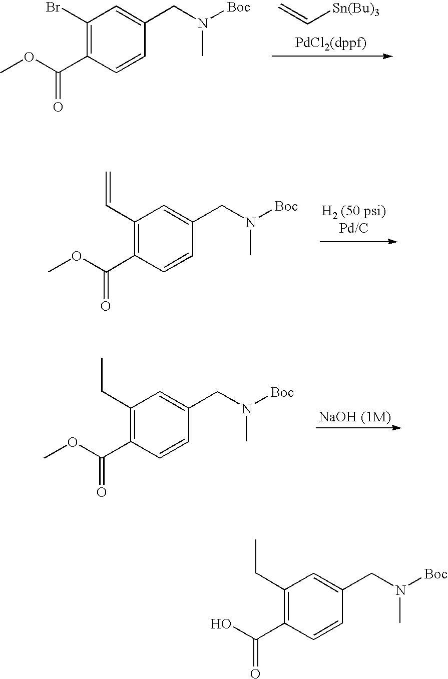

### Step 1. Methyl 4-{[{[(1,1-dimethyl ethyl)oxy]carbonyl}(methyl)amino]methyl}-2-ethynylbenzoate

A mixture of methyl 2-bromo-4-{[{[(1,1-dimethylethyl)oxy]carbonyl}(methyl)amino]methyl}benzoate (357 mg, 1 mmol), PdCl₂(dppf) (82 mg, 0.1 mmol) and tributyl-vinyl-stannane (1.90 g, 6 mmol) in dry DMF (5 mL) was heated at 125° C. for 6 h. After cooling, the reaction mixture was filtered through Celite and the filtrate was concentrated in vacuo. The residue was dissolved in EtOAc and washed with brine, dried, filtered, and concentrated to give 440 mg of crude material as a dark brown oil. Purification by column chromatography (18 g silica gel 60, 230-400 mesh, 5-10% EtOAc/hexane) gave methyl 4-{[{[(1,1-dimethylethyl)oxy]carbonyl}(methyl)amino]methyl}-2-ethynylbenzoate (57.2 mg, 18%) as a clear oil. MS (m/z) 306.4 (M+H⁺).

### Step 2. Methyl 4-{[{[(1,1-dimethylethyl)oxy]carbonyl}(methyl)amino]methyl}-2-ethylbenzoate

A mixture of methyl 4-{[{[(1,1-dimethylethyl)oxy]carbonyl}(methyl)amino]methyl}-2-ethynylbenzoate (55 mg, 0.18 mmol) and palladium on carbon (10%, 50 mg) in EtOH (5 mL) was hydrogenated at 50 psi at rt for 5 h. The catalyst was removed via filtration and filtrate concentrated to provide methyl 4-{[{[(1,1-dimethylethyl)oxy]carbonyl}(methyl)amino]methyl}-2-ethylbenzoate (44 mg, 80%). MS (m/z) 307.8 (M+H⁺).

### Step 3. 4-{[{[(1,1-dimethylethyl)oxy]carbonyl}(methyl)amino]methyl}-2-ethylbenzoic acid

NaOH (1N, 0.28 ml) was added to a solution of methyl 4-{[{[(1,1-dimethylethyl)oxy]carbonyl}(methyl)amino]methyl}-2-ethylbenzoate (42 mg, 0.14 mmol) in EtOH (5 mL). The resulting mixture was stirred at rt over the weekend. The reaction mixture was then extracted with ethyl acetate (2×3 mL) and the aqueous layer acidified by HCl (1M) and then extracted with ethyl acetate (3×3 mL). The organic extracts were washed with brine, dried over Na₂SO₄, filtered and concentrated to afford 4-{[{[(1,1-dimethylethyl)oxy]carbonyl}(methyl)amino]methyl}-2-ethylbenzoic acid (17.6 mg, 43%) as a clear oil. MS (m/z) 294.4 (M+H⁺)

### d.) 2-cyano-4-{[{[(1,1-dimethylethyl)oxy]carbonyl}(methyl)amino]methyl}benzoic acid

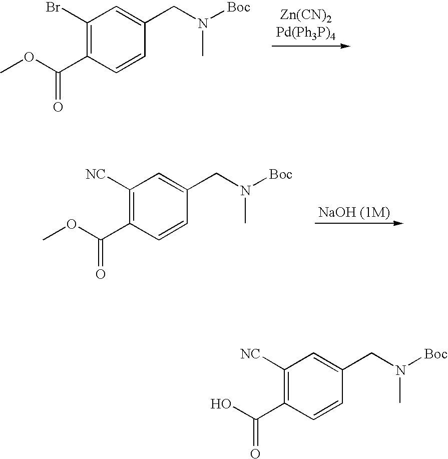

### Step 1. Methyl 2-cyano-4-{[{[(1,1-dimethylethyl)oxy]carbonyl}(methyl)amino]methyl}benzoate

A mixture of methyl 2-bromo-4-{[{[(1,1-dimethylethyl)oxy]carbonyl}(methyl)amino]methyl}benzoate (357 mg, 1 mmol), and Zinc cyanide (70 mg, 0.6 mmol) in DMF (3 mL) was deoxygenated, then Pd(Ph₃P)₄ (58 mg, 0.05 mmol) was added. The resulting mixture was heated at 100° C. under argon for 42 h. After cooling, another 70 mg of zinc cyanide was added, the reaction was heated for an additional 20 h. The reaction mixture was concentrated in vacuo and the residue dissolved in ethyl acetate and washed with 2 N NH₄OH, brine, dried filtered and concentrated to give 267 mg of crude material which was purified by preparatory TLC (Kieselge 60 F₂₅₄, 0.5 mm, 20×20 cm, 2 plates, 40% EtOAc/Hexane) to provide methyl 2-cyano-4-{[{[(1,1-dimethylethyl)oxy]carbonyl}(methyl)amino]methyl}benzoate (38 mg, 12.7%). MS (m/z) 305.1 (M+H⁺).

### Step 2. 2-cyano-4-{[{[(1,1-dimethylethyl)oxy]carbonyl}(methyl)amino]methyl}benzoic acid

To a solution of methyl 2-cyano-4-{[{[(1,1-dimethylethyl)oxy]carbonyl}(methyl)amino]methyl}benzoate (38 mg, 0.12 mmol) in EtOH (5 mL) was added NaOH (1N, 0.24 mL). The resulting mixture was stirred at rt for 24 h. LCMS as well as HPLC indicated the reaction was complete at this time. The reaction mixture was extracted with ethyl acetate (2×5 mL) and the aqueous acidified by HCl (1M) and then extracted with ethyl acetate (3×3 mL). The organic extracts were washed with brine, dried over Na₂SO₄, filtered, and concentrated to afford 2-cyano-4-{[{[(1,1-dimethylethyl)oxy]carbonyl}(methyl)amino]methyl}benzoic acid (31.2 mg, 90%) as a clear oil. MS (m/z) 291.2 (M+H⁺).

### e.) 2-(cyanomethyl)-4-{[{[(1,1-dimethylethyl)oxy]carbonyl}(methyl)amino]methyl}benzoic acid

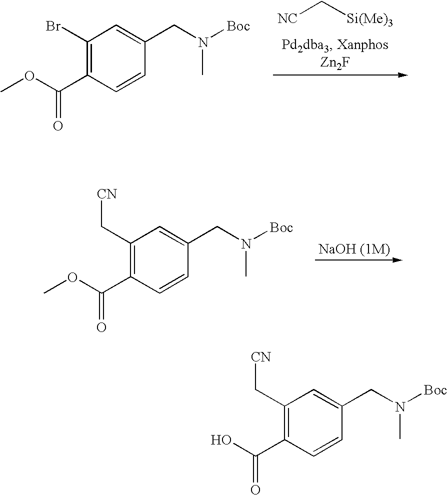

### Step 1. Methyl 2-(cyanomethyl)-4-{[{[(1,1-dimethylethyl)oxy]carbonyl}(methyl)amino]methyl}benzoate

To a mixture containing Xantphos (49 mg, 0.084 mmol), Pd₂ dba₃ (77 mg, 0.084 mmol) and methyl 2-bromo-4-{[{[(1,1-dimethylethyl)oxy]carbonyl}(methyl)amino]methyl}benzoate (1.50 g, 4.2 mmol) in DMF (4.2 mL) was added trimethylsilylacetonitrile (526 mg, 1.18 mmol) followed by zinc fluoride (260 mg, 2.52 mmol). The resulting mixture was heated in a sealed tube at 90° C. for 50 h. After cooling, the reaction was diluted with ether (100 mL) and washed with water and brine, dried, filtered and concentrated to give 1.2 g of crude material which was purified by column chromatography (48 g silica gel 60, 230-400 mesh, 5-15% EtOAc/hexane) to afford methyl 2-(cyanomethyl)-4-{[{[(1,1-dimethylethyl)oxy]carbonyl}(methyl)amino]methyl}benzoate (55 mg, 4%) as a white solid. MS (m/z) 319.3 (M+H⁺).

### Step 2. 2-(cyanomethyl)-4-{[{[(1,1-dimethylethyl)oxy]carbonyl}(methyl)amino]methyl}benzoic acid

To a solution of methyl 2-(cyanomethyl)-4-{[{[(1,1-dimethylethyl)oxy]carbonyl}(methyl)amino]methyl}benzoate (55 mg, 0.17 mmol) in EtOH (5 mL) was added NaOH (1N, 0.34 ml. The resulting mixture was stirred at rt for 20 h. LCMS as well as HPLC indicated the reaction was completed. The reaction mixture was extracted with ethyl acetate (2×5 mL) and the aqueous acidified with HCl (1M) and then extracted with ethyl acetate (2×5 mL). The organic extracts were washed with brine, dried over Na₂SO₄, filtered, and concentrated to provide 2-(cyanomethyl)-4-{[{[(1,1-dimethylethyl)oxy]carbonyl}(methyl)amino]methyl}benzoic acid (48 mg, 92%) as an oil. MS (m/z) 304.8 (M+H⁺).

### Preparation 27

### 1-(6-chloro-3′-ethyl-2-biphenylyl)-1-[(3R)-3-piperidinyl]-1,6-hexanediol

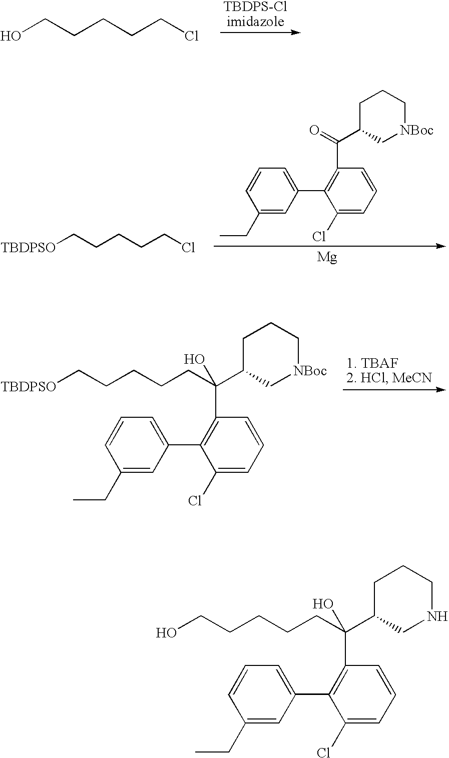

### Step 1. 5-(t-butyldimethylsilyloxy)-1-chloropentane

A solution of 5-chloro-1-pentanol (10 g, 80 mmol) in DMF (100 mL) at 0° C. was treated with imidazole (19 g, 185 mmol) and TBDPS-Cl (26 g, 95 mmol), and the mixture was stirred and allowed to warm to room temperature over 2 hours before being quenched with the addition of water and extracted with EtOAc. The organic extract was washed with aqueous 1N HCl, saturated aqueous NaHCO₃, saturated aqueous NaCl, dried (Na₂SO₄), concentrated under reduced pressure, and subjected to flash chromatography to give 5-(t-butyldimethylsilyloxy)-1-chloropentane as a colorless liquid (11 g, 38%).

### Step 2. (3R)-tert-butyl 3-(6-(tert-butyldiphenylsilyloxy)-1-(6-chloro-3′-ethylbiphenyl-2-yl)-1-hydroxyhexyl)piperidine-1-carboxylate

Magnesium (0.451 g, 18.6 mmol) was treated with a small crystal of iodine and THF (5 ml) and the mixture was heated at 65° C. for 5 minutes while the red color faded to yellow. 1 drop of dibromoethane was added and the mixture began to bubble and turned colorless. A solution of 5-(t-butyldimethylsilyloxy)-1-chloropentane (5.5 g, 15.2 mmol) in THF 10 mL was added dropwise over 10 minutes and the resulting yellow mixture was stirred at 65° C. for 30 minutes. The resulting cloudy, gray mixture was added to a solution of 1,1-dimethylethyl (3R)-3-[(6-chloro-3′-ethyl-2-biphenylyl)carbonyl]-1-piperidinecarboxylate (1.5 g, 3.5 mmol) in THF (10 ml) at −20° C. The mixture was warmed to 25° C. over 3 hours before being quenched with the addition of saturated aqueous NH₄Cl. The mixture was extracted with Et₂O and the organic extract was dried (Na₂SO₄), concentrated under reduced pressure, and subjected to flash chromatography to provide (3R)-tert-butyl 3-(6-(tert-butyldiphenylsilyloxy)-1-(6-chloro-3′-ethylbiphenyl-2-yl)-1-hydroxyhexyl)piperidine-1-carboxylate as a colorless oil (1.1 g, 40%): ESI-MS (m/z): 654.3 (M+H⁺-Boc).

### Step 3. (3R)-tert-butyl 3-(1-(6-chloro-3′-ethylbiphenyl-2-yl)-1,6-dihydroxyhexyl)piperidine-1-carboxylate

A solution of the (3R)-tert-butyl 3-(1-(6-(tert-butyldiphenylsilyloxy)-1-(6-chloro-3′-ethylbiphenyl-2-yl)-1-hydroxyhexyl)piperidine-1-carboxylate (200 mg, 0.27 mmol) in THF (3 ml) at 25° C. was treated with TBAF (0.32 ml of 1M in THF, 0.32 mmol) and stirred for 24 hr before being quenched with the addition of saturated aqueous NH₄Cl. The mixture was extracted with CH₂Cl₂ and the organic extracts were dried (Na₂SO₄), concentrated, and subjected to flash chromatography to give (3R)-tert-butyl 3-(1-(6-chloro-3′-ethylbiphenyl-2-yl)-1,6-dihydroxyhexyl)piperidine-1-carboxylate as a colorless oil (60 mg, 44%): ESI-MS (m/z): 538.2 (M+Na⁺).

### Step 4. 1-(6-chloro-3′-ethyl-2-biphenylyl)-1-[(3R)-3-piperidinyl]-1,6-hexanediol

A solution of (3R)-tert-butyl 3-(1-(6-chloro-3′-ethylbiphenyl-2-yl)-1,6-dihydroxyhexyl)piperidine-1-carboxylate (60 mg, 0.12 mmol) in CH₃CN (3 ml) at 25° C. was treated with aqueous 2N HCl (3 ml, 6.0 mmol) and allowed to stand overnight. The mixture was concentrated to provide 1-(6-chloro-3′-ethyl-2-biphenylyl)-1-[(3R)-3-piperidinyl]-1,6-hexanediol as a colorless oil (50 mg, 96%): ESI-MS (m/z): 416.2 (M+H⁺).

### Preparation 28

(R)-1-(6-chloro-3′-ethylbiphenyl-2-yl)-1-((R)-morpholin-2-yl)pent-4-en-1-ol

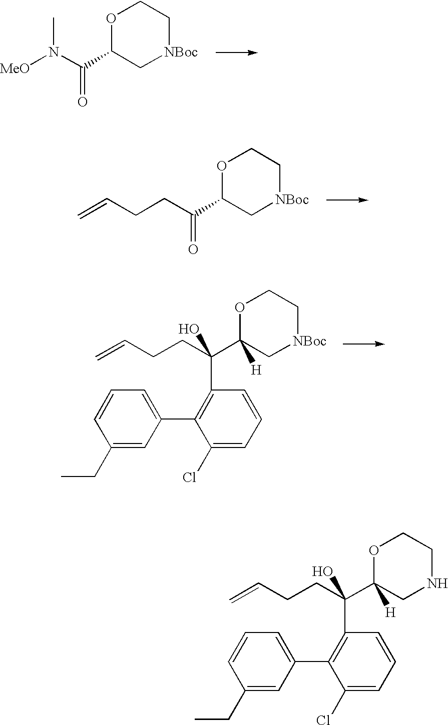

### Step 1. (R)-tert-butyl 2-pent-4-enoylmorpholine-4-carboxylate

To a solution of (R)-tert-butyl 2-(methoxy(methyl)carbamoyl)morpholine-4-carboxylate (1.2 g, 4.38 mmol) in 50 mL of THF at −78° C. under a nitrogen atmosphere was slowly added 26 mL (13.3 mmol, 0.5M) of (4-penten-1-yl)magnesium bromide in THF using a syringe. The solution was stirred overnight, allowing it to slowly warm to rt. A saturated solution of NH₄Cl in water (50 mL) was added to the reaction flask. The solution was extracted using EtOAc (3×25 mL). The combined organic extracts were dried over Na₂SO₄ and filtered, followed by concentration under reduced pressure to give 810 mg of (R)-tert-butyl 2-pent-4-enoylmorpholine-4-carboxylate.

### Step 2. (R)-tert-butyl 2-((R)-1-(6-chloro-3′-ethylbiphenyl-2-yl)-1-hydroxypent-4-enyl)morpholine-4-carboxylate

To a solution of 2-bromo-6-chloro-3′-ethylbiphenyl, 2.2 g (7.44 mmol) in 20 mL of THF at −78° C. under a nitrogen atmosphere was slowly added a hexane solution of n-BuLi (3.7 ml, 2.5M) using a syringe. The resulting solution was stirred for 0.5 h. 1,1-dimethylethyl (2R)-2-(4-pentenoyl)-4-morpholinecarboxylate (0.8 g, 2.97 mmol) in 20 mL of THF was slowly added to the above solution using a syringe.

The reaction was then allowed to stir and warm to rt overnight. A saturated solution of NH₄Cl in water (50 mL) was added to the reaction flask. The solution was extracted using EtOAc (3×25 mL). The combined organic extracts were dried over Na₂SO₄ and filtered, followed by concentration under reduced pressure. This afforded 550 mg of (R)-tert-butyl 2-((R)-1-(6-chloro-3′-ethylbiphenyl-2-yl)-1-hydroxypent-4-enyl)morpholine-4-carboxylate which was used without purification. LC-MS t_{R}=3.74 min, (m/z) 508.2 (M+H⁺).

### Step 3. (R)-1-(6-chloro-3′-ethylbiphenyl-2-yl)-1-((R)-morpholin-2-yl)pent-4-en-1-ol

To a solution of 1,1-dimethylethyl (2R)-2-[(1R)-1-(6-chloro-3′-ethyl-2-biphenylyl)-1-hydroxy-4-penten-1-yl]-4-morpholinecarboxylate (73 mg, 0.15 mmol) in 5 ml of acetonitrile was added 5 ml of 2N aqueous HCl. The reaction was stirred overnight. It was basified with 10N aqueous NaOH to pH=14 and extracted with DCM (3×10 ml). The combined organic extracts were dried over Na₂SO₄ and filtered, followed by concentration under reduced pressure. This afforded (R)-1-(6-chloro-3′-ethylbiphenyl-2-yl)-1-((R)-morpholin-2-yl)pent-4-en-1-ol which was used without purification.

### Preparation 29

### (R)-1-(6-chloro-3′-ethylbiphenyl-2-yl)-1-((R)-morpholin-2-yl)pent-4-en-1-ol

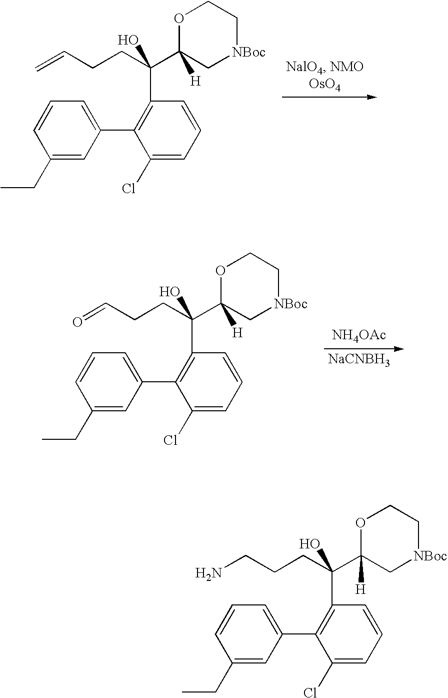

### Step 1. (R)-tert-butyl 2-((R)-1-(6-chloro-3′-ethylbiphenyl-2-yl)-1-hydroxy-4-oxobutyl)morpholine-4-carboxylate

To a solution of (R)-tert-butyl 2-((R)-1-(6-chloro-3′-ethylbiphenyl-2-yl)-1-hydroxypent-4-enyl)morpholine-4-carboxylate (350 mg, 0.72 mmol) in 10 mL of THF and 5 mL of water was added NMO (255 mg, 2.18 mmol), followed by NaIO₄ (310 mg, 1.44 mmol) and a few small crystals of OsO₄. The reaction was stirred overnight. The solution was diluted with 10 mL of water and extracted with CH₂Cl₂ (3×10 ml). The combined organic extracts were dried over Na₂SO₄ and filtered, followed by concentration under reduced pressure. This afforded (R)-tert-butyl 2-((R)-1-(6-chloro-3′-ethylbiphenyl-2-yl)-1-hydroxy-4-oxobutyl)morpholine-4-carboxylate which was used without purification. LC-MS t_{R}=3.36 min, (m/z) 510.2 (M+Na⁺).

### Step 2. (R)-tert-butyl 2-((R)-4-amino-1-(6-chloro-3′-ethylbiphenyl-2-yl)-1-hydroxybutyl)morpholine-4-carboxylate

To a refluxing solution of (R)-tert-butyl 2-((R)-1-(6-chloro-3′-ethylbiphenyl-2-yl)-1-hydroxy-4-oxobutyl)morpholine-4-carboxylate (350 mg, 0.7 mmol) in 20 mL of MeOH was added NH₃.AcOH (550 mg, 7.2 mmol), followed by NaCNBH₃ (135 mg, 2.2 mmol). After a few h at reflux the reaction was cooled to rt and diluted with 20 mL of water. The solution was extracted using EtOAc (3×10 ml). The combined organic extracts were dried over Na₂SO₄ and filtered, followed by concentration under reduced pressure. This afforded (R)-tert-butyl 2-((R)-4-amino-1-(6-chloro-3′-ethylbiphenyl-2-yl)-1-hydroxybutyl)morpholine-4-carboxylate which was used without purification. LC-MS t_{R}=2.56 min, (m/z) 489.2 (M+H⁺).

### Preparation 30

### 1-(3′-ethyl-6-fluorobiphenyl-2-yl)-5-methoxy-1-(piperidin-4-yl)pentan-1-ol

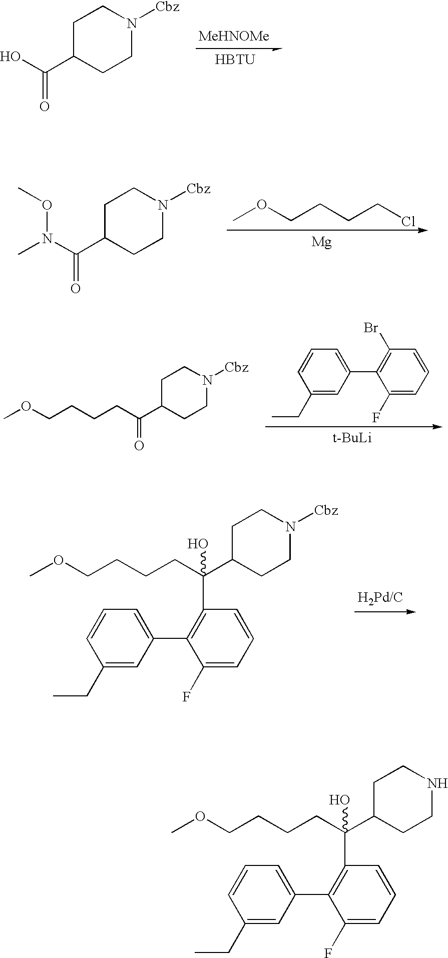

### Step 1. Benzyl 4-(methoxy(methyl)carbamoyl)piperidine-1-carboxylate

A solution of 1-(benzyloxycarbonyl)piperidine-4-carboxylic acid (2.1 g, 8.0 mmol) in 20 mL of DMF at 0° C. was treated with N,O-dimethylhydroxylamine hydrochloride (0.84 g, 8.6 mmol), i-Pr₂NEt (7 mL, 40.0 mmol), HBTU (3.3 g, 8.8 mmol), and HOBt (1.2 g, 8.8 mmol) and the mixture was stirred and warmed to 25° C. After 16 h, H₂O (50 mL) was added and the mixture was extracted with EtOAc (3×50 mL). The combined organic extracts were washed (1N HCl, 1N NaOH, H₂O, and brine), dried (Na₂SO₄), and concentrated to provide benzyl 4-(methoxy(methyl)carbamoyl)piperidine-1-carboxylate as a yellow oil (2.1 g, 89%).

### Step 2. Benzyl 4-(5-methoxypentanoyl)piperidine-1-carboxylate

A solution of benzyl 4-(methoxy(methyl)carbamoyl)piperidine-1-carboxylate (0.7 g, 2.3 mmol) in 4 mL of THF at −20° C. was treated with a solution of 4-(methyloxy)butyl magnesium chloride (7 mL of 1.28 M in THF, 9.0 mmol) and the mixture was stirred and warmed to 25° C. over 2 h before being quenched with the addition of aqueous 1N HCl and extracted with Et₂O. The combined organic extracts were dried (Na₂SO₄), concentrated, and subjected to flash chromatography to provide benzyl 4-(5-methoxypentanoyl)piperidine-1-carboxylate as a colorless oil (0.67 g, 88%). MS (m/z) 334.2 (M+H⁺).

### Step 3. benzyl 4-(143′-ethyl-6-fluorobiphenyl-2-yl)-1-hydroxy-5-methoxypentyl)piperidine-1-carboxylate

A solution of 2-bromo-3′-ethyl-6-fluorobiphenyl (0.5 mg, 1.8 mmol) in 2 mL of Et₂O at −78° C. was treated with t-BuLi (2.1 mL of 1.7 M in pentane, 3.6 mmol). After 5 min, a solution of benzyl 4-(5-methoxypentanoyl)piperidine-1-carboxylate (0.3 g, 0.9 mmol) in 2 mL of THF was added and the mixture was stirred for 1 h before being quenched with the addition of saturated aqueous NH₄Cl and extracted with Et₂O. The combined organic extracts were dried (Na₂SO₄), concentrated, and subjected to flash chromatography to provide benzyl 441-(3′-ethyl-6-fluorobiphenyl-2-yl)-1-hydroxy-5-methoxypentyl)piperidine-1-carboxylate as a colorless oil (0.15 g, 31%). MS (m/z) 556.2 (M+Na⁺).

### Step 4. 1-(3′-ethyl-6-fluorobiphenyl-2-yl)-5-methoxy-1-(piperidin-4-yl)pentan-1-ol

A solution of benzyl 4-(143′-ethyl-6-fluorobiphenyl-2-yl)-1-hydroxy-5-methoxypentyl)piperidine-1-carboxylate (70 mg, 0.13 mmol) in 2 mL of MeOH at 25° C. was treated with 10% Pd/C (20 mg) and stirred under an atmosphere of hydrogen. After 2 h, the mixture was filtered and concentrated to provide 1-(3′-ethyl-6-fluorobiphenyl-2-yl)-5-methoxy-1-(piperidin-4-yl)pentan-1-ol as a colorless oil (53 mg, quantitative). MS (m/z) 400.3 (M+H⁺).

The following procedures describe preparation of compounds of Formula I.

### Example 1

### N-{4-(6-chloro-3′-ethyl-2-biphenylyl)-4-hydr oxy-4-[(3R)-1-({4-[(methylamino)methyl]phenyl}carbonyl)-3-piperidinyl]butyl}acetamide (#30)

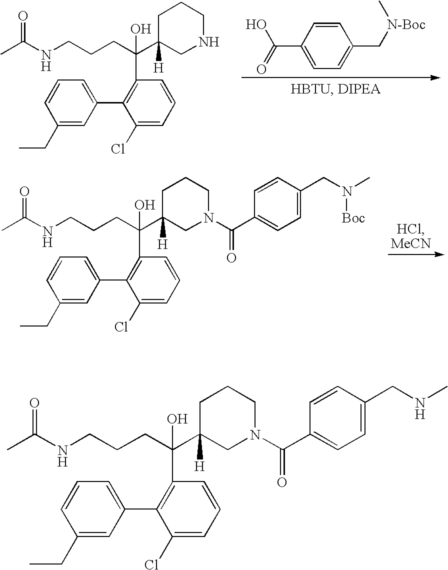

### Step 1. 1,1-dimethylethyl {[4-({(3R)-3-[4-(acetylamino)-1-(6-chloro-3′-ethyl-2-biphenylyl)-1-hydroxybutyl]-1-piperidinyl}carbonyl)phenyl]methyl}methylcarbamate

A solution of N-{4-(6-chloro-3′-ethyl-2-biphenylyl)-4-hydr oxy-4-[(3R)-3-piperidinyl]butyl}acetamide (48 mg, 0.10 mmol) in 1 mL of DMF at 25° C. was treated with 4-((tert-butoxycarbonyl(methyl)amino)methyl)benzoic acid (33 mg, 0.12 mmol), i-Pr₂NEt (0.089 mL, 0.5 mmol), and HBTU (47 mg, 0.12 mmol). After 24 h, H₂O was added and the mixture was extracted with EtOAc. The organic extracts were washed (1N aq HCl, 1N aq NaOH, H₂O, brine), dried (Na₂SO₄), concentrated under reduced pressure, and subjected to flash chromatography to provide 1,1-dimethylethyl {[4-({(3R)-3-[4-(acetylamino)-1-(6-chloro-3′-ethyl-2-biphenylyl)-1-hydroxybutyl]-1-piperidinyl}carbonyl)phenyl]methyl}methylcarbamate as a colorless oil (50 mg, 71%). MS (m/z) 676.3 (M+H⁺).

### Step 2. N-{4-(6-chloro-3′-ethyl-2-biphenylyl)-4-hydroxy-4-[(3R)-1-({4-[(methylamino)methyl]phenyl}carbonyl)-3-piperidinyl]butyl}acetamide

A solution of 1,1-dimethylethyl {[4-({(3R)-3-[4-(acetylamino)-1-(6-chloro-3′-ethyl-2-biphenylyl)-1-hydroxybutyl]-1-piperidinyl}carbonyl)phenyl]methyl}methylcarbamate (50 mg, 0.074 mmol) in 3 mL of CH₃CN at 25° C. was treated with 3 mL of aqueous 2N HCl. After 24 h, the mixture was concentrated under reduced pressure to provide N-{4-(6-chloro-3′-ethyl-2-biphenylyl)-4-hydroxy-4-[(3R)-1-({4-[(methylamino)methyl]phenyl}carbonyl)-3-piperidinyl]butyl}acetamide as a white solid (39 mg, quantitative). MS (m/z) 576.2 (M+H⁺).

### Example 2

The following compounds were prepared following procedures analogous to those described in Example 1.

| | |
|---|---|
| #19 | methyl 4-hydroxy-4-((R)-1-(4-((methylamino)methyl)benzoyl)piperidin-3-yl)- |
| | 4-(2-(o-tolyloxy)phenyl)butylcarbamate |
| #20 | N-4-(6-chloro-3′-ethylbiphenyl-2-yl)-4-hydroxy-4-((R)-1-(4- |
| | ((methylamino)methyl)benzoyl)piperidin-3-yl)butyl)formamide |
| #22 | ((3R)-3-(1-(3-chloro-2-(2-methylbenzyl)phenyl)-1-hydroxy-5- |
| | methoxypentyl)piperidin-1-yl)(4-((methylamino)methyl)phenyl)methanone |
| #31 | methyl 4-(6-fluoro-3′-methoxybiphenyl-2-yl)-4-hydroxy-4-((R)-1-(4- |
| | ((methylamino)methyl)benzoyl)piperidin-3-yl)butylcarbamate |
| #33 | methyl 4-(6-chloro-3′-methylbiphenyl-2-yl)-4-hydroxy-4-((R)-1-(4- |
| | ((methylamino)methyl)benzoyl)piperidin-3-yl)butylcarbamate |
| #37 | N-(4-(6-chloro-3′-ethylbiphenyl-2-yl)-4-hydroxy-4-((R)-1-(4- |
| | ((methylamino)methyl)benzoyl)piperidin-3-yl)butyl)propionamide |
| #41 | N-(4-(6-chloro-3′-ethylbiphenyl-2-yl)-4-hydroxy-4-((R)-1-(4- |
| | ((methylamino)methyl)benzoyl)piperidin-3-yl)butyl)-2-hydroxyacetamide |
| #42 | methyl 4-(6-chloro-3′-methoxybiphenyl-2-yl)-4-hydroxy-4-((R)-1-(4- |
| | ((methylamino)methyl)benzoyl)piperidin-3-yl)butylcarbamate |
| #47 | N-(4-(6-chloro-3′-ethylbiphenyl-2-yl)-4-hydroxy-4-((R)-1-(4- |
| | ((methylamino)methyl)benzoyl)piperidin-3-yl)butyl)-2,2,2-trifluoroacetamide |
| #48 | methyl {4-(3′-ethyl-6-fluoro-2-biphenylyl)-4-hydroxy-4-[(3R)-1-({4- |
| | [(methylamino)methyl]phenyl}carbonyl)-3-piperidinyl]butyl}carbamate |
| #49 | methyl {4-(2′,6-difluoro-5′-methyl-2-biphenylyl)-4-hydroxy-4-[(3R)-1-({4- |
| | [(methylamino)methyl]phenyl}carbonyl)-3-piperidinyl]butyl}carbamate |
| #50 | N-{4-(2′,6-difluoro-5′-methyl-2-biphenylyl)-4-hydroxy-4-[(3R)-1-({4- |
| | [(methylamino)methyl]phenyl}carbonyl)-3-piperidinyl]butyl}-2-hydroxyacetamide |
| #52 | (1R)-1-(6-chloro-3′-ethyl-2-biphenylyl)-1-[(2R)-4-({4- |
| | [(methylamino)methyl]phenyl}carbonyl)-2-morpholinyl]-5-(methyloxy)-1-pentanol |
| #53 | 1-(6-chloro-3′-ethyl-2-biphenylyl)-1-[(3R)-1-({4- |
| | [(methylamino)methyl]phenyl}carbonyl)-3-piperidinyl]-5-(methyloxy)-1-pentanol |
| #55 | N-{4-(6-chloro-3′-methyl-2-biphenylyl)-4-hydroxy-4-[(3R)-1-({4- |
| | [(methylamino)methyl]phenyl}carbonyl)-3-piperidinyl]butyl}acetamide |
| #58 | N-{4-(6-chloro-3′-methyl-2-biphenylyl)-4-[(3R)-1-({2-fluoro-4- |
| | [(methylamino)methyl]phenyl}carbonyl)-3-piperidinyl]-4-hydroxybutyl}acetamide |
| #63 | methyl [4-hydroxy-4-[(3R)-1-({4-[(methylamino)methyl]phenyl}carbonyl)-3- |
| | piperidinyl]-4-(2′,4,6-trifluoro-5′-methyl-2-biphenylyl)butyl]carbamate |
| #69 | methyl {4-(6-fluoro-3′-methyl-2-biphenylyl)-4-hydroxy-4-[(3R)-1-({4- |
| | [(methylamino)methyl]phenyl}carbonyl)-3-piperidinyl]butyl}carbamate |
| #70 | N-{4-(6-fluoro-3′-methyl-2-biphenylyl)-4-hydroxy-4-[(3R)-1-({4- |
| | [(methylamino)methyl]phenyl}carbonyl)-3-piperidinyl]butyl}-2-hydroxyacetamide |
| #71 | 2-chloro-N-{(4S)-4-(6-chloro-3′-ethyl-2-biphenylyl)-4-hydroxy-4-[(3R)-1-({4- |
| | [(methylamino)methyl]phenyl}carbonyl)-3-piperidinyl]butyl}acetamide |
| #87 | methyl {4-[6-fluoro-3′-methyl-5-(methyloxy)-2-biphenylyl]-4-hydroxy-4-[(3R)- |
| | 1-({4-[(methylamino)methyl]phenyl}carbonyl)-3-piperidinyl]butyl}carbamate |
| #91 | methyl {(4R)-4-(3′-ethyl-6-fluoro-2-biphenylyl)-4-hydroxy-4-[(2R)-4-({4- |
| | [(methylamino)methyl]phenyl}carbonyl)-2-morpholinyl]butyl}carbamate |
| #94 | methyl {4-[3-chloro-2-(3-quinolinyl)phenyl]-4-hydroxy-4-[(3R)-1-({4- |
| | [(methylamino)methyl]phenyl}carbonyl)-3-piperidinyl]butyl}carbamate |
| #95 | (1R)-1-[4-chloro-3-(3-ethylphenyl)-2-pyridinyl]-1-[(2R)-4-({4- |
| | [(methylamino)methyl]phenyl}carbonyl)-2-morpholinyl]-5-(methyloxy)-1-pentanol |
| #96 | methyl {4-{3-chloro-2-[4-(1-methylethyl)-2-quinazolinyl]phenyl}-4-hydroxy- |
| | 4-[(3R)-1-({4-[(methylamino)methyl]phenyl}carbonyl)-3- |
| | piperidinyl]butyl}carbamate |
| #97 | methyl {4-[2-chloro-3-(3-ethylphenyl)-4-pyridinyl]-4-hydroxy-4-[(3R)-1-({4- |
| | [(methylamino)methyl]phenyl}carbonyl)-3-piperidinyl]butyl}carbamate |
| #98 | methyl {(4R)-4-(6-chloro-3′-methyl-2-biphenylyl)-4-hydroxy-4-[(2R)-4-({4- |
| | [(methylamino)methyl]phenyl}carbonyl)-2-morpholinyl]butyl}carbamate |
| #102 | methyl {4-{3-chloro-2-[8-(1-methylethyl)-2-quinolinyl]phenyl}-4-hydroxy-4- |
| | [(3R)-1-({4-[(methylamino)methyl]phenyl}carbonyl)-3- |
| | piperidinyl]butyl}carbamate |
| #111 | methyl [2-({(R)-(6-chloro-3′-ethyl-2-biphenylyl)[(3R)-1-({4- |
| | [(methylamino)methyl]phenyl}carbonyl)-3- |
| | piperidinyl]methyl}oxy)ethyl]carbamate |
| #112 | methyl [2-({(S)-(6-chloro-3′-ethyl-2-biphenylyl)[(2R)-4-({4- |
| | [(methylamino)methyl]phenyl}carbonyl)-2- |
| | morpholinyl]methyl}oxy)ethyl]carbamate |
| #128 | methyl {4-[3-chloro-2-(8-methyl-2-quinolinyl)phenyl]-4-hydroxy-4-[(3R)-1- |
| | ({4-[(methylamino)methyl]phenyl}carbonyl)-3-piperidinyl]butyl}carbamate |
| #131 | methyl {4-[3-fluoro-2-(3-quinolinyl)phenyl]-4-hydroxy-4-[(3R)-1-({4- |
| | [(methylamino)methyl]phenyl}carbonyl)-3-piperidinyl]butyl}carbamate |
| #132 | methyl {4-{2-chloro-3-[3-(1-methylethyl)phenyl]-4-pyridinyl}-4-hydroxy-4- |
| | [(3R)-1-({4-[(methylamino)methyl]phenyl}carbonyl)-3- |
| | piperidinyl]butyl}carbamate |
| #133 | methyl {4-[3-chloro-2-(5-methyl-2-furanyl)phenyl]-4-hydroxy-4-[(3R)-1-({4- |
| | [(methylamino)methyl]phenyl}carbonyl)-3-piperidinyl]butyl}carbamate |
| #134 | (1R)-1-{2-chloro-3-[3-(1-methylethyl)phenyl]-4-pyridinyl}-1-[(2R)-4-({4- |
| | [(methylamino)methyl]phenyl}carbonyl)-2-morpholinyl]-5-(methyloxy)-1-pentanol |
| #135 | methyl {4-[5-chloro-4-(3-ethylphenyl)-3-pyridinyl]-4-hydroxy-4-[(3R)-1-({4- |
| | [(methylamino)methyl]phenyl}carbonyl)-3-piperidinyl]butyl}carbamate |
| #136 | methyl {4-{5-chloro-4-[3-(1-methylethyl)phenyl]-3-pyridinyl}-4-hydroxy-4- |
| | [(3R)-1-({4-[(methylamino)methyl]phenyl}carbonyl)-3- |
| | piperidinyl]butyl}carbamate |
| #142 | 1-(6-chloro-3′-ethyl-2-biphenylyl)-1-[(3R)-1-({4- |
| | [(methylamino)methyl]phenyl}carbonyl)-3-piperidinyl]-1,6-hexanediol |
| #144 | N-{(4S)-4-[6-fluoro-3′-(methyloxy)-2-biphenylyl]-4-hydroxy-4-[(3R)-1-({4- |
| | [(methylamino)methyl]phenyl}carbonyl)-3-piperidinyl]butyl}-2-hydroxyacetamide |
| #147 | N-{4-[6-chloro-3′-(methyloxy)-2-biphenylyl]-4-hydroxy-4-[(3R)-1-({4- |
| | [(methylamino)methyl]phenyl}carbonyl)-3-piperidinyl]butyl}-2-hydroxyacetamide |
| #150 | methyl {4-(6-chloro-3′-fluoro-5′-methyl-2-biphenylyl)-4-hydroxy-4-[(3R)-1- |
| | ({4-[(methylamino)methyl]phenyl}carbonyl)-3-piperidinyl]butyl}carbamate |
| #153 | methyl {4-[3-chloro-2-(2,3-dihydro-1-benzofuran-6-yl)phenyl]-4-hydroxy-4- |
| | [(3R)-1-({4-[(methylamino)methyl]phenyl}carbonyl)-3- |
| | piperidinyl]butyl}carbamate |
| #155 | methyl {(4S)-4-[6-chloro-2′-(methyloxy)-5′-(trifluoromethyl)-2-biphenylyl]-4- |
| | hydroxy-4-[(3R)-1-({4-[(methylamino)methyl]phenyl}carbonyl)-3- |
| | piperidinyl]butyl}carbamate |
| #158 | methyl {4-(3′-ethyl-6-fluoro-2-biphenylyl)-4-hydroxy-4-[(3R)-1-({4- |
| | [(methylamino)methyl]phenyl}carbonyl)-3-piperidinyl]butyl}carbamate |
| #159 | N-{4-(2′,6-difluoro-5′-methyl-2-biphenylyl)-4-hydroxy-4-[(3R)-1-({4- |
| | [(methylamino)methyl]phenyl}carbonyl)-3-piperidinyl]butyl}acetamide |
| #160 | N-{4-(6-fluoro-3′-methyl-2-biphenylyl)-4-hydroxy-4-[(3R)-1-({4- |
| | [(methylamino)methyl]phenyl}carbonyl)-3-piperidinyl]butyl}acetamide |
| #162 | N-{4-(3′-ethyl-6-fluoro-2-biphenylyl)-4-hydroxy-4-[(3R)-1-({4- |
| | [(methylamino)methyl]phenyl}carbonyl)-3-piperidinyl]butyl}acetamide |
| #166 | N-{4-(2′,6-difluoro-5′-methyl-2-biphenylyl)-4-hydroxy-4-[(3R)-1-({4- |
| | [(methylamino)methyl]phenyl}carbonyl)-3-piperidinyl]butyl}-2,2,2- |
| | trifluoroacetamide |
| #167 | methyl {4-[6-fluoro-3′-(1-methylethyl)-2-biphenylyl]-4-hydroxy-4-[(3R)-1-({4- |
| | [(methylamino)methyl]phenyl}carbonyl)-3-piperidinyl]butyl}carbamate |
| #171 | methyl {4-{3-chloro-2-[(3-ethylphenyl)oxy]phenyl}-4-hydroxy-4-[(3R)-1-({4- |
| | [(methylamino)methyl]phenyl}carbonyl)-3-piperidinyl]butyl}carbamate |
| #172 | 1-{3-chloro-2-[(3-methylphenyl)methyl]phenyl}-1-[(3R)-1-({4- |
| | [(methylamino)methyl]phenyl}carbonyl)-3-piperidinyl]-5-(methyloxy)-1-pentanol |
| #173 | methyl {4-{3-chloro-2-[(3-methylphenyl)methyl]phenyl}-4-hydroxy-4-[(3R)-1- |
| | ({4-[(methylamino)methyl]phenyl}carbonyl)-3-piperidinyl]butyl}carbamate |

### Example 3

### Methyl (S)-4-(6-chloro-3′-ethylbiphenyl-2-yl)-4-hydroxy-4-((R)-1-(4-((methylamino)methyl)benzoyl)piperidin-3-yl)butylcarbamate (#39)

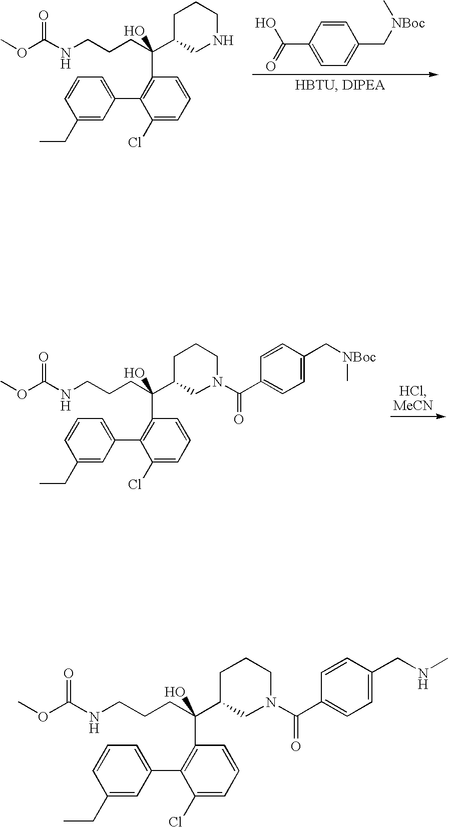

### Step 1. Methyl (S)-4-(6-chloro-3′-ethylbiphenyl-2-yl)-4-hydroxy-4-((R)-1-(4-(((N-t-butoxycarbonyl-N-methyl)amino)methyl)benzoyl)piperidin-3-yl)butylcarbamate

A solution of methyl (S)-4-(6-chloro-3′-ethylbiphenyl-2-yl)-4-hydroxy-4-((R)-piperidin-3-yl)butylcarbamate (30 mg, 0.07 mmol) in 1 mL of DMF at 25° C. was treated with 4-((tert-butoxycarbonyl(methyl)amino)methyl)benzoic acid (21 mg, 0.08 mmol), i-Pr₂NEt (0.063 mL, 0.37 mmol), and HBTU (30 mg, 0.08 mmol). After 1 h, H₂O was added and the mixture was extracted with EtOAc. The organic extracts were washed (1N HCl, 1N NaOH, H₂O, brine), dried (Na₂SO₄), concentrated under reduced pressure, and subjected to flash chromatography to provide methyl (S)-4-(6-chloro-3′-ethylbiphenyl-2-yl)-4-hydroxy-4-((R)-1-(4-(((N-t-butoxycarbonyl-N-methyl)amino)methyl)benzoyl)piperidin-3-yl)butylcarbamate as a colorless oil (24 mg, 51%). MS (m/z) 692.3 (M+H⁺).

### Step 2. methyl {(45)-4-(6-chloro-3′-ethyl-2-biphenylyl)-4-hydroxy-4-[(3R)-1-({4-[(methylamino)methyl]phenyl}carbonyl)-3-piperidinyl]butyl}carbamate

A solution of methyl (S)-4-(6-chloro-3′-ethylbiphenyl-2-yl)-4-hydroxy-4-((R)-1-(4-(((N-t-butoxycarbonyl-N-methyl)amino)methyl)benzoyl)piperidin-3-yl)butylcarbamate (24 mg, 0.034 mmol) in 3 mL of CH₃CN at 25° C. was treated with 3 mL of aqueous 2N HCl. After 24 h, the mixture was concentrated under reduced pressure to provide methyl {(4S)-4-(6-chloro-3′-ethyl-2-biphenylyl)-4-hydroxy-4-[(3R)-1-({4-[(methylamino)methyl]phenyl}carbonyl)-3-piperidinyl]butyl}carbamate as a white solid (17 mg, 81%). MS (m/z) 592.2 (M+H⁺).

### Example 4

The following piperidines were prepared following procedures analogous to those described in Example 3 using the appropriate amine intermediate and the indicated acid in place of 4-((tert-butoxycarbonyl(methyl)amino)methyl)benzoic acid in Step 1:

| Cpd. # | Product | Acid used in Step 1 |
|---|---|---|
| #1 | N-(4-((R)-1-(4- | 4-[({[(1,1- |
| | (aminomethyl)benzoyl)piperidin-3- | dimethylethyl)oxy]carbonyl}amino)methyl]benzoic |
| | yl)-4-hydroxy-4-(2-(o- | acid |
| | tolyloxy)phenyl)butyl)acetamide | |
| #2 | (4-(aminomethyl)phenyl)((3R)-3-(1- | 4-[({[(1,1- |
| | (3′-ethyl-6-fluorobiphenyl-2-yl)-1- | dimethylethyl)oxy]carbonyl}amino)methyl]benzoic |
| | hydroxy-5-methoxypentyl)piperidin- | acid |
| | 1-yl)methanone | |
| #4 | (4-(aminomethyl)phenyl)((R)-2-((R)- | 4-[({[(1,1- |
| | 1-(4′,6-difluoro-3′-methylbiphenyl-2- | dimethylethyl)oxy]carbonyl}amino)methyl]benzoic |
| | yl)-1-hydroxy-5- | acid |
| | methoxypentyl)morpholino)methanone | |
| #5 | methyl 4-((R)-1-(4- | 4-[({[(1,1- |
| | (aminomethyl)benzoyl)piperidin-3- | dimethylethyl)oxy]carbonyl}amino)methyl]benzoic |
| | yl)-4-hydroxy-4-(2-(o- | acid |
| | tolyloxy)phenyl)butylcarbamate | |
| #7 | (4-(aminomethyl)phenyl)((3R)-3-(1- | 4-[({[(1,1- |
| | (6-fluoro-3′-methoxy-5′- | dimethylethyl)oxy]carbonyl}amino)methyl]benzoic |
| | methylbiphenyl-2-yl)-1-hydroxy-5- | acid |
| | methoxypentyl)piperidin-1- | |
| | yl)methanone | |
| #8 | 1-((3R)-1-{[4- | 4-[({[(1,1- |
| | (aminomethyl)phenyl]carbonyl}-3- | dimethylethyl)oxy]carbonyl}amino)methyl]benzoic |
| | piperidinyl)-1-(6-chloro-3′-ethyl-2- | acid |
| | biphenylyl)-5-(methyloxy)-1-pentanol | |
| #9 | (4-(aminomethyl)phenyl)((3R)-3-(1-(6- | 4-[({[(1,1- |
| | chloro-3′-ethylbiphenyl-2-yl)-1-hydroxy- | dimethylethyl)oxy]carbonyl}amino)methyl]benzoic |
| | 5-methoxypentyl)piperidin-1- | acid |
| | yl)methanone | |
| #10 | (4-(aminomethyl)phenyl)((3R)-3-(1- | 4-[({[(1,1- |
| | (3-chloro-2-(3-methylbenzyl)phenyl)- | dimethylethyl)oxy]carbonyl}amino)methyl]benzoic |
| | 1-hydroxy-5- | acid |
| | methoxypentyl)piperidin-1- | |
| | yl)methanone | |
| #11 | ((R)-3-(1-(6-chloro-3′-ethylbiphenyl-2- | 4-[(4-{[(1,1- |
| | yl)-1-hydroxy-5- | dimethylethyl)oxy]carbonyl}-1- |
| | methoxypentyl)piperidin-1-yl)(4- | piperazinyl)methyl]benzoic acid |
| | (piperazin-1-ylmethyl)phenyl)methanone | |
| #12 | (1R)-1-((2R)-4-{[4- | 4-[({[(1,1- |
| | (aminomethyl)phenyl]carbonyl}-2- | dimethylethyl)oxy]carbonyl}amino)methyl]benzoic |
| | morpholinyl)-1-[6-fluoro-3′-methyl- | acid |
| | 5′-(methyloxy)-2-biphenylyl]-5- | |
| | (methyloxy)-1-pentanol | |
| #13 | (3-(aminomethyl)phenyl)((R)-2-((R)-1- | 3-[({[(1,1- |
| | (6-chloro-3′-ethylbiphenyl-2-yl)-1- | dimethylethyl)oxy]carbonyl}amino)methyl]benzoic |
| | hydroxy-5- | acid |
| | methoxypentyl)morpholino)methanone | |
| #14 | (4-(aminomethyl)phenyl)((R)-2-((R)- | 4-[({[(1,1- |
| | 1-(6-chloro-3′-ethylbiphenyl-2-yl)-1- | dimethylethyl)oxy]carbonyl}amino)methyl]benzoic |
| | hydroxy-5- | acid |
| | methoxypentyl)morpholino)methanone | |
| #15 | (4-(aminomethyl)phenyl)((3R)-3-(1- | 4-[({[(1,1- |
| | (3-chloro-2-(o-tolyloxy)phenyl)-1- | dimethylethyl)oxy]carbonyl}amino)methyl]benzoic |
| | hydroxy-5-methoxypentyl)piperidin- | acid |
| | 1-yl)methanone | |
| #16 | (4-(aminomethyl)phenyl)((3R)-3-(1- | 4-[({[(1,1- |
| | (2-(2-chloro-6- | dimethylethyl)oxy]carbonyl}amino)methyl]benzoic |
| | methylphenoxy)phenyl)-1-hydroxy-5- | acid |
| | methoxypentyl)piperidin-1- | |
| | yl)methanone | |
| #17 | methyl 4-(6-chloro-3′-ethylbiphenyl-2- | 4-[(dimethylamino)methyl]benzoic acid |
| | yl)-4-((R)-1-(4- | |
| | ((dimethylamino)methyl)benzoyl)piperidin- | |
| | 3-yl)-4-hydroxybutylcarbamate | |
| #18 | (4-(aminomethyl)phenyl)((2R)-2- | 4-[({[(1,1- |
| | ((1R)-1-(6-chloro-2′-fluoro-5′- | dimethylethyl)oxy]carbonyl}amino)methyl]benzoic |
| | methylbiphenyl-2-yl)-1-hydroxy-5- | acid |
| | methoxypentyl)morpholino)methanone | |
| #21 | (4-(2-aminoethyl)phenyl)((3R)-3-(1-(6- | 4-[2-({[(1,1- |
| | chloro-3′-ethylbiphenyl-2-yl)-1-hydroxy- | dimethylethyl)oxy]carbonyl}amino)ethyl]benzoic |
| | 5-methoxypentyl)piperidin-1- | acid |
| | yl)methanone | |
| #23 | methyl 4-((R)-1-(4- | 4-[({[(1,1- |
| | (aminomethyl)benzoyl)piperidin-3- | dimethylethyl)oxy]carbonyl}amino)methyl]benzoic |
| | yl)-4-(6-fluoro-3′-methoxybiphenyl-2- | acid |
| | yl)-4-hydroxybutylcarbamate | |
| #24 | (4-(aminomethyl)phenyl)((3R)-3-(1- | 4-[({[(1,1- |
| | (3-chloro-2-(2-ethylphenoxy)phenyl)- | dimethylethyl)oxy]carbonyl}amino)methyl]benzoic |
| | 1-hydroxy-5- | acid |
| | methoxypentyl)piperidin-1- | |
| | yl)methanone | |
| #25 | (4-(aminomethyl)phenyl)((3R)-3-(1- | 4-[({[(1,1- |
| | (3-chloro-2-(3-ethylphenoxy)phenyl)- | dimethylethyl)oxy]carbonyl}amino)methyl]benzoic |
| | 1-hydroxy-5- | acid |
| | methoxypentyl)piperidin-1- | |
| | yl)methanone | |
| #26 | (4-(aminomethyl)phenyl)((R)-2-((R)- | 4-[({[(1,1- |
| | 1-(6-chloro-3′- | dimethylethyl)oxy]carbonyl}amino)methyl]benzoic |
| | (methoxymethyl)biphenyl-2-yl)-1- | acid |
| | hydroxy-5- | |
| | methoxypentyl)morpholino)methanone | |
| #27 | (4-(aminomethyl)phenyl)(3-((R)-1-(3- | 4-[({[(1,1- |
| | chloro-2-(quinolin-3-yl)phenyl)-1- | dimethylethyl)oxy]carbonyl}amino)methyl]benzoic |
| | hydroxy-5-methoxypentyl)piperidin- | acid |
| | 1-yl)methanone | |
| #28 | (4-(aminomethyl)phenyl)((R)-2-((R)- | 4-[({[(1,1- |
| | 1-(3-chloro-2-(naphthalen-2- | dimethylethyl)oxy]carbonyl}amino)methyl]benzoic |
| | yl)phenyl)-1-hydroxy-5- | acid |
| | methoxypentyl)morpholino)methanone | |
| #29 | (1R)-1-((2R)-4-{[4- | 4-[({[(1,1- |
| | (aminomethyl)phenyl]carbonyl}-2- | dimethylethyl)oxy]carbonyl}amino)methyl]benzoic |
| | morpholinyl)-1-[3-chloro-2-(3- | acid |
| | quinolinyl)phenyl]-5-(methyloxy)-1- | |
| | pentanol | |
| #32 | methyl 4-((R)-1-(4- | 4-[({[(1,1- |
| | (aminomethyl)benzoyl)piperidin-3- | dimethylethyl)oxy]carbonyl}amino)methyl]benzoic |
| | yl)-4-(6-chloro-3′-ethylbiphenyl-2- | acid |
| | yl)-4-hydroxybutylcarbamate | |
| #34 | (4-(2-aminoethoxy)phenyl)((3R)-3-(1-(6- | 4-{[2-({[(1,1- |
| | chloro-3′-ethylbiphenyl-2-yl)-1-hydroxy- | dimethylethyl)oxy]carbonyl}amino)ethyl]oxy}benzoic |
| | 5-methoxypentyl)piperidin-1- | acid |
| | yl)methanone | |
| #35 | methyl (R)-4-((R)-4-(4- | 4-[({[(1,1- |
| | (aminomethyl)benzoyl)morpholin-2- | dimethylethyl)oxy]carbonyl}amino)methyl]benzoic |
| | yl)-4-(6-chloro-3′-ethylbiphenyl-2- | acid |
| | yl)-4-hydroxybutylcarbamate | |
| #36 | methyl 4-((R)-1-(4- | 4-[({[(1,1- |
| | (aminomethyl)benzoyl)piperidin-3- | dimethylethyl)oxy]carbonyl}amino)methyl]benzoic |
| | yl)-4-(6-chloro-3′-methoxybiphenyl- | acid |
| | 2-yl)-4-hydroxybutylcarbamate | |
| #38 | ethyl 4-((R)-1-(4- | 4-[({[(1,1- |
| | (aminomethyl)benzoyl)piperidin-3- | dimethylethyl)oxy]carbonyl}amino)methyl]benzoic |
| | yl)-4-(6-chloro-3′-ethylbiphenyl-2- | acid |
| | yl)-4-hydroxybutylcarbamate | |
| #40 | methyl 4-((R)-1-(4- | 4-[({[(1,1- |
| | (aminomethyl)benzoyl)piperidin-3- | dimethylethyl)oxy]carbonyl}amino)methyl]benzoic |
| | yl)-4-(6-chloro-3′-isopropylbiphenyl- | acid |
| | 2-yl)-4-hydroxybutylcarbamate | |
| #43 | methyl 4-((R)-1-(4-(aminomethyl)-2- | 4-[({[(1,1- |
| | fluorobenzoyl)piperidin-3-yl)-4-(6- | dimethylethyl)oxy]carbonyl}amino)methyl]- |
| | chloro-3′-ethylbiphenyl-2-yl)-4- | 2-fluorobenzoic acid |
| | hydroxybutylcarbamate | |
| #44 | isopropyl 4-((R)-1-(4- | 4-[({[(1,1- |
| | (aminomethyl)benzoyl)piperidin-3- | dimethylethyl)oxy]carbonyl}amino)methyl]- |
| | yl)-4-(6-chloro-3′-ethylbiphenyl-2- | 2-fluorobenzoic acid |
| | yl)-4-hydroxybutylcarbamate | |
| #45 | methyl 4-(6-chloro-3′-ethylbiphenyl-2- | 4-{[{[(1,1- |
| | yl)-4-((R)-1-(4- | dimethylethyl)oxy]carbonyl}(ethyl)amino]methyl}benzoic |
| | ((ethylamino)methyl)benzoyl)piperidin- | acid |
| | 3-yl)-4-hydroxybutylcarbamate | |
| #46 | methyl 4-(6-chloro-3′-ethylbiphenyl-2- | 4-{[{[(1,1- |
| | yl)-4-hydroxy-4-((R)-1-(4- | dimethylethyl)oxy]carbonyl}(1- |
| | ((isopropylamino)methyl)benzoyl)piperidin- | methylethyl)amino]methyl}benzoic acid |
| | 3-yl)butylcarbamate | |
| #51 | methyl 4-(6-chloro-3′-ethylbiphenyl-2- | 4-[((cyclohexylmethyl){[(1,1- |
| | yl)-4-((R)-1-(4- | dimethylethyl)oxy]carbonyl}amino)methyl]benzoic |
| | ((cyclohexylmethylamino)methyl)benzoyl)piperidin- | acid |
| | 3-yl)-4- | |
| | hydroxybutylcarbamate | |
| #54 | methyl [4-((3R)-1-{[4-(aminomethyl)- | 4-[({[(1,1- |
| | 3-(methyloxy)phenyl]carbonyl}-3- | dimethylethyl)oxy]carbonyl}amino)methyl]- |
| | piperidinyl)-4-(6-chloro-3′-ethyl-2- | 3-(methyloxy)benzoic acid |
| | biphenylyl)-4- | |
| | hydroxybutyl]carbamate | |
| #56 | N-[4-((3R)-1-{[4-(aminomethyl)-2- | 4-[({[(1,1- |
| | fluorophenyl]carbonyl}-3- | dimethylethyl)oxy]carbonyl}amino)methyl]- |
| | piperidinyl)-4-(6-chloro-3′-methyl-2- | 2-fluorobenzoic acid |
| | biphenylyl)-4- | |
| | hydroxybutyl]acetamide | |
| #57 | N-[4-((3R)-1-{[4- | 4-[({[(1,1- |
| | (aminomethyl)phenyl]carbonyl}-3- | dimethylethyl)oxy]carbonyl}amino)methyl]benzoic |
| | piperidinyl)-4-(6-chloro-3′-methyl-2- | acid |
| | biphenylyl)-4- | |
| | hydroxybutyl]acetamide | |
| #59 | N-{4-(6-chloro-3′-methyl-2- | 4-[2,2,2-trifluoro-1-hydroxy-1- |
| | biphenylyl)-4-hydroxy-4-[(3R)-1-({4- | (trifluoromethyl)ethyl]benzoic acid |
| | [2,2,2-trifluoro-1-hydroxy-1- | |
| | (trifluoromethyl)ethyl]phenyl}carbonyl)- | |
| | 3-piperidinyl]butyl}acetamide | |
| #60 | 4-{[(3R)-3-(1-(6-chloro-3′-ethyl-2- | 1,4-benzenedicarboxylic acid |
| | biphenylyl)-1-hydroxy-4- | |
| | {[(methyloxy)carbonyl]amino}butyl)- | |
| | 1-piperidinyl]carbonyl}benzoic acid | |
| #61 | methyl {4-(6-chloro-3′-methyl-2- | 4-[(methylsulfonyl)methyl]benzoic |
| | biphenylyl)-4-hydroxy-4-[(3R)-1-({4- | acid |
| | [(methylsulfonyl)methyl]phenyl}carbonyl)- | |
| | 3-piperidinyl]butyl}carbamate | |
| #64 | methyl [4-((3R)-1-{[4-(2- | 4-[2-({[(1,1- |
| | aminoethyl)phenyl]carbonyl}-3- | dimethylethyl)oxy]carbonyl}amino)ethyl]benzoic |
| | piperidinyl)-4-(6-chloro-3′-methyl-2- | acid |
| | biphenylyl)-4- | |
| | hydroxybutyl]carbamate | |
| #65 | methyl [4-[(3R)-1-({2-fluoro-4- | 4-{[{[(1,1- |
| | [(methylamino)methyl]phenyl}carbonyl)- | dimethylethyl)oxy]carbonyl}(methyl)amino]methyl}- |
| | 3-piperidinyl]-4-hydroxy-4- | 2-fluorobenzoic acid |
| | (2′,4,6-trifluoro-5′-methyl-2- | |
| | biphenylyl)butyl]carbamate | |
| #66 | methyl [4-[(3R)-1-({4-[(2- | 4-{[2-({[(1,1- |
| | aminoethyl)oxy]phenyl}carbonyl)-3- | dimethylethyl)oxy]carbonyl}amino)ethyl]oxy}benzoic |
| | piperidinyl]-4-(6-chloro-3′-methyl-2- | acid |
| | biphenylyl)-4- | |
| | hydroxybutyl]carbamate | |
| #67 | methyl [4-((3R)-1-{[4- | 4-[({[(1,1- |
| | (aminomethyl)phenyl]carbonyl}-3- | dimethylethyl)oxy]carbonyl}amino)methyl]benzoic |
| | piperidinyl)-4-(6-chloro-3′-methyl-2- | acid |
| | biphenylyl)-4- | |
| | hydroxybutyl]carbamate | |
| #68 | methyl [4-((3R)-1-{[3- | {3-[({[(1,1- |
| | (aminomethyl)phenyl]acetyl}-3- | dimethylethyl)oxy]carbonyl}amino)methyl]phenyl}acetic |
| | piperidinyl)-4-(6-chloro-3′-methyl-2- | acid |
| | biphenylyl)-4- | |
| | hydroxybutyl]carbamate | |
| #72 | methyl {4-(6-chloro-3′-ethyl-2- | 4-[(cyclohexyl{[(1,1- |
| | biphenylyl)-4-[(3R)-1-({4- | dimethylethyl)oxy]carbonyl}amino)methyl]benzoic |
| | [(cyclohexylamino)methyl]phenyl}carbonyl)- | acid |
| | 3-piperidinyl]-4- | |
| | hydroxybutyl}carbamate | |
| #73 | methyl {4-(6-chloro-3′-ethyl-2- | 4-{[{[(1,1- |
| | biphenylyl)-4-hydroxy-4-[(3R)-1-({4- | dimethylethyl)oxy]carbonyl}(tetrahydro- |
| | [(tetrahydro-2H-pyran-4- | 2H-pyran-4- |
| | ylamino)methyl]phenyl}carbonyl)-3- | yl)amino]methyl}benzoic acid |
| | piperidinyl]butyl}carbamate | |
| #74 | methyl [4-[(3R)-1-({2-bromo-4- | 2-bromo-4-{[{[(1,1- |
| | [(methylamino)methyl]phenyl}carbonyl)- | dimethylethyl)oxy]carbonyl}(methyl)amino]methyl}benzoic |
| | 3-piperidinyl]-4-(6-chloro-3′- | acid |
| | ethyl-2-biphenylyl)-4- | |
| | hydroxybutyl]carbamate | |
| #75 | methyl {4-(6-chloro-3′-ethyl-2- | 5-{[{[(1,1- |
| | biphenylyl)-4-hydroxy-4-[(3R)-1-({5- | dimethylethyl)oxy]carbonyl}(methyl)amino]methyl}- |
| | [(methylamino)methyl]-2- | 2-biphenylcarboxylic |
| | biphenylyl}carbonyl)-3- | acid |
| | piperidinyl]butyl}carbamate | |
| #76 | methyl {4-(6-chloro-3′-ethyl-2- | 4-{[{[(1,1- |
| | biphenylyl)-4-hydroxy-4-[(3R)-1-({4- | dimethylethyl)oxy]carbonyl}(methyl)amino]methyl}- |
| | [(methylamino)methyl]-1- | 1- |
| | naphthalenyl}carbonyl)-3- | naphthalenecarboxylic acid |
| | piperidinyl]butyl}carbamate | |
| #77 | methyl [4-[(3R)-1-({2-butyl-4- | 2-butyl-4-{[{[(1,1- |
| | [(methylamino)methyl]phenyl}carbonyl)- | dimethylethyl)oxy]carbonyl}(methyl)amino]methyl}benzoic |
| | 3-piperidinyl]-4-(6-chloro-3′- | acid |
| | ethyl-2-biphenylyl)-4- | |
| | hydroxybutyl]carbamate | |
| #78 | methyl {4-(6-chloro-3′-ethyl-2- | 4-{[{[(1,1- |
| | biphenylyl)-4-[(3R)-1-({2-ethyl-4- | dimethylethyl)oxy]carbonyl}(methyl)amino]methyl}- |
| | [(methylamino)methyl]phenyl}carbonyl)- | 2-ethylbenzoic acid |
| | 3-piperidinyl]-4- | |
| | hydroxybutyl}carbamate | |
| #79 | methyl {4-(6-chloro-3′-ethyl-2- | 4-{[{[(1,1- |
| | biphenylyl)-4-hydroxy-4-[(3R)-1-({2- | dimethylethyl)oxy]carbonyl}(methyl)amino]methyl}- |
| | methyl-4- | 2-methylbenzoic acid |
| | [(methylamino)methyl]phenyl}carbonyl)- | |
| | 3-piperidinyl]butyl}carbamate | |
| #80 | methyl [4-(6-chloro-3′-ethyl-2- | 4-{[{[(1,1- |
| | biphenylyl)-4-hydroxy-4-((3R)-1-{[4- | dimethylethyl)oxy]carbonyl}(methyl)amino]methyl}- |
| | [(methylamino)methyl]-2-(2- | 2-(2- |
| | methylpropyl)phenyl]carbonyl}-3- | methylpropyl)benzoic acid |
| | piperidinyl)butyl]carbamate | |
| #81 | methyl {4-(6-chloro-3′-ethyl-2- | 4-{[{[(1,1- |
| | biphenylyl)-4-[(3R)-1-({2-[2-(1,3- | dimethylethyl)oxy]carbonyl}(methyl)amino]methyl}- |
| | dioxolan-2-yl)ethyl]-4- | 2-[2-(1,3-dioxolan-2- |
| | [(methylamino)methyl]phenyl}carbonyl)- | yl)ethyl]benzoic acid |
| | 3-piperidinyl]-4- | |
| | hydroxybutyl}carbamate | |
| #82 | methyl {4-(6-chloro-3′-ethyl-2- | 2-(2-cyanoethyl)-4-{[{[(1,1- |
| | biphenylyl)-4-[(3R)-1-({2-(2- | dimethylethyl)oxy]carbonyl}(methyl)amino]methyl}benzoic |
| | cyanoethyl)-4- | acid |
| | [(methylamino)methyl]phenyl}carbonyl)- | |
| | 3-piperidinyl]-4- | |
| | hydroxybutyl}carbamate | |
| #85 | methyl {4-(6-chloro-3′-ethyl-2- | 2-cyano-4-{[{[(1,1- |
| | biphenylyl)-4-[(3R)-1-({2-cyano-4- | dimethylethyl)oxy]carbonyl}(methyl)amino]methyl}benzoic |
| | [(methylamino)methyl]phenyl}carbonyl)- | acid |
| | 3-piperidinyl]-4- | |
| | hydroxybutyl}carbamate | |
| #88 | methyl [4-(6-chloro-3′-ethyl-2- | 4-{[{[(1,1- |
| | biphenylyl)-4-hydroxy-4-((3R)-1-{[4- | dimethylethyl)oxy]carbonyl}(methyl)amino]methyl}- |
| | [(methylamino)methyl]-2- | 2-(methyloxy)benzoic |
| | (methyloxy)phenyl]carbonyl}-3- | acid |
| | piperidinyl)butyl]carbamate | |
| #92 | (1R)-1-((2R)-4-{[4- | 4-[({[(1,1- |
| | (aminomethyl)phenyl]carbonyl}-2- | dimethylethyl)oxy]carbonyl}amino)methyl]benzoic |
| | morpholinyl)-1-[2-chloro-3-(3- | acid |
| | ethylphenyl)-4-pyridinyl]-5- | |
| | (methyloxy)-1-pentanol | |
| #93 | 1-((2R)-4-{[4- | 4-[({[(1,1- |
| | (aminomethyl)phenyl]carbonyl}-2- | dimethylethyl)oxy]carbonyl}amino)methyl]benzoic |
| | morpholinyl)-1-[2-(1-benzothien-3- | acid |
| | 3-chlorophenyl]-5-(methyloxy)-1- | |
| | pentanol | |
| #99 | 1-((3R)-1-{[4- | 4-[({[(1,1- |
| | (aminomethyl)phenyl]carbonyl}-3- | dimethylethyl)oxy]carbonyl}amino)methyl]benzoic |
| | piperidinyl)-1-{3-chloro-2-[(3- | acid |
| | methylphenyl)oxy]phenyl}-5- | |
| | (methyloxy)-1-pentanol | |
| #100 | methyl (4-((3R)-1-{[4- | 4-[({[(1,1- |
| | (aminomethyl)phenyl]carbonyl}-3- | dimethylethyl)oxy]carbonyl}amino)methyl]benzoic |
| | piperidinyl)-4-{3-chloro-2-[(3- | acid |
| | methylphenyl)oxy]phenyl}-4- | |
| | hydroxybutyl)carbamate | |
| #101 | methyl (4-((3R)-1-{[4- | 4-[({[(1,1- |
| | (aminomethyl)phenyl]carbonyl}-3- | dimethylethyl)oxy]carbonyl}amino)methyl]benzoic |
| | piperidinyl)-4-{2-[(2,6- | acid |
| | dimethylphenyl)oxy]phenyl}-4- | |
| | hydroxybutyl)carbamate | |
| #118 | methyl [4-[(3R)-1-({4- | 4-[({[(1,1- |
| | [amino(imino)methyl]phenyl}carbonyl)- | dimethylethyl)oxy]carbonyl}amino)(imino)methyl]benzoic |
| | 3-piperidinyl]-4-(6-fluoro-3′- | acid |
| | methyl-2-biphenylyl)-4- | |
| | hydroxybutyl]carbamate | |
| #130 | methyl {4-((3R)-1-{[4- | 4-[({[(1,1- |
| | (aminomethyl)-2- | dimethylethyl)oxy]carbonyl}amino)methyl]- |
| | fluorophenyl]carbonyl}-3- | 2-fluorobenzoic acid |
| | piperidinyl)-4-[3-chloro-2-(3- | |
| | quinolinyl)phenyl]-4- | |
| | hydroxybutyl}carbamate | |
| #143 | N-{4-((3R)-1-{[4- | 4-[({[(1,1- |
| | (aminomethyl)phenyl]carbonyl}-3- | dimethylethyl)oxy]carbonyl}amino)methyl]benzoic |
| | piperidinyl)-4-[6-fluoro-3′- | acid |
| | (methyloxy)-2-biphenylyl]-4- | |
| | hydroxybutyl}-2-hydroxyacetamide | |
| #145 | N-{4-((3R)-1-{[4-(aminomethyl)-2- | 4-[({[(1,1- |
| | fluorophenyl]carbonyl}-3- | dimethylethyl)oxy]carbonyl}amino)methyl]- |
| | piperidinyl)-4-[6-fluoro-3′- | 2-fluorobenzoic acid |
| | (methyloxy)-2-biphenylyl]-4- | |
| | hydroxybutyl}-2-hydroxyacetamide | |
| #146 | N-{4-((3R)-1-{[4- | 4-[({[(1,1- |
| | (aminomethyl)phenyl]carbonyl}-3- | dimethylethyl)oxy]carbonyl}amino)methyl]benzoic |
| | piperidinyl)-4-[6-chloro-3′- | acid |
| | (methyloxy)-2-biphenylyl]-4- | |
| | hydroxybutyl}-2-hydroxyacetamide | |
| #148 | N-{4-((3R)-1-{[4-(aminomethyl)-2- | 4-[({[(1,1- |
| | fluorophenyl]carbonyl}-3- | dimethylethyl)oxy]carbonyl}amino)methyl]- |
| | piperidinyl)-4-[6-chloro-3′- | 2-fluorobenzoic acid |
| | (methyloxy)-2-biphenylyl]-4- | |
| | hydroxybutyl}-2-hydroxyacetamide | |
| #149 | methyl {4-(6-chloro-3′-fluoro-5′- | 4-{[{[(1,1- |
| | methyl-2-biphenylyl)-4-[(3R)-1-({2- | dimethylethyl)oxy]carbonyl}(methyl)amino]methyl}- |
| | fluoro-4- | 2-fluorobenzoic acid |
| | [(methylamino)methyl]phenyl}carbonyl)- | |
| | 3-piperidinyl]-4- | |
| | hydroxybutyl}carbamate | |
| #151 | methyl {4-(3′,6-difluoro-5′-methyl-2- | 4-{[{[(1,1- |
| | biphenylyl)-4-[(3R)-1-({2-fluoro-4- | dimethylethyl)oxy]carbonyl}(methyl)amino]methyl}- |
| | [(methylamino)methyl]phenyl}carbonyl)- | 2-fluorobenzoic acid |
| | 3-piperidinyl]-4- | |
| | hydroxybutyl}carbamate | |
| #152 | methyl {4-[3-chloro-2-(2,3-dihydro-1- | 4-{[{[(1,1- |
| | benzofuran-6-yl)phenyl]-4-[(3R)-1- | dimethylethyl)oxy]carbonyl}(methyl)amino]methyl}- |
| | ({2-fluoro-4- | 2-fluorobenzoic acid |
| | [(methylamino)methyl]phenyl}carbonyl)- | |
| | 3-piperidinyl]-4- | |
| | hydroxybutyl}carbamate | |
| #154 | methyl {4-[6-chloro-2′-(methyloxy)- | 4-{[{[(1,1- |
| | 5′-(trifluoromethyl)-2-biphenylyl]-4- | dimethylethyl)oxy]carbonyl}(methyl)amino]methyl}- |
| | [(3R)-1-({2-fluoro-4- | 2-fluorobenzoic acid |
| | [(methylamino)methyl]phenyl}carbonyl)- | |
| | 3-piperidinyl]-4- | |
| | hydroxybutyl}carbamate | |
| #156 | methyl (4-((3R)-1-{[4- | 4-[({[(1,1- |
| | (aminomethyl)phenyl]carbonyl}-3- | dimethylethyl)oxy]carbonyl}amino)methyl]benzoic |
| | piperidinyl)-4-{3-chloro-2-[(2- | acid |
| | ethylphenyl)oxy]phenyl}-4- | |
| | hydroxybutyl)carbamate | |
| #157 | methyl (4-((3R)-1-{[4- | 4-[({[(1,1- |
| | (aminomethyl)phenyl]carbonyl}-3- | dimethylethyl)oxy]carbonyl}amino)methyl]benzoic |
| | piperidinyl)-4-{3-chloro-2-[(2- | acid |
| | methylphenyl)oxy]phenyl}-4- | |
| | hydroxybutyl)carbamate | |
| #161 | methyl {(4S)-4-(3′-ethyl-6-fluoro-2- | 4-{[{[(1,1- |
| | biphenylyl)-4-[(3R)-1-({2-fluoro-4- | dimethylethyl)oxy]carbonyl}(methyl)amino]methyl}- |
| | [(methylamino)methyl]phenyl}carbonyl)- | 2-fluorobenzoic acid |
| | 3-piperidinyl]-4- | |
| | hydroxybutyl}carbamate | |
| #163 | methyl [4-[(3R)-1-({2-fluoro-4- | 4-{[{[(1,1- |
| | [(methylamino)methyl]phenyl}carbonyl)- | dimethylethyl)oxy]carbonyl}(methyl)amino]methyl}- |
| | 3-piperidinyl]-4-(6-fluoro-3′- | 2-fluorobenzoic acid |
| | methyl-2-biphenylyl)-4- | |
| | hydroxybutyl]carbamate | |
| #164 | N-{4-(2′,6-difluoro-5′-methyl-2- | 4-{[{[(1,1- |
| | biphenylyl)-4-[(3R)-1-({2-fluoro-4- | dimethylethyl)oxy]carbonyl}(methyl)amino]methyl}- |
| | [(methylamino)methyl]phenyl}carbonyl)- | 2-fluorobenzoic acid |
| | 3-piperidinyl]-4-hydroxybutyl}-2- | |
| | hydroxyacetamid | |
| #165 | methyl {4-(2′,6-difluoro-5′-methyl-2- | 4-{[{[(1,1- |
| | biphenylyl)-4-[(3R)-1-({2-fluoro-4- | dimethylethyl)oxy]carbonyl}(methyl)amino]methyl}- |
| | [(methylamino)methyl]phenyl}carbonyl)- | 2-fluorobenzoic acid |
| | 3-piperidinyl]-4- | |
| | hydroxybutyl}carbamate | |
| #168 | methyl {4-(2′,6-difluoro-5′-methyl-2- | 4-[({[(1,1- |
| | biphenylyl)-4-[(3R)-1-({2-fluoro-4- | dimethylethyl)oxy]carbonyl}amino)methyl]benzoic |
| | [(methylamino)methyl]phenyl}carbonyl)- | acid |
| | 3-piperidinyl]-4- | |
| | hydroxybutyl}carbamate | |
| #169 | methyl (4-((3R)-1-{[4- | 4-[({[(1,1- |
| | (aminomethyl)phenyl]carbonyl}-3- | dimethylethyl)oxy]carbonyl}amino)methyl]benzoic |
| | piperidinyl)-4-{3-chloro-2-[(3- | acid |
| | ethylphenyl)oxy]phenyl}-4- | |
| | hydroxybutyl)carbamate | |
| #170 | methyl (4-((3R)-1-{[4- | 4-[({[(1,1- |
| | (aminomethyl)phenyl]carbonyl}-3- | dimethylethyl)oxy]carbonyl}amino)methyl]benzoic |
| | piperidinyl)-4-{3-fluoro-2-[(3- | acid |
| | methylphenyl)oxy]phenyl}-4- | |
| | hydroxybutyl)carbamate | |
| #174 | methyl {4-{3-chloro-2-[(3- | 4-{[{[(1,1- |
| | methylphenyl)methyl]phenyl}-4- | dimethylethyl)oxy]carbonyl}(methyl)amino]methyl}- |
| | [(3R)-1-({2-fluoro-4- | 2-fluorobenzoic acid |
| | [(methylamino)methyl]phenyl}carbonyl)- | |
| | 3-piperidinyl]-4- | |
| | hydroxybutyl}carbamate | |

### Example 5

The following piperidines were prepared following procedures analogous to those described in Example 3 using the appropriate amine intermediate and the indicated acid in place of 4-((tert-butoxycarbonyl(methyl)amino)methyl)benzoic acid in Step 1 and omitting Step 2:

| Cpd. # | Product | Acid used in Step 1 |
|---|---|---|
| #120 | methyl [4-((3R)-1-{[4- | 4-(aminocarbonyl)- |
| | (aminocarbonyl)phenyl]carbonyl}-3- | benzoic acid for 4- |
| | piperidinyl)-4-(6-fluoro-3′-methyl-2- | formylbenzoic acid |
| | biphenylyl)-4-hydroxybutyl]carbamate | |
| #103 | methyl [4-(6-chloro-3′-ethyl-2- | 4-(hydroxymethyl)- |
| | biphenylyl)-4-hydroxy-4-((3R)-1-{[4- | benzoic acid |
| | (hydroxymethyl)phenyl]carbonyl}-3- | |
| | piperidinyl)butyl]carbamate | |

### Example 6

### (3-(aminomethyl)phenyl)(4-(1-(3′-ethyl-6-fluorobiphenyl-2-yl)-1-hydroxy-5-methoxypentyl)piperidin-1-yl)methanone (#3)

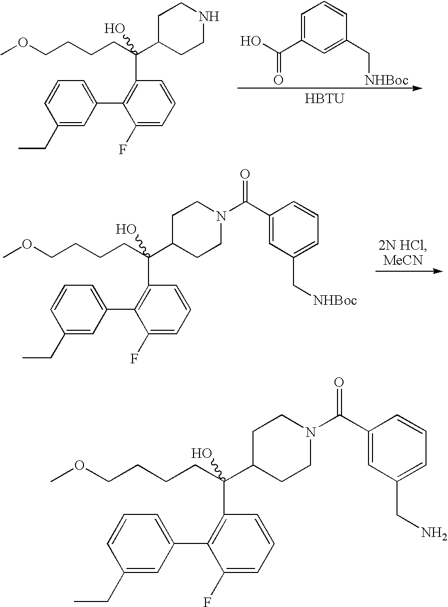

### Step 1. tert-butyl 3-(4-(143′-ethyl-6-fluorobiphenyl-2-yl)-1-hydroxy-5-methoxypentyl)piperidine-1-carbonyl)benzylcarbamate

A solution of 1-(3′-ethyl-6-fluorobiphenyl-2-yl)-5-methoxy-1-(piperidin-4-yl)pentan-1-ol (15 mg, 0.038 mmol) in 0.3 mL of DMF at 25° C. was treated with 33-((tert-butoxycarbonylamino)methyl)benzoic acid (11 mg, 0.042 mmol), i-Pr₂NEt (0.03 mL, 0.17 mmol), HBTU (16 mg, 0.042 mmol) and HOBt (6 mg, 0.042 mmol). After 20 h, H₂O was added and the mixture was extracted with EtOAc. The organic extracts were washed (1N HCl, 1N NaOH, H₂O, brine), dried (Na₂SO₄), concentrated under reduced pressure, and subjected to flash chromatography to provide tert-butyl 3-(4-(1-(3′-ethyl-6-fluorobiphenyl-2-yl)-1-hydroxy-5-methoxypentyl)piperidine-1-carbonyl)benzylcarbamate as a colorless oil (10 mg, 42%). MS (m/z) 633.3 (M+H⁺).

### Step 2. (3-(aminomethyl)phenyl)(4-(143′-ethyl-6-fluorobiphenyl-2-yl)-1-hydroxy-5-methoxypentyl)piperidin-1-yl)methanone

A solution of tert-butyl 3-(4-(143′-ethyl-6-fluorobiphenyl-2-yl)-1-hydroxy-5-methoxypentyl)piperidine-1-carbonyl)benzylcarbamate (10 mg, 0.016 mmol) in 1 mL of CH₃CN at 25° C. was treated with 1 mL of aqueous 2N HCl. After 24 h, the mixture was concentrated under reduced pressure to provide (3-(aminomethyl)phenyl)(4-(143′-ethyl-6-fluorobiphenyl-2-yl)-1-hydroxy-5-methoxypentyl)piperidin-1-yl)methanone as a white solid (8 mg, quantitative). MS (m/z) 533.3 (M+H⁺).

The following piperidines were prepared following procedures analogous to those described above by using the indicated acid in place of 3-[({[(1,1-dimethylethyl)oxy]carbonyl}amino)methyl]benzoic acid in Step 1:

| Structure | Name | Step 1 Acid |
|---|---|---|
| 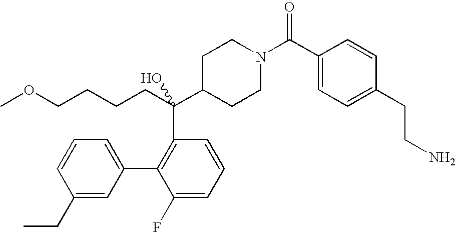 | 1-(1-{[4-(2- aminoethyl)phenyl]carbonyl}- 4-piperidinyl)-1-(3′-ethyl-6- fluoro-2-biphenylyl)-5- (methyloxy)-1-pentanol | 4-[2-({[(1,1- dimethylethyl)oxy]carbonyl} amino)ethyl]benzoic acid |

### Example 7

### (4-{[3-(1-(6-chloro-3′-ethyl-2-biphenylyl)-1-hydroxy-4-{[(methyloxy)carbonyl]amino}butyl)-1-piperidinyl]carbonyl}phenyl)-N,N,N-trimethylmethanaminium trifluoroacetate (#62)

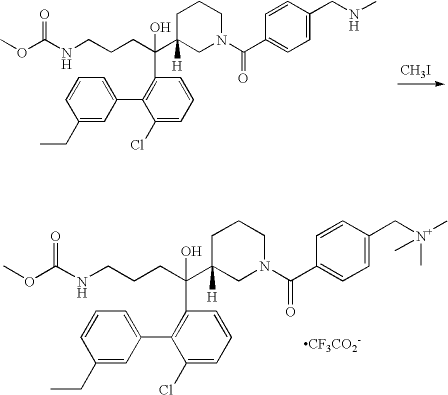

### Step 1. (4-{[3-(1-(6-chloro-3′-ethyl-2-biphenylyl)-1-hydroxy-4-{[(methyloxy)carbonyl]amino}butyl)-1-piperidinyl]carbonyl}phenyl)-N,N,N-trimethylmethanaminium trifluoroacetate

To methyl 4-(6-chloro-3′-ethylbiphenyl-2-yl)-4-hydroxy-4-((R)-1-(4-((methylamino)methyl)benzoyl)piperidin-3-yl)butylcarbamate (0.08 mmol, 57 mg) in a solution of dry DCM (5 ml) was added triethylamine (0.64 mmol, 88 uL) and methyl iodide (0.4 mmol, 24 uL). After stirring at room temperature overnight, the mixture was diluted with water and extracted into ethyl acetate. The combined organic layers were dried and evaporated. The crude product was purified by reverse phase HPLC to give 45 mg of the quaternary ammonium TFA salt. MS (m/z) 620.3 (M⁺).

### Example 8

### Methyl {4-(6-chloro-3′-ethyl-2-biphenylyl)-4-hydroxy-4-[(3R)-1-({4-[(methylamino)methyl]-2-[2-(1H-tetrazol-5-yl)ethyl]phenyl}carbonyl)-3-piperidinyl]butyl}carbamate (#83)

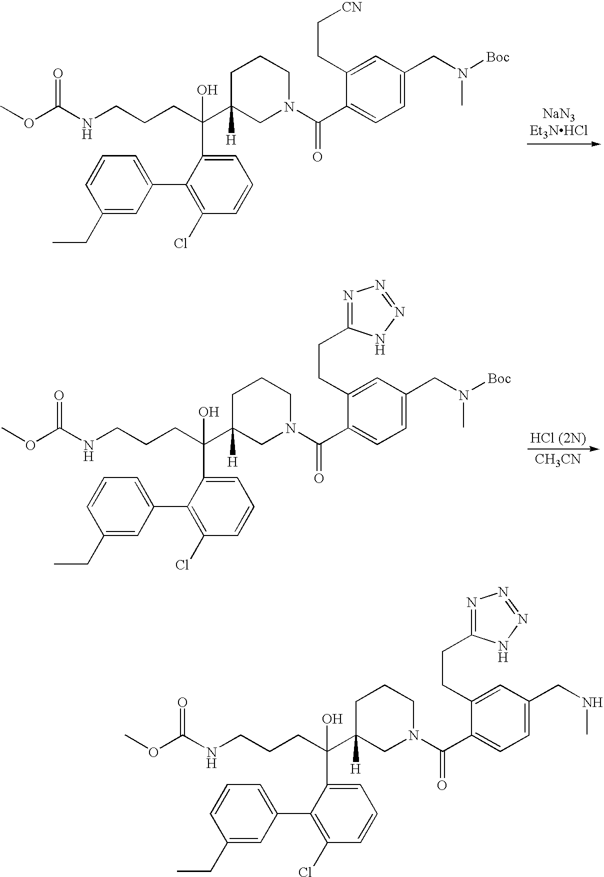

### Step 1. Methyl {4-(6-chloro-3′-ethyl-2-biphenylyl)-4-[(3R)-1-({4-{[{[(1,1-dimethylethyl)oxy]carbonyl}(methyl)amino]methyl}-2-[2-(1H-tetrazol-5-yl)ethyl]phenyl}carbonyl)-3-piperidinyl]-4-hydroxybutyl}carbamate

To a mixture of methyl (4-(6-chloro-3′-ethyl-2-biphenylyl)-4-{(3R)-1-[(2-(2-cyanoethyl)-4-{[{[(1,1-dimethylethyl)oxy]carbonyl}(methyl)amino]methyl}phenyl)carbonyl]-3-piperidinyl}-4-hydroxybutyl)carbamate (104 mg, 0.14 mmol) and sodium azide (45.5 mg, 0.70 mmol) in DMF (3 mL) was added triethylamine hydrochloride (96 mg, 0.70 mmol).

The mixture was heated at 100° C. for 18 h. HPLC analysis showed only about 15% conversion at this time. Another 45.5 mg of sodium azide and 96 mg of triethylamine hydrochloride were added and the reaction continued to heat at 100° C. for another 48 h. HPLC analysis showed no further conversion at this time. The reaction mixture was then heated at 120° C. for 20 h and HPLC analysis showed about 20% conversion at this time. The reaction was concentrated and the residue dissolved in EtOAc and washed with brine, dried, filtered and concentrated to give 100 mg of crude material which was purified by preparatory TLC (Kieselge 60 F₂₅₄, 1.0 mm, 20×20 cm, 90% EtOAc/Hexane) to provide methyl {4-(6-chloro-3′-ethyl-2-biphenylyl)-4-[(3R)-1-({4-{[{[(1,1-dimethylethyl)oxy]carbonyl}(methyl)amino]methyl}-2-[2-(1H-tetrazol-5-yl)ethyl]phenyl}carbonyl)-3-piperidinyl]-4-hydroxybutyl}carbamate (13.7 mg, 12.4%) as a clear oil. MS (m/z) 787.8 (M+H⁺).

### Step 2. Methyl {4-(6-chloro-3′-ethyl-2-biphenylyl)-4-hydroxy-4-[(3R)-1-({4-[(methylamino)methyl]-2-[2-(1H-tetrazol-5-yl)ethyl]phenyl}carbonyl)-3-piperidinyl]butyl}carbamate

To a solution methyl {4-(6-chloro-3′-ethyl-2-biphenylyl)-4-[(3R)-1-({4-{[{[(1,1-dimethylethyl)oxy]carbonyl}(methyl)amino]methyl}-2-[2-(1H-tetrazol-5-yl)ethyl]phenyl}carbonyl)-3-piperidinyl]-4-hydroxybutyl}carbamate (13 mg, 0.017 mmol) in acetonitrile (3 mL) at 0° C. was added 2N HCl (3 mL). The resulting solution was stirred at rt and monitored by HPLC and LCMS. After 48 h, the reaction mixture was concentrated and lyophilized to give methyl {4-(6-chloro-3′-ethyl-2-biphenylyl)-4-hydroxy-4-[(3R)-1-({4-[(methylamino)methyl]-2-[2-(1H-tetrazol-5-yl)ethyl]phenyl}carbonyl)-3-piperidinyl]butyl}carbamate (7.8 mg, 66%) as a light amber solid. MS (m/z) 687.8 (M+H⁺).

### Example 9

### Methyl [4-[(3R)-1-({2-(3-amino-3-oxopropyl)-4-[(methylamino)methyl]phenyl}carbonyl)-3-piperidinyl]-4-(6-chloro-3′-ethyl-2-biphenylyl)-4-hydroxybutyl]carbamate (#84)

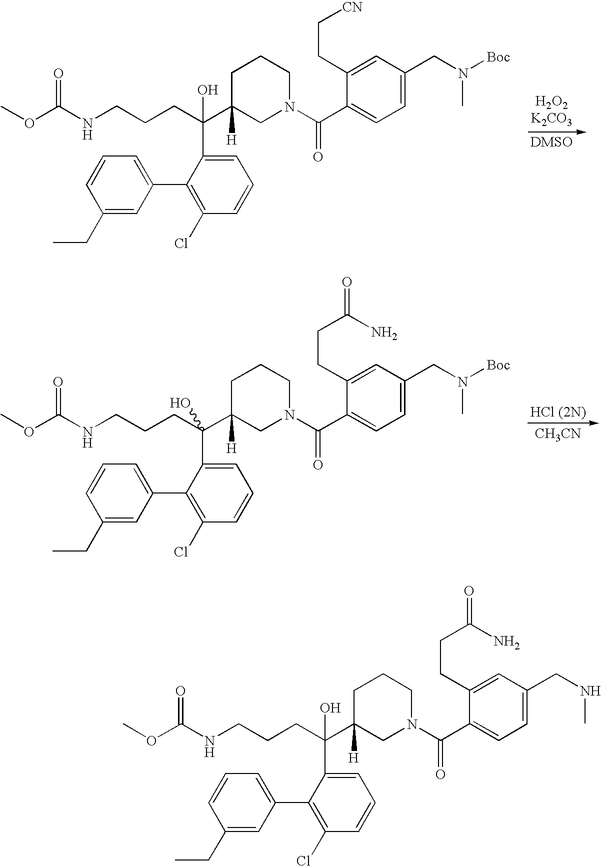

### Step 1. 1,1-dimethylethyl [(3-(3-amino-3-oxopropyl)-4-{[(3R)-3-(1-(6-chloro-3′-ethyl-2-biphenylyl)-1-hydroxy-4-{[(methyloxy)carbonyl]amino}butyl)-1-piperidinyl]carbonyl}phenyl)methyl]methylcarbamate

To solution of methyl (4-(6-chloro-3′-ethyl-2-biphenylyl)-4-{(3R)-1-[(2-(2-cyanoethyl)-4-{[{[(1,1-dimethylethyl)oxy]carbonyl}(methyl)amino]methyl}phenyl)carbonyl]-3-piperidinyl}-4-hydroxybutyl)carbamate (23 mg, 0.03 mmol) in DMSO (9 mL) were added hydrogen peroxide (30%, 250 μl) and potassium carbonate (10% aqueous, 375 μl). The mixture was then stirred at rt for 2 h. The solvent was removed in vacuo and the residue dissolved in EtOAc (5 ml) and washed with water, brine, dried, filtered and concentrated to afford 1,1-dimethylethyl [(3-(3-amino-3-oxopropyl)-4-{[(3R)-3-(1-(6-chloro-3′-ethyl-2-biphenylyl)-1-hydroxy-4-{[(methyloxy)carbonyl]amino}butyl)-1-piperidinyl]carbonyl}phenyl)methyl]methylcarbamate (21 mg, 89%) as an oil. MS (m/z) 762.8 (M+H⁺).

### Step 2. Methyl [4-[(3R)-1-({2-(3-amino-3-oxopropyl)-4-[(methylamino)methyl]phenyl}carbonyl)-3-piperidinyl]-4-(6-chloro-3′-ethyl-2-biphenylyl)-4-hydroxybutyl]carbamate

To a solution 1,1-dimethylethyl [(3-(3-amino-3-oxopropyl)-4-{[(3R)-3-(1-(6-chloro-3′-ethyl-2-biphenylyl)-1-hydroxy-4-{[(methyloxy)carbonyl]amino}butyl)-1-piperidinyl]carbonyl}phenyl)methyl]methylcarbamate (21 mg, 0.028 mmol) in acetonitrile (3 mL) at 0° C. was added 2N HCl (3 mL). The resulting solution was stirred at rt and monitored by HPLC and LCMS. After 48 h, the reaction mixture was concentrated and lyophilized to give methyl [4-[(3R)-1-({2-(3-amino-3-oxopropyl)-4-[(methylamino)methyl]phenyl}carbonyl)-3-piperidinyl]-4-(6-chloro-3′-ethyl-2-biphenylyl)-4-hydroxybutyl]carbamate (19.4 mg, 99%) as a white powder. MS (m/z) 663.6 (M+H⁺).

### Example 10

### Methyl [4-[(3R)-1-({2-(aminocarbonyl)-4-[(methylamino)methyl]phenyl}carbonyl)-3-piperidinyl]-4-(6-chloro-3′-ethyl-2-biphenylyl)-4-hydroxybutyl]carbamate (#86)

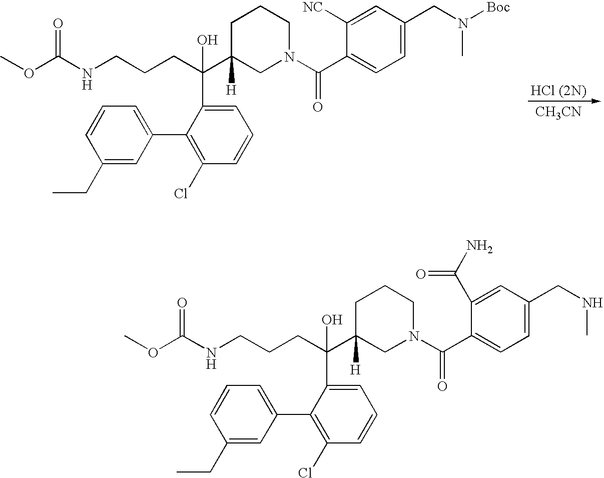

### Step 1. Methyl [4-[(3R)-1-({2-(aminocarbonyl)-4-[(methylamino)methyl]phenyl}carbonyl)-3-piperidinyl]-4-(6-chloro-3′-ethyl-2-biphenylyl)-4-hydroxybutyl]carbamate

To a solution of methyl (4-(6-chloro-3′-ethyl-2-biphenylyl)-4-{(3R)-1-[(2-cyano-4-{[{[(1,1-dimethylethyl)oxy]carbonyl}(methyl)amino]methyl}phenyl)carbonyl]-3-piperidinyl}-4-hydroxybutyl)carbamate (41 mg, 0.057 mmol) in acetonitrile (5 mL) was added 2N HCl (5 mL) at 0° C. The resulting solution was stirred at room temperature and monitored by LCMS and HPLC. The solvent was removed in vacuo to give a white solid, which appeared to be a mixture of two compounds by HPLC and LCMS analysis. The mixture was purified via preparatory HPLC (Sunfire Prep C₁₈OBD, 5 μm, 30×150 mm, 25 mL/min, B: CH₃CN (0.1% TFA), A: water (0.1% TFA), 40-80% B in 15 min). The fractions were combined and lyophilized to afford methyl [4-[(3R)-1-({2-(aminocarbonyl)-4-[(methylamino)methyl]phenyl}carbonyl)-3-piperidinyl]-4-(6-chloro-3′-ethyl-2-biphenylyl)-4-hydroxybutyl]carbamate (4.5 mg, 12%). MS (m/z) 653.3 (M+H⁺).

### Example 11

### 2-{[(3R)-3-(1-(6-chloro-3′-ethyl-2-biphenylyl)-1-hydroxy-4-{[(methyloxy)carbonyl]amino}butyl)-1-piperidinyl]carbonyl}-5-[(methylamino)methyl]benzoic acid (#89)

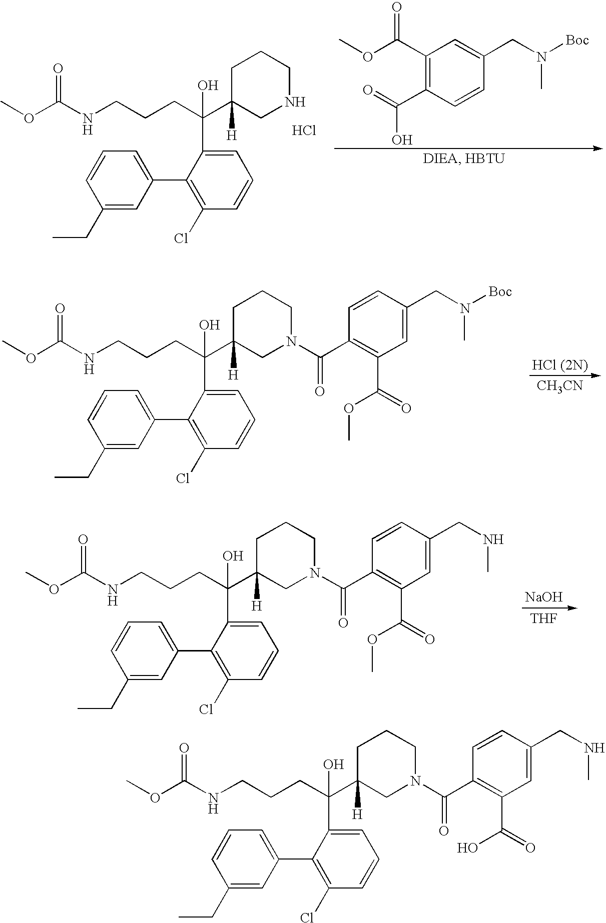

### Step 1. Methyl 2-{[(3R)-3-(1-(6-chloro-3′-ethyl-2-biphenylyl)-1-hydroxy-4-{[(methyloxy)carbonyl]amino}butyl)-1-piperidinyl]carbonyl}-5-{[{[(1,1-dimethylethyl)oxy]carbonyl}(methyl)amino]methyl}benzoate

To a solution of 4-{[{[(1,1-dimethylethyl)oxy]carbonyl}(methyl)amino]methyl}-2-[(methyloxy)carbonyl]benzoic acid (168 mg, 0.52 mmol) in DMF (5 mL) was added DIEA (181 μl, 1.04 mmol) at 0° C., followed by HBTU (217 mg, 0.57 mmol). Methyl {4-(6-chloro-3′-ethyl-2-biphenylyl)-4-hydroxy-4-[(3R)-3-piperidinyl]butyl}carbamate hydrochloride (231 mg, 0.52 mmol) was then added and the resulting solution was warmed to rt and stirred overnight. The solvent was removed in vacuo and the residue diluted with water (8 mL) and extracted with EtOAc (2×8 mL). The combined organic extracts were washed with 1N HCl (8 mL), then 1N NaOH (2×8 mL), water (8 mL) and brine (8 mL), and dried over Na₂SO₄, filtered, and concentrated to provide 387 mg of crude material which was purified by column chromatography (16 g silica gel 60, 230-400 mesh, 50-70% EtOAc/hexanes) to give methyl 2-{[(3R)-3-(1-(6-chloro-3′-ethyl-2-biphenylyl)-1-hydroxy-4-{[(methyloxy)carbonyl]amino}butyl)-1-piperidinyl]carbonyl}-5-{[{[(1,1-dimethylethyl)oxy]carbonyl}(methyl)amino]methyl}benzoate (237.4 mg, 61%) as a white solid. MS (m/z) 749.8 (M+H⁺).

### Step 2. Methyl 2-{[(3R)-3-(1-(6-chloro-3′-ethyl-2-biphenylyl)-1-hydroxy-4-{[(methyloxy)carbonyl]amino}butyl)-1-piperidinyl]carbonyl}-5-[(methylamino)methyl]benzoate

To a solution methyl 2-{[(3R)-3-(1-(6-chloro-3′-ethyl-2-biphenylyl)-1-hydroxy-4-{[(methyloxy)carbonyl]amino}butyl)-1-piperidinyl]carbonyl}-5-{[{[(1,1-dimethylethyl)oxy]carbonyl}(methyl)amino]methyl}benzoate (60 mg, 0.08 mmol) in acetonitrile (5 mL) was added 2N aqueous HCl (5 mL) at 0° C. The resulting solution was stirred at rt over the weekend at which time HPLC analysis as well as LCMS indicated the reaction was complete. The reaction mixture was concentrated to give methyl 2-{[(3R)-3-(1-(6-chloro-3′-ethyl-2-biphenylyl)-1-hydroxy-4-{[(methyloxy)carbonyl]amino}butyl)-1-piperidinyl]carbonyl}-5-[(methylamino)methyl]benzoate (48 mg) as an off white solid. MS (m/z) 650.8 (M+H⁺).

### Step 3. 2-{[(3R)-3-(1-(6-chloro-3′-ethyl-2-biphenylyl)-1-hydroxy-4-{[(methyloxy)carbonyl]amino}butyl)-1-piperidinyl]carbonyl}-5-[(methylamino)methyl]benzoic acid

To a solution of methyl 2-{[(3R)-3-(1-(6-chloro-3′-ethyl-2-biphenylyl)-1-hydroxy-4-{[(methyloxy)carbonyl]amino}butyl)-1-piperidinyl]carbonyl}-5-[(methylamino)methyl]benzoate (48 mg, 0.074 mmol) in THF (5 mL) was added NaOH (1N, 148 μl). The resulting mixture was stirred at rt. After 16 h, LCMS as well as HPLC analysis indicated the reaction was complete. The solvent was removed in vacuo to afford 2-{[(3R)-3-(1-(6-chloro-3′-ethyl-2-biphenylyl)-1-hydroxy-4-{[(methyloxy)carbonyl]amino}butyl)-1-piperidinyl]carbonyl}-5-[(methylamino)methyl]benzoic acid as a white solid. MS (m/z) 636.7 (M+H⁺).

### Example 12

### Methyl [4-[(3R)-1-({2-(2-amino-2-oxoethyl)-4-[(methylamino)methyl]phenyl}carbonyl)-3-piperidinyl]-4-(6-chloro-3′-ethyl-2-biphenylyl)-4-hydroxybutyl]carbamate (#90)

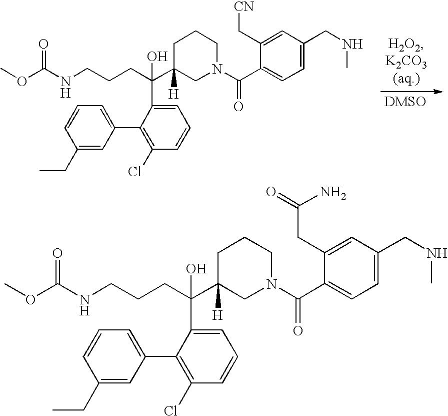

### Step 1. Methyl [4-[(3R)-1-({2-(2-amino-2-oxoethyl)-4-[(methylamino)methyl]phenyl}carbonyl)-3-piperidinyl]-4-(6-chloro-3′-ethyl-2-biphenylyl)-4-hydroxybutyl]carbamate

To a solution of methyl {4-(6-chloro-3′-ethyl-2-biphenylyl)-4-[(3R)-1-({2-(cyanomethyl)-4-[(methylamino)methyl]phenyl}carbonyl)-3-piperidinyl]-4-hydroxybutyl}carbamate (42 mg, 0.067 mmol) in DMSO (10 mL) was added hydrogen peroxide (30%, 500 μl), followed by potassium carbonate (10% aqueous, 750 μl). The resulting mixture was stirred at rt for 2 h. The reaction mixture was diluted with EtOAc (20 mL) and washed with water (3×10 mL). The organic layer was dried, filtered and concentrated to give 40 mg of crude material which was purified by Prep HPLC: Sunfire™ Prep C₁₈OBD, 5 μm, 30×150 mm, 25 ml/min, B: CH₃CN (0.1% TFA), A: water (0.1% TFA), 30-60% B in 15 min. The fractions were combined and lyophilized to afford methyl [4-[(3R)-1-({2-(2-amino-2-oxoethyl)-4-[(methylamino)methyl]phenyl}carbonyl)-3-piperidinyl]-4-(6-chloro-3′-ethyl-2-biphenylyl)-4-hydroxybutyl]carbamate (8 mg) as a white powder. MS (m/z) 649.2 (M+H⁺).

### Example 13

### Methyl (4-(6-chloro-3′-ethyl-2-biphenylyl)-4-hydroxy-4-{(3R)-1-[(4-{[(2-hydroxyethyl)amino]methyl}phenyl)carbonyl]-3-piperidinyl}butyl)carbamate (#104)

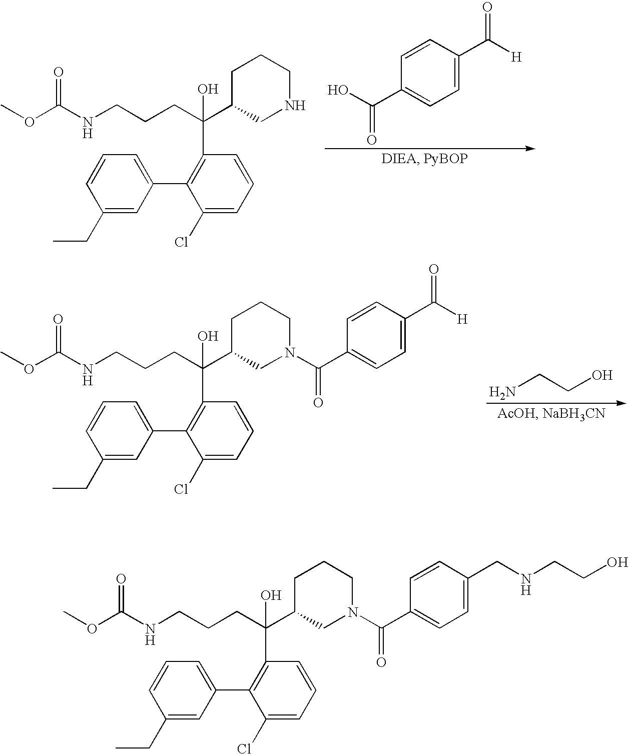

### Step 1. Methyl (4-(6-chloro-3′-ethyl-2-biphenylyl)-4-{(3R)-1-[(4-formylphenyl)carbonyl]-3-piperidinyl}-4-hydroxybutyl)carbamate

To a suspension of 4-formylbenzoic acid (0.093 g, 0.618 mmol) in CH₂Cl₂ (10 mL) was added diisopropylethylamine (0.2 mL, 1.12 mmol) and PyBOP (0.29 g, 0.562 mmol). The resulting mixture was stirred for 10 minutes before methyl {4-(6-chloro-3′-ethyl-2-biphenylyl)-4-hydroxy-4-[(3R)-3-piperidinyl]butyl}carbamate (0.250 g, 0.562 mmol) was added over 5 minutes as a solution in CH₂Cl₂ (2 mL. After 30 minutes, the reaction mixture was concentrated in vacuo. The residue was purified via silica gel chromatography (25-95% ethyl acetate/hexanes) to afford methyl (4-(6-chloro-3′-ethyl-2-biphenylyl)-4-{(3R)-1-[(4-formylphenyl)carbonyl]-3-piperidinyl}-4-hydroxybutyl)carbamate (0.324 g, 100%) as a light yellow foam. MS (m/z) 577.3 (M+H⁺).

### Step 2. Methyl (4-(6-chloro-3′-ethyl-2-biphenylyl)-4-hydroxy-4-{(3R)-1-[(4-{[(2-hydroxyethyl)amino]methyl}phenyl)carbonyl]-3-piperidinyl}butyl)carbamate

A solution of 2-aminoethanol (0.023 mL, 0.385 mmol) in MeOH (1 mL) was acidified to pH 6 with glacial acetic acid (5-10 drops). This solution was combined with methyl (4-(6-chloro-3′-ethyl-2-biphenylyl)-4-{(3R)-1-[(4-formylphenyl)carbonyl]-3-piperidinyl}-4-hydroxybutyl)carbamate (55.5 mg, 0.0962 mmol) and the resulting mixture stirred for 10 minutes before sodium cyanoborohydride (6 mg, 0.0962 mmol) was added. After 19 hours, the reaction was filtered through an Acrodisc CR13 mm syringe filter with 0.45 μM PTFE membrane. The mixture was purified by HPLC (20-65% CH₃CN/H₂O, 0.1% TFA, 30×150 mm Sunfire C18, 25 mL/minute, 15 minutes) to afford methyl (4-(6-chloro-3′-ethyl-2-biphenylyl)-4-hydroxy-4-{(3R)-1-[(4-{[(2-hydroxyethyl)amino]methyl}phenyl)carbonyl]-3-piperidinyl}butyl)carbamate (55.2 mg, 80%) as a white foam. MS (m/z) 622.2 (M+H⁺).

The following piperidines were prepared following procedures analogous to those described in Example 13 using the appropriate aldehyde intermediate and the indicated amine in place of 2-aminoethanol in Step 2:

| Cpd. # | Product | Amine Used in Step 2 |
|---|---|---|
| #105 | methyl (4-(6-chloro-3′-ethyl-2-biphenylyl)-4- | 3-amino-1-propanol |
| | hydroxy-4-{(3R)-1-[(4-{[(3- | |
| | hydroxypropyl)amino]methyl}phenyl)carbonyl]- | |
| | 3-piperidinyl}butyl)carbamate | |
| #106 | methyl [4-(6-chloro-3′-ethyl-2-biphenylyl)-4- | [2- |
| | hydroxy-4-((3R)-1-{[4-({[2- | (methyloxy)ethyl]amine |
| | (methyloxy)ethyl]amino}methyl)phenyl]carbonyl}- | |
| | 3-piperidinyl)butyl]carbamate | |
| #107 | methyl [4-(6-chloro-3′-ethyl-2-biphenylyl)-4- | [2-(ethyloxy)ethyl]amine |
| | ((3R)-1-{[4-({[2- | |
| | (ethyloxy)ethyl]amino}methyl)phenyl]carbonyl}- | |
| | 3-piperidinyl)-4-hydroxybutyl]carbamate | |
| #108 | methyl [4-(6-chloro-3′-ethyl-2-biphenylyl)-4- | [1-methyl-2- |
| | hydroxy-4-((3R)-1-{[4-({[1-methyl-2- | (methyloxy)ethyl]amine |
| | (methyloxy)ethyl]amino}methyl)phenyl]carbonyl}- | |
| | 3-piperidinyl)butyl]carbamate | |
| #109 | methyl {4-(6-chloro-3′-ethyl-2-biphenylyl)-4- | {2-[(1- |
| | hydroxy-4-[(3R)-1-({4-[({2-[(1- | methylethyl)oxy]ethyl}amine |
| | methylethyl)oxy]ethyl}amino)methyl]phenyl}carbonyl)- | |
| | 3-piperidinyl]butyl}carbamate | |
| #110 | methyl (4-(6-chloro-3′-ethyl-2-biphenylyl)-4- | (tetrahydro-2- |
| | hydroxy-4-{(3R)-1-[(4-{[(tetrahydro-2- | furanylmethyl)amine |
| | furanylmethyl)amino]methyl}phenyl)carbonyl]- | |
| | 3-piperidinyl}butyl)carbamate | |
| #113 | methyl [4-(6-chloro-3′-ethyl-2-biphenylyl)-4- | [2- |
| | hydroxy-4-((3R)-1-{[4-({[2- | (methylthio)ethyl]amine |
| | (methylthio)ethyl]amino}methyl)phenyl]carbonyl}- | |
| | 3-piperidinyl)butyl]carbamate | |
| #116 | methyl [4-{(3R)-1-[(4-{[(2- | 3-aminopropanenitrile |
| | cyanoethyl)amino]methyl}phenyl)carbonyl]- | |
| | 3-piperidinyl}-4-(6-fluoro-3′-methyl-2- | |
| | biphenylyl)-4-hydroxybutyl]carbamate | |
| #123 | methyl (4-(6-fluoro-3′-methyl-2-biphenylyl)- | 4-amino-1-butanol |
| | 4-hydroxy-4-{(3R)-1-[(4-{[(4- | |
| | hydroxybutyl)amino]methyl}phenyl)carbonyl]- | |
| | 3-piperidinyl}butyl)carbamate | |
| #124 | methyl [4-((3R)-1-{[4-({[2- | (2- |
| | (dimethylamino)ethyl]amino}methyl)phenyl]carbonyl}- | aminoethyl)dimethylamine |
| | 3-piperidinyl)-4-(6-fluoro-3′- | |
| | methyl-2-biphenylyl)-4- | |
| | hydroxybutyl]carbamate | |
| #125 | methyl [4-((3R)-1-{[4-({[3- | (3- |
| | (dimethylamino)propyl]amino}methyl)phenyl]carbonyl}- | aminopropyl)dimethylamine |
| | 3-piperidinyl)-4-(6-fluoro-3′- | |
| | methyl-2-biphenylyl)-4- | |
| | hydroxybutyl]carbamate | |
| #127 | methyl [4-{(3R)-1-[(4- | aminoacetonitrile |
| | {[(cyanomethyl)amino]methyl}phenyl)carbonyl]- | |
| | 3-piperidinyl}-4-(6-fluoro-3′-methyl-2- | |
| | biphenylyl)-4-hydroxybutyl]carbamate | |
| #137 | methyl [4-{(3R)-1-[(4-{[(4-amino-4- | 4-aminobutanamide |
| | oxobutyl)amino]methyl}phenyl)carbonyl]-3- | hydrochloride |
| | piperidinyl}-4-(6-fluoro-3′-methyl-2- | |
| | biphenylyl)-4-hydroxybutyl]carbamate | |
| #138 | methyl {4-(6-fluoro-3′-methyl-2-biphenylyl)- | N-(2- |
| | 4-hydroxy-4-[(3R)-1-({4-[({2- | aminoethyl)methanesulfonamide |
| | [(methylsulfonyl)amino]ethyl}amino)methyl]phenyl}carbonyl)- | hydrochloride |
| | 3- | |
| | piperidinyl]butyl}carbamate | |
| #139 | methyl [4-(6-fluoro-3′-methyl-2-biphenylyl)- | [3-(1H-tetrazol-5- |
| | 4-hydroxy-4-((3R)-1-{[4-({[3-(1H-tetrazol-5- | yl)propyl]amine |
| | yl)propyl]amino}methyl)phenyl]carbonyl}-3- | hydrochloride |
| | piperidinyl)butyl]carbamate | |

### Example 14

### Methyl [4-{(3R)-1-[(4-{[(2-amino-2-oxoethyl)amino]methyl}phenyl)carbonyl]-3-piperidinyl}-4-(6-fluoro-3′-methyl-2-biphenylyl)-4-hydroxybutyl]carbamate (#140)

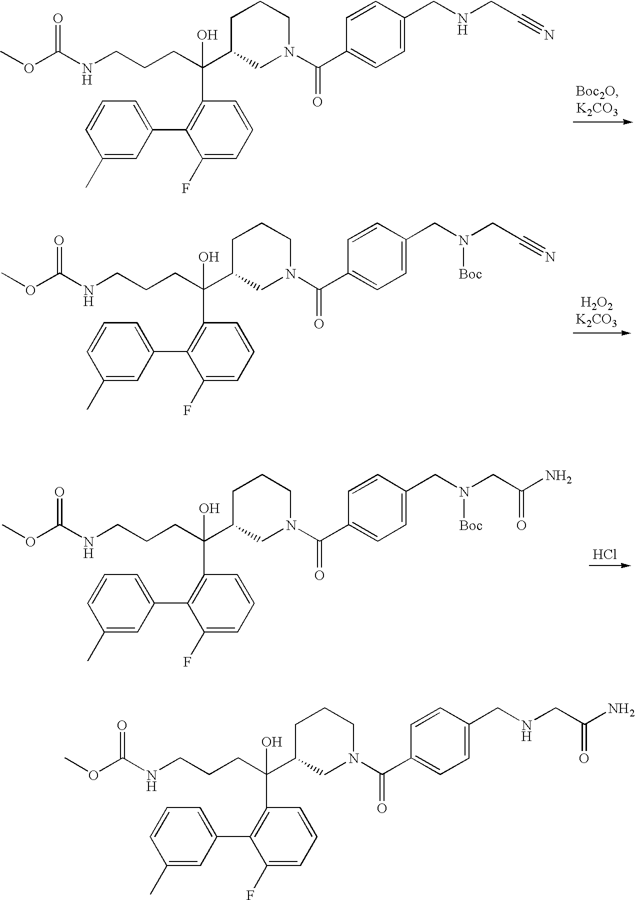

### Step 1. 1,1-Dimethylethyl (cyanomethyl)[(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl]carbamate

To a solution of methyl [4-{(3R)-1-[(4-{[(cyanomethyl)amino]methyl}phenyl)carbonyl]-3-piperidinyl}-4-(6-fluoro-3′-methyl-2-biphenylyl)-4-hydroxybutyl]carbamate (57.5 mg, 0.0821 mmol) and potassium carbonate (34 mg, 0.246 mmol) in acetone (1 mL) and water (0.25 mL) was added di-tert-butyl dicarbonate (20 mg, 0.0903 mmol). The resulting mixture was stirred 15 hours and additional di-tert-butyl dicarbonate (20 mg, 0.0903 mmol) was added. The reaction mixture was stirred for 2 additional days. At this time the acetone was removed in vacuo, the residue diluted with saturated NaCl (2 mL) and CH₂Cl₂ (5 mL), and the layers were separated using a hydrophobic frit phase separator. The organic layer was concentrated to afford 1,1-dimethylethyl (cyanomethyl)[(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl]carbamate and excess tert-butyl components (67.2 mg, 119%) as white foam. MS (m/z) 686.8 (M+H⁺).

### Step 2. 1,1-Dimethylethyl (2-amino-2-oxoethyl)[(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl]carbamate

To a solution of 1,1-Dimethylethyl (cyanomethyl)[(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl]carbamate (56.4 g, 0.082 mmol) in dimethyl sulfoxide (2 ml) was added hydrogen peroxide (0.3 mL, 9.79 mmol) (over 5 minutes) and potassium carbonate (3.4 mg, 0.025 mmol). The resulting mixture was stirred for 15 hours. Additional hydrogen peroxide (0.3 mL, 9.79 mmol) and potassium carbonate (3.4 mg, 0.025 mmol) were added at this time. The resulting mixture was stirred and additional 4 hours and then water (10 mL) and ethyl acetate (20 mL) were added. The mixture was stirred for 10 minutes until the bubbling stopped, and then the layers were separated. The organic layer was washed with water (2×10 mL). The organic layer was dried over Na₂SO₄, filtered, and concentrated to the afford 1,1-dimethylethyl (2-amino-2-oxoethyl)[(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl]carbamate (53 mg, 82%) as white foam. MS (m/z) 704.8 (M+H⁺).

### Step 3. Methyl [4-{(3R)-1-[(4-{[(2-amino-2-oxoethyl)amino]methyl}phenyl)carbonyl]-3-piperidinyl}-4-(6-fluoro-3′-methyl-2-biphenylyl)-4-hydroxybutyl]carbamate

To a solution of 1,1-dimethylethyl (2-amino-2-oxoethyl)[(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl]carbamate (47.8 mg, 0.068 mmol) in acetonitrile (1 mL) was added HCl (4 N in dioxane, 0.085 mL, 0.339 mmol). The resulting mixture was stirred for 90 minutes and then the mixture was purified by HPLC (15-55% CH₃CN/H₂O, 0.1% TFA, 30×150 mm Sunfire C18, 25 mL/minute, 15 minutes) to afford methyl [4-{(3R)-1-[(4-{[(2-amino-2-oxoethyl)amino]methyl}phenyl)carbonyl]-3-piperidinyl}-4-(6-fluoro-3′-methyl-2-biphenylyl)-4-hydroxybutyl]carbamate (28.7 g, 57%) as a white foam. MS (m/z) 605.3 (M+H⁺).

The following piperidines were prepared following procedures analogous to those described in Example 14:

| Cpd. # | Product |
|---|---|
| #121 | methyl [4-{(3R)-1-[(4-{[(3-amino-3- |
| | oxopropyl)amino]methyl}phenyl)carbonyl]-3-piperidinyl}-4-(6- |
| | fluoro-3′-methyl-2-biphenylyl)-4-hydroxybutyl]carbamate |

### Example 15

### Methyl (4-(6-fluoro-3′-methyl-2-biphenylyl)-4-hydroxy-4-{(3R)-1-[(4-{[(1H-tetrazol-5-ylmethyl)amino]methyl}phenyl)carbonyl]-3-piperidinyl}butyl)carbamate (#141)

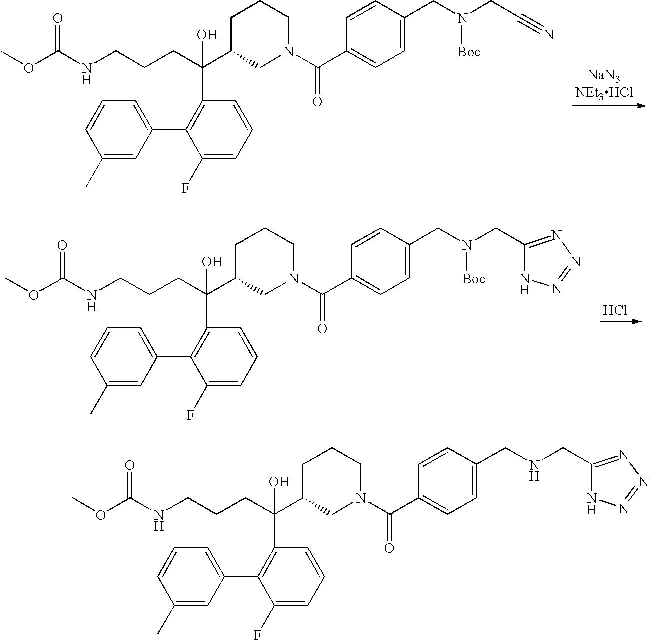

### Step 1. 1,1-dimethylethyl [(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl](1H-tetrazol-5-ylmethyl)carbamate

To a solution of 1,1-dimethylethyl (cyanomethyl)[(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl]carbamate (87 mg, 0.127 mmol) in DMF (3 mL) was added triethylamine hydrochloride (0.349 g, 2.53 mmol) and sodium azide (0.165 g, 2.53 mmol). The reaction mixture was heated at 120° C. for 24 hours. After cooling to room temperature, 10 mL water was added, and the mixture was acidified to pH 3-4 with 1 N HCl. The aqueous layer was extracted with ethyl acetate (2×10 mL). The combined organic layers were dried over Na₂SO₄, filtered, and concentrated to afford 1,1-dimethylethyl [(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl] (1H-tetrazol-5-ylmethyl)carbamate, which was used in the subsequent step without further purification. MS (m/z) 729.8 (M+H⁺).

### Step 2. Methyl (4-(6-fluoro-3′-methyl-2-biphenylyl)-4-hydroxy-4-{(3R)-1-[(4-{[(1H-tetrazol-5-ylmethyl)amino]methyl}phenyl)carbonyl]-3-piperidinyl}butyl)carbamate

The Boc protecting group was removed using the protocol described in Example 14, step 3. MS (m/z) 629.8 (M+H⁺).

The following piperidines were prepared following procedures analogous to those described in Example 15:

| Cpd. | |
|---|---|
| # | Product |
| #122 | methyl [4-(6-fluoro-3′-methyl-2-biphenylyl)-4-hydroxy-4-((3R)-1- |
| | {[4-({[2-(1H-tetrazol-5-yl)ethyl]amino}methyl)phenyl]carbonyl}- |
| | 3-piperidinyl)butyl]carbamate |

### Example 16

### N-[(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl]glycine (#119)

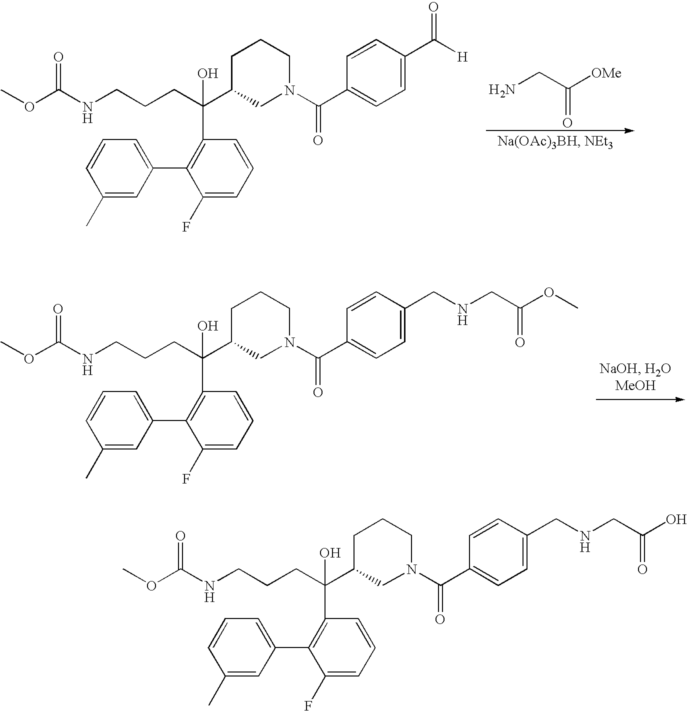

### Step 1. Methyl N-[(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl]glycinate

To a suspension of methyl (4-(6-fluoro-3′-methyl-2-biphenylyl)-4-{(3R)-1-[(4-formylphenyl)carbonyl]-3-piperidinyl}-4-hydroxybutyl)carbamate (70 mg, 0.128 mmol) in 1,2-dichloroethane (4 mL) was added triethylamine (0.054 mL, 0.384 mmol), glycine methyl ester hydrochloride (48 mg, 0.384 mmol), and sodium triacetoxyborohydride (109 mg, 0.512 mmol). The resulting mixture was stirred for 19 hours before additional triethylamine (0.054 mL, 0.384 mmol), glycine methyl ester hydrochloride (48 mg, 0.384 mmol), and sodium triacetoxyborohydride (109 mg, 0.512 mmol) were added. After stirring 2 days total, 10 mL of water was added and the aqueous layer was adjusted to pH 11-12 with 2 N NaOH. CH₂Cl₂ (15 mL) was added and the layers were separated using a hydrophobic frit phase separator. The organic layer was concentrated to afford methyl N-[(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl]glycinate, which was used directly in the next step.

### Step 2. N-[(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl]glycine

To a solution of methyl N-[(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl]glycinate in MeOH (4 mL) was added NaOH (1N, 0.85 mL). The resulting mixture was stirred for 16 hours, before 3 N HCl was added dropwise to obtain a pH 2-3 mixture. The mixture was filtered through an Acrodisc CR13 mm syringe filter with 0.45 μM PTFE membrane and purified by HPLC (15-55% CH₃CN/H₂O, 0.1% TFA, 30×150 mm Sunfire C18, 25 mL/minute, 20 minutes) to afford N-[(4-{[(3R)-3-(1-(6-fluoro-3′-methyl-2-biphenylyl)-1-hydroxy-4-{[(methyloxy)carbonyl]amino}butyl)-1-piperidinyl]carbonyl}phenyl)methyl]glycine (58 mg, 63%) as a white foam. MS (m/z) 606.1 (M+H⁺).

The following piperidines were prepared following procedures analogous to those described above using the appropriate aldehyde intermediate and the indicated amine in place of 2-aminoethanol in Step 1:

| Cpd. | | Amine Used |
|---|---|---|
| # | Product | in Step 1 |
| #117 | N-[(4-{[(3R)-3-(1-(6-fluoro-3′-methyl-2-biphenylyl)- | beta-alanine |
| | 1-hydroxy-4-{[(methyloxy)carbonyl]amino}butyl)- | ethyl ester |
| | 1-piperidinyl]carbonyl}phenyl)methyl]-β-alanine | hydro- |
| | | chloride |

### Example 17

### Methyl [4-(6-chloro-3′-ethyl-2-biphenylyl)-4-hydroxy-4-((3R)-1-{[4-({[2-(methylsulfonyl)ethyl]amino}methyl)phenyl]carbonyl}-3-piperidinyl)butyl]carbamate (#114)

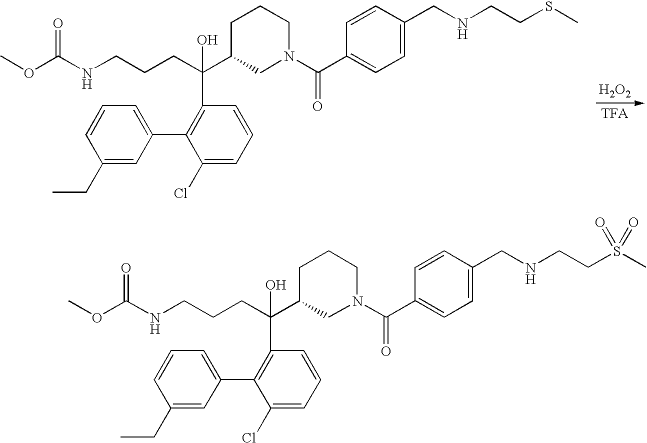

To a solution of trifluoroacetic acid (1 mL) and 30% hydrogen peroxide (0.5 mL) cooled to 0° C. was added methyl [4-(6-chloro-3′-ethyl-2-biphenylyl)-4-hydroxy-4-((3R)-1-{[4-({[2-(methylthio)ethyl]amino}methyl)phenyl]carbonyl}-3-piperidinyl)butyl]carbamate (79.4 mg, 0.122 mmol). After stirring for one hour, 5 mL of water and solid sodium bicarbonate were carefully added to make a pH 9 mixture. The aqueous layer was extracted with ethyl acetate (2×5 mL). The combined organic layers were dried over Na₂SO₄, filtered, and concentrated. The residue was purified by HPLC (20-60% CH₃CN/H₂O, 0.1% TFA, 30×150 mm Sunfire C18, 25 mL/minute, 20 minutes) to afford methyl [4-(6-chloro-3′-ethyl-2-biphenylyl)-4-hydroxy-4-((3R)-1-{[4-({[2-(methylsulfonyl)ethyl]amino}methyl)phenyl]carbonyl}-3-piperidinyl)butyl]carbamate (30 mg, 31%) as a light yellow foam. MS (m/z) 684.1 (M+H⁺).

### Example 18

### Methyl [4-((3R)-1-{[4-({[amino(imino)methyl]amino}methyl)phenyl]carbonyl}-3-piperidinyl)-4-(6-fluoro-3′-methyl-2-biphenylyl)-4-hydroxybutyl]carbamate (#126)

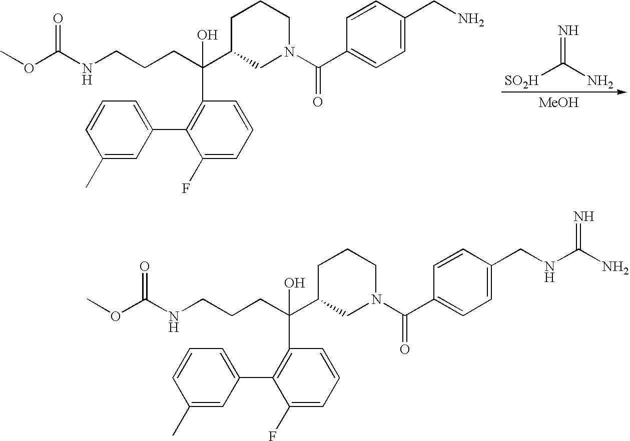

To a solution of methyl [4-((3R)-1-{[4-(aminomethyl)phenyl]carbonyl}-3-piperidinyl)-4-(6-fluoro-3′-methyl-2-biphenylyl)-4-hydroxybutyl]carbamate (44 mg, 0.0803 mmol) in MeOH (1 mL) was added aminoiminomethanesulfonic acid (50 mg, 0.402 mmol). After 18 hours, the reaction was filtered through an Acrodisc CR13 mm syringe filter with 0.45 μM PTFE membrane. The mixture was purified by HPLC (15-55% CH₃CN/H₂O, 0.1% TFA, 30×150 mm Sunfire C18, 25 mL/minute, 15 minutes) to afford methyl [4-((3R)-1-{[4-({[amino(imino)methyl]amino}methyl)phenyl]carbonyl}-3-piperidinyl)-4-(6-fluoro-3′-methyl-2-biphenylyl)-4-hydroxybutyl]carbamate (10 mg, 18%) as a light yellow glassy solid. MS (m/z) 589.8 (M+H⁺).

### Example 19

### Methyl {4-(6-fluoro-3′-methyl-2-biphenylyl)-4-hydroxy-4-[(3R)-1-({4-[(methylamino)methyl]phenyl}sulfonyl)-3-piperidinyl]butyl}carbamate (#115)

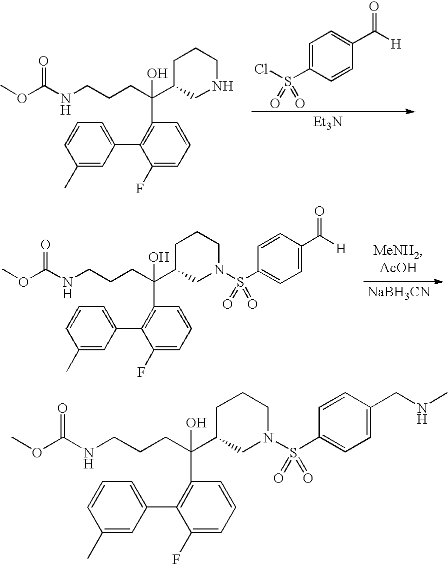

### Step 1. Methyl (4-(6-fluoro-3′-methyl-2-biphenylyl)-4-{(3R)-1-[(4-formylphenyl)sulfonyl]-3-piperidinyl}-4-hydroxybutyl)carbamate

To a solution of methyl {4-(6-fluoro-3′-methyl-2-biphenylyl)-4-hydroxy-4-[(3R)-3-piperidinyl]butyl}carbamate (75 mg, 0.181 mmol) and triethylamine (0.038 mL, 0.272 mmol) in CH₂Cl₂ (3.6 mL) was added 4-formylbenzenesulfonyl chloride (41 mg, 0.199 mmol). After one hour the reaction was concentrated. The residue was purified via silica gel chromatography (5-50% ethyl acetate/hexanes) to afford methyl (4-(6-fluoro-3′-methyl-2-biphenylyl)-4-{(3R)-1-[(4-formylphenyl)sulfonyl]-3-piperidinyl}-4-hydroxybutyl)carbamate (78.7 mg, 75%) as a white foam. MS (m/z) 582.8 (M+H⁺).

### Step 2. Methyl {4-(6-fluoro-3′-methyl-2-biphenylyl)-4-hydroxy-4-[(3R)-1-({4-[(methylamino)methyl]phenyl}sulfonyl)-3-piperidinyl]butyl}carbamate

This compound was made according in a manner analogous to the procedure outlined in Example 13, step 2, starting with methyl (4-(6-fluoro-3′-methyl-2-biphenylyl)-4-{(3R)-1-[(4-formylphenyl)sulfonyl]-3-piperidinyl}-4-hydroxybutyl)carbamate for methyl (4-(6-chloro-3′-ethyl-2-biphenylyl)-4-{(3R)-1-[(4-formylphenyl)carbonyl]-3-piperidinyl}-4-hydroxybutyl)carbamate and substituting methylamine for 2-aminoethanol. MS (m/z) 598.3 (M+H⁺).

The following are compounds of the invention:

| | | Synthetic | | | |
|---|---|---|---|---|---|
| Cpd. | | Method | | t_{R} | Mass |
| #. | Name | Example No. | LC_MS Method | (min) | observed |
| 1 | N-(4-((R)-1-(4-(aminomethyl)benzoyl)piperidin-3-yl)-4- | 4 | 1 | 2.08 | 530.2 |
| | hydroxy-4-(2-(o-tolyloxy)phenyl)butyl)acetamide | | | | |
| 2 | (4-(aminomethyl)phenyl)((3R)-3-(1-(3′-ethyl-6- | 4 | 1 | 2.45 | 533.3 |
| | fluorobiphenyl-2-yl)-1-hydroxy-5-methoxypentyl)piperidin-1- | | | | |
| | yl)methanone | | | | |
| 3 | (3-(aminomethyl)phenyl)(4-(1-(3′-ethyl-6-fluorobiphenyl-2- | 5 | 1 | 2.53 | 533.3 |
| | yl)-1-hydroxy-5-methoxypentyl)piperidin-1-yl)methanone | | | | |
| 4 | (4-(aminomethyl)phenyl)((R)-2-((R)-1-(4′,6-difluoro-3′- | 4 | 1 | 2.34 | 539.2 |
| | methylbiphenyl-2-yl)-1-hydroxy-5-methoxypentyl)morpholino)methanone | | | | |
| 5 | methyl 4-((R)-1-(4-(aminomethyl)benzoyl)piperidin-3-yl)-4- | 4 | | | |
| | hydroxy-4-(2-(o-tolyloxy)phenyl)butylcarbamate | | | | |
| 6 | 1-(1-{[4-(2-aminoethyl)phenyl]carbonyl}-4-piperidinyl)-1-(3′- | 6 | 1 | 2.54 | 547.3 |
| | ethyl-6-fluoro-2-biphenylyl)-5-(methyloxy)-1-pentanol | | | | |
| 7 | (4-(aminomethyl)phenyl)((3R)-3-(1-(6-fluoro-3′-methoxy-5′- | 4 | 1 | 2.36 | 549.3 |
| | methylbiphenyl-2-yl)-1-hydroxy-5-methoxypentyl)piperidin-1- | | | | |
| | yl)methanone | | | | |
| 8 | 1-((3R)-1-{[4-(aminomethyl)phenyl]carbonyl}-3-piperidinyl)- | 4 | 1 | 2.54 | 549.2 |
| | 1-(6-chloro-3′-ethyl-2-biphenylyl)-5-(methyloxy)-1-pentanol | | | | |
| 9 | (4-(aminomethyl)phenyl)((3R)-3-(1-(6-chloro-3′- | 4 | 1 | 2.48 | 549.2 |
| | ethylbiphenyl-2-yl)-1-hydroxy-5-methoxypentyl)piperidin-1- | | | | |
| | yl)methanone | | | | |
| 10 | (4-(aminomethyl)phenyl)((3R)-3-(1-(3-chloro-2-(3- | 4 | 1 | 2.43 | 549.2 |
| | methylbenzyl)phenyl)-1-hydroxy-5-methoxypentyl)piperidin-1- | | | | |
| | yl)methanone | | | | |
| 11 | ((R)-3-(1-(6-chloro-3′-ethylbiphenyl-2-yl)-1-hydroxy-5- | 4 | 1 | 2.31 | 618.2 |
| | methoxypentyl)piperidin-1-yl)(4-(piperazin-1- | | | | |
| | ylmethyl)phenyl)methanone | | | | |
| 12 | (1R)-1-((2R)-4-{[4-(aminomethyl)phenyl]carbonyl}-2- | 4 | 1 | 2.29 | 551.2 |
| | morpholinyl)-1-[6-fluoro-3′-methyl-5′-(methyloxy)-2-biphenylyl]-5- | | | | |
| | (methyloxy)-1-pentanol | | | | |
| 13 | (3-(aminomethyl)phenyl)((R)-2-((R)-1-(6-chloro-3′- | 4 | 1 | 2.44 | 551.2 |
| | ethylbiphenyl-2-yl)-1-hydroxy-5- | | | | |
| | methoxypentyl)morpholino)methanone | | | | |
| 14 | (4-(aminomethyl)phenyl)((R)-2-((R)-1-(6-chloro-3′- | 4 | 1 | 2.41 | 551.2 |
| | ethylbiphenyl-2-yl)-1-hydroxy-5- | | | | |
| | methoxypentyl)morpholino)methanone | | | | |
| 15 | (4-(aminomethyl)phenyl)((3R)-3-(1-(3-chloro-2-(o- | 4 | 3 | 2.90; | 551.7 |
| | tolyloxy)phenyl)-1-hydroxy-5-methoxypentyl)piperidin-1- | | | 3.0 | |
| | yl)methanone | | | | |
| 16 | (4-(aminomethyl)phenyl)((3R)-3-(1-(2-(2-chloro-6- | 4 | | | 551.1 |
| | methylphenoxy)phenyl)-1-hydroxy-5-methoxypentyl)piperidin-1- | | | | |
| | yl)methanone | | | | |
| 17 | methyl 4-(6-chloro-3′-ethylbiphenyl-2-yl)-4-((R)-1-(4- | 4 | 2 | 1.76 | 606.3 |
| | ((dimethylamino)methyl)benzoyl)piperidin-3-yl)-4- | | | | |
| | hydroxybutylcarbamate | | | | |
| 18 | (4-(aminomethyl)phenyl)((2R)-2-((1R)-1-(6-chloro-2′-fluoro- | 4 | 1 | 2.37 | 555.3 |
| | 5′-methylbiphenyl-2-yl)-1-hydroxy-5- | | | | |
| | methoxypentyl)morpholino)methanone | | | | |
| 19 | methyl 4-hydroxy-4-((R)-1-(4- | 2 | 1 | 2.49 | 560.3 |
| | ((methylamino)methyl)benzoyl)piperidin-3-yl)-4-(2-(o- | | | | |
| | tolyloxy)phenyl)butylcarbamate | | | | |
| 20 | N-4-(6-chloro-3′-ethylbiphenyl-2-yl)-4-hydroxy-4-((R)-1-(4- | 2 | 1 | 2.42 | 562.2 |
| | ((methylamino)methyl)benzoyl)piperidin-3-yl)butyl)formamide | | | | |
| 21 | (4-(2-aminoethyl)phenyl)((3R)-3-(1-(6-chloro-3′- | 4 | 1 | 2.55 | 563.2 |
| | ethylbiphenyl-2-yl)-1-hydroxy-5-methoxypentyl)piperidin-1- | | | | |
| | yl)methanone | | | | |
| 22 | ((3R)-3-(1-(3-chloro-2-(2-methylbenzyl)phenyl)-1-hydroxy-5- | 2 | 1 | 2.49 | 563.3 |
| | methoxypentyl)piperidin-1-yl)(4- | | | | |
| | ((methylamino)methyl)phenyl)methanone | | | | |
| 23 | methyl 4-((R)-1-(4-(aminomethyl)benzoyl)piperidin-3-yl)-4- | 4 | 1 | 2.18 | 564.1 |
| | (6-fluoro-3′-methoxybiphenyl-2-yl)-4-hydroxybutylcarbamate | | | | |
| 24 | (4-(aminomethyl)phenyl)((3R)-3-(1-(3-chloro-2-(2- | 4 | 3 | 3.06; | 565.3 |
| | ethylphenoxy)phenyl)-1-hydroxy-5-methoxypentyl)piperidin-1- | | | 3.15 | |
| | yl)methanone | | | | |
| 25 | (4-(aminomethyl)phenyl)((3R)-3-(1-(3-chloro-2-(3- | 4 | 3 | 1.83 | 565.3 |
| | ethylphenoxy)phenyl)-1-hydroxy-5-methoxypentyl)piperidin-1- | | | | |
| | yl)methanone | | | | |
| 26 | (4-(aminomethyl)phenyl)((R)-2-((R)-1-(6-chloro-3′- | 4 | 2 | 1.61 | 567.5 |
| | (methoxymethyl)biphenyl-2-yl)-1-hydroxy-5- | | | | |
| | methoxypentyl)morpholino)methanone | | | | |
| 27 | (4-(aminomethyl)phenyl)(3-((R)-1-(3-chloro-2-(quinolin-3- | 4 | 1 | 2.02; | 572.2 |
| | yl)phenyl)-1-hydroxy-5-methoxypentyl)piperidin-1-yl)methanone | | | 2.16 | |
| 28 | (4-(aminomethyl)phenyl)((R)-2-((R)-1-(3-chloro-2- | 4 | 1 | 2.42 | 573.2 |
| | (naphthalen-2-yl)phenyl)-1-hydroxy-5- | | | | |
| | methoxypentyl)morpholino)methanone | | | | |
| 29 | (1R)-1-((2R)-4-{[4-(aminomethyl)phenyl]carbonyl}-2- | 4 | 1 | 2.07 | 524.3 |
| | morpholinyl)-1-[3-chloro-2-(3-quinolinyl)phenyl]-5-(methyloxy)-1- | | | | |
| | pentanol | | | | |
| 30 | N-{4-(6-chloro-3′-ethyl-2-biphenylyl)-4-hydroxy-4-[(3R)-1- | 1 | 1 | 2.45 | 576.2 |
| | ({4-[(methylamino)methyl]phenyl}carbonyl)-3- | | | | |
| | piperidinyl]butyl}acetamide | | | | |
| 31 | methyl 4-(6-fluoro-3′-methoxybiphenyl-2-yl)-4-hydroxy-4- | 2 | 1 | 2.34 | 578.2 |
| | ((R)-1-(4-((methylamino)methyl)benzoyl)piperidin-3- | | | | |
| | yl)butylcarbamate | | | | |
| 32 | methyl 4-((R)-1-(4-(aminomethyl)benzoyl)piperidin-3-yl)-4- | 4 | 1 | 2.44 | 578.3 |
| | (6-chloro-3′-ethylbiphenyl-2-yl)-4-hydroxybutylcarbamate | | | | |
| 33 | methyl 4-(6-chloro-3′-methylbiphenyl-2-yl)-4-hydroxy-4-((R)- | 2 | 1 | 2.50 | 578.2 |
| | 1-(4-((methylamino)methyl)benzoyl)piperidin-3-yl)butylcarbamate | | | | |
| 34 | (4-(2-aminoethoxy)phenyl)((3R)-3-(1-(6-chloro-3′- | 4 | 1 | 2.54 | 579.2 |
| | ethylbiphenyl-2-yl)-1-hydroxy-5-methoxypentyl)piperidin-1- | | | | |
| | yl)methanone | | | | |
| 35 | methyl (R)-4-((R)-4-(4-(aminomethyl)benzoyl)morpholin-2- | 4 | 1 | 2.31 | 580.2 |
| | yl)-4-(6-chloro-3′-ethylbiphenyl-2-yl)-4-hydroxybutylcarbamate | | | | |
| 36 | methyl 4-((R)-1-(4-(aminomethyl)benzoyl)piperidin-3-yl)-4- | 4 | 1 | 2.37 | 580.2 |
| | (6-chloro-3′-methoxybiphenyl-2-yl)-4-hydroxybutylcarbamate | | | | |
| 37 | N-(4-(6-chloro-3′-ethylbiphenyl-2-yl)-4-hydroxy-4-((R)-1-(4- | 2 | 1 | 2.48 | 590.3 |
| | ((methylamino)methyl)benzoyl)piperidin-3-yl)butyl)propionamide | | | | |
| 38 | ethyl 4-((R)-1-(4-(aminomethyl)benzoyl)piperidin-3-yl)-4-(6- | 4 | 2 | 1.73 | 592.3 |
| | chloro-3′-ethylbiphenyl-2-yl)-4-hydroxybutylcarbamate | | | | |
| 39 | Methyl (S)-4-(6-chloro-3′-ethylbiphenyl-2-yl)-4-hydroxy-4- | 3 | 2 | 1.77 | 592.3 |
| | ((R)-1-(4-((methylamino)methyl)benzoyl)piperidin-3- | | | | |
| | yl)butylcarbamate | | | | |
| 40 | methyl 4-((R)-1-(4-(aminomethyl)benzoyl)piperidin-3-yl)-4- | 4 | 1 | 2.45 | 592.3 |
| | (6-chloro-3′-isopropylbiphenyl-2-yl)-4-hydroxybutylcarbamate | | | | |
| 41 | N-(4-(6-chloro-3′-ethylbiphenyl-2-yl)-4-hydroxy-4-((R)-1-(4- | 2 | 1 | 2.40 | 592.2 |
| | ((methylamino)methyl)benzoyl)piperidin-3-yl)butyl)-2- | | | | |
| | hydroxyacetamide | | | | |
| I-42 | methyl 4-(6-chloro-3′-methoxybiphenyl-2-yl)-4-hydroxy-4- | 2 | 1 | 2.39 | 594.2 |
| | ((R)-1-(4-((methylamino)methyl)benzoyl)piperidin-3- | | | | |
| | yl)butylcarbamate | | | | |
| 43 | methyl 4-((R)-1-(4-(aminomethyl)-2-fluorobenzoyl)piperidin- | 4 | 1 | 2.47 | 596.2 |
| | 3-yl)-4-(6-chloro-3′-ethylbiphenyl-2-yl)-4-hydroxybutylcarbamate | | | | |
| 44 | isopropyl 4-((R)-1-(4-(aminomethyl)benzoyl)piperidin-3-yl)- | 4 | 2 | 1.87 | 606.4 |
| | 4-(6-chloro-3′-ethylbiphenyl-2-yl)-4-hydroxybutylcarbamate | | | | |
| 45 | methyl 4-(6-chloro-3′-ethylbiphenyl-2-yl)-4-((R)-1-(4- | 4 | 2 | 1.78 | 606.3 |
| | ((ethylamino)methyl)benzoyl)piperidin-3-yl)-4- | | | | |
| | hydroxybutylcarbamate | | | | |
| 46 | methyl 4-(6-chloro-3′-ethylbiphenyl-2-yl)-4-hydroxy-4-((R)-1- | 4 | 2 | 1.86 | 620.4 |
| | (4-((isopropylamino)methyl)benzoyl)piperidin-3-yl)butylcarbamate | | | | |
| 47 | N-(4-(6-chloro-3′-ethylbiphenyl-2-yl)-4-hydroxy-4-((R)-1-(4- | 2 | 1 | 2.49 | 630.2 |
| | ((methylamino)methyl)benzoyl)piperidin-3-yl)butyl)-2,2,2- | | | | |
| | trifluoroacetamide | | | | |
| 48 | methyl {4-(3′-ethyl-6-fluoro-2-biphenylyl)-4-hydroxy-4-[(3R)- | 2 | 1 | 2.45 | 576.3 |
| | 1-({4-[(methylamino)methyl]phenyl}carbonyl)-3- | | | | |
| | piperidinyl]butyl}carbamate | | | | |
| 49 | methyl {4-(2′,6-difluoro-5′-methyl-2-biphenylyl)-4-hydroxy-4- | 2 | 1 | 2.43 | 580.2 |
| | [(3R)-1-({4-[(methylamino)methyl]phenyl}carbonyl)-3- | | | | |
| | piperidinyl]butyl}carbamate | | | | |
| 50 | N-{4-(2′,6-difluoro-5′-methyl-2-biphenylyl)-4-hydroxy-4-[(3R)- | 2 | 1 | 2.48 | 580.2 |
| | 1-({4-[(methylamino)methyl]phenyl}carbonyl)-3-piperidinyl]butyl}- | | | | |
| | 2-hydroxyacetamide | | | | |
| 51 | methyl 4-(6-chloro-3′-ethylbiphenyl-2-yl)-4-((R)-1-(4- | 4 | 2 | 2.07 | 674.3 |
| | ((cyclohexylmethylamino)methyl)benzoyl)piperidin-3-yl)-4- | | | | |
| | hydroxybutylcarbamate | | | | |
| 52 | (1R)-1-(6-chloro-3′-ethyl-2-biphenylyl)-1-[(2R)-4-({4- | 2 | 1 | 2.46 | 565.3 |
| | [(methylamino)methyl]phenyl}carbonyl)-2-morpholinyl]-5- | | | | |
| | (methyloxy)-1-pentanol | | | | |
| 53 | 1-(6-chloro-3′-ethyl-2-biphenylyl)-1-[(3R)-1-({4- | 2 | 1 | 2.43 | 563.3 |
| | [(methylamino)methyl]phenyl}carbonyl)-3-piperidinyl]-5- | | | | |
| | (methyloxy)-1-pentanol | | | | |
| 54 | methyl [4-((3R)-1-{[4-(aminomethyl)-3- | 4 | 1 | 2.47 | 608.2 |
| | (methyloxy)phenyl]carbonyl}-3-piperidinyl)-4-(6-chloro-3′-ethyl-2- | | | | |
| | biphenylyl)-4-hydroxybutyl]carbamate | | | | |
| 55 | N-{4-(6-chloro-3′-methyl-2-biphenylyl)-4-hydroxy-4-[(3R)-1- | 2 | 1 | 2.5 | 562.3 |
| | ({4-[(methylamino)methyl]phenyl}carbonyl)-3- | | | | |
| | piperidinyl]butyl}acetamide | | | | |
| 56 | N-[4-((3R)-1-{[4-(aminomethyl)-2-fluorophenyl]carbonyl}-3- | 4 | 1 | 2.58 | 566.2 |
| | piperidinyl)-4-(6-chloro-3′-methyl-2-biphenylyl)-4- | | | | |
| | hydroxybutyl]acetamide | | | | |
| 57 | N-[4-((3R)-1-{[4-(aminomethyl)phenyl]carbonyl}-3- | 4 | 1 | 2.48 | 548.2 |
| | piperidinyl)-4-(6-chloro-3′-methyl-2-biphenylyl)-4- | | | | |
| | hydroxybutyl]acetamide | | | | |
| 58 | N-{4-(6-chloro-3′-methyl-2-biphenylyl)-4-[(3R)-1-({2-fluoro-4- | 2 | 1 | 2.4 | 580.2 |
| | [(methylamino)methyl]phenyl}carbonyl)-3-piperidinyl]-4- | | | | |
| | hydroxybutyl}acetamide | | | | |
| 59 | N-{4-(6-chloro-3′-methyl-2-biphenylyl)-4-hydroxy-4-[(3R)-1- | 4 | 1 | 2.96 | 685.2 |
| | ({4-[2,2,2-trifluoro-1-hydroxy-1- | | | | |
| | (trifluoromethyl)ethyl]phenyl}carbonyl)-3- | | | | |
| | piperidinyl]butyl}acetamide | | | | |
| 60 | 4-{[(3R)-3-(1-(6-chloro-3′-ethyl-2-biphenylyl)-1-hydroxy-4- | 4 | 1 | 2.76 | 593.2 |
| | {[(methyloxy)carbonyl]amino}butyl)-1-piperidinyl]carbonyl}benzoic | | | | |
| | acid | | | | |
| 61 | methyl {4-(6-chloro-3′-methyl-2-biphenylyl)-4-hydroxy-4- | 4 | 1 | 2.73 | 627.2 |
| | [(3R)-1-({4-[(methylsulfonyl)methyl]phenyl}carbonyl)-3- | | | | |
| | piperidinyl]butyl}carbamate | | | | |
| 62 | (4-{[3-(1-(6-chloro-3′-ethyl-2-biphenylyl)-1-hydroxy-4- | 7 | 1 | 2.56 | 620.3 |
| | {[(methyloxy)carbonyl]amino}butyl)-1-piperidinyl]carbonyl}phenyl)- | | | | |
| | N,N,N-trimethylmethanaminium trifluoroacetate | | | | |
| 63 | methyl [4-hydroxy-4-[(3R)-1-({4- | 2 | 1 | 2.5 | 598.2 |
| | [(methylamino)methyl]phenyl}carbonyl)-3-piperidinyl]-4-(2′,4,6- | | | | |
| | trifluoro-5′-methyl-2-biphenylyl)butyl]carbamate | | | | |
| 64 | methyl [4-((3R)-1-{[4-(2-aminoethyl)phenyl]carbonyl}-3- | 4 | 1 | 2.49 | 578.2 |
| | piperidinyl)-4-(6-chloro-3′-methyl-2-biphenylyl)-4- | | | | |
| | hydroxybutyl]carbamate | | | | |
| 65 | methyl [4-[(3R)-1-({2-fluoro-4- | 4 | 1 | 2.48 | 616.2 |
| | [(methylamino)methyl]phenyl}carbonyl)-3-piperidinyl]-4-hydroxy-4- | | | | |
| | (2′,4,6-trifluoro-5′-methyl-2-biphenylyl)butyl]carbamate | | | | |
| 66 | methyl [4-[(3R)-1-({4-[(2-aminoethyl)oxy]phenyl}carbonyl)-3- | 4 | 1 | 2.4 | 594.2 |
| | piperidinyl]-4-(6-chloro-3′-methyl-2-biphenylyl)-4- | | | | |
| | hydroxybutyl]carbamate | | | | |
| 67 | methyl [4-((3R)-1-{[4-(aminomethyl)phenyl]carbonyl}-3- | 4 | 1 | 2.44 | 564.2 |
| | piperidinyl)-4-(6-chloro-3′-methyl-2-biphenylyl)-4- | | | | |
| | hydroxybutyl]carbamate | | | | |
| 68 | methyl [4-((3R)-1-{[3-(aminomethyl)phenyl]acetyl}-3- | 4 | 1 | 2.5 | 578.2 |
| | piperidinyl)-4-(6-chloro-3′-methyl-2-biphenylyl)-4- | | | | |
| | hydroxybutyl]carbamate | | | | |
| 69 | methyl {4-(6-fluoro-3′-methyl-2-biphenylyl)-4-hydroxy-4- | 2 | 1 | 2.39 | 562.3 |
| | [(3R)-1-({4-[(methylamino)methyl]phenyl}carbonyl)-3- | | | | |
| | piperidinyl]butyl}carbamate | | | | |
| 70 | N-{4-(6-fluoro-3′-methyl-2-biphenylyl)-4-hydroxy-4-[(3R)-1- | 2 | 1 | 2.38 | 562.3 |
| | ({4-[(methylamino)methyl]phenyl}carbonyl)-3-piperidinyl]butyl}-2- | | | | |
| | hydroxyacetamide | | | | |
| 71 | 2-chloro-N-{(4S)-4-(6-chloro-3′-ethyl-2-biphenylyl)-4- | 2 | 1 | 2.43 | 610.2 |
| | hydroxy-4-[(3R)-1-({4-[(methylamino)methyl]phenyl}carbonyl)-3- | | | | |
| | piperidinyl]butyl}acetamide | | | | |
| 72 | methyl {4-(6-chloro-3′-ethyl-2-biphenylyl)-4-[(3R)-1-({4- | 4 | 2 | 2.05 | 660.6 |
| | [(cyclohexylamino)methyl]phenyl}carbonyl)-3-piperidinyl]-4- | | | | |
| | hydroxybutyl}carbamate | | | | |
| 73 | methyl {4-(6-chloro-3′-ethyl-2-biphenylyl)-4-hydroxy-4-[(3R)- | 4 | 2 | 1.83 | 662.7 |
| | 1-({4-[(tetrahydro-2H-pyran-4-ylamino)methyl]phenyl}carbonyl)-3- | | | | |
| | piperidinyl]butyl}carbamate | | | | |
| 74 | methyl [4-[(3R)-1-({2-bromo-4- | 4 | 2 | 1.84 | 670.4 |
| | [(methylamino)methyl]phenyl}carbonyl)-3-piperidinyl]-4-(6-chloro- | | | | |
| | 3′-ethyl-2-biphenylyl)-4-hydroxybutyl]carbamate | | | | |
| 75 | methyl {4-(6-chloro-3′-ethyl-2-biphenylyl)-4-hydroxy-4-[(3R)- | 4 | 2 | 1.96 | 668.4 |
| | 1-({5-[(methylamino)methyl]-2-biphenylyl}carbonyl)-3- | | | | |
| | piperidinyl]butyl}carbamate | | | | |
| 76 | methyl {4-(6-chloro-3′-ethyl-2-biphenylyl)-4-hydroxy-4-[(3R)- | 4 | 2 | 1.89 | 642.5 |
| | 1-({4-[(methylamino)methyl]-1-naphthalenyl}carbonyl)-3- | | | | |
| | piperidinyl]butyl}carbamate | | | | |
| 77 | methyl [4-[(3R)-1-({2-butyl-4- | 4 | 2 | 2.01 | 648.7 |
| | [(methylamino)methyl]phenyl}carbonyl)-3-piperidinyl]-4-(6-chloro- | | | | |
| | 3′-ethyl-2-biphenylyl)-4-hydroxybutyl]carbamate | | | | |
| 78 | methyl {4-(6-chloro-3′-ethyl-2-biphenylyl)-4-[(3R)-1-({2-ethyl- | 4 | 2 | 1.87 | 620.6 |
| | 4-[(methylamino)methyl]phenyl}carbonyl)-3-piperidinyl]-4- | | | | |
| | hydroxybutyl}carbamate | | | | |
| 79 | methyl {4-(6-chloro-3′-ethyl-2-biphenylyl)-4-hydroxy-4-[(3R)- | 4 | 2 | 1.8 | 606.5 |
| | 1-({2-methyl-4-[(methylamino)methyl]phenyl}carbonyl)-3- | | | | |
| | piperidinyl]butyl}carbamate | | | | |
| 80 | methyl [4-(6-chloro-3′-ethyl-2-biphenylyl)-4-hydroxy-4-((3R)- | 4 | 2 | 1.96 | 648.7 |
| | 1-{[4-[(methylamino)methyl]-2-(2-methylpropyl)phenyl]carbonyl}-3- | | | | |
| | piperidinyl)butyl]carbamate | | | | |
| 81 | methyl {4-(6-chloro-3′-ethyl-2-biphenylyl)-4-[(3R)-1-({2-[2- | 4 | 2 | 1.39 | 692.5 |
| | (1,3-dioxolan-2-yl)ethyl]-4-[(methylamino)methyl]phenyl}carbonyl)- | | | | |
| | 3-piperidinyl]-4-hydroxybutyl}carbamate | | | | |
| 82 | methyl {4-(6-chloro-3′-ethyl-2-biphenylyl)-4-[(3R)-1-({2-(2- | 4 | 2 | 1.35 | 645.6 |
| | cyanoethyl)-4-[(methylamino)methyl]phenyl}carbonyl)-3- | | | | |
| | piperidinyl]-4-hydroxybutyl}carbamate | | | | |
| 83 | Methyl {4-(6-chloro-3′-ethyl-2-biphenylyl)-4-hydroxy-4-[(3R)- | 8 | 2 | 1.35 | 688.8 |
| | 1-({4-[(methylamino)methyl]-2-[2-(1H-tetrazol-5- | | | | |
| | yl)ethyl]phenyl}carbonyl)-3-piperidinyl]butyl}carbamate | | | | |
| 84 | Methyl [4-[(3R)-1-({2-(3-amino-3-oxopropyl)-4- | 9 | 2 | 1.27 | 663.5 |
| | [(methylamino)methyl]phenyl}carbonyl)-3-piperidinyl]-4-(6-chloro- | | | | |
| | 3′-ethyl-2-biphenylyl)-4-hydroxybutyl]carbamate | | | | |
| 85 | methyl {4-(6-chloro-3′-ethyl-2-biphenylyl)-4-[(3R)-1-({2- | 4 | 2 | 1.59 | 617.1 |
| | cyano-4-[(methylamino)methyl]phenyl}carbonyl)-3-piperidinyl]-4- | | | | |
| | hydroxybutyl}carbamate | | | | |
| 86 | Methyl [4-[(3R)-1-({2-(aminocarbonyl)-4- | 10 | 2 | 1.17 | 635.3 |
| | [(methylamino)methyl]phenyl}carbonyl)-3-piperidinyl]-4-(6-chloro- | | | | |
| | 3′-ethyl-2-biphenylyl)-4-hydroxybutyl]carbamate | | | | |
| 87 | methyl {4-[6-fluoro-3′-methyl-5-(methyloxy)-2-biphenylyl]-4- | 2 | 2 | 1.05 | 592.2 |
| | hydroxy-4-[(3R)-1-({4-[(methylamino)methyl]phenyl}carbonyl)-3- | | | | |
| | piperidinyl]butyl}carbamate | | | | |
| 88 | methyl [4-(6-chloro-3′-ethyl-2-biphenylyl)-4-hydroxy-4-((3R)- | 4 | 2 | 1.33 | 622.6 |
| | 1-{[4-[(methylamino)methyl]-2-(methyloxy)phenyl]carbonyl}-3- | | | | |
| | piperidinyl)butyl]carbamate | | | | |
| 89 | 2-{[(3R)-3-(1-(6-chloro-3′-ethyl-2-biphenylyl)-1-hydroxy-4- | 11 | 2 | 1.62 | 636.8 |
| | {[(methyloxy)carbonyl]amino}butyl)-1-piperidinyl]carbonyl}-5- | | | | |
| | [(methylamino)methyl]benzoic acid | | | | |
| 90 | Methyl [4-[(3R)-1-({2-(2-amino-2-oxoethyl)-4- | 12 | 2 | 1.15 | 649.3 |
| | [(methylamino)methyl]phenyl}carbonyl)-3-piperidinyl]-4-(6-chloro- | | | | |
| | 3′-ethyl-2-biphenylyl)-4-hydroxybutyl]carbamate | | | | |
| 91 | methyl {(4R)-4-(3′-ethyl-6-fluoro-2-biphenylyl)-4-hydroxy-4- | 2 | 1 | 2.17 | 578.3 |
| | [(2R)-4-({4-[(methylamino)methyl]phenyl}carbonyl)-2- | | | | |
| | morpholinyl]butyl}carbamate | | | | |
| 92 | (1R)-1-((2R)-4-{[4-(aminomethyl)phenyl]carbonyl}-2- | 4 | 1 | 2.44 | 552.2 |
| | morpholinyl)-1-[2-chloro-3-(3-ethylphenyl)-4-pyridinyl]-5- | | | | |
| | (methyloxy)-1-pentanol | | | | |
| 93 | 1-((2R)-4-{[4-(aminomethyl)phenyl]carbonyl}-2-morpholinyl)- | 4 | 1 | 2.46 | 579.2 |
| | 1-[2-(1-benzothien-3-yl)-3-chlorophenyl]-5-(methyloxy)-1-pentanol | | | | |
| 94 | methyl {4-[3-chloro-2-(3-quinolinyl)phenyl]-4-hydroxy-4- | 2 | 1 | 2.36 | 615.2 |
| | [(3R)-1-({4-[(methylamino)methyl]phenyl}carbonyl)-3- | | | | |
| | piperidinyl]butyl}carbamate | | | | |
| 95 | (1R)-1-[4-chloro-3-(3-ethylphenyl)-2-pyridinyl]-1-[(2R)-4-({4- | 2 | 1 | 2.15 | 566.3 |
| | [(methylamino)methyl]phenyl}carbonyl)-2-morpholinyl]-5- | | | | |
| | (methyloxy)-1-pentanol | | | | |
| 96 | methyl {4-{3-chloro-2-[4-(1-methylethyl)-2- | 2 | 1 | 2.39 | 658.2 |
| | quinazolinyl]phenyl}-4-hydroxy-4-[(3R)-1-({4- | | | | |
| | [(methylamino)methyl]phenyl}carbonyl)-3- | | | | |
| | piperidinyl]butyl}carbamate | | | | |
| 97 | methyl {4-[2-chloro-3-(3-ethylphenyl)-4-pyridinyl]-4-hydroxy- | 2 | 1 | 2.44 | 593.3 |
| | 4-[(3R)-1-({4-[(methylamino)methyl]phenyl}carbonyl)-3- | | | | |
| | piperidinyl]butyl}carbamate | | | | |
| 98 | methyl {(4R)-4-(6-chloro-3′-methyl-2-biphenylyl)-4-hydroxy- | 2 | 1 | 2.4 | 580.2 |
| | 4-[(2R)-4-({4-[(methylamino)methyl]phenyl}carbonyl)-2- | | | | |
| | morpholinyl]butyl}carbamate | | | | |
| 99 | 1-((3R)-1-{[4-(aminomethyl)phenyl]carbonyl}-3-piperidinyl)- | 4 | 3 | 2.94 | 551.2 |
| | 1-{3-chloro-2-[(3-methylphenyl)oxy]phenyl}-5-(methyloxy)-1- | | | | |
| | pentanol | | | | |
| 100 | methyl (4-((3R)-1-{[4-(aminomethyl)phenyl]carbonyl}-3- | 4 | 3 | 3.33 | 580.1 |
| | piperidinyl)-4-{3-chloro-2-[(3-methylphenyl)oxy]phenyl}-4- | | | | |
| | hydroxybutyl)carbamate | | | | |
| 101 | methyl (4-((3R)-1-{[4-(aminomethyl)phenyl]carbonyl}-3- | 4 | 3 | 3.48 | 560.2 |
| | piperidinyl)-4-{2-[(2,6-dimethylphenyl)oxy]phenyl}-4- | | | | |
| | hydroxybutyl)carbamate | | | | |
| 102 | methyl {4-{3-chloro-2-[8-(1-methylethyl)-2-quinolinyl]phenyl}- | 2 | 3 | 1.88 | 657.2 |
| | 4-hydroxy-4-[(3R)-1-({4-[(methylamino)methyl]phenyl}carbonyl)-3- | | | | |
| | piperidinyl]butyl}carbamate | | | | |
| 103 | methyl [4-(6-chloro-3′-ethyl-2-biphenylyl)-4-hydroxy-4-((3R)- | 5 | 2 | 1.88 | 579.5 |
| | 1-{[4-(hydroxymethyl)phenyl]carbonyl}-3- | | | | |
| | piperidinyl)butyl]carbamate | | | | |
| 104 | Methyl (4-(6-chloro-3′-ethyl-2-biphenylyl)-4-hydroxy-4-{(3R)- | 13 | 2 | 1.24 | 622.2 |
| | 1-[(4-{[(2-hydroxyethyl)amino]methyl}phenyl)carbonyl]-3- | | | | |
| | piperidinyl}butyl)carbamate | | | | |
| 105 | methyl (4-(6-chloro-3′-ethyl-2-biphenylyl)-4-hydroxy-4-{(3R)- | 13 | 2 | 1.2 | 636.3 |
| | 1-[(4-{[(3-hydroxypropyl)amino]methyl}phenyl)carbonyl]-3- | | | | |
| | piperidinyl}butyl)carbamate | | | | |
| 106 | methyl [4-(6-chloro-3′-ethyl-2-biphenylyl)-4-hydroxy-4-((3R)- | 13 | 2 | 1.26 | 636.3 |
| | 1-{[4-({[2-(methyloxy)ethyl]amino}methyl)phenyl]carbonyl}-3- | | | | |
| | piperidinyl)butyl]carbamate | | | | |
| 107 | methyl [4-(6-chloro-3′-ethyl-2-biphenylyl)-4-((3R)-1-{[4-({[2- | 13 | 2 | 1.31 | 650.4 |
| | (ethyloxy)ethyl]amino}methyl)phenyl]carbonyl}-3-piperidinyl)-4- | | | | |
| | hydroxybutyl]carbamate | | | | |
| 108 | methyl [4-(6-chloro-3′-ethyl-2-biphenylyl)-4-hydroxy-4-((3R)- | 13 | 2 | 1.29 | 650.4 |
| | 1-{[4-({[1-methyl-2- | | | | |
| | (methyloxy)ethyl]amino}methyl)phenyl]carbonyl}-3- | | | | |
| | piperidinyl)butyl]carbamate | | | | |
| 109 | methyl {4-(6-chloro-3′-ethyl-2-biphenylyl)-4-hydroxy-4-[(3R)- | 13 | 2 | 1.37 | 664.3 |
| | 1-({4-[({2-[(1- | | | | |
| | methylethyl)oxy]ethyl}amino)methyl]phenyl}carbonyl)-3- | | | | |
| | piperidinyl]butyl}carbamate | | | | |
| 110 | methyl (4-(6-chloro-3′-ethyl-2-biphenylyl)-4-hydroxy-4-{(3R)- | 13 | 2 | 1.3 | 662.3 |
| | 1-[(4-{[(tetrahydro-2-furanylmethyl)amino]methyl}phenyl)carbonyl]- | | | | |
| | 3-piperidinyl}butyl)carbamate | | | | |
| 111 | methyl [2-({(R)-(6-chloro-3′-ethyl-2-biphenylyl)[(3R)-1-({4- | 2 | 2 | 1.41 | 578.6 |
| | [(methylamino)methyl]phenyl}carbonyl)-3- | | | | |
| | piperidinyl]methyl}oxy)ethyl]carbamate | | | | |
| 112 | methyl [2-({(S)-(6-chloro-3′-ethyl-2-biphenylyl)[(2R)-4-({4- | 2 | 2 | 1.68 | 580.7 |
| | [(methylamino)methyl]phenyl}carbonyl)-2- | | | | |
| | morpholinyl]methyl}oxy)ethyl]carbamate | | | | |
| 113 | methyl [4-(6-chloro-3′-ethyl-2-biphenylyl)-4-hydroxy-4-((3R)- | 13 | 2 | 1.31 | 652.2 |
| | 1-{[4-({[2-(methylthio)ethyl]amino}methyl)phenyl]carbonyl}-3- | | | | |
| | piperidinyl)butyl]carbamate | | | | |
| 114 | Methyl [4-(6-chloro-3′-ethyl-2-biphenylyl)-4-hydroxy-4-((3R)- | 17 | 2 | 1.25 | 684.1 |
| | 1-{[4-({[2-(methylsulfonyl)ethyl]amino}methyl)phenyl]carbonyl}-3- | | | | |
| | piperidinyl)butyl]carbamate | | | | |
| 115 | methyl {4-(6-fluoro-3′-methyl-2-biphenylyl)-4-hydroxy-4- | 19 | 2 | 1.27 | 598.3 |
| | [(3R)-1-({4-[(methylamino)methyl]phenyl}sulfonyl)-3- | | | | |
| | piperidinyl]butyl}carbamate | | | | |
| 116 | methyl [4-{(3R)-1-[(4-{[(2- | 13 | 2 | 1.12 | 601.3 |
| | cyanoethyl)amino]methyl}phenyl)carbonyl]-3-piperidinyl}-4-(6- | | | | |
| | fluoro-3′-methyl-2-biphenylyl)-4-hydroxybutyl]carbamate | | | | |
| 117 | N-[(4-{[(3R)-3-(1-(6-fluoro-3′-methyl-2-biphenylyl)-1- | 16 | 2 | 1.14 | 620.3 |
| | hydroxy-4-{[(methyloxy)carbonyl]amino}butyl)-1- | | | | |
| | piperidinyl]carbonyl}phenyl)methyl]-$$-alanine | | | | |
| 118 | methyl [4-[(3R)-1-({4-[amino(imino)methyl]phenyl}carbonyl)- | 4 | 2 | 1.09 | 561.1 |
| | 3-piperidinyl]-4-(6-fluoro-3′-methyl-2-biphenylyl)-4- | | | | |
| | hydroxybutyl]carbamate | | | | |
| 119 | N-[(4-{[(3R)-3-(1-(6-fluoro-3′-methyl-2-biphenylyl)-1- | 16 | 2 | 1.18 | 606.1 |
| | hydroxy-4-{[(methyloxy)carbonyl]amino}butyl)-1- | | | | |
| | piperidinyl]carbonyl}phenyl)methyl]glycine | | | | |
| 120 | methyl [4-((3R)-1-{[4-(aminocarbonyl)phenyl]carbonyl}-3- | 5 | 2 | 1.42 | 562.1 |
| | piperidinyl)-4-(6-fluoro-3′-methyl-2-biphenylyl)-4- | | | | |
| | hydroxybutyl]carbamate | | | | |
| 121 | methyl [4-{(3R)-1-[(4-{[(3-amino-3- | 14 | 2 | 1.08 | 619.3 |
| | oxopropyl)amino]methyl}phenyl)carbonyl]-3-piperidinyl}-4-(6- | | | | |
| | fluoro-3′-methyl-2-biphenylyl)-4-hydroxybutyl]carbamate | | | | |
| 122 | methyl [4-(6-fluoro-3′-methyl-2-biphenylyl)-4-hydroxy-4- | 15 | 2 | 1.17 | 644.6 |
| | ((3R)-1-{[4-({[2-(1H-tetrazol-5- | | | | |
| | yl)ethyl]amino}methyl)phenyl]carbonyl}-3- | | | | |
| | piperidinyl)butyl]carbamate | | | | |
| 123 | methyl (4-(6-fluoro-3′-methyl-2-biphenylyl)-4-hydroxy-4- | 13 | 2 | 1.15 | 620.3 |
| | {(3R)-1-[(4-{[(4-hydroxybutyl)amino]methyl}phenyl)carbonyl]-3- | | | | |
| | piperidinyl}butyl)carbamate | | | | |
| 124 | methyl [4-((3R)-1-{[4-({[2- | 13 | 2 | 0.89 | 619.3 |
| | (dimethylamino)ethyl]amino}methyl)phenyl]carbonyl}-3- | | | | |
| | piperidinyl)-4-(6-fluoro-3′-methyl-2-biphenylyl)-4- | | | | |
| | hydroxybutyl]carbamate | | | | |
| 125 | methyl [4-((3R)-1-{[4-({[3- | 13 | 2 | 0.97 | 633.3 |
| | (dimethylamino)propyl]amino}methyl)phenyl]carbonyl}-3- | | | | |
| | piperidinyl)-4-(6-fluoro-3′-methyl-2-biphenylyl)-4- | | | | |
| | hydroxybutyl]carbamate | | | | |
| 126 | Methyl [4-((3R)-1-{[4- | 18 | 2 | 1.21 | 590.5 |
| | ({[amino(imino)methyl]amino}methyl)phenyl]carbonyl}-3- | | | | |
| | piperidinyl)-4-(6-fluoro-3′-methyl-2-biphenylyl)-4- | | | | |
| | hydroxybutyl]carbamate | | | | |
| 127 | methyl [4-{(3R)-1-[(4- | 13 | 2 | 1.26 | 587.1 |
| | {[(cyanomethyl)amino]methyl}phenyl)carbonyl]-3-piperidinyl}-4-(6- | | | | |
| | fluoro-3′-methyl-2-biphenylyl)-4-hydroxybutyl]carbamate | | | | |
| 128 | methyl {4-[3-chloro-2-(8-methyl-2-quinolinyl)phenyl]-4- | 2 | 3 | 1.66 | 629.1 |
| | hydroxy-4-[(3R)-1-({4-[(methylamino)methyl]phenyl}carbonyl)-3- | | | | |
| | piperidinyl]butyl}carbamate | | | | |
| 130 | methyl {4-((3R)-1-{[4-(aminomethyl)-2- | 4 | 1 | 2.37 | 619.2 |
| | fluorophenyl]carbonyl}-3-piperidinyl)-4-[3-chloro-2-(3- | | | | |
| | quinolinyl)phenyl]-4-hydroxybutyl}carbamate | | | | |
| 131 | methyl {4-[3-fluoro-2-(3-quinolinyl)phenyl]-4-hydroxy-4- | 2 | 1 | 2.3 | 599.3 |
| | [(3R)-1-({4-[(methylamino)methyl]phenyl}carbonyl)-3- | | | | |
| | piperidinyl]butyl}carbamate | | | | |
| 132 | methyl {4-{2-chloro-3-[3-(1-methylethyl)phenyl]-4-pyridinyl}- | 2 | 1 | 2.36 | 607.2 |
| | 4-hydroxy-4-[(3R)-1-({4-[(methylamino)methyl]phenyl}carbonyl)-3- | | | | |
| | piperidinyl]butyl}carbamate | | | | |
| 133 | methyl {4-[3-chloro-2-(5-methyl-2-furanyl)phenyl]-4-hydroxy- | 2 | 1 | 2.38 | 568.2 |
| | 4-[(3R)-1-({4-[(methylamino)methyl]phenyl}carbonyl)-3- | | | | |
| | piperidinyl]butyl}carbamate | | | | |
| 134 | (1R)-1-{2-chloro-3-[3-(1-methylethyl)phenyl]-4-pyridinyl}-1- | 2 | 1 | 2.26 | 580.2 |
| | [(2R)-4-({4-[(methylamino)methyl]phenyl}carbonyl)-2-morpholinyl]- | | | | |
| | 5-(methyloxy)-1-pentanol | | | | |
| 135 | methyl {4-[5-chloro-4-(3-ethylphenyl)-3-pyridinyl]-4-hydroxy- | 2 | 1 | 2.14 | 593.2 |
| | 4-[(3R)-1-({4-[(methylamino)methyl]phenyl}carbonyl)-3- | | | | |
| | piperidinyl]butyl}carbamate | | | | |
| 136 | methyl {4-{5-chloro-4-[3-(1-methylethyl)phenyl]-3-pyridinyl}- | 2 | 1 | 2.2 | 607.2 |
| | 4-hydroxy-4-[(3R)-1-({4-[(methylamino)methyl]phenyl}carbonyl)-3- | | | | |
| | piperidinyl]butyl}carbamate | | | | |
| 137 | methyl [4-{(3R)-1-[(4-{[(4-amino-4- | 13 | 2 | 1.5 | 633.6 |
| | oxobutyl)amino]methyl}phenyl)carbonyl]-3-piperidinyl}-4-(6-fluoro- | | | | |
| | 3′-methyl-2-biphenylyl)-4-hydroxybutyl]carbamate | | | | |
| 138 | methyl {4-(6-fluoro-3′-methyl-2-biphenylyl)-4-hydroxy-4- | 13 | 2 | 1.11 | 669.2 |
| | [(3R)-1-({4-[({2- | | | | |
| | [(methylsulfonyl)amino]ethyl}amino)methyl]phenyl}carbonyl)-3- | | | | |
| | piperidinyl]butyl}carbamate | | | | |
| 139 | methyl [4-(6-fluoro-3′-methyl-2-biphenylyl)-4-hydroxy-4- | 13 | 2 | 1.12 | 658.3 |
| | ((3R)-1-{[4-({[3-(1H-tetrazol-5- | | | | |
| | yl)propyl]amino}methyl)phenyl]carbonyl}-3- | | | | |
| | piperidinyl)butyl]carbamate | | | | |
| 140 | Methyl [4-{(3R)-1-[(4-{[(2-amino-2- | 14 | 2 | 1.02 | 605.3 |
| | oxoethyl)amino]methyl}phenyl)carbonyl]-3-piperidinyl}-4-(6-fluoro- | | | | |
| | 3′-methyl-2-biphenylyl)-4-hydroxybutyl]carbamate | | | | |
| 141 | Methyl (4-(6-fluoro-3′-methyl-2-biphenylyl)-4-hydroxy-4- | 15 | 2 | 1.58 | 630.7 |
| | {(3R)-1-[(4-{[(1H-tetrazol-5- | | | | |
| | ylmethyl)amino]methyl}phenyl)carbonyl]-3- | | | | |
| | piperidinyl}butyl)carbamate | | | | |
| 142 | 1-(6-chloro-3′-ethyl-2-biphenylyl)-1-[(3R)-1-({4- | 2 | 1 | 2.4 | 563.3 |
| | [(methylamino)methyl]phenyl}carbonyl)-3-piperidinyl]-1,6- | | | | |
| | hexanediol | | | | |
| 143 | N-{4-((3R)-1-{[4-(aminomethyl)phenyl]carbonyl}-3- | 4 | 1 | 2.32 | 564.2 |
| | piperidinyl)-4-[6-fluoro-3′-(methyloxy)-2-biphenylyl]-4- | | | | |
| | hydroxybutyl}-2-hydroxyacetamide | | | | |
| 144 | N-{(4S)-4-[6-fluoro-3′-(methyloxy)-2-biphenylyl]-4-hydroxy-4- | 2 | 1 | 2.33 | 578.2 |
| | [(3R)-1-({4-[(methylamino)methyl]phenyl}carbonyl)-3- | | | | |
| | piperidinyl]butyl}-2-hydroxyacetamide | | | | |
| 145 | N-{4-((3R)-1-{[4-(aminomethyl)-2-fluorophenyl]carbonyl}-3- | 4 | 1 | 2.33 | 582.2 |
| | piperidinyl)-4-[6-fluoro-3′-(methyloxy)-2-biphenylyl]-4- | | | | |
| | hydroxybutyl}-2-hydroxyacetamide | | | | |
| 146 | N-{4-((3R)-1-{[4-(aminomethyl)phenyl]carbonyl}-3- | 4 | 1 | 2.62 | 581.2 |
| | piperidinyl)-4-[6-chloro-3′-(methyloxy)-2-biphenylyl]-4- | | | | |
| | hydroxybutyl}-2-hydroxyacetamide | | | | |
| 147 | N-{4-[6-chloro-3′-(methyloxy)-2-biphenylyl]-4-hydroxy-4- | 2 | 1 | 2.43 | 596.2 |
| | [(3R)-1-({4-[(methylamino)methyl]phenyl}carbonyl)-3- | | | | |
| | piperidinyl]butyl}-2-hydroxyacetamide | | | | |
| 148 | N-{4-((3R)-1-{[4-(aminomethyl)-2-fluorophenyl]carbonyl}-3- | 4 | 1 | 2.32 | 599.2 |
| | piperidinyl)-4-[6-chloro-3′-(methyloxy)-2-biphenylyl]-4- | | | | |
| | hydroxybutyl}-2-hydroxyacetamide | | | | |
| 149 | methyl {4-(6-chloro-3′-fluoro-5′-methyl-2-biphenylyl)-4-[(3R)- | 4 | 1 | 2.42 | 614.3 |
| | 1-({2-fluoro-4-[(methylamino)methyl]phenyl}carbonyl)-3- | | | | |
| | piperidinyl]-4-hydroxybutyl}carbamate | | | | |
| 150 | methyl {4-(6-chloro-3′-fluoro-5′-methyl-2-biphenylyl)-4- | 2 | 1 | 2.4 | 597.2 |
| | hydroxy-4-[(3R)-1-({4-[(methylamino)methyl]phenyl}carbonyl)-3- | | | | |
| | piperidinyl]butyl}carbamate | | | | |
| 151 | methyl {4-(3′,6-difluoro-5′-methyl-2-biphenylyl)-4-[(3R)-1-({2- | 4 | 1 | 1.7 | 598.3 |
| | fluoro-4-[(methylamino)methyl]phenyl}carbonyl)-3-piperidinyl]-4- | | | | |
| | hydroxybutyl}carbamate | | | | |
| 152 | methyl {4-[3-chloro-2-(2,3-dihydro-1-benzofuran-6- | 4 | 1 | 1.7 | 624.2 |
| | yl)phenyl]-4-[(3R)-1-({2-fluoro-4- | | | | |
| | [(methylamino)methyl]phenyl}carbonyl)-3-piperidinyl]-4- | | | | |
| | hydroxybutyl}carbamate | | | | |
| 153 | methyl {4-[3-chloro-2-(2,3-dihydro-1-benzofuran-6- | 2 | 1 | 1.66 | 606.3 |
| | yl)phenyl]-4-hydroxy-4-[(3R)-1-({4- | | | | |
| | [(methylamino)methyl]phenyl}carbonyl)-3- | | | | |
| | piperidinyl]butyl}carbamate | | | | |
| 154 | methyl {4-[6-chloro-2′-(methyloxy)-5′-(trifluoromethyl)-2- | 4 | 1 | 2.39 | 680.2 |
| | biphenylyl]-4-[(3R)-1-({2-fluoro-4- | | | | |
| | [(methylamino)methyl]phenyl}carbonyl)-3-piperidinyl]-4- | | | | |
| | hydroxybutyl}carbamate | | | | |
| 155 | methyl {(4S)-4-[6-chloro-2′-(methyloxy)-5′-(trifluoromethyl)- | 2 | 1 | 2.34 | 662.2 |
| | 2-biphenylyl]-4-hydroxy-4-[(3R)-1-({4- | | | | |
| | [(methylamino)methyl]phenyl}carbonyl)-3- | | | | |
| | piperidinyl]butyl}carbamate | | | | |
| 156 | methyl (4-((3R)-1-{[4-(aminomethyl)phenyl]carbonyl}-3- | 4 | 3 | 2.81 | 594.2 |
| | piperidinyl)-4-{3-chloro-2-[(2-ethylphenyl)oxy]phenyl}-4- | | | | |
| | hydroxybutyl)carbamate | | | | |
| 157 | methyl (4-((3R)-1-{[4-(aminomethyl)phenyl]carbonyl}-3- | 4 | 3 | 2.69 | 580.2 |
| | piperidinyl)-4-{3-chloro-2-[(2-methylphenyl)oxy]phenyl}-4- | | | | |
| | hydroxybutyl)carbamate | | | | |
| 158 | methyl {4-(3′-ethyl-6-fluoro-2-biphenylyl)-4-hydroxy-4-[(3R)- | 2 | 1 | 2.45 | 576.3 |
| | 1-({4-[(methylamino)methyl]phenyl}carbonyl)-3- | | | | |
| | piperidinyl]butyl}carbamate | | | | |
| 159 | N-{4-(2′,6-difluoro-5′-methyl-2-biphenylyl)-4-hydroxy-4-[(3R)- | 2 | 1 | 2.4 | 564.2 |
| | 1-({4-[(methylamino)methyl]phenyl}carbonyl)-3- | | | | |
| | piperidinyl]butyl}acetamide | | | | |
| 160 | N-{4-(6-fluoro-3′-methyl-2-biphenylyl)-4-hydroxy-4-[(3R)-1- | 2 | 1 | 2.43 | 546.2 |
| | ({4-[(methylamino)methyl]phenyl}carbonyl)-3- | | | | |
| | piperidinyl]butyl}acetamide | | | | |
| 161 | methyl {(4S)-4-(3′-ethyl-6-fluoro-2-biphenylyl)-4-[(3R)-1-({2- | 4 | 1 | 2.44 | 594.3 |
| | fluoro-4-[(methylamino)methyl]phenyl}carbonyl)-3-piperidinyl]-4- | | | | |
| | hydroxybutyl}carbamate | | | | |
| 162 | N-{4-(3′-ethyl-6-fluoro-2-biphenylyl)-4-hydroxy-4-[(3R)-1-({4- | 2 | 1 | 2.45 | 560.3 |
| | [(methylamino)methyl]phenyl}carbonyl)-3- | | | | |
| | piperidinyl]butyl}acetamide | | | | |
| 163 | methyl [4-[(3R)-1-({2-fluoro-4- | 4 | 1 | 2.42 | 580.2 |
| | [(methylamino)methyl]phenyl}carbonyl)-3-piperidinyl]-4-(6-fluoro- | | | | |
| | 3′-methyl-2-biphenylyl)-4-hydroxybutyl]carbamate | | | | |
| 164 | N-{4-(2′,6-difluoro-5′-methyl-2-biphenylyl)-4-[(3R)-1-({2- | 4 | 1 | 2.35 | 598.3 |
| | fluoro-4-[(methylamino)methyl]phenyl}carbonyl)-3-piperidinyl]-4- | | | | |
| | hydroxybutyl}-2-hydroxyacetamid | | | | |
| 165 | methyl {4-(2′,6-difluoro-5′-methyl-2-biphenylyl)-4-[(3R)-1-({2- | 4 | 1 | 2.47 | 598.3 |
| | fluoro-4-[(methylamino)methyl]phenyl}carbonyl)-3-piperidinyl]-4- | | | | |
| | hydroxybutyl}carbamate | | | | |
| 166 | N-{4-(2′,6-difluoro-5′-methyl-2-biphenylyl)-4-hydroxy-4-[(3R)- | 2 | 1 | 2.5 | 618.2 |
| | 1-({4-[(methylamino)methyl]phenyl}carbonyl)-3-piperidinyl]butyl}- | | | | |
| | 2,2,2-trifluoroacetamide | | | | |
| 167 | methyl {4-[6-fluoro-3′-(1-methylethyl)-2-biphenylyl]-4- | 2 | 1 | 2.46 | 590.3 |
| | hydroxy-4-[(3R)-1-({4-[(methylamino)methyl]phenyl}carbonyl)-3- | | | | |
| | piperidinyl]butyl}carbamate | | | | |
| 168 | methyl {4-(2′,6-difluoro-5′-methyl-2-biphenylyl)-4-[(3R)-1-({2- | 4 | 3 | 1.68 | 535.2 |
| | fluoro-4-[(methylamino)methyl]phenyl}carbonyl)-3-piperidinyl]-4- | | | | |
| | hydroxybutyl}carbamate | | | | |
| 169 | methyl (4-((3R)-1-{[4-(aminomethyl)phenyl]carbonyl}-3- | 4 | 3 | 1.74 | 594.2 |
| | piperidinyl)-4-{3-chloro-2-[(3-ethylphenyl)oxy]phenyl}-4- | | | | |
| | hydroxybutyl)carbamate | | | | |
| 170 | methyl (4-((3R)-1-{[4-(aminomethyl)phenyl]carbonyl}-3- | 4 | 3 | 1.61 | 564.2 |
| | piperidinyl)-4-{3-fluoro-2-[(3-methylphenyl)oxy]phenyl}-4- | | | | |
| | hydroxybutyl)carbamate | | | | |
| 171 | methyl {4-{3-chloro-2-[(3-ethylphenyl)oxy]phenyl}-4-hydroxy- | 2 | 3 | 2.3 | 608.3 |
| | 4-[(3R)-1-({4-[(methylamino)methyl]phenyl}carbonyl)-3- | | | | |
| | piperidinyl]butyl}carbamate | | | | |
| 172 | 1-{3-chloro-2-[(3-methylphenyl)methyl]phenyl}-1-[(3R)-1-({4- | 2 | 1 | 2.48 | 563.3 |
| | [(methylamino)methyl]phenyl}carbonyl)-3-piperidinyl]-5- | | | | |
| | (methyloxy)-1-pentanol | | | | |
| 173 | methyl {4-{3-chloro-2-[(3-methylphenyl)methyl]phenyl}-4- | 2 | 1 | 2.51 | 592.2 |
| | hydroxy-4-[(3R)-1-({4-[(methylamino)methyl]phenyl}carbonyl)-3- | | | | |
| | piperidinyl]butyl}carbamate | | | | |
| 174 | methyl {4-{3-chloro-2-[(3-methylphenyl)methyl]phenyl}-4- | | 1 | 2.45 | 610.2 |
| | [(3R)-1-({2-fluoro-4-[(methylamino)methyl]phenyl}carbonyl)-3- | | | | |
| | piperidinyl]-4-hydroxybutyl}carbamate | | | | |

^{a}Minor isomer separated by chromatography

### Example 20

### In Vitro Activity Studies

The compounds of the invention have enzyme-inhibiting properties. In particular, they inhibit the action of the natural enzyme renin. The latter passes from the kidneys into the blood where it affects the cleavage of angiotensinogen, releasing the decapeptide angiotensin I which is then cleaved in the blood, lungs, the kidneys and other organs by angiotensin converting enzyme to form the octapeptide angiotensin II. The octapeptide increases blood pressure both directly by binding to its receptor, causing arterial vasoconstriction, and indirectly by liberating from the adrenal glands the sodium-ion-retaining hormone aldosterone, accompanied by an increase in extracellular fluid volume. That increase can be attributed to the action of angiotensin II. Inhibitors of the enzymatic activity of renin bring about a reduction in the formation of angiotensin I. As a result a smaller amount of angiotensin II is produced. The reduced concentration of that active peptide hormone is the direct cause of the hypotensive effect of renin inhibitors.

The action of renin inhibitors in vitro is demonstrated experimentally by means of a test which measures the increase in fluorescence of an internally quenched peptide substrate. The sequence of this peptide corresponds to the sequence of human angiotensinogen. The following test protocol is used: All reactions are carried out in a flat bottom white opaque microtiter plate. A 4 μL aliquot of 400 μM renin substrate (DABCYL-γ-Abu-Ile-His-Pro-Phe-His-Leu-Val-Ile-His-Thr-EDANS) in 192 μL assay buffer (50 mM BES, 150 mM NaCl, 0.25 mg/mL bovine serum albumin, pH7.0) is added to 4 μL of test compound in DMSO at various concentrations ranging from 10 μM to 1 nM final concentrations. Next, 100 μL of trypsin-activated recombinant human renin (final enzyme concentration of 0.2-2 nM) in assay buffer is added, and the solution is mixed by pipetting. The increase in fluorescence at 495 nm (excitation at 340 nm) is measured for 60-360 min at rt using a Perkin-Elmer Fusion microplate reader. The slope of a linear portion of the plot of fluorescence increase as a function of time is then determined, and the rate is used for calculating percent inhibition in relation to uninhibited control. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor. (Wang G. T. et al. Anal. Biochem. 1993, 210, 351; Nakamura, N. et al. J. Biochem. (Tokyo) 1991, 109, 741; Murakami, K. et al. Anal Biochem. 1981, 110, 232).

In this in vitro systems the compounds of the invention exhibit 50% inhibition at concentrations of from approximately 5000 nM to approximately 0.01 nM. Preferred compounds of the invention exhibit 50% inhibition at concentrations of from approximately 50 n M to approximately 0.01 nM. More preferred compounds of the invention exhibit 50% inhibition at concentrations of from approximately 5 nM to approximately 0.01 nM. Highly preferred compounds of the invention exhibit 50% inhibition at concentrations of from approximately 5 nM to approximately 0.01 nM and exhibit 50% inhibition at concentrations of from approximately 10 nM to approximately 0.01 nM in the in vitro assay in the presence of human plasma described below.

### Example 21

### In Vitro Activity Studies

All reactions are carried out in a low volume, black, 384 well microtiter plate (greiner bio-one). Compounds were diluted in 100% DMSO, and a 100 nL aliquot of each compound concentration was stamped into the plate using a Hummingbird (Genomic Solutions). 5 μL of 600 pM renin (trypsin-activated recombinant human renin) was then added to the plate, followed by 5 μL of 2 μM substrate (Arg-Glu-Lys(5-FAM)-Ile-His-Pro-Phe-His-Leu-Val-Ile-His-Thr-Lys(5,6-TAMRA)-Arg-CONH₂). Both renin and substrate were made up in buffer containing 50 mM HEPES, 125 mM NaCl, 0.1% CHAPS, with the pH adjusted to 7.4. After 2 hours of reaction at room temperature, the plates were read on a Viewlux (PerkinElmer) with an excitation/emission of 485/530 nm, and using a 505 nm cutoff filter. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor. In the in vitro systems the compounds of the invention exhibit inhibiting activities at minimum concentrations of from approximately 5×10⁻⁵ M to approximately 10⁻¹² M. Preferred compounds of the invention exhibit inhibiting activities at minimum concentrations of from approximately 10⁻⁷ M to approximately 10⁻¹² M.

### Example 22

### In Vitro Activity Studies

The potency of renin inhibitors was measured using an in vitro renin assay. In this assay, renin-catalyzed proteolysis of a fluorescently labeled peptide converts the peptide from a weakly fluorescent to a strongly fluorescent molecule. The following test protocol was used. Substrate solution (5 μl; 2 μM Arg-Glu-Lys(5-Fam)-Ile-His-Pro-Phe-His-Leu-Val-Ile-His-Thr-Lys(5,6 Tamra)-Arg-CONH₂ in 50 mM Hepes, 125 mM NaCl, 0.1% CHAPS, pH 7.4) then trypsin-activated recombinant human renin (Scott, Martin J. et. al. Protein Expression and Purification 2007, 52(1), 104-116; 5 μL; 600 pM renin in 50 mM Hepes, 125 mM NaCl, 0.1% CHAPS, pH 7.4) were added sequentially to a black Greiner low volume 384-well plate (cat.#784076) pre-stamped with a 100 nl DMSO solution of compound at the desired concentration. The assay plates were incubated at room temperature for 2 hours with a cover plate then quenched by the addition of a stop solution (2 μL; 5 μM of Bachem C-3195 in 50 mM Hepes, 125 mM NaCl, 0.1% CHAPS, pH 7.4, 10% DMSO). The assay plates were read on an LJL Acquest using a 485 nm excitation filter, a 530 nm emission filter, and a 505 nm dichroic filter. Compounds were initially prepared in neat DMSO at a concentration of 10 mM. For inhibition curves, compounds were diluted using a three fold serial dilution and tested at 11 concentrations (e.g. 50 μM-0.8 nM or 25 μM-0.42 nM or 2.5 μM to 42 μM). Curves were analyzed using ActivityBase and XLfit, and results were expressed as pIC₅₀ values. In the in vitro systems the compounds of the invention exhibit inhibiting activities at minimum concentrations of from approximately 5×10⁻⁵ M to approximately 10⁻¹² M. Preferred compounds of the invention exhibit inhibiting activities at minimum concentrations of from approximately 10⁻⁷ M to approximately 10⁻¹² M.

The following compounds were found to not to demonstrate in vitro renin inhibition (IC₅₀) at concentrations below 5×10⁻⁵ M:
1-(1-{[4-(aminomethyl)phenyl]carbonyl}-3-piperidinyl)-1-{2-[(2,6-dimethylphenyl)oxy]phenyl}-5-(methyloxy)-1-pentanol;
1-(1-{[4-(aminomethyl)phenyl]carbonyl}-3-piperidinyl)-1-{3-chloro-2-[(2-methylphenyl)methyl]phenyl}-5-(methyloxy)-1-pentanol;
1-{3-chloro-2-[(3-methylphenyl)methyl]phenyl}-1-[1-({4-[(methylamino)methyl]phenyl}carbonyl)-3-piperidinyl]-5-(methyloxy)-1-pentanol;
methyl {4-[6-fluoro-3′-methyl-5′-(methyloxy)-2-biphenyl)-1]-4-hydroxy-4-[4-({4-[(methylamino)methyl]phenyl}carbonyl)-2-morpholinyl]butyl}carbamate;

methyl {4-[6-fluoro-3′-methyl-5′-(methyloxy)-2-biphenyl)-1]-4-hydroxy-4-[4-({4-[(methylamino)methyl]phenyl}carbonyl)-2-morpholinyl]butyl}carbamate;
1-{3-chloro-2-[4-(1-methylethyl)-2-quinazolinyl]phenyl}-1-[4-({4-[(methylamino)methyl]phenyl}carbonyl)-2-morpholinyl]-5-(methyloxy)-1-pentanol;
methyl {4-[3-fluoro-2-(2-thienyl)phenyl]-4-hydroxy-4-[1-({4-[(methylamino)methyl]phenyl}carbonyl)-3-piperidinyl]butyl}carbamate;
an isomer or isomeric mixture of methyl {4-(4-{[3-aminocyclopentyl]carbonyl}-2-morpholinyl)-4-[6-fluoro-3′-methyl-5′-(methyloxy)-2-biphenylyl]-4-hydroxybutyl}carbamate and methyl [2-({(6-chloro-3′-ethyl-2-biphenylyl)[4-({4-[(methylamino)methyl]phenyl}carbonyl)-2-morpholinyl]methyl}oxy)ethyl]carbamate.

### Example 23

### In Vitro Activity of the Disclosed Compounds In Human Plasma

The action of renin inhibitors in vitro in human plasma is demonstrated experimentally by the decrease in plasma renin activity (PRA) levels observed in the presence of the compounds. Incubations mixtures contain in the final volume of 250 μL 95.5 mM N,N-bis(2-hydroxyethyl)-2-aminoethanesulfonic acid, pH 7.0, 8 mM EDTA, 0.1 mM neomycin sulfate, 1 mg/ml sodium azide, 1 mM phenylmethanesulfonyl fluoride, 2% DMSO and 87.3% of pooled mixed-gender human plasma stabilized with EDTA. For plasma batches with low PRA (less than 1 ng/ml/hr) ˜2 pM of recombinant human renin IS added to achieve PRA of 3-4 ng/ml/hr. The cleavage of endogenous angiotensinogen in plasma is carried out at 37° C. for 90 min and the product angiotensin I is measured by competitive radioimmunoassay using DiaSorin PRA kit. Uninhibited incubations containing 2% DMSO and fully inhibited controls with 2 μM of isovaleryl-Phe-Nle-Sta-Ala-Sta-OH are used for deriving percent of inhibition for each concentration of inhibitors and fitting dose-response data into a four parametric model from which IC₅₀ values, defined as concentrations of inhibitors at which 50% inhibition occurs, is determined.

### Example 24

### Efficacy of the Disclosed Inhibitors in a Transgenic Rat Model

The efficacy of the renin inhibitors is also evaluated in vivo in double transgenic rats engineered to express human renin and human angiotensinogen (Bohlender J, Fukamizu A, Lippoldt A, Nomura T, Dietz R, Menard J, Murakami K, Luft F C, Ganten D. High human renin hypertension in transgenic rats. Hypertension 1997, 29, 428-434).

Experiments are conducted in 5-10 week-old double transgenic rats (dTGRs). The model has been described in detail earlier. Briefly, the human renin construct are used to generate transgenic animals (hRen) made up the entire genomic human renin gene (10 exons and 9 introns), with 3.0 kB of the 5′-promoter region and 1.2 kB of 3′ additional sequences. The human angiotensinogen construct made up the entire human angiotensinogen gene (5 exons and 4 introns), with 1.3 kB of 5′-flanking and 2.4 kB of 3′-flanking sequences are used to generate rats producing human angiotensinogen (hAogen). The hRen and hAogen rats are rederived using embryo transfer from breeding pairs obtained under license from Ascencion Gmbh (Germany). The hAogen and hRen are then crossed to produce the double transgenic dTGR) off-spring. The dTGr rats are maintained on irradiated rodent chow (5VO2, Purina Mills Inc) and normal water. Radio telemetry transmitters (TA11PAC40, Data Sciences International) are surgically implanted at 5-6 weeks of age. The telemetry system provided 24-h recordings of systolic, mean, diastolic arterial pressure (SAP, MAP, DAP, respectively) and heart rate (HR). Prior to dosing, baseline hemodynamic measures are obtained for 24 hours. Rats are then dosed orally with vehicle or drug and monitored up to 48 hours post-dose.

While this invention has been particularly shown and described with references to preferred embodiments thereof, it will be understood by those skilled in the art that various changes in form and details may be made therein without departing from the scope of the invention encompassed by the appended claims.

1. A compound represented by Formula I: 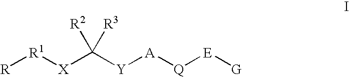
wherein
R is:
a) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cycloalkoxy, (C₅-C₇)cyclo-alkenyloxy, (C₃-C₇)cycloalkoxy(C₁-C₃)alkyl, (C₃-C₇)cycloalkyl(C₁-C₃)alkoxy, (C₅-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁-C₃)alkylthio, (C₅-C₇)cycloalkenyl(C₁-C₃)alkylthio, (C₁-C₈)alkylamino, di(C₁-C₈)alkylamino, azepano, azetidino, piperidino, pyrrolidino, (C₃-C₇)cycloalkylamino, ((C₃-C₇)cycloalkyl(C₁-C₃)alkyl)amino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy and oxo;
b) aryl, heteroaryl, arylheterocyclyl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃))alkenyl, or heteroaryl(C₂-C₃))alkynyl, each optionally substituted with up to three substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkyl, halo(C₁-C₆)alkoxy(C₁-C₆)alkyl, (C₃-C₆)cycloalkoxy(C₁-C₆)alkyl, (C₄-C₇)cycloalkylalkoxy(C₁-C₆)alkyl, halo(C₃-C₆)cycloalkoxy(C₁-C₆)alkyl, halo(C₄-C₇)cycloalkylalkoxy(C₁-C₆)alkyl, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or
c) a divalent radical selected from —(CH₂)₃—, —(CH₂)₄—, —(CH₂)₅— and —(CH₂)₆—, which is attached to R¹ to form a fused or spiro-fused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo;
R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally substituted with up to four substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylaminocarbonyl and di(C₁-C₆)alkylaminocarbonyl;
X and Y are each independently CH₂ or a single bond;
R² is a) —H; or b) (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, aminocarbonylamino(C₁-C₁₂)alkylamino, (C₁-C₆)-alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxy-carbonyl(C₁-C₆)alkylamino, (C₁-C₆)acyloxy(C₁-C₆)alkyl, (C₁-C₆) acyloxy(C₁-C₆)alkoxy, (C₁-C₆) acyloxy(C₁-C₆)alkylthio, (C₁-C₆)acyloxy(C₁-C₆)alkylamino, aminosulfonylamino(C₁-C₁₂)alkyl, aminosulfonylamino(C₁-C₁₂)alkoxy, aminosulfonylamino(C₁-C₁₂)alkylthio, aminosulfonyl-amino(C₁-C₁₂)alkylamino, (C₁-C₆)alkanesulfonylamino(C₁-C₆)alkyl, (C₁-C₆)alkanesulfonyl-amino(C₁-C₆)alkoxy, (C₁-C₆)alkanesulfonylamino(C₁-C₆)alkylthio, (C₁-C₆)alkanesulfonyl-amino(C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkyl, (C₁-C₆)alkylaminocarbonylamino(C₁-C₆)alkoxy, (C₁-C₆)alkylaminocarbonyl-amino(C₁-C₆)alkylthio, (C₁-C₆)alkylaminocarbonylamino(C₁-C₆)alkylamino, aminocarbonyl(C₁-C₆)alkyl, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkyl, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkyl, (C₁-C₆)alkylamino-carboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:
1) 1 to 5 halogen atoms; and
2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy;
the divalent sulfur atoms in R² are independently optionally oxidized to sulfoxide or sulfone and wherein the carbonyl groups are optionally independently changed to a thiocarbonyl groups;
R³ is —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)-alkoxycarbonylamino, (C₁-C₆)alkylamino-carbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonyl-amino, phenylamino or heteroarylamino in which each phenylamino or heteroarylamino group is optionally substituted with 1 to 5 groups independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₁-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)-cycloalkylalkanesulfinyl, C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)-cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl, provided that
i) R² and R³ are not both hydrogen; and
ii) when R³ is hydroxyl, halogen, or optionally substituted phenylamino or heteroarylamino, R² is not (C₁-C₁₂)alkoxy, (C₁-C₁₂)alkylthio, (C₁-C₁₂)alkylamino, oxo(C₁-C₁₂)alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)-alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, aminocarbonylamino(C₁-C₁₂)alkoxy, aminocarbonyl-amino(C₁-C₁₂)alkylthio, aminocarbonylamino(C₁-C₁₂)alkylamino, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl-(C₁-C₆)alkylamino, (C₁-C₆) acyloxy(C₁-C₆)alkoxy, (C₁-C₆) acyloxy(C₁-C₆)alkylthio, (C₁-C₆)-acyloxy(C₁-C₆)alkylamino, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkoxy, (C₁-C₆)alkylaminocarbonylamino(C₁-C₆)alkylthio, (C₁-C₆)alkylamino-carbonylamino(C₁-C₆)alkylamino, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkylamino, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:
1) 1 to 5 halogen atoms; and
2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy;
the divalent sulfur atoms in R³ are independently optionally oxidized to sulfoxide or sulfone and wherein the carbonyl groups in R³ are optionally independently changed to thiocarbonyl groups;
A is a saturated or unsaturated 4-, 5-, 6-, or 7-membered ring which is optionally bridged by (CH₂)ₘ, via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally substituted with up to four independently selected halogen atoms, (C₁-C₆)alkyl groups, halo(C₁-C₆)alkyl groups or oxo groups such that when there is substitution with one oxo group on a carbon atom it forms a carbonyl group and when there is substitution of one or two oxo groups on sulfur it forms sulfoxide or sulfone groups, respectively, where m is 1 to 3;
Q and Y are attached to carbon or nitrogen atoms in ring A in a 1, 2 or 1,3, or 1,4 relationship;
Q is a divalent radical: 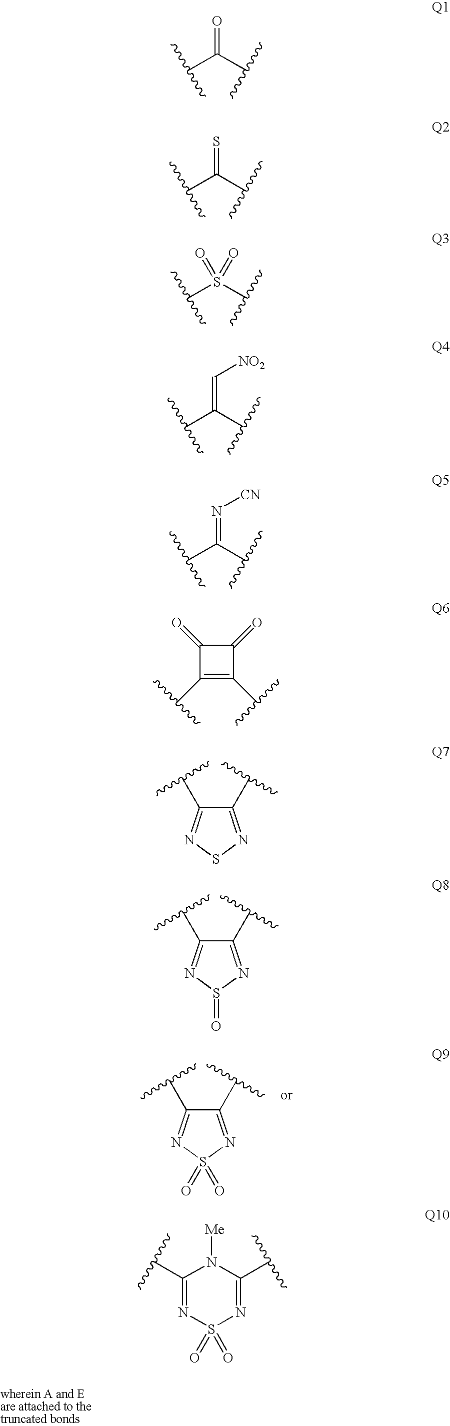
E is an optionally substituted (C₁-C₄)alkyl naphthyl, (C₁-C₄)alkyl phenyl, naphthyl or phenyl group, wherein said group is optionally substituted with up to four groups independently selected from halogen, hydroxy, aryl, (C₁-C₆)alkyl, halo(C₁-C₆)alkyl, hydroxy(C₁-C₆)alkyl, heterocyclyl(C₁-C₆)alkyl, (C₄-C₆)cycloalkyl(C₁-C₆)alkyl, cyano(C₁-C₆)alkyl, NH₂C(═O)—(C₁-C₆)alkyl, (C₁-C₆)alkoxy, —C(═O)(C₁-C₆)alkyl, —C(═O)OH, —C(═O)O(C₁-C₆)alkyl, —C(═O)NH₂, and —CN; when E is an optionally substituted (C₁-C₄)alkyl naphthyl or (C₁-C₄)alkyl phenyl, the bonding arrangement of Q to E is via the (C₁-C₄)alkyl moiety, for example—Q-(C₁-C₄)alkyl-phenyl-G;
G is hydroxy, —NR⁴R^{4a}, —O(C₂-C₆)alkyl-NR⁴R^{4a}, heterocyclyl, —(C₁-C₆)alkyl-OH, —(C₁-C₆)haloalkyl-OH, —(C₁-C₆)alkyl-NR⁴R^{4a}, —(C₁-C₆)alkyl-N⁺(C₁-C₆)alkylR⁴R^{4a}, —(C₁-C₆)alkylSO₂(C₁-C₆)alkyl, —C(═O)(C₁-C₆)alkyl-NR⁴R^{4a}, —C(═O)OH, —C(═O)NH₂, —C(═NH)NR⁴R^{4a}, —NHC(═NH)NR⁴R^{4a}, —C(═O)(C₁-C₄)alkylaryl, —C(═O)(C₁-C₄)alkyl(C₄-C₇)heterocyclyl, —(C₁-C₄)alkyl(C₃-C₈)cycloalkyl, or —(C₁-C₄)alkyl(C₄-C₇)heterocyclyl, wherein the (C₁-C₄)alkyl moiety is optionally substituted by amino, hydroxy, or (C₁-C₃)alkylamino, where R^{4a} is H or (C₁-C₃)alkyl and R⁴ is selected from H, optionally substituted (C₁-C₆)alkyl, —C(═NH)NH₂, (C₃-C₇)cycloalkyl, (C₄-C₇)heterocyclyl, (C₃-C₇)cycloalkyl(C₁-C₆)alkyl, and (C₄-C₇)heterocyclyl(C₁-C₆)alkyl, wherein the optionally substituted (C₁-C₆)alkyl is optionally substituted by hydroxy, (C₁-C₆)alkoxy, —NH₂, —NH(C₁-C₆)alkyl, —N(C₁-C₆)alkyl(C₁-C₆)alkyl, —NHSO₂(C₁-C₆)alkyl, (C₁-C₆)alkylthio, (C₁-C₆)alkanesulfonyl, —C(═O)OH, —C(═O)NH₂, or —CN, or R⁴ and R^{4a}, taken together with the nitrogen atom to which they are attached, form a 5-6 membered saturated heterocyclic ring composed of carbon atoms and 2-3 nitrogen atoms, said ring being optionally substituted with up to four groups independently selected from halogen, hydroxy, amino, (C₁-C₆)alkyl, (C₁-C₆)alkylamino, halo(C₁-C₆)alkyl, hydroxy(C₁-C₆)alkyl, amino(C₁-C₆)alkyl, and oxo groups such that when there is substitution with one oxo group on a carbon atom it forms a carbonyl group;
or a salt thereof.

2. The compound or salt according to claim 1 wherein the compound of Formula I is represented by the following structural formula: 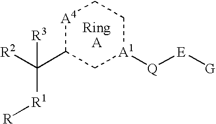
wherein when Ring A is a benzene ring, A¹ and A⁴ are CH and the bonds in ring A are aromatic bonds; when Ring A is a piperidinyl ring, A¹ is N, A⁴ is CH₂ and the bonds in ring A are single bonds; and when Ring A is a morpholinyl ring, A¹ is N, A⁴ is O and the bonds in ring A are single bonds.

3. The compound or salt according to claim 2, wherein the compound of Formula I is represented by the following structural formula:

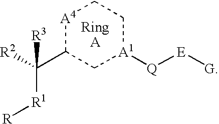

4. The compound or salt according to claim 2, wherein the compound of Formula I is represented by the following structural formula:

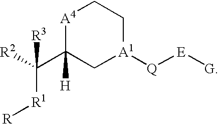

5. The compound or salt according to claim 1, wherein: E is a phenyl, methyl-phenyl, or naphthyl group, optionally substituted on the phenyl or naphthyl moiety thereof with 1-2 groups independently selected from halogen, hydroxy, (C₁-C₆)alkyl, halo(C₁-C₆)alkyl, hydroxy(C₁-C₆)alkyl, cyano(C₁-C₆)alkyl, NH₂C(═O)—(C₁-C₆)alkyl, (C₁-C₆)alkoxy, aryl, heterocyclyl(C₁-C₆)alkyl, (C₄-C₆)cycloalkyl(C₁-C₆)alkyl, —C(═O)OH, —C(═O)O(C₁-C₆)alkyl, —C(═O)NH₂, and —CN.

6. The compound or salt according to claim 1, wherein: A or Ring A is a piperidinyl ring or a morpholinyl ring.

7. The compound or salt according to claim 1, wherein: Q is Q1, Q2, Q3 or Q6.

8. The compound or salt according to claim 1, wherein: Q is Q1 or Q3.

9. The compound or salt according to claim 1, wherein: G is, —(C₁-C₄)alkyl-OH, —(C₁-C₆)haloalkyl-OH, —(C₁-C₆)alkyl-N⁺(C₁-C₆)alkylR⁴R^{4a}, —(C₁-C₆)alkylSO₂(C₁-C₆)alkyl, —C(═O)OH, —C(═O)NH₂, —C(═NH)NR⁴R^{4a}, —(C₁-C₄)alkyl-NR⁴R^{4a}, —O(C₂-C₄)alkyl-NR⁴R^{4a}, —C(═O)(C₁-C₄)alkyl-NR⁴R^{4a}, —C(═O)(C₁-C₄)alkylphenyl, —C(═O)(C₁-C₄)alkyl(C₄-C₆)heterocyclyl, —(C₁-C₄)alkyl(C₃-C₆)cycloalkyl, or —(C₁-C₄)alkyl(C₄-C₆)heterocyclyl, wherein the (C₁-C₄)alkyl moiety of said —C(═O)(C₁-C₄)alkylphenyl, —C(═O)(C₁-C₄)alkyl(C₄-C₆)heterocyclyl, —(C₁-C₄)alkyl(C₃-C₆)cycloalkyl and —(C₁-C₄)alkyl(C₄-C₆)heterocyclyl is optionally substituted by amino, hydroxy, or (C₁-C₃)alkylamino, and where R^{4a} is H or (C₁-C₃)alkyl and R⁴ is selected from H, optionally substituted (C₁-C₆)alkyl, —C(═NH)NH₂, (C₃-C₆)cycloalkyl, (C₄-C₆)heterocyclyl, (C₃-C₆)cycloalkyl(C₁-C₄)alkyl, and (C₄-C₆)heterocyclyl(C₁-C₄)alkyl, wherein the optionally substituted (C₁-C₆)alkyl is optionally substituted by hydroxy, (C₁-C₄)alkoxy, —NH₂, —NH(C₁-C₄)alkyl, —N(C₁-C₄)alkyl(C₁-C₄)alkyl, —NHSO₂(C₁-C₄)alkyl, (C₁-C₄)alkylthio, (C₁-C₄)alkanesulfonyl, —C(═O)OH, —C(═O)NH₂, —CN, or R⁴ and R^{4a}, taken together with the nitrogen atom to which they are attached, form a 5-6 membered saturated heterocyclic ring composed of carbon atoms and 2 nitrogen atoms.

10. The compound or salt according to claim 1, wherein: G is —(C₁-C₃)alkyl-NR⁴R^{4a}, —O(C₂-C₃)alkyl-NR⁴R^{4a}, —(C₁-C₃)haloalkyl-OH, —(C₁-C₃)alkyl-N⁺(C₁-C₃)alkylR⁴R^{4a}, —(C₁-C₃)alkylSO₂(C₁-C₃)alkyl, —C(═O)OH, —C(═O)NH₂, or —C(═NH)NR⁴R^{4a}, where R^{4a} is H or (C₁-C₃)alkyl and R⁴ is selected from H, optionally substituted (C₁-C₄)alkyl, —C(═NH)NH₂, (C₅-C₆)cycloalkyl, (C₅-C₆)heterocyclyl, and (C₅-C₆)heterocyclyl(C₁-C₃)alkyl wherein the optionally substituted (C₁-C₄)alkyl is optionally substituted by hydroxy, (C₁-C₄)alkoxy, —NH₂, —NH(C₁-C₄)alkyl, —N(C₁-C₄)alkyl(C₁-C₄)alkyl, —NHSO₂(C₁-C₄)alkyl, (C₁-C₄)alkylthio, (C₁-C₄)alkanesulfonyl, —C(═O)OH, —C(═O)NH₂, —CN, or R⁴ and R^{4a}, taken together with the nitrogen atom to which they are attached, form a 5-6 membered saturated heterocyclic ring composed of carbon atoms and 2 nitrogen atoms.

11. The compound or salt according to claim 1, wherein:
R is aryl, monocyclic heteroaryl, arylheterocyclyl, bicyclic heteroaryl, phenoxy, monocyclic heteroaryloxy, phenyl(C₁-C₃)alkoxy, phenyl(C₁-C₃)alkyl, and monocyclic heteroaryl(C₁-C₃)alkoxy, each optionally substituted with up to 3 substituents independently selected from fluorine, chlorine, (C₁-C₃)alkyl, (C₃-C₆)cycloalkyl, halo(C₁-C₃)alkyl, (C₁-C₃)alkoxy(C₁-C₃)alkyl, and (C₁-C₃)alkoxy;
R¹ is a phenyl ring or a pyridinyl ring, optionally substituted with up to 1-2 substituents independently selected from: halogen, (C₁-C₃)alkyl, halo(C₁-C₃)alkyl, (C₁-C₃)alkoxy, and halo(C₁-C₃)alkoxy;
R² is hydroxy(C₁-C₅)alkyl, (C₁-C₃)alkoxy(C₁-C₅)alkyl, (C₁-C₃)alkoxy(C₁-C₅)alkoxy, (C₃-C₆)cycloalkyl(C₁-C₅)alkyl, (C₃-C₆)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, halo(C₁-C₃)alkanoylamino(C₁-C₅)alkyl, hydroxy-(C₁-C₃)alkanoylamino(C₁-C₅)alkyl, formylamino(C₁-C₅)alkyl, (C₁-C₃)-alkanoylamino(C₁-C₅)alkoxy, (C₁-C₃)alkylaminocarbonyl(C₁-C₅)alkyl or (C₁-C₃)alkylaminocarbonyl(C₁-C₅)alkoxy; and
R³ is hydrogen, OH, (C₁-C₄)alkanoylamino, or (C₁-C₃)alkoxy; provided that when R³ is OH, R² is not (C₃-C₆)cycloalkyl(C₁-C₅)alkoxy, (C₁-C₃)alkoxy(C₁-C₅)alkoxy, (C₁-C₃)alkanoylamino(C₁-C₅)alkoxy, (C₁-C₃)alkoxycarbonylamino(C₁-C₅)alkoxy, or (C₁-C₃)alkylaminocarbonyl(C₁-C₅)alkoxy.

12. The compound or salt according to claim 1, wherein:
R is phenyl, naphthyl, monocyclic heteroaryl, bicyclic heteroaryl, phenoxy, monocyclic heteroaryloxy, phenyl(C₁-C₃)alkoxy, phenyl(C₁-C₃)alkyl, and monocyclic heteroaryl(C₁-C₃)alkoxy, each optionally substituted with up to 3 substituents independently selected from fluorine, chlorine, (C₁-C₃)alkyl, (C₃-C₆)cycloalkyl, halo(C₁-C₃)alkyl, and (C₁-C₃)alkoxy;
R¹ is a phenyl ring, optionally substituted with up to 1-2 substituents independently selected from: halogen, (C₁-C₃)alkyl, halo(C₁-C₃)alkyl, (C₁-C₃)alkoxy, and halo(C₁-C₃)alkoxy;
R² is (C₁-C₃)alkoxy(C₁-C₅)alkyl, (C₁-C₃)alkoxy(C₁-C₅)alkoxy, (C₃-C₆)cycloalkyl(C₁—O₅)alkyl, (C₃-C₆)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, fluoro(C₁-C₃)alkanoylamino(C₁-C₅)alkyl, hydroxy-(C₁-C₃)alkanoylamino(C₁-C₅)alkyl, formylamino(C₁-C₅)alkyl, (C₁-C₃)-alkanoylamino(C₁-C₅)alkoxy, (C₁-C₃)alkylaminocarbonyl(C₁-C₅)alkyl or (C₁-C₃)alkylaminocarbonyl(C₁-C₅)alkoxy; and
R³ is OH, (C₁-C₄)alkanoylamino, or (C₁-C₃)alkoxy; provided that when R³ is OH, R² is not (C₃-C₆)cycloalkyl(C₁-C₅)alkoxy, (C₁-C₃)alkoxy(C₁-C₅)alkoxy, (C₁-C₃)-alkanoylamino(C₁-C₅)alkoxy, (C₁-C₃)alkoxycarbonylamino(C₁-C₅)alkoxy, or (C₁-C₃)alkylaminocarbonyl(C₁-C₅)alkoxy;
A or Ring A is a piperidinyl ring or a morpholinyl ring;
Q is Q1;
E is phenyl, optionally substituted with 1-2 groups independently selected from halogen, hydroxy, (C₁-C₆)alkyl, halo(C₁-C₆)alkyl and hydroxy(C₁-C₆)alkyl;
G is —(C₁-C₃)alkyl-NR⁴R^{4a} or —O(C₂-C₃)alkyl-NR⁴R^{4a}, where R⁴ is H or (C₁-C₃)alkyl and R^{4a} is selected from H, (C₁-C₃)alkyl, and (C₅-C₆)cycloalkyl(C₁-C₃)alkyl, or R⁴ and R^{4a}, taken together with the nitrogen atom to which they are attached, form a 5-6 membered saturated heterocyclic ring composed of carbon atoms and 1-2 nitrogen atoms.

13. The compound or salt according to claim 1, wherein R is 3-ethylphenyl, 3-methylphenyl, 3-isopropylphenyl, 3-methoxyphenyl, 3-methoxymethyl-phenyl, 3-methoxy-5-methyl-phenyl, 2-methoxy-5-trifluoromethyl-phenyl, phenoxy, 2-methyl-phenoxy, 2-ethyl-phenoxy, 3-methyl-phenoxy, 3-ethyl-phenoxy, 2,6-dimethyl-phenoxy, 2-chloro-6-methyl-phenoxy, 3-methyl-4-fluoro-phenyl, 2-fluoro-5-methyl-phenyl, 3-fluoro-5-methyl-phenyl, 3-methylbenzyl-, 2-methylbenzyl-, 8-methylquinolin-2-yl, 8-isopropylquinoline-2-yl, quinolin-3-yl, 4-isopropylquinazoline-2-yl, 1-benzothien-3-yl, 5-methyl-furan-2-yl, 2,3-dihydro-1-benzofuran-6-yl, or naphthalene-2-yl; R¹ is phenyl, 3-fluorophenyl, 3-chlorophenyl, 3,5-difluorophenyl, 3-fluoro-4-methoxyphenyl, 2-chloropyridinyl, 3-chloropyridinyl, or 4-chloropyridinyl; R² is 3-(acetamido)propyl-, 3-(trifluoro-acetamido)propyl-, 3-(hydroxy-acetamido)propyl-, 3-(chloro-acetamido)propyl-, 3-(propanamido)propyl-, 3-(formamido)propyl-, 3-(methoxycarbonylamino)propyl-, 3-(ethoxycarbonylamino)propyl-, 3-(isopropoxycarbonylamino)propyl-, 2-(methoxycarbonylamino)ethoxy, 5-hydroxypentyl-, or 4-methoxybutyl-; R³ is hydrogen or hydroxyl; Ring A is a piperidinyl ring or a morpholinyl ring; Q is Q1 or Q3; E is phenyl, 2-fluorophenyl, 2-bromophenyl, 2-phenylphenyl, 2-methylphenyl, 2-ethylphenyl, 2-n-butylphenyl, 2-iso-butylphenyl, 2-methoxyphenyl, 3-methoxyphenyl, 2-cyanophenyl, 2-carboxyphenyl, 2-[H₂N(C═O)]phenyl, 2-[H₂N(C═O)CH₂]phenyl, 2-[H₂N(C═O)(CH₂)₂]phenyl, 2-(cyanoethyl)phenyl, 2-[(1H-tetrazol-5-yl)ethyl]phenyl, 2-[2-(1,3-dioxolan-2-yl)ethyl]phenyl, naphthyl, or benzyl; G is —CH₂NH₂, —CH₂NHCH₃, —CH₂N(CH₃)₂, —CH₂N⁺(CH₃)₃, —CH₂NHCH₂CH₃, —CH₂NHCH(CH₃)₂, —CH₂NH-cyclohexyl, —CH₂NH-(tetrahydro-2H-pyran-4-yl), —CH₂NHCH₂-cyclohexyl, —CH₂NHCH₂— (tetrahydro-2-furanyl), —(CH₂)₂NH₂, —CH₂NH(CH₂)₂OH, —CH₂NH(CH₂)₃OH, —CH₂NH(CH₂)₄OH, —CH₂NH(CH₂)₂OCH₃, —CH₂NH(CH₂)₂OCH₂CH₃, —CH₂NH(CH₂)₂OCH(CH₃)₂, —CH₂NHCH(CH₃)CH₂OCH₃, —CH₂NH(CH₂)₂N(CH₃)₂, —CH₂NH(CH₂)₃N(CH₃)₂, —CH₂NH(CH₂)₂SCH₃, —CH₂NH(CH₂)₂SO₂CH₃, —CH₂NHCH₂CO₂H, —CH₂NH(CH₂)₂CO₂H, —CH₂NHCH₂CN, —CH₂NH(CH₂)₂CN, —CH₂NHCH₂CONH₂, —CH₂NH(CH₂)₂CONH₂, —CH₂NH(CH₂)₃CONH₂, —CH₂NHCH₂-(1H-tetrazol-5-yl), —CH₂NH(CH₂)₂-(1H-tetrazol-5-yl), —CH₂NH(CH₂)₃-(1H-tetrazol-5-yl), —CH₂NHC(NH)NH₂, —CH₂NH(CH₂)₂NHSO₂CH₃, —C(NH)NH₂, —O(CH₂)₂NH₂, —CO₂H, —CONH₂, —CH₂SO₂CH₃, —CH₂— (piperazin-1-yl), C(CF₃)₂OH.

14. The compound or salt according to claim 1, wherein R is 3-ethylphenyl, 3-methylphenyl, 3-isopropylphenyl, 3-methoxyphenyl, 3-methoxymethyl-phenyl, 2-ethoxyphenyl, 3-methoxy-5-methyl-phenyl, phenoxy, 2-methyl-phenoxy, 2-ethyl-phenoxy, 3-methyl-phenoxy, 2-chloro-6-methyl-phenoxy, 3-methyl-4-fluoro-phenyl, 2-fluoro-5-methyl-phenyl, 3-methylbenzyl-, 2-methylbenzyl-, quinolin-3-yl, or naphthalene-2-yl; R¹ is phenyl, 3-fluorophenyl or 3-chlorophenyl; R² is 3-(acetamido)propyl-, 3-(trifluoro-acetamido)propyl-, 3-(hydroxy-acetamido)propyl-, 3-(propanamido)propyl-, 3-(formamido)propyl-, 3-(methoxycarbonylamino)propyl, 3-(ethoxycarbonylamino)propyl, 3-(isopropoxycarbonylamino)propyl, or 4-methoxybutyl; R³ is hydroxyl; Ring A is a piperidinyl ring or a morpholinyl ring; Q is Q1; E is phenyl or fluoro-phenyl; G is —CH₂NH₂, —CH₂CH₂NH₂, —OCH₂CH₂NH₂, —CH₂NHCH₃, —CH₂NHCH₂CH₃, —CH₂NHCH(CH₃)₂, —CH₂N(CH₃)₂, —CH₂NHCH₂-cyclohexyl, —CH₂— (piperazin-1-yl.

15. The compound or salt according to claim 1, which is selected from Compounds I-1-I-174.

16. A pharmaceutical composition comprising the compound or salt of claim 1 and a pharmaceutically acceptable carrier therefore.

17. The pharmaceutical composition of claim 16, further comprising an additional agent selected from the group consisting of an α-blocker, a β-blocker, a calcium channel blocker, a diuretic, an angiotensin converting enzyme inhibitor, a dual angiotensin converting enzyme-neutral endopeptidase inhibitor, an angiotensin-receptor blocker, an aldosterone synthase inhibitor, an aldosterone-receptor antagonist, and an endothelin receptor antagonist.

18. A method of inhibiting an aspartic protease, wherein the aspartic protease is renin, in a subject in need thereof comprising administering to the subject a therapeutically effective amount of the compound or salt of claim 1.

19. A method for treating or ameliorating an aspartic protease mediated disorder in a subject in need thereof comprising administering to said subject a therapeutically effective amount of the compound or salt of claim 1.

20. The method of claim 19, wherein the aspartic protease at least one of β-secretase, plasmepsin and HIV protease.

21. A method for treating or ameliorating a renin mediated disorder in a subject in need thereof comprising administering to the subject an effective amount of the compound or salt of claim 1.

22. The method of claim 21, wherein the renin mediated disorder is hypertension, congestive heart failure, cardiac hypertrophy, cardiac fibrosis, cardiomyopathy post-infarction, complications resulting from diabetes, such as nephropathy, vasculopathy and neuropathy, diseases of the coronary vessels, post-surgical hypertension, restenosis following angioplasty, raised intra-ocular pressure, glaucoma, abnormal vascular growth, hyperaldosteronism, anxiety states, or a cognitive disorder.

23. A method for the treatment of hypertension in a subject in need thereof comprising administering to the subject the compound or salt of claim 1 in combination therapy with one or more additional agents, wherein each of said additional agents is independently selected from the group consisting of an α-blocker, a β-blocker, a calcium channel blocker, a diuretic, an angiotensin converting enzyme inhibitor, a dual angiotensin converting enzyme-neutral endopeptidase inhibitor, an angiotensin-receptor blocker, an aldosterone synthase inhibitor, an aldosterone-receptor antagonist, and an endothelin receptor antagonist.

24. The method of claim 23, wherein: the α-blocker is selected from the group consisting of doxazosin, prazosin, tamsulosin, and terazosin; the β-blocker is selected from the group consisting of atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, and carvedilol, or pharmaceutically acceptable salts thereof; the calcium channel blocker is selected from the group consisting of dihydropyridines (DHPs) and non-DHPs, wherein the DHPs are selected from the group consisting of amlodipine, felodipine, ryosidine, isradipine, lacidipine, nicardipine, nifedipine, nigulpidine, nimodiphine, nisoldipine, nitrendipine, and nivaldipine and their pharmaceutically acceptable salts and the non-DHPs are selected from the group consisting of flunarizine, prenylamine, diltiazem, fendiline, gallopamil, mibefradil, anipamil, tiapamil, and verampimil, or pharmaceutically acceptable salts thereof; the diuretic is a thiazide derivative selected from the group consisting of an amiloride, chlorothiazide, hydrochlorothiazide, methylchlorothiazide, and chlorothalidon; the ACE inhibitor is selected from the group consisting of alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril; the dual angiotensin converting enzyme-neutral endopeptidase inhibitor is selected from the group consisting of include omapatrilat, fasidotril, and fasidotrilat; the angiotensin-receptor blocker is selected from the group consisting of candesartan, eprosartan, irbesartan, losartan, olmesartan, tasosartan, telmisartan, and valsartan; the aldosterone synthase inhibitor is selected from the group consisting of anastrozole, fadrozole, and exemestane; the aldosterone-receptor antagonist is selected from the group consisting of spironolactone and eplerenone; and the endothelin antagonist is selected from the group consisting of bosentan, enrasentan, atrasentan, darusentan, sitaxentan, and tezosentan, or pharmaceutically acceptable salts thereof.

25. The method of claim 24, wherein the compound or salt and the additional agents are administered by sequential administration or simultaneous administration.

26-29. (canceled)
